(19) United States
(12) Patent Application Publication
(10) Pub. No.: US 2024/0122182 A1
(43) Pub. Date: Apr. 18, 2024

(54) PESTICIDALLY ACTIVE HETEROCYCLIC DERIVATIVES WITH SULFUR CONTAINING SUBSTITUENTS

(71) Applicant: SYNGENTA CROP PROTECTION AG, Basel (CH)

(72) Inventors: Vikas SIKERVAR, Bracknell, Berkshire (GB); Sebastian RENDLER, Basel (CH); Michel MUEHLEBACH, Stein (CH); Swarnendu SASMAL, Corlim, Ilhas (IN); Andre STOLLER, Stein (CH); André JEANGUENAT, Stein (CH); Daniel EMERY, Stein (CH); Benedikt KURTZ, Stein (CH)

(73) Assignee: SYNGENTA CROP PROTECTION AG, Basel (CH)

(21) Appl. No.: 18/262,407

(22) PCT Filed: Jan. 21, 2022
(86) PCT No.: PCT/EP2022/051396

§ 371 Date: Jul. 21, 2023

(30) Foreign Application Priority Data
Jan. 21, 2021 (EP) 20211002976
Jan. 21, 2021 (IN) 202111002976

(51) Int. Cl. A01N 43/90; A01P 5/00; A01P 7/02; A01P 7/04; A01P 9/00

(52) U.S. Cl. CPC A01N 43/90; A01P 5/00; A01P 7/02; A01P 7/04; A01P 9/00

(57) ABSTRACT

Compounds of the formula (I) wherein the substituents are as defined in claim 1. Furthermore, the present invention relates to agrochemical compositions which comprise compounds of formula (I), to preparation of these compositions, and to the use of the compounds or compositions in agriculture or horticulture for combating, preventing or controlling animal pests, including arthropods and in particular insects, nematodes, molluscs or representatives of the order Acarina.

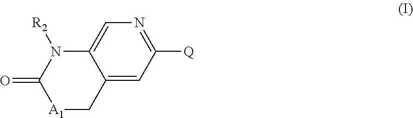

The present invention relates to pesticidally active, in particular insecticidally active heterocyclic derivatives containing sulfur substituents, to processes for their preparation, to compositions comprising those compounds, and to their use for controlling animal pests, including arthropods and in particular insects or representatives of the order Acarina.

Heterocyclic compounds with pesticidal action are known and described, for example, in WO2013191112, WO2021136722 and WO2021224409.

It has now surprisingly been found that certain novel pesticidally active derivatives with sulfur containing substitutents have favourable properties as pesticides.

The present invention therefore provides compounds of formula I,

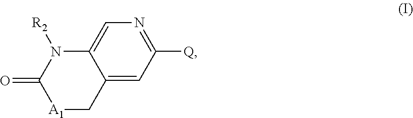

wherein
R₂ is C₁-C₆haloalkyl;
A₁ is CH₂ or O;
Q is a radical selected from the group consisting of formula Qa and Qb

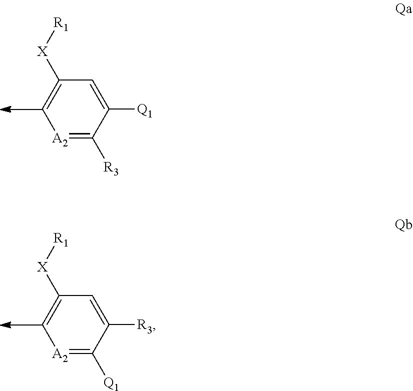

wherein the arrow denotes the point of attachment to the carbon atom of the bicyclic ring; and wherein A₂ represents CH or N;
X is S, SO, or SO₂;
R₁ is C₁-C₄alkyl or C₃-C₆cycloalkyl-C₁-C₄alkyl;
Q₁ is hydrogen, halogen, C₁-C₆haloalkyl, C₃-C₆cycloalkyl, C₃-C₆cycloalkyl monosubstituted by cyano, C₁-C₆cyanoalkyl, C₁-C₆cyanoalkoxy, C₁-C₆haloalkoxy, —N(R₄)₂, —N(R₄)COR₅, —N(R₄)CON(R₄)₂, (oxazolidin-2-one)-3-yl or 2-pyridyloxy; or
Q₁ is a five- to six-membered aromatic ring system linked via a ring carbon atom to the ring which contains the substituent A₂, said ring system is unsubstituted or is mono- or polysubstituted by substituents selected from the group consisting of halogen, cyano, C₁-C₄alkyl, C₁-C₄haloalkyl, C₁-C₄alkoxy, C₁-C₄haloalkoxy, C₁-C₄alkylsulfanyl, C₁-C₄alkylsulfinyl and C₁-C₄alkylsulfonyl; and said ring system can contain 1, 2 or 3 ring heteroatoms selected from the group consisting of nitrogen, oxygen and sulphur, where said ring system may not contain more than one ring oxygen atom and not more than one ring sulfur atom; or
Q₁ is a five-membered aromatic ring system linked via a ring nitrogen atom to the ring which contains the substituent A₂, said ring system is unsubstituted or is mono- or polysubstituted by substituents selected from the group consisting of halogen, cyano, C₁-C₄alkyl, C₁-C₄haloalkyl, C₁-C₄alkoxy, C₁-C₄haloalkoxy, C₁-C₄alkylsulfanyl, C₁-C₄alkylsulfinyl and C₁-C₄alkylsulfonyl; and said ring system contains 1, 2 or 3 ring heteroatoms selected from the group consisting of nitrogen, oxygen and sulphur, where said ring system contains at least one ring nitrogen atom and may not contain more than one ring oxygen atom and not more than one ring sulfur atom;
R₃ is hydrogen, halogen or C₁-C₄alkyl;
Each R₄ is independently hydrogen, C₁-C₄alkyl or C₃-C₆cycloalkyl; and
R₅ is C₁-C₆alkyl, C₁-C₆haloalkyl or C₃-C₆cycloalkyl.

The present invention also provides agrochemically acceptable salts, stereoisomers, enantiomers, tautomers and N-oxides of the compounds of formula I.

Compounds of formula I which have at least one basic centre can form, for example, acid addition salts, for example with strong inorganic acids such as mineral acids, for example perchloric acid, sulfuric acid, nitric acid, nitrous acid, a phosphorus acid or a hydrohalic acid, with strong organic carboxylic acids, such as C₁-C₄alkanecarboxylic acids which are unsubstituted or substituted, for example by halogen, for example acetic acid, such as saturated or unsaturated dicarboxylic acids, for example oxalic acid, malonic acid, succinic acid, maleic acid, fumaric acid or phthalic acid, such as hydroxycarboxylic acids, for example ascorbic acid, lactic acid, malic acid, tartaric acid or citric acid, or such as benzoic acid, or with organic sulfonic acids, such as C₁-C₄alkane- or arylsulfonic acids which are unsubstituted or substituted, for example by halogen, for example methane- or p-toluenesulfonic acid. Compounds of formula I which have at least one acidic group can form, for example, salts with bases, for example mineral salts such as alkali metal or alkaline earth metal salts, for example sodium, potassium or magnesium salts, or salts with ammonia or an organic amine, such as morpholine, piperidine, pyrrolidine, a mono-, di- or tri-lower-alkylamine, for example ethyl-, diethyl-, triethyl- or dimethylpropylamine, or a mono-, di- or trihydroxy-lower-alkylamine, for example mono-, di- or triethanolamine.

In each case, the compounds of formula I according to the invention are in free form, in oxidized form as a N-oxide or in salt form, e.g. an agronomically usable salt form.

N-oxides are oxidized forms of tertiary amines or oxidized forms of nitrogen containing heteroaromatic compounds. They are described for instance in the book “Heterocyclic N-oxides” by A. Albini and S. Pietra, CRC Press, Boca Raton 1991.

The compounds of formula I according to the invention also include hydrates which may be formed during the salt formation.

Where substituents are indicated as being itself further substituted, this means that they carry one or more identical or different substituents, e.g. one to four substituents. Normally not more than three such optional substituents are present at the same time. Preferably not more than two such substituents are present at the same time (i.e. the group is substituted by one or two of the substituents indicated). Where the additional substituent group is a larger group, such as cycloalkyl or phenyl, it is most preferred that only one such optional substituent is present. Where a group is indicated as being substituted, e.g. alkyl, this includes those groups that are part of other groups, e.g. the alkyl in alkylthio.

The term “C₁-Cₙalkyl” as used herein refers to a saturated straight-chain or branched hydrocarbon radical attached via any of the carbon atoms having 1 to n carbon atoms, for example, any one of the radicals methyl, ethyl, n-propyl, 1-methylbutyl, 2-methylbutyl, 3-methylbutyl, 2,2-dimethylpropyl, 1-ethylpropyl, n-hexyl, n-pentyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, or 1-ethyl-2-methylpropyl.

The term “C₁-Cₙhaloalkyl” as used herein refers to a straight-chain or branched saturated alkyl radical attached via any of the carbon atoms having 1 to n carbon atoms (as mentioned above), where some or all of the hydrogen atoms in these radicals may be replaced by fluorine, chlorine, bromine and/or iodine, i.e., for example, any one of chloromethyl, dichloromethyl, trichloromethyl, fluoromethyl, difluoromethyl, trifluoromethyl, chlorofluoromethyl, dichlorofluoromethyl, chlorodifluoromethyl, 2-fluoroethyl, 2-chloroethyl, 2-bromoethyl, 2-iodoethyl, 2,2-difluoroethyl, 2,2,2-trifluoroethyl, 2-chloro-2-fluoroethyl, 2-chloro-2,2-difluoroethyl, 2,2-dichloro-2-fluoroethyl, 2,2,2-trichloroethyl, pentafluoroethyl, 2-fluoropropyl, 3-fluoropropyl, 2,2-difluoropropyl, 2,3-difluoropropyl, 2-chloropropyl, 3-chloropropyl, 2,3-dichloropropyl, 2-bromopropyl, 3-bromopropyl, 3,3,3-trifluoropropyl, 3,3,3-trichloropropyl, 2,2,3,3,3-pentafluoropropyl, heptafluoropropyl, 1-(fluoromethyl)-2-fluoroethyl, 1-(chloromethyl)-2-chloroethyl, 1-(bromomethyl)-2-bromoethyl, 4-fluorobutyl, 4-chlorobutyl, 4-bromobutyl or nonafluorobutyl. According a term “C₁-C₂-fluoroalkyl” would refer to a C₁-C₂-alkyl radical which carries 1, 2, 3, 4, or 5 fluorine atoms, for example, any one of difluoromethyl, trifluoromethyl, 1-fluoroethyl, 2-fluoroethyl, 2,2-difluoroethyl, 2,2,2-trifluoroethyl, 1,1,2,2-tetrafluoroethyl or penta-fluoroethyl.

The term “C₁-Cₙalkoxy” as used herein refers to a straight-chain or branched saturated alkyl radical having 1 to n carbon atoms (as mentioned above) which is attached via an oxygen atom, i.e., for example, any one of methoxy, ethoxy, n-propoxy, 1-methylethoxy, n-butoxy, 1-methylpropoxy, 2-methylpropoxy or 1,1-dimethylethoxy.

The term “C₁-Cₙhaloalkoxy” as used herein refers to a C₁-Cₙalkoxy radical as mentioned above which is partially or fully substituted by fluorine, chlorine, bromine and/or iodine, i.e., for example, any one of chloromethoxy, dichloromethoxy, trichloromethoxy, fluoromethoxy, difluoromethoxy, trifluoromethoxy, chlorofluoromethoxy, dichlorofluoromethoxy, chlorodifluoromethoxy, 2-fluoroethoxy, 2-chloroethoxy, 2-bromoethoxy, 2-iodoethoxy, 2,2-difluoroethoxy, 2,2,2-trifluoroethoxy, 2-chloro-2-fluoroethoxy, 2-chloro-2,2-difluoroethoxy, 2,2-dichloro-2-fluoroethoxy, 2,2,2-trichloroethoxy, pentafluoroeth-oxy, 2-fluoropropoxy, 3-fluoropropoxy, 2,2-difluoropropoxy, 2,3-difluoropropoxy, 2-chloropropoxy, 3-chloropropoxy, 2,3-dichloropropoxy, 2-bromopropoxy, 3-bromopropoxy, 3,3,3-trifluoropropoxy, 3,3,3-trichloropropoxy, 2,2,3,3,3-pentafluoropropoxy, heptafluoropropoxy, 1-(fluoromethyl)-2-fluoroethoxy, 1-(chloromethyl)-2-chloroethoxy, 1-(bromomethyl)-2-bromoethoxy, 4-fluorobutoxy, 4-chlorobutoxy, or 4-bromobutoxy.

The term “C₁-Cₙ-alkylsulfanyl” as used herein refers to a straight chain or branched saturated alkyl radical having 1 to n carbon atoms (as mentioned above) which is attached via a sulfur atom, i.e., for example, any one of methylthio, ethylthio, n-propylthio, 1-methylethylthio, butylthio, 1-methylpropylthio, 2-methylpropylthio or 1,1-dimethylethylthio.

The term “C₁-Cₙalkylsulfinyl” as used herein refers to a straight chain or branched saturated alkyl radical having 1 to n carbon atoms (as mentioned above) which is attached via the sulfur atom of the sulfinyl group, i.e., for example, any one of methylsulfinyl, ethylsulfinyl, n-propylsulfinyl, 1-methylethyl-sulfinyl, n-butylsulfinyl, 1-methylpropylsulfinyl, 2-methylpropylsulfinyl, 1,1-dimethyl-ethylsulfinyl, n-pentylsulfinyl, 1-methylbutylsulfinyl, 2-methylbutylsulfinyl, 3-methyl-butylsulfinyl, 1,1-dimethylpropylsulfinyl, 1,2-dimethylpropylsulfinyl, 2,2-dimethylpropylsulfinyl or 1-ethylpropylsulfinyl.

The term “C₁-Cₙalkylsulfonyl” as used herein refers to a straight chain or branched saturated alkyl radical having 1 to n carbon atoms (as mentioned above) which is attached via the sulfur atom of the sulfonyl group, i.e., for example, any one of methylsulfonyl, ethylsulfonyl, n-propylsulfonyl, isopropylsulfonyl, n-butylsulfonyl, 1-methylpropylsulfonyl, 2-methylpropylsulfonyl ort-butylsulphonyl.

The term “C₁-Cₙcyanoalkyl” as used herein refers to a straight chain or branched saturated alkyl radicals having 1 to n carbon atoms (as mentioned above) which is substituted by a cyano group, for example cyanomethylene, cyanoethylene, 1,1-dimethylcyanomethyl, cyanomethyl, cyanoethyl, and 1-dimethylcyanomethyl.

The term “C₁-Cₙcyanoalkoxy” refers to the groups above but which is attached via an oxygen atom.

The suffix “—C₁-Cₙalkyl” after terms such as “C₃-Cₙcycloalkyl”, wherein n is an integer from 1-6, as used herein refers to a straight chain or branched saturated alkyl radicals which is substituted by C₃-Cₙcycloalkyl. An example of C₃-Cₙcycloalkyl-C₁-Cₙalkyl is for example, cyclopropylmethyl.

The term “C₃-C₆cycloalkyl” as used herein refers to 3-6 membered cycloylkyl groups such as cyclopropane, cyclobutane, cyclopropane, cyclopentane and cyclohexane.

Halogen is generally fluorine, chlorine, bromine or iodine. This also applies, correspondingly, to halogen in combination with other meanings, such as haloalkyl.

In the context of this invention “mono- or polysubstituted” in the definition of the substituents, means typically, depending on the chemical structure of the substituents, monosubstituted to five-times substituted, more preferably mono-, double- or triple-substituted.

In the context of the this invention, the phrases “Q₁ is a five- to six-membered aromatic ring system, linked via a ring carbon atom to the ring which contains the substituent A₂ . . . ” and “Q₁ is a five-membered aromatic ring system linked via a ring nitrogen atom to the ring which contains the substituent A₂ . . . ”, as the case may be, refer to the manner of attachment of particular embodiments of the substituent Q₁ to the radical Q as represented by either formula Qa or formula Qb, as the case may be.

In the context of this invention, examples of “Q₁ is a five- to six-membered aromatic ring system, linked via a ring carbon atom . . . ; and said ring system can contain 1, 2 or 3 heteroatoms” are, but not limited to, phenyl, pyrazolyl, triazolyl, pyridinyl and pyrimidinyl; preferably phenyl, 2-pyridyl, 3-pyridyl, 4-pyridyl, pyrimidin-2-yl, pyrimidin-4-yl, and pyrimidin-5-yl.

In the context of this invention, examples of “Q₁ is a five-membered aromatic ring system linked via a ring nitrogen atom . . . ; and said ring system contains 1, 2 or 3 heteroatoms” are, but not limited to, pyrazolyl, pyrrolyl, imidazolyl and triazolyl; preferably pyrrol-1-yl, pyrazol-1-yl, triazol-2-yl, 1,2,4-triazol-1-yl, triazol-1-yl, and imidazol-1-yl.

Certain embodiments according to the invention are provided as set out below.

Embodiment 1 provides compounds of formula I, or an agrochemically acceptable salt, stereoisomer, enantiomer, tautomer or N-oxide thereof, as defined above.

Embodiment 2 provides compounds, or an agrochemically acceptable salt, stereoisomer, enantiomer, tautomer or N-oxide thereof, according to embodiment 1 wherein Q is Qa and having preferred values of R₂, A₁, A₂, X, R₁, Q₁, R₄, R₅ and R₃ as set out below.

Embodiment 3 provides compounds, or an agrochemically acceptable salt, stereoisomer, enantiomer, tautomer or N-oxide thereof, according to embodiment 1 wherein Q is Qb and having preferred values of R₂, A₁, A₂, X, R₁, Q₁, R₄, R₅ and R₃ as set out below.

With respect to embodiments 1-3, preferred values of R₂, A₁, A₂, X, R₁, Q₁, R₄, R₅ and R₃ are, in any combination thereof, as set out below:

Preferably R₂ is C₁-C₆haloalkyl.

More preferably R₂ is C₁-C₆fluoroalkyl.

Even more preferably R₂ is —CH₂CF₂CF₃, —CH₂CF₂CHF₂, —CH₂CF₃, —CH₂CHF₂ or —CH₂CF₂CHFCF₃.

Most preferably R₂ is —CH₂CF₃, —CH₂CF₂CHF₂ or —CH₂CF₂CF₃.

Preferably A₁ is CH₂ or O.

Preferably A₂ is N.

Preferably X is S or SO₂.

Most preferably X is SO₂.

Preferably R₁ is C₁-C₄alkyl or cyclopropyl-C₁-C₄alkyl.

More preferably R₁ is ethyl or cyclopropylmethyl.

Most preferably R₁ is ethyl.

Preferably Q₁ is hydrogen, halogen, C₁-C₆haloalkyl, C₃-C₆cycloalkyl, C₃-C₆cycloalkyl monosubstituted by cyano, C₁-C₆cyanoalkyl, C₁-C₆cyanoalkoxy, C₁-C₆haloalkoxy, —N(R₄)₂, —N(R₄)COR₅, or —N(R₄)CON(R₄)₂, (oxazolidin-2-one)-3-yl or 2-pyridyloxy; or
Q₁ is a five- to six-membered aromatic ring system linked via a ring carbon atom to the ring which contains the substituent A₂, said ring system is unsubstituted or is mono-substituted by a substituent selected from the group consisting of halogen, cyano and C₁-C₄haloalkyl; and said ring system can contain 1 or 2 ring nitrogen atoms; or
Q₁ is a five-membered aromatic ring system linked via a ring nitrogen atom to the ring which contains the substituent A₂, said ring system is unsubstituted or is mono-substituted by a substituent selected from the group consisting of halogen, cyano and C₁-C₄haloalkyl; and said ring system contains 2 or 3 ring nitrogen atoms.

More preferably Q₁ is hydrogen, halogen, trifluoromethyl, fluoroisopropyl, cyclopropyl, cyanocyclopropyl, cyanoisopropyl, cyanoisopropoxy, trifluoroethoxy, difluoropropoxy, —N(R₄)₂, —N(R₄)COR₅, or —N(R₄)CON(R₄)₂, in each of which R₄ is independently either hydrogen or methyl and R₅ is either methyl or cyclopropyl, or Q₁ is (oxazolidin-2-one)-3-yl, 2-pyridyloxy, N-linked pyrazolyl which is unsubstituted or is mono-substituted by chloro, cyano or trifluoromethyl, or Q₁ is N-linked triazolyl or C-linked pyrimidinyl.

More preferably Q₁ is hydrogen, chlorine, bromine, trifluoromethyl, 1-fluoro-1-methyl-ethyl, cyclopropyl, 1-cyanocyclopropyl, 1-cyano-1-methyl-ethyl, 1-cyano-1-methyl-ethoxy, 2,2,2-trifluoroethoxy, 2,2-difluoropropoxy, —NH₂, —NH(CH₃), —N(CH₃)₂, —NHCOCH₃, —N(CH₃)COCH₃, —N(CH₃)COCH₂CH₃, —NHCO(cyclopropyl), —N(CH₃)CO(cyclopropyl), —N(H)CONH₂, —N(H)CONH(CH₃), —N(H)CON(CH₃)₂, —N(CH₃)CONH₂, —N(CH₃)CONH(CH₃), —N(CH₃)CON(CH₃)₂, (oxazolidin-2-one)-3-yl, 2-pyridyloxy, pyrazol-1-yl, 3-chloro-pyrazol-1-yl, 3-cyano-pyrazol-1-yl, 3-trifluoromethyl-pyrazol-1-yl, 1,2,4-triazol-1-yl or pyrimidin-2-yl.

Most preferably Q₁ is hydrogen, chlorine, bromine, trifluoromethyl, 1-fluoro-1-methyl-ethyl, cyclopropyl, 1-cyanocyclopropyl, 1-cyano-1-methyl-ethyl, 1-cyano-1-methyl-ethoxy, 2,2,2-trifluoroethoxy, 2,2-difluoropropoxy, —NH(CH₃), —N(CH₃)COCH₃, —N(CH₃)CO(cyclopropyl), —N(H)CONH(CH₃), —N(CH₃)CONH(CH₃), (oxazolidin-2-one)-3-yl, 2-pyridyloxy, pyrazol-1-yl, 3-chloro-pyrazol-1-yl, 3-cyano-pyrazol-1-yl, 3-trifluoromethyl-pyrazol-1-yl, 1,2,4-triazol-1-yl or pyrimidin-2-yl.

Also preferred is when Q₁ is chlorine, bromine, trifluoromethyl, 1-fluoro-1-methyl-ethyl, cyclopropyl, 1-cyanocyclopropyl, 1-cyano-1-methyl-ethyl, 1-cyano-1-methyl-ethoxy, 2,2,2-trifluoroethoxy, 2,2-difluoropropoxy, —NH(CH₃), —N(CH₃)COCH₃, —N(CH₃)CO(cyclopropyl), —N(H)CONH(CH₃), —N(CH₃)CONH(CH₃), (oxazolidin-2-one)-3-yl, 2-pyridyloxy, pyrazol-1-yl, 3-chloro-pyrazol-1-yl, 3-cyano-pyrazol-1-yl, 3-trifluoromethyl-pyrazol-1-yl, 1,2,4-triazol-1-yl or pyrimidin-2-yl.

Preferably each R₄ is independently hydrogen or C₁-C₄alkyl.

Most preferably each R₄ is independently hydrogen or methyl.

Preferably R₅ is C₁-C₆alkyl or C₃-C₆cycloalkyl.

More preferably R₅ is methyl, ethyl or cyclopropyl.

More preferably R₅ is methyl or cyclopropyl.

Most preferably R₅ is methyl.

Preferably R₃ is hydrogen or C₁-C₄alkyl.

More preferably R₃ is hydrogen or methyl.

Most preferably R₃ is hydrogen.

One group of compounds according to the invention are those of formula I-1

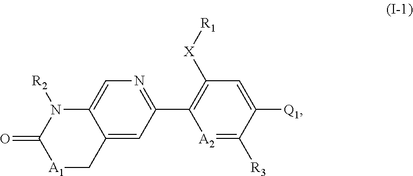

wherein A₁, A₂, X, R₁, and R₂ are as defined for compounds of formula I (above), or an agrochemically acceptable salt, stereoisomer, enantiomer, tautomer or N-oxide thereof, and wherein Q₁ is preferably hydrogen, halogen, C₁-C₆haloalkyl, C₃-C₆cycloalkyl, C₃-C₆cycloalkyl monosubstituted by cyano, C₁-C₆cyanoalkyl, C₁-C₆cyanoalkoxy, C₁-C₆haloalkoxy, —N(R₄)₂, —N(R₄)COR₅, or —N(R₄)CON(R₄)₂, (oxazolidin-2-one)-3-yl or 2-pyridyloxy; or
Q₁ is a five- to six-membered aromatic ring system linked via a ring carbon atom to the ring which contains the substituent A₂, said ring system is unsubstituted or is mono-substituted by a substituent selected from the group consisting of halogen, cyano and C₁-C₄haloalkyl; and said ring system can contain 1 or 2 ring nitrogen atoms; or
Q₁ is a five-membered aromatic ring system linked via a ring nitrogen atom to the ring which contains the substituent A₂, said ring system is unsubstituted or is mono-substituted by a substituent selected from the group consisting of halogen, cyano and C₁-C₄haloalkyl; and said ring system contains 2 or 3 ring nitrogen atoms;
R₃ is preferably hydrogen or C₁-C₄alkyl;
Preferably each R₄ is independently hydrogen or C₁-C₄alkyl; and
R₅ is preferably C₁-C₆alkyl or C₃-C₆cycloalkyl.

Preferred definitions of A₁, A₂, X, R₁, and R₂ in the compounds of formula I-1 are as defined for compounds of formula I (above), and more preferably Q₁ is hydrogen, halogen, fluoroisopropyl, trifluoromethyl, cyclopropyl, cyanocyclopropyl, cyanoisopropyl, cyanoisopropoxy, trifluoroethoxy, difluoropropoxy, —N(R₄)₂, —N(R₄)COR₅, or —N(R₄)CON(R₄)₂, in each of which R₄ is independently either hydrogen or methyl and R₅ is methyl, ethyl or cyclopropyl; preferably methyl or cyclopropyl, or Q₁ is (oxazolidin-2-one)-3-yl, 2-pyridyloxy, N-linked pyrazolyl which is unsubstituted or is mono-substituted by chloro, cyano or trifluoromethyl, or Q₁ is N-linked triazolyl or C-linked pyrimidinyl; and R₃ is hydrogen or methyl, preferably hydrogen.

In one group of compounds of formula I-1, preferably Q₁ is hydrogen, chlorine, bromine, trifluoromethyl, 1-fluoro-1-methyl-ethyl, cyclopropyl, 1-cyanocyclopropyl, 1-cyano-1-methyl-ethyl, 1-cyano-1-methyl-ethoxy, 2,2,2-trifluoroethoxy, 2,2-difluoropropoxy, —NH(CH₃), —N(CH₃)COCH₃, —N(CH₃)CO(cyclopropyl), —N(H)CONH(CH₃), —N(CH₃)CONH(CH₃), (oxazolidin-2-one)-3-yl, 2-pyridyloxy, pyrazol-1-yl, 3-chloro-pyrazol-1-yl, 3-cyano-pyrazol-1-yl, 3-trifluoromethyl-pyrazol-1-yl, 1,2,4-triazol-1-yl or pyrimidin-2-yl.

In another group of compounds of formula I-1, also preferred is when Q₁ is chlorine, bromine, trifluoromethyl, 1-fluoro-1-methyl-ethyl, cyclopropyl, 1-cyanocyclopropyl, 1-cyano-1-methyl-ethyl, 1-cyano-1-methyl-ethoxy, 2,2,2-trifluoroethoxy, 2,2-difluoropropoxy, —NH(CH₃), —N(CH₃)COCH₃, —N(CH₃)CO(cyclopropyl), —N(H)CONH(CH₃), —N(CH₃)CONH(CH₃), (oxazolidin-2-one)-3-yl, 2-pyridyloxy, pyrazol-1-yl, 3-chloro-pyrazol-1-yl, 3-cyano-pyrazol-1-yl, 3-trifluoromethyl-pyrazol-1-yl, 1,2,4-triazol-1-yl or pyrimidin-2-yl.

One group of compounds according to this embodiment are compounds of formula (I-1a) which are compounds of formula (I-1) wherein A₂ is N.

Another group of compounds according to this embodiment are compounds of formula (I-1 b) which are compounds of formula (I-1) wherein A₂ is CH.

One group of compounds according to this embodiment are compounds of formula (I-1c) which are compounds of formula (I-1) wherein R₂ is C₁-C₆fluoroalkyl; preferably R₂ is —CH₂CF₂CF₃, —CH₂CF₂CHF₂, —CH₂CF₃, —CH₂CHF₂ or —CH₂CF₂CHFCF₃; more preferably R₂ is —CH₂CF₃, —CH₂CF₂CHF₂ or —CH₂CF₂CF₃.

Another group of compounds according to this embodiment are compounds of formula (I-1d) which are compounds of formula (I-1) wherein X is S or SO₂; preferably X is SO₂.

Another group of compounds according to this embodiment are compounds of formula (I-1e) which are compounds of formula (I-1) wherein R₁ is C₁-C₄alkyl or cyclopropyl-C₁-C₄alkyl; preferably R₁ is ethyl or cyclopropylmethyl; more preferably R₁ is ethyl.

One group of compounds according to this embodiment are compounds of formula (I-1f) which are compounds of formula (I-1) wherein A₁ is CH₂.

Another group of compounds according to this embodiment are compounds of formula (I-1g) which are compounds of formula (I-1) wherein A₁ is O.

Another group of compounds according to the invention are those of formula I-2

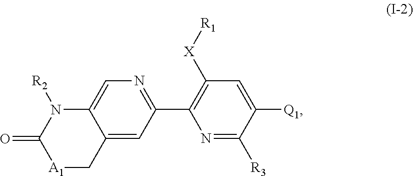

wherein A₁, X, R₁ and R₂ are as defined for compounds of formula I (above), or an agrochemically acceptable salt, stereoisomer, enantiomer, tautomer or N-oxide thereof, and wherein Q₁ is preferably hydrogen, halogen, C₁-C₆haloalkyl, C₃-C₆cycloalkyl, C₃-C₆cycloalkyl monosubstituted by cyano, C₁-C₆cyanoalkyl, C₁-C₆cyanoalkoxy, C₁-C₆haloalkoxy, —N(R₄)₂, —N(R₄)COR₅, or —N(R₄)CON(R₄)₂, (oxazolidin-2-one)-3-yl or 2-pyridyloxy; or
Q₁ is a five- to six-membered aromatic ring system linked via a ring carbon atom to the pyridyl ring substituted by X—R₁, said ring system is unsubstituted or is mono-substituted by a substituent selected from the group consisting of halogen, cyano and C₁-C₄haloalkyl; and said ring system can contain 1 or 2 ring nitrogen atoms; or
Q₁ is a five-membered aromatic ring system linked via a ring nitrogen atom to the pyridyl ring substituted by X—R₁, said ring system is unsubstituted or is mono-substituted by a substituent selected from the group consisting of halogen, cyano and C₁-C₄haloalkyl; and said ring system contains 2 or 3 ring nitrogen atoms;
R₃ is preferably hydrogen or C₁-C₄alkyl;
Preferably each R₄ is independently hydrogen or C₁-C₄alkyl; and
R₅ is preferably C₁-C₆alkyl or C₃-C₆cycloalkyl.

Preferred definitions of A₁, X, R₁ and R₂ in the compounds of formula I-2 are as defined for compounds of formula I (above), and more preferably Q₁ is hydrogen, halogen, trifluoromethyl, fluoroisopropyl, cyclopropyl, cyanocyclopropyl, cyanoisopropyl, cyanoisopropoxy, trifluoroethoxy, difluoropropoxy, —N(R₄)₂, —N(R₄)COR₅, or —N(R₄)CON(R₄)₂, in each of which R₄ is independently either hydrogen or methyl and R₅ is methyl, ethyl or cyclopropyl; preferably methyl or cyclopropyl, or Q₁ is (oxazolidin-2-one)-3-yl, 2-pyridyloxy, N-linked pyrazolyl which is unsubstituted or is mono-substituted by chloro, cyano or trifluoromethyl, or Q₁ is N-linked triazolyl or C-linked pyrimidinyl; and R₃ is hydrogen or methyl, preferably hydrogen.

In one group of compounds of formula I-2, preferably Q₁ is hydrogen, chlorine, bromine, trifluoromethyl, 1-fluoro-1-methyl-ethyl, cyclopropyl, 1-cyanocyclopropyl, 1-cyano-1-methyl-ethyl, 1-cyano-1-methyl-ethoxy, 2,2,2-trifluoroethoxy, 2,2-difluoropropoxy, —NH(CH₃), —N(CH₃)COCH₃, —N(CH₃)CO(cyclopropyl), —N(H)CONH(CH₃), —N(CH₃)CONH(CH₃), (oxazolidin-2-one)-3-yl, 2-pyridyloxy, pyrazol-1-yl, 3-chloro-pyrazol-1-yl, 3-cyano-pyrazol-1-yl, 3-trifluoromethyl-pyrazol-1-yl, 1,2,4-triazol-1-yl or pyrimidin-2-yl.

In another group of compounds of formula I-2, also preferred is when Q₁ is chlorine, bromine, trifluoromethyl, 1-fluoro-1-methyl-ethyl, cyclopropyl, 1-cyanocyclopropyl, 1-cyano-1-methyl-ethyl, 1-cyano-1-methyl-ethoxy, 2,2,2-trifluoroethoxy, 2,2-difluoropropoxy, —NH(CH₃), —N(CH₃)COCH₃, —N(CH₃)CO(cyclopropyl), —N(H)CONH(CH₃), —N(CH₃)CONH(CH₃), (oxazolidin-2-one)-3-yl, 2-pyridyloxy, pyrazol-1-yl, 3-chloro-pyrazol-1-yl, 3-cyano-pyrazol-1-yl, 3-trifluoromethyl-pyrazol-1-yl, 1,2,4-triazol-1-yl or pyrimidin-2-yl.

One group of compounds according to this embodiment are compounds of formula (I-2a) which are compounds of formula (I-2) wherein X is S or SO₂, preferably X is SO₂.

Another group of compounds according to this embodiment are compounds of formula (I-2b) which are compounds of formula (I-2) wherein R₁ is C₁-C₄alkyl or cyclopropyl-C₁-C₄alkyl, preferably R₁ is ethyl or cyclopropylmethyl; more preferably R₁ is ethyl.

Another group of compounds according to this embodiment are compounds of formula (I-2c) which are compounds of formula (I-2) wherein R₂ is C₁-C₆fluoroalkyl; preferably R₂ is —CH₂CF₂CF₃, —CH₂CF₂CHF₂, —CH₂CF₃, —CH₂CHF₂ or —CH₂CF₂CHFCF₃; more preferably R₂ is —CH₂CF₃, —CH₂CF₂CHF₂ or —CH₂CF₂CF₃.

One group of compounds according to this embodiment are compounds of formula (I-2d) which are compounds of formula (I-2) wherein A₁ is CH₂.

Another group of compounds according to this embodiment are compounds of formula (I-2e) which are compounds of formula (I-2) wherein A₁ is O.

Another group of compounds according to the invention are those of formula I-3

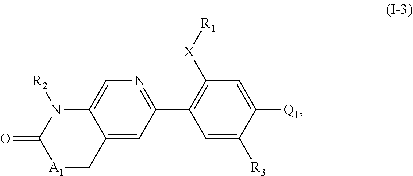

wherein A₁, X, R₁ and R₂ are as defined for compounds of formula I (above), or an agrochemically acceptable salt, stereoisomer, enantiomer, tautomer or N-oxide thereof, and wherein Q₁ is preferably hydrogen, halogen, C₁-C₆haloalkyl, C₃-C₆cycloalkyl, C₃-C₆cycloalkyl monosubstituted by cyano, C₁-C₆cyanoalkyl, C₁-C₆cyanoalkoxy, C₁-C₆haloalkoxy, —N(R₄)₂, —N(R₄)COR₅, or —N(R₄)CON(R₄)₂, (oxazolidin-2-one)-3-yl or 2-pyridyloxy; or
Q₁ is a five- to six-membered aromatic ring system linked via a ring carbon atom to the phenyl ring substituted by X—R₁, said ring system is unsubstituted or is mono-substituted by a substituent selected from the group consisting of halogen, cyano and C₁-C₄haloalkyl; and said ring system can contain 1 or 2 ring nitrogen atoms; or
Q₁ is a five-membered aromatic ring system linked via a ring nitrogen atom to the phenyl ring substituted by X—R₁, said ring system is unsubstituted or is mono-substituted by a substituent selected from the group consisting of halogen, cyano and C₁-C₄haloalkyl; and said ring system contains 2 or 3 ring nitrogen atoms;
R₃ is preferably hydrogen or C₁-C₄alkyl;
Preferably each R₄ is independently hydrogen or C₁-C₄alkyl; and
R₅ is preferably C₁-C₆alkyl or C₃-C₆cycloalkyl.

Preferred definitions of A₁, X, R₁ and R₂ in the compounds of formula I-3 are as defined for compounds of formula I (above), and more preferably Q₁ is hydrogen, halogen, trifluoromethyl, fluoroisopropyl, cyclopropyl, cyanocyclopropyl, cyanoisopropyl, cyanoisopropoxy, trifluoroethoxy, difluoropropoxy, —N(R₄)₂, —N(R₄)COR₅, or —N(R₄)CON(R₄)₂, in each of which R₄ is independently either hydrogen or methyl and R₅ is methyl, ethyl or cyclopropyl; preferably methyl or cyclopropyl, or Q₁ is (oxazolidin-2-one)-3-yl, 2-pyridyloxy, N-linked pyrazolyl which is unsubstituted or is mono-substituted by chloro, cyano or trifluoromethyl, or Q₁ is N-linked triazolyl or C-linked pyrimidinyl; and R₃ is hydrogen or methyl, preferably hydrogen.

In one group of compounds of formula I-3, preferably Q₁ is hydrogen, chlorine, bromine, trifluoromethyl, 1-fluoro-1-methyl-ethyl, cyclopropyl, 1-cyanocyclopropyl, 1-cyano-1-methyl-ethyl, 1-cyano-1-methyl-ethoxy, 2,2,2-trifluoroethoxy, 2,2-difluoropropoxy, —NH(CH₃), —N(CH₃)COCH₃, —N(CH₃)CO(cyclopropyl), —N(H)CONH(CH₃), —N(CH₃)CONH(CH₃), (oxazolidin-2-one)-3-yl, 2-pyridyloxy, pyrazol-1-yl, 3-chloro-pyrazol-1-yl, 3-cyano-pyrazol-1-yl, 3-trifluoromethyl-pyrazol-1-yl, 1,2,4-triazol-1-yl or pyrimidin-2-yl.

In another group of compounds of formula I-3, also preferred is when Q₁ is chlorine, bromine, trifluoromethyl, 1-fluoro-1-methyl-ethyl, cyclopropyl, 1-cyanocyclopropyl, 1-cyano-1-methyl-ethyl, 1-cyano-1-methyl-ethoxy, 2,2,2-trifluoroethoxy, 2,2-difluoropropoxy, —NH(CH₃), —N(CH₃)COCH₃, —N(CH₃)CO(cyclopropyl), —N(H)CONH(CH₃), —N(CH₃)CONH(CH₃), (oxazolidin-2-one)-3-yl, 2-pyridyloxy, pyrazol-1-yl, 3-chloro-pyrazol-1-yl, 3-cyano-pyrazol-1-yl, 3-trifluoromethyl-pyrazol-1-yl, 1,2,4-triazol-1-yl or pyrimidin-2-yl.

One group of compounds according to this embodiment are compounds of formula (I-3a) which are compounds of formula (I-3) wherein X is S or SO₂, preferably X is SO₂.

Another group of compounds according to this embodiment are compounds of formula (I-3b) which are compounds of formula (I-3) wherein R₁ is C₁-C₄alkyl or cyclopropyl-C₁-C₄alkyl, preferably R₁ is ethyl or cyclopropylmethyl; more preferably R₁ is ethyl.

Another group of compounds according to this embodiment are compounds of formula (I-3c) which are compounds of formula (I-3) wherein R₂ is C₁-C₆fluoroalkyl; preferably R₂ is —CH₂CF₂CF₃, —CH₂CF₂CHF₂, —CH₂CF₃, —CH₂CHF₂ or —CH₂CF₂CHFCF₃; more preferably R₂ is —CH₂CF₃, —CH₂CF₂CHF₂ or —CH₂CF₂CF₃.

One group of compounds according to this embodiment are compounds of formula (I-3d) which are compounds of formula (I-3) wherein A₁ is CH₂.

Another group of compounds according to this embodiment are compounds of formula (I-3e) which are compounds of formula (I-3) wherein A₁ is O.

Another group of compounds according to the invention are those of formula I-4

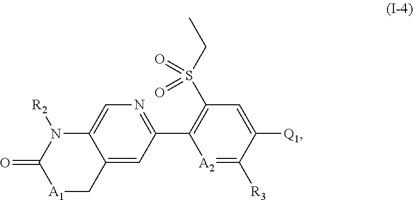

wherein
A₁ is CH₂ or O;
A₂ is CH or N, preferably N;
R₂ is C₁-C₆haloalkyl, preferably R₂ is C₁-C₆fluoroalkyl, more preferably R₂ is —CH₂CF₂CF₃, —CH₂CF₂CHF₂, —CH₂CF₃, —CH₂CHF₂ or —CH₂CF₂CHFCF₃;
R₃ is hydrogen or C₁-C₄alkyl, preferably hydrogen or methyl;
Q₁ is hydrogen, halogen, C₁-C₆haloalkyl, C₃-C₆cycloalkyl, C₃-C₆cycloalkyl monosubstituted by cyano, C₁-C₆cyanoalkyl, C₁-C₆cyanoalkoxy, C₁-C₆haloalkoxy, —N(R₄)₂, —N(R₄)COR₅, or —N(R₄)CON(R₄)₂, (oxazolidin-2-one)-3-yl or 2-pyridyloxy; or
Q₁ is a five- to six-membered aromatic ring system linked via a ring carbon atom to the ring which contains the substituent A₂, said ring system is unsubstituted or is mono-substituted by a substituent selected from the group consisting of halogen, cyano and C₁-C₄haloalkyl; and said ring system can contain 1 or 2 ring nitrogen atoms; or
Q₁ is a five-membered aromatic ring system linked via a ring nitrogen atom to the ring which contains the substituent A₂, said ring system is unsubstituted or is mono-substituted by a substituent selected from the group consisting of halogen, cyano and C₁-C₄haloalkyl; and said ring system contains 2 or 3 ring nitrogen atoms;
Each R₄ is independently hydrogen or C₁-C₄alkyl, preferably hydrogen or methyl; and
R₅ is C₁-C₆alkyl or C₃-C₆cycloalkyl, preferably methyl, ethyl or cyclopropyl, more preferably methyl or cyclopropyl; or
an agrochemically acceptable salt, stereoisomer, enantiomer, tautomer or N-oxide thereof.

One group of compounds according to this embodiment are compounds of formula (I-4a) which are compounds of formula (I-4) wherein A₁ is CH₂.

Another preferred group of compounds according to this embodiment are compounds of formula (I-4b) which are compounds of formula (I-4) wherein A₁ is O.

One further preferred group of compounds according to this embodiment are compounds of formula (I-4c) which are compounds of formula (I-4) wherein A₂ is N.

Another preferred group of compounds according to this embodiment are compounds of formula (I-4d) which are compounds of formula (I-4) wherein A₂ is CH.

One further preferred group of compounds according to this embodiment are compounds of formula (I-4e) which are compounds of formula (I-4) wherein R₃ is hydrogen.

Another preferred group of compounds according to this embodiment are compounds of formula (I-40 which are compounds of formula (I-4) wherein R₃ is C₁-C₄alkyl, preferably methyl.

One further preferred group of compounds according to this embodiment are compounds of formula (I-4g) which are compounds of formula (I-4) wherein A₂ is N and R₃ is hydrogen.

One further preferred group of compounds according to this embodiment are compounds of formula (I-4h) which are compounds of formula (I-4) wherein A₁ is CH₂, A₂ is N and R₃ is hydrogen.

One further preferred group of compounds according to this embodiment are compounds of formula (I-4i) which are compounds of formula (I-4) wherein A₁ is O, A₂ is N and R₃ is hydrogen.

One further preferred group of compounds according to this embodiment are compounds of formula (I-4j) which are compounds of formula (I-4) wherein Q₁ is hydrogen, halogen, trifluoromethyl, fluoroisopropyl, cyclopropyl, cyanocyclopropyl, cyanoisopropyl, cyanoisopropoxy, trifluoroethoxy, difluoropropoxy, —N(R₄)₂, —N(R₄)COR₅, or —N(R₄)CON(R₄)₂, in each of which R₄ is independently either hydrogen or methyl and R₅ is either methyl, ethyl or cyclopropyl, or Q₁ is (oxazolidin-2-one)-3-yl or 2-pyridyloxy; preferably Q₁ is hydrogen, chlorine, bromine, trifluoromethyl, 1-fluoro-1-methyl-ethyl, cyclopropyl, 1-cyanocyclopropyl, 1-cyano-1-methyl-ethyl, 1-cyano-1-methyl-ethoxy, 2,2,2-trifluoroethoxy, 2,2-difluoropropoxy, —NH₂, —NH(CH₃), —N(CH₃)₂, —NHCOCH₃, —N(CH₃)COCH₃, —N(CH₃)COCH₂CH₃, —NHCO(cyclopropyl), —N(CH₃)CO(cyclopropyl), —N(H)CONH₂, —N(H)CONH(CH₃), —N(H)CON(CH₃)₂, —N(CH₃)CONH₂, —N(CH₃)CONH(CH₃), —N(CH₃)CON(CH₃)₂, (oxazolidin-2-one)-3-yl, or 2-pyridyloxy; more preferably Q₁ is hydrogen, chlorine, bromine, trifluoromethyl, 1-fluoro-1-methyl-ethyl, cyclopropyl, 1-cyanocyclopropyl, 1-cyano-1-methyl-ethyl, 1-cyano-1-methyl-ethoxy, 2,2,2-trifluoroethoxy, 2,2-difluoropropoxy, —NH(CH₃), —N(CH₃)COCH₃, —N(CH₃)CO(cyclopropyl), —N(H)CONH(CH₃), —N(CH₃)CONH(CH₃), (oxazolidin-2-one)-3-yl, or 2-pyridyloxy.

One further preferred group of compounds according to this embodiment are compounds of formula (I-4k) which are compounds of formula (I-4) wherein Q₁ is a five- to six-membered aromatic ring system linked via a ring carbon atom to the ring which contains the substituent A₂, said ring system is unsubstituted or is mono-substituted by a substituent selected from the group consisting of halogen, cyano and C₁-C₄haloalkyl; and said ring system can contain 1 or 2 ring nitrogen atoms; preferably Q₁ is C-linked pyrimidinyl; more preferably Q₁ is pyrimidin-2-yl.

One further preferred group of compounds according to this embodiment are compounds of formula (I-4l) which are compounds of formula (I-4) wherein Q₁ is a five-membered aromatic ring system linked via a ring nitrogen atom to the ring which contains the substituent A₂, said ring system is unsubstituted or is mono-substituted by a substituent selected from the group consisting of halogen, cyano and C₁-C₄haloalkyl; and said ring system contains 2 ring nitrogen atoms; preferably Q₁ is N-linked pyrazolyl which is unsubstituted or is mono-substituted by chloro, cyano or trifluoromethyl; more preferably Q₁ is pyrazol-1-yl, 3-chloro-pyrazol-1-yl, 3-cyano-pyrazol-1-yl, 3-trifluoromethyl-pyrazol-1-yl or 1,2,4-triazol-1-yl.

One further preferred group of compounds according to this embodiment are compounds of formula (I-4m) which are compounds of formula (I-4) wherein
A₁ is CH₂ or O;
A₂ is N;
R₂ is C₁-C₆fluoroalkyl, preferably —CH₂CF₂CHF₂ or —CH₂CF₂CF₃;
R₃ is hydrogen or C₁-C₄alkyl, preferably hydrogen or methyl; and
Q₁ is hydrogen, halogen, trifluoromethyl, fluoroisopropyl, cyclopropyl, cyanocyclopropyl, cyanoisopropyl, cyanoisopropoxy, trifluoroethoxy, difluoropropoxy, —N(R₄)₂, —N(R₄)COR₅, or —N(R₄)CON(R₄)₂, in each of which R₄ is independently either hydrogen or methyl and R₅ is methyl, ethyl or cyclopropyl, or Q₁ is (oxazolidin-2-one)-3-yl, 2-pyridyloxy, N-linked pyrazolyl which is unsubstituted or is mono-substituted by chloro, cyano or trifluoromethyl, or Q₁ is N-linked triazolyl or C-linked pyrimidinyl.

Another preferred group of compounds according to this embodiment are compounds of formula (I-4n) which are compounds of formula (I-4) wherein
A₁ is CH₂ or O;
A₂ is N;
R₂ is —CH₂CF₂CHF₂ or —CH₂CF₂CF₃;
R₃ is hydrogen; and
Q₁ is hydrogen, chlorine, bromine, trifluoromethyl, 1-fluoro-1-methyl-ethyl, cyclopropyl, 1-cyanocyclopropyl, 1-cyano-1-methyl-ethyl, 1-cyano-1-methyl-ethoxy, 2,2,2-trifluoroethoxy, 2,2-difluoropropoxy, —NH₂, —NH(CH₃), —N(CH₃)₂, —NHCOCH₃, —N(CH₃)COCH₃, —N(CH₃)COCH₂CH₃, —NHCO(cyclopropyl), —N(CH₃)CO(cyclopropyl), —N(H)CONH₂, —N(H)CONH(CH₃), —N(H)CON(CH₃)₂, —N(CH₃)CONH₂, —N(CH₃)CONH(CH₃), —N(CH₃)CON(CH₃)₂, (oxazolidin-2-one)-3-yl, 2-pyridyloxy, pyrazol-1-yl, 3-chloro-pyrazol-1-yl, 3-cyano-pyrazol-1-yl, 3-trifluoromethyl-pyrazol-1-yl, 1,2,4-triazol-1-yl or pyrimidin-2-yl; preferably Q₁ is hydrogen, chlorine, bromine, trifluoromethyl, 1-fluoro-1-methyl-ethyl, cyclopropyl, 1-cyanocyclopropyl, 1-cyano-1-methyl-ethyl, 1-cyano-1-methyl-ethoxy, 2,2,2-trifluoroethoxy, 2,2-difluoropropoxy, —NH(CH₃), —N(CH₃)COCH₃, —N(CH₃)CO(cyclopropyl), —N(H)CONH(CH₃), —N(CH₃)CONH(CH₃), (oxazolidin-2-one)-3-yl, 2-pyridyloxy, pyrazol-1-yl, 3-chloro-pyrazol-1-yl, 3-cyano-pyrazol-1-yl, 3-trifluoromethyl-pyrazol-1-yl. 1,2,4-triazol-1-yl or pyrimidin-2-yl.

One group of compounds according to the invention are those of formula I-5

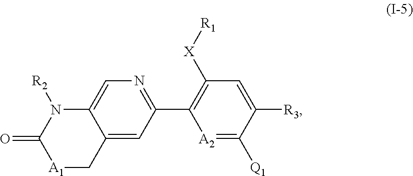

wherein A₁, A₂, X, R₁, and R₂ are as defined for compounds of formula I (above), or an agrochemically acceptable salt, stereoisomer, enantiomer, tautomer or N-oxide thereof, and wherein Q₁ is preferably hydrogen, halogen, C₁-C₆haloalkyl, C₃-C₆cycloalkyl, C₃-C₆cycloalkyl monosubstituted by cyano, C₁-C₆cyanoalkyl, C₁-C₆cyanoalkoxy, C₁-C₆haloalkoxy, —N(R₄)₂, —N(R₄)COR₅, or —N(R₄)CON(R₄)₂, (oxazolidin-2-one)-3-yl or 2-pyridyloxy; or
Q₁ is a five- to six-membered aromatic ring system linked via a ring carbon atom to the ring which contains the substituent A₂, said ring system is unsubstituted or is mono-substituted by a substituent selected from the group consisting of halogen, cyano and C₁-C₄haloalkyl; and said ring system can contain 1 or 2 ring nitrogen atoms; or
Q₁ is a five-membered aromatic ring system linked via a ring nitrogen atom to the ring which contains the substituent A₂, said ring system is unsubstituted or is mono-substituted by a substituent selected from the group consisting of halogen, cyano and C₁-C₄haloalkyl; and said ring system contains 2 or 3 ring nitrogen atoms;
R₃ is preferably hydrogen or C₁-C₄alkyl;
Preferably each R₄ is independently hydrogen or C₁-C₄alkyl; and
R₅ is preferably C₁-C₆alkyl or C₃-C₆cycloalkyl.

Preferred definitions of A₁, A₂, X, R₁, and R₂ in the compounds of formula I-5 are as defined for compounds of formula I (above), and more preferably Q₁ is hydrogen, halogen, trifluoromethyl, fluoroisopropyl, cyclopropyl, cyanocyclopropyl, cyanoisopropyl, cyanoisopropoxy, trifluoroethoxy, difluoropropoxy, —N(R₄)₂, —N(R₄)COR₅, or —N(R₄)CON(R₄)₂, in each of which R₄ is independently either hydrogen or methyl and R₅ is methyl, ethyl or cyclopropyl; preferably methyl or cyclopropyl, or Q₁ is (oxazolidin-2-one)-3-yl, 2-pyridyloxy, N-linked pyrazolyl which is unsubstituted or is mono-substituted by chloro, cyano or trifluoromethyl, or Q₁ is N-linked triazolyl or C-linked pyrimidinyl; and R₃ is hydrogen or methyl, preferably hydrogen.

One group of compounds according to this embodiment are compounds of formula (I-5a) which are compounds of formula (I-5) wherein A₂ is N.

Another group of compounds according to this embodiment are compounds of formula (I-5b) which are compounds of formula (I-5) wherein A₂ is CH.

One group of compounds according to this embodiment are compounds of formula (I-5c) which are compounds of formula (I-5) wherein R₂ is C₁-C₆fluoroalkyl; preferably R₂ is —CH₂CF₂CF₃, —CH₂CF₂CHF₂, —CH₂CF₃, —CH₂CHF₂ or —CH₂CF₂CHFCF₃; more preferably R₂ is —CH₂CF₃, —CH₂CF₂CHF₂ or —CH₂CF₂CF₃.

Another group of compounds according to this embodiment are compounds of formula (I-5d) which are compounds of formula (I-5) wherein X is S or SO₂, preferably X is SO₂.

Another group of compounds according to this embodiment are compounds of formula (I-5e) which are compounds of formula (I-5) wherein R₁ is C₁-C₄alkyl or cyclopropyl-C₁-C₄alkyl; preferably R₁ is ethyl or cyclopropylmethyl; more preferably R₁ is ethyl.

One group of compounds according to this embodiment are compounds of formula (I-5f which are compounds of formula (I-5) wherein A₁ is CH₂.

Another group of compounds according to this embodiment are compounds of formula (I-5g) which are compounds of formula (I-5) wherein A₁ is O.

Another group of compounds according to the invention are those of formula I-6

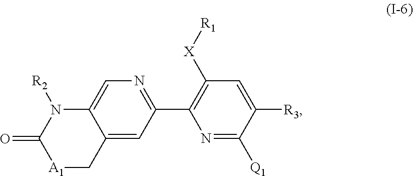

wherein A₁, X, R₁ and R₂ are as defined for compounds of formula I (above), or an agrochemically acceptable salt, stereoisomer, enantiomer, tautomer or N-oxide thereof, and wherein Q₁ is preferably hydrogen, halogen, C₁-C₆haloalkyl, C₃-C₆cycloalkyl, C₃-C₆cycloalkyl monosubstituted by cyano, C₁-C₆cyanoalkyl, C₁-C₆cyanoalkoxy, C₁-C₆haloalkoxy, —N(R₄)₂, —N(R₄)COR₅, or —N(R₄)CON(R₄)₂, (oxazolidin-2-one)-3-yl or 2-pyridyloxy; or
Q₁ is a five- to six-membered aromatic ring system linked via a ring carbon atom to the pyridyl ring substituted by X—R₁, said ring system is unsubstituted or is mono-substituted by a substituent selected from the group consisting of halogen, cyano and C₁-C₄haloalkyl; and said ring system can contain 1 or 2 ring nitrogen atoms; or
Q₁ is a five-membered aromatic ring system linked via a ring nitrogen to the pyridyl ring substituted by X—R₁, said ring system is unsubstituted or is mono-substituted by a substituent selected from the group consisting of halogen, cyano and C₁-C₄haloalkyl; and said ring system contains 2 or 3 ring nitrogen atoms;
R₃ is preferably hydrogen or C₁-C₄alkyl;
Preferably each R₄ is independently hydrogen or C₁-C₄alkyl; and
R₅ is preferably C₁-C₆alkyl or C₃-C₆cycloalkyl.

Preferred definitions of A₁, X, R₁ and R₂ in the compounds of formula I-6 are as defined for compounds of formula I (above), and more preferably Q₁ is hydrogen, halogen, trifluoromethyl, fluoroisopropyl, cyclopropyl, cyanocyclopropyl, cyanoisopropyl, cyanoisopropoxy, trifluoroethoxy, difluoropropoxy, —N(R₄)₂, —N(R₄)COR₅, or —N(R₄)CON(R₄)₂, in each of which R₄ is independently either hydrogen or methyl and R₅ is methyl, ethyl or cyclopropyl; preferably methyl or cyclopropyl, or Q₁ is (oxazolidin-2-one)-3-yl, 2-pyridyloxy, N-linked pyrazolyl which is unsubstituted or is mono-substituted by chloro, cyano or trifluoromethyl, or Q₁ is N-linked triazolyl or C-linked pyrimidinyl; and R₃ is hydrogen or methyl, preferably hydrogen.

In one group of compounds of formula I-6, preferably Q₁ is hydrogen, chlorine, bromine, trifluoromethyl, cyclopropyl, 1-cyanocyclopropyl, 1-cyano-1-methyl-ethyl, 2,2,2-trifluoroethoxy, —NH(CH₃), —N(CH₃)COCH₃, —N(CH₃)CO(cyclopropyl), —N(H)CONH(CH₃), —N(CH₃)CONH(CH₃), (oxazolidin-2-one)-3-yl, 2-pyridyloxy, pyrazol-1-yl, 3-chloro-pyrazol-1-yl, 3-cyano-pyrazol-1-yl, 3-trifluoromethyl-pyrazol-1-yl, 1,2,4-triazol-1-yl or pyrimidin-2-yl.

In another group of compounds of formula I-6, also preferred is when Q₁ is chlorine, bromine, trifluoromethyl, 1-fluoro-1-methyl-ethyl, cyclopropyl, 1-cyanocyclopropyl, 1-cyano-1-methyl-ethyl, 1-cyano-1-methyl-ethoxy, 2,2,2-trifluoroethoxy, 2,2-difluoropropoxy, —NH(CH₃), —N(CH₃)COCH₃, —N(CH₃)CO(cyclopropyl), —N(H)CONH(CH₃), —N(CH₃)CONH(CH₃), (oxazolidin-2-one)-3-yl, 2-pyridyloxy, pyrazol-1-yl, 3-chloro-pyrazol-1-yl, 3-cyano-pyrazol-1-yl, 3-trifluoromethyl-pyrazol-1-yl, 1,2,4-triazol-1-yl or pyrimidin-2-yl.

One group of compounds according to this embodiment are compounds of formula (I-6a) which are compounds of formula (I-6) wherein X is S or SO₂, preferably X is SO₂.

Another group of compounds according to this embodiment are compounds of formula (I-6b) which are compounds of formula (I-6) wherein R₁ is C₁-C₄alkyl or cyclopropyl-C₁-C₄alkyl, preferably R₁ is ethyl or cyclopropylmethyl; more preferably R₁ is ethyl.

Another group of compounds according to this embodiment are compounds of formula (I-6c) which are compounds of formula (I-6) wherein R₂ is C₁-C₆fluoroalkyl; preferably R₂ is —CH₂CF₂CF₃, —CH₂CF₂CHF₂, —CH₂CF₃, —CH₂CHF₂ or —CH₂CF₂CHFCF₃; more preferably R₂ is —CH₂CF₃, —CH₂CF₂CHF₂ or —CH₂CF₂CF₃.

One group of compounds according to this embodiment are compounds of formula (I-6d) which are compounds of formula (I-6) wherein A₁ is CH₂.

Another group of compounds according to this embodiment are compounds of formula (I-6e) which are compounds of formula (I-6) wherein A₁ is O.

Another group of compounds according to the invention are those of formula I-7

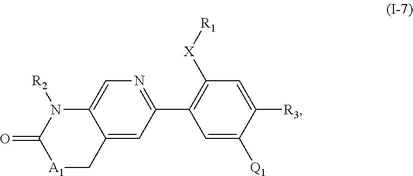

wherein A₁, X, R₁ and R₂ are as defined for compounds of formula I (above), or an agrochemically acceptable salt, stereoisomer, enantiomer, tautomer or N-oxide thereof, and wherein Q₁ is preferably hydrogen, halogen, C₁-C₆haloalkyl, C₃-C₆cycloalkyl, C₃-C₆cycloalkyl monosubstituted by cyano, C₁-C₆cyanoalkyl, C₁-C₆cyanoalkoxy, C₁-C₆haloalkoxy, —N(R₄)₂, —N(R₄)COR₅, or —N(R₄)CON(R₄)₂, (oxazolidin-2-one)-3-yl or 2-pyridyloxy; or
Q₁ is a five- to six-membered aromatic ring system linked via a carbon atom to the phenyl ring substituted by X—R₁, said ring system is unsubstituted or is mono-substituted by a substituent selected from the group consisting of halogen, cyano and C₁-C₄haloalkyl; and said ring system can contain 1 or 2 ring nitrogen atoms; or
Q₁ is a five-membered aromatic ring system linked via a nitrogen atom to the phenyl ring substituted by X—R₁, said ring system is unsubstituted or is mono-substituted by a substituent selected from the group consisting of halogen, cyano and C₁-C₄haloalkyl; and said ring system contains 2 or 3 ring nitrogen atoms;
R₃ is preferably hydrogen or C₁-C₄alkyl;
Preferably each R₄ is independently hydrogen or C₁-C₄alkyl; and
R₅ is preferably C₁-C₆alkyl or C₃-C₆cycloalkyl.

Preferred definitions of A₁, X, R₁ and R₂ in the compounds of formula I-7 are as defined for compounds of formula I (above), and more preferably Q₁ is hydrogen, halogen, trifluoromethyl, fluoroisopropyl, cyclopropyl, cyanocyclopropyl, cyanoisopropyl, cyanoisopropoxy, trifluoroethoxy, difluoropropoxy, —N(R₄)₂, —N(R₄)COR₅, or —N(R₄)CON(R₄)₂, in each of which R₄ is independently either hydrogen or methyl and R₅ is methyl, ethyl or cyclopropyl; preferably methyl or cyclopropyl, or Q₁ is (oxazolidin-2-one)-3-yl, 2-pyridyloxy, N-linked pyrazolyl which is unsubstituted or is mono-substituted by chloro, cyano or trifluoromethyl, or Q₁ is N-linked triazolyl or C-linked pyrimidinyl; and R₃ is hydrogen or methyl, preferably hydrogen.

In one group of compounds of formula I-7, preferably Q₁ is hydrogen, chlorine, bromine, trifluoromethyl, 1-fluoro-1-methyl-ethyl, cyclopropyl, 1-cyanocyclopropyl, 1-cyano-1-methyl-ethyl, 1-cyano-1-methyl-ethoxy, 2,2,2-trifluoroethoxy, 2,2-difluoropropoxy, —NH(CH₃), —N(CH₃)COCH₃, —N(CH₃)CO(cyclopropyl), —N(H)CONH(CH₃), —N(CH₃)CONH(CH₃), (oxazolidin-2-one)-3-yl, 2-pyridyloxy, pyrazol-1-yl, 3-chloro-pyrazol-1-yl, 3-cyano-pyrazol-1-yl, 3-trifluoromethyl-pyrazol-1-yl, 1,2,4-triazol-1-yl or pyrimidin-2-yl.

In another group of compounds of formula I-7, also preferred is when Q₁ is chlorine, bromine, trifluoromethyl, 1-fluoro-1-methyl-ethyl, cyclopropyl, 1-cyanocyclopropyl, 1-cyano-1-methyl-ethyl, 1-cyano-1-methyl-ethoxy, 2,2,2-trifluoroethoxy, 2,2-difluoropropoxy, —NH(CH₃), —N(CH₃)COCH₃, —N(CH₃)CO(cyclopropyl), —N(H)CONH(CH₃), —N(CH₃)CONH(CH₃), (oxazolidin-2-one)-3-yl, 2-pyridyloxy, pyrazol-1-yl, 3-chloro-pyrazol-1-yl, 3-cyano-pyrazol-1-yl, 3-trifluoromethyl-pyrazol-1-yl, 1,2,4-triazol-1-yl or pyrimidin-2-yl.

One group of compounds according to this embodiment are compounds of formula (I-7a) which are compounds of formula (I-7) wherein X is S or SO₂, preferably X is SO₂.

Another group of compounds according to this embodiment are compounds of formula (I-7b) which are compounds of formula (I-7) wherein R₁ is C₁-C₄alkyl or cyclopropyl-C₁-C₄alkyl, preferably R₁ is ethyl or cyclopropylmethyl; more preferably R₁ is ethyl.

Another group of compounds according to this embodiment are compounds of formula (I-7c) which are compounds of formula (I-7) wherein R₂ is C₁-C₆fluoroalkyl; preferably R₂ is —CH₂CF₂CF₃, —CH₂CF₂CHF₂, —CH₂CF₃, —CH₂CHF₂ or —CH₂CF₂CHFCF₃; more preferably R₂ is —CH₂CF₃, —CH₂CF₂CHF₂ or —CH₂CF₂CF₃.

One group of compounds according to this embodiment are compounds of formula (I-7d) which are compounds of formula (I-7) wherein A₁ is CH₂.

Another group of compounds according to this embodiment are compounds of formula (I-7e) which are compounds of formula (I-7) wherein A₁ is O.

Another group of compounds according to the invention are those of formula I-8

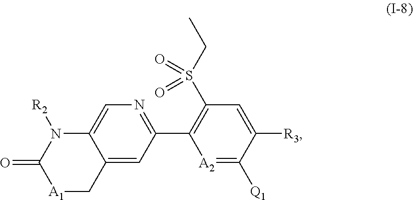

wherein
A₁ is CH₂ or O;
A₂ is CH or N, preferably N;
R₂ is C₁-C₆haloalkyl, preferably R₂ is C₁-C₆fluoroalkyl, more preferably R₂ is —CH₂CF₂CF₃, —CH₂CF₂CHF₂, —CH₂CF₃, —CH₂CHF₂ or —CH₂CF₂CHFCF₃;
R₃ is hydrogen or C₁-C₄alkyl, preferably hydrogen or methyl;
Q₁ is hydrogen, halogen, C₁-C₆haloalkyl, C₃-C₆cycloalkyl, C₃-C₆cycloalkyl monosubstituted by cyano, C₁-C₆cyanoalkyl, C₁-C₆cyanoalkoxy, C₁-C₆haloalkoxy, —N(R₄)₂, —N(R₄)COR₅, or —N(R₄)CON(R₄)₂, (oxazolidin-2-one)-3-yl or 2-pyridyloxy; or
Q₁ is a five- to six-membered aromatic ring system linked via a ring carbon atom to the ring which contains the substituent A₂, said ring system is unsubstituted or is mono-substituted by a substituent selected from the group consisting of halogen, cyano and C₁-C₄haloalkyl; and said ring system can contain 1 or 2 ring nitrogen atoms; or
Q₁ is a five-membered aromatic ring system linked via a ring nitrogen atom to the ring which contains the substituent A₂, said ring system is unsubstituted or is mono-substituted by a substituent selected from the group consisting of halogen, cyano and C₁-C₄haloalkyl; and said ring system contains 2 or 3 ring nitrogen atoms;
Each R₄ is independently hydrogen or C₁-C₄alkyl, preferably hydrogen or methyl; and
R₅ is C₁-C₆alkyl or C₃-C₆cycloalkyl; preferably methyl, ethyl or cyclopropyl, more preferably methyl or cyclopropyl; or
an agrochemically acceptable salt, stereoisomer, enantiomer, tautomer or N-oxide thereof.

One group of compounds according to this embodiment are compounds of formula (I-8a) which are compounds of formula (I-8) wherein A₁ is CH₂.

Another preferred group of compounds according to this embodiment are compounds of formula (I-8b) which are compounds of formula (I-8) wherein A₁ is O.

One further preferred group of compounds according to this embodiment are compounds of formula (I-8c) which are compounds of formula (I-8) wherein A₂ is N.

Another preferred group of compounds according to this embodiment are compounds of formula (I-8d) which are compounds of formula (I-8) wherein A₂ is CH.

One further preferred group of compounds according to this embodiment are compounds of formula (I-8e) which are compounds of formula (I-8) wherein R₃ is hydrogen.

Another preferred group of compounds according to this embodiment are compounds of formula (I-80 which are compounds of formula (I-8) wherein R₃ is C₁-C₄alkyl, preferably methyl.

One further preferred group of compounds according to this embodiment are compounds of formula (I-8g) which are compounds of formula (I-8) wherein A₂ is N and R₃ is hydrogen.

One further preferred group of compounds according to this embodiment are compounds of formula (I-8h) which are compounds of formula (I-8) wherein A₁ is CH₂, A₂ is N and R₃ is hydrogen.

One further preferred group of compounds according to this embodiment are compounds of formula (I-8i) which are compounds of formula (I-8) wherein A₁ is O, A₂ is N and R₃ is hydrogen.

One further preferred group of compounds according to this embodiment are compounds of formula (I-8j) which are compounds of formula (I-8) wherein Q₁ is hydrogen, halogen, trifluoromethyl, fluoroisopropyl, cyclopropyl, cyanocyclopropyl, cyanoisopropyl, cyanoisopropoxy, trifluoroethoxy, difluoropropoxy, —N(R₄)₂, —N(R₄)COR₅, or —N(R₄)CON(R₄)₂, in each of which R₄ is independently either hydrogen or methyl and R₅ is methyl, ethyl or cyclopropyl, or Q₁ is (oxazolidin-2-one)-3-yl or 2-pyridyloxy; preferably Q₁ is hydrogen, chlorine, bromine, trifluoromethyl, 1-fluoro-1-methyl-ethyl, cyclopropyl, 1-cyanocyclopropyl, 1-cyano-1-methyl-ethyl, 1-cyano-1-methyl-ethoxy, 2,2,2-trifluoroethoxy, 2,2-difluoropropoxy, —NH₂, —NH(CH₃), —N(CH₃)₂, —NHCOCH₃, —N(CH₃)COCH₃, —N(CH₃)COCH₂CH₃, —NHCO(cyclopropyl), —N(CH₃)CO(cyclopropyl), —N(H)CONH₂, —N(H)CONH(CH₃), —N(H)CON(CH₃)₂, —N(CH₃)CONH₂, —N(CH₃)CONH(CH₃), —N(CH₃)CON(CH₃)₂, (oxazolidin-2-one)-3-yl, or 2-pyridyloxy; more preferably Q₁ is hydrogen, chlorine, bromine, trifluoromethyl, 1-fluoro-1-methyl-ethyl, cyclopropyl, 1-cyanocyclopropyl, 1-cyano-1-methyl-ethyl, 1-cyano-1-methyl-ethoxy, 2,2,2-trifluoroethoxy, 2,2-difluoropropoxy, —NH(CH₃), —N(CH₃)COCH₃, —N(CH₃)CO(cyclopropyl), —N(H)CONH(CH₃), —N(CH₃)CONH(CH₃), (oxazolidin-2-one)-3-yl, or 2-pyridyloxy.

One further preferred group of compounds according to this embodiment are compounds of formula (I-8k) which are compounds of formula (I-8) wherein Q₁ is a five- to six-membered aromatic ring system linked via a ring carbon atom to the ring which contains the substituent A₂, said ring system is unsubstituted or is mono-substituted by a substituent selected from the group consisting of halogen, cyano and C₁-C₄haloalkyl; and said ring system can contain 1 or 2 ring nitrogen atoms; preferably Q₁ is C-linked pyrimidinyl; more preferably Q₁ is pyrimidin-2-yl.

One further preferred group of compounds according to this embodiment are compounds of formula (I-81) which are compounds of formula (I-8) wherein Q₁ is a five-membered aromatic ring system linked via a ring nitrogen atom to the ring which contains the substituent A₂, said ring system is unsubstituted or is mono-substituted by a substituent selected from the group consisting of halogen, cyano and C₁-C₄haloalkyl; and said ring system contains 2 ring nitrogen atoms; preferably Q₁ is N-linked pyrazolyl which is unsubstituted or is mono-substituted by chloro, cyano or trifluoromethyl; more preferably Q₁ is pyrazol-1-yl, 3-chloro-pyrazol-1-yl, 3-cyano-pyrazol-1-yl, 3-trifluoromethyl-pyrazol-1-yl or 1,2,4-triazol-1-yl.

One further preferred group of compounds according to this embodiment are compounds of formula (I-8m) which are compounds of formula (I-8) wherein
A₁ is CH₂ or O;
A₂ is N;
R₂ is C₁-C₆fluoroalkyl, preferably —CH₂CF₃, —CH₂CF₂CHF₂ or —CH₂CF₂CF₃;
R₃ is hydrogen or C₁-C₄alkyl, preferably hydrogen or methyl; and
Q₁ is hydrogen, halogen, trifluoromethyl, cyclopropyl, cyanocyclopropyl, cyanoisopropyl, trifluoroethoxy, —N(R₄)₂, —N(R₄)COR₅, or —N(R₄)CON(R₄)₂, in each of which R₄ is independently either hydrogen or methyl and R₅ is either methyl or cyclopropyl, or Q₁ is (oxazolidin-2-one)-3-yl, 2-pyridyloxy, N-linked pyrazolyl which is unsubstituted or is mono-substituted by chloro, cyano or trifluoromethyl, or Q₁ is N-linked triazolyl or C-linked pyrimidinyl.

Another preferred group of compounds according to this embodiment are compounds of formula (I-8n) which are compounds of formula (I-8) wherein
A₁ is CH₂ or O;
A₂ is N;
R₂ is —CH₂CF₃, —CH₂CF₂CHF₂ or —CH₂CF₂CF₃;
R₃ is hydrogen; and
Q₁ is hydrogen, chlorine, bromine, trifluoromethyl, 1-fluoro-1-methyl-ethyl, cyclopropyl, 1-cyanocyclopropyl, 1-cyano-1-methyl-ethyl, 1-cyano-1-methyl-ethoxy, 2,2,2-trifluoroethoxy, 2,2-difluoropropoxy, —NH₂, —NH(CH₃), —N(CH₃)₂, —NHCOCH₃, —N(CH₃)COCH₃, —N(CH₃)COCH₂CH₃, —NHCO(cyclopropyl), —N(CH₃)CO(cyclopropyl), —N(H)CONH₂, —N(H)CONH(CH₃), —N(H)CON(CH₃)₂, —N(CH₃)CONH₂, —N(CH₃)CONH(CH₃), —N(CH₃)CON(CH₃)₂, (oxazolidin-2-one)-3-yl, 2-pyridyloxy, pyrazol-1-yl, 3-chloro-pyrazol-1-yl, 3-cyano-pyrazol-1-yl, 3-trifluoromethyl-pyrazol-1-yl, 1,2,4-triazol-1-yl or pyrimidin-2-yl; preferably Q₁ is hydrogen, chlorine, bromine, trifluoromethyl, 1-fluoro-1-methyl-ethyl, cyclopropyl, 1-cyanocyclopropyl, 1-cyano-1-methyl-ethyl, 2,2,2-trifluoroethoxy, 2,2-difluoropropoxy, —NH(CH₃), —N(CH₃)COCH₃, —N(CH₃)CO(cyclopropyl), —N(H)CONH(CH₃), —N(CH₃)CONH(CH₃), (oxazolidin-2-one)-3-yl, 2-pyridyloxy, pyrazol-1-yl, 3-chloro-pyrazol-1-yl, 3-cyano-pyrazol-1-yl, 3-trifluoromethyl-pyrazol-1-yl, 1,2,4-triazol-1-yl or pyrimidin-2-yl.

An outstanding group of compounds according to the invention are those of formula I-9

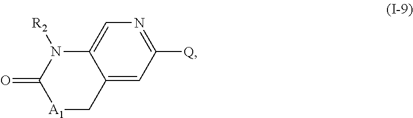

wherein
A₁ is CH₂ or O;
R₂ is C₁-C₆haloalkyl, preferably R₂ is C₁-C₆fluoroalkyl, more preferably R₂ is —CH₂CF₂CF₃, —CH₂CF₂CHF₂, —CH₂CF₃, —CH₂CHF₂ or —CH₂CF₂CHFCF₃;
Q is a radical selected from the group consisting of formula Qa1 and Qb1

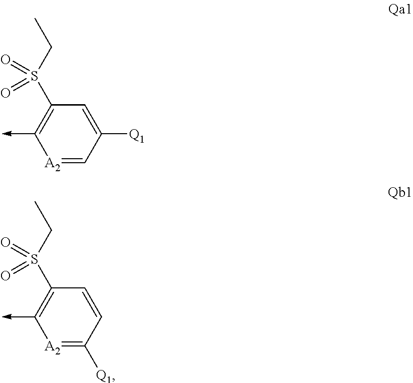

wherein the arrow denotes the point of attachment to the carbon atom of the bicyclic ring; and wherein
A₂ is CH or N, preferably N; and
Q₁ is hydrogen, halogen, trifluoromethyl, fluoroisopropyl, cyclopropyl, cyanocyclopropyl, cyanoisopropyl, cyanoisopropoxy, trifluoroethoxy, difluoropropoxy, —N(R₄)₂, —N(R₄)COR₅, or —N(R₄)CON(R₄)₂, in each of which R₄ is independently either hydrogen or methyl and R₅ is methyl, ethyl or cyclopropyl; preferably R₅ is methyl or cyclopropyl, or Q₁ is (oxazolidin-2-one)-3-yl, 2-pyridyloxy, N-linked pyrazolyl which is unsubstituted or is mono-substituted by chloro, cyano or trifluoromethyl, or Q₁ is N-linked triazolyl or C-linked pyrimidinyl;
preferably Q₁ is hydrogen, chlorine, bromine, trifluoromethyl, 1-fluoro-1-methyl-ethyl, cyclopropyl, 1-cyanocyclopropyl, 1-cyano-1-methyl-ethyl, 1-cyano-1-methyl-ethoxy, 2,2,2-trifluoroethoxy, 2,2-difluoropropoxy, —NH₂, —NH(CH₃), —N(CH₃)₂, —NHCOCH₃, —N(CH₃)COCH₃, —N(CH₃)COCH₂CH₃, —NHCO(cyclopropyl), —N(CH₃)CO(cyclopropyl), —N(H)CONH₂, —N(H)CONH(CH₃), —N(H)CON(CH₃)₂, —N(CH₃)CONH₂, —N(CH₃)CONH(CH₃), —N(CH₃)CON(CH₃)₂, (oxazolidin-2-one)-3-yl, 2-pyridyloxy, pyrazol-1-yl, 3-chloro-pyrazol-1-yl, 3-cyano-pyrazol-1-yl, 3-trifluoromethyl-pyrazol-1-yl, 1,2,4-triazol-1-yl or pyrimidin-2-yl;
more preferably Q₁ is hydrogen, chlorine, bromine, trifluoromethyl, 1-fluoro-1-methyl-ethyl, cyclopropyl, 1-cyanocyclopropyl, 1-cyano-1-methyl-ethyl, 1-cyano-1-methyl-ethoxy, 2,2,2-trifluoroethoxy, 2,2-difluoropropoxy, —NH(CH₃), —N(CH₃)COCH₃, —N(CH₃)CO(cyclopropyl), —N(H)CONH(CH₃), —N(CH₃)CONH(CH₃), (oxazolidin-2-one)-3-yl, 2-pyridyloxy, pyrazol-1-yl, 3-chloro-pyrazol-1-yl, 3-cyano-pyrazol-1-yl, 3-trifluoromethyl-pyrazol-1-yl, 1,2,4-triazol-1-yl or pyrimidin-2-yl.

One group of compounds according to this embodiment are compounds of formula (I-9a) which are compounds of formula (I-9) and any of the preferred embodiments of formula (I-9) wherein A₁ is CH₂.

Another preferred group of compounds according to this embodiment are compounds of formula (I-9b) which are compounds of formula (I-9) and any of the preferred embodiments of formula (I-9) wherein A₁ is O.

One further preferred group of compounds according to this embodiment are compounds of formula (I-9c) which are compounds of formula (I-9) and any of the preferred embodiments of formula (I-9) wherein A₂ is N.

Another group of compounds according to this embodiment are compounds of formula (I-9d) which are compounds of formula (I-9) and any of the preferred embodiments of formula (I-9) wherein A₂ is CH.

One group of compounds according to this embodiment are compounds of formula (I-9e) which are compounds of formula (I-9) and any of the preferred embodiments of formula (I-9) wherein R₂ is —CH₂CF₃, —CH₂CF₂CHF₂ or —CH₂CF₂CF₃.

One further preferred group of compounds according to this embodiment are compounds of formula (I-9f) which are compounds of formula (I-9) and any of the preferred embodiments of formula (I-9) wherein A₁ is CH₂, A₂ is N and R₂ is —CH₂CF₃, —CH₂CF₂CHF₂ or —CH₂CF₂CF₃.

One further preferred group of compounds according to this embodiment are compounds of formula (I-9g) which are compounds of formula (I-9) and any of the preferred embodiments of formula (I-9) wherein A₁ is O, A₂ is N and R₂ is —CH₂CF₃, —CH₂CF₂CHF₂ or —CH₂CF₂CF₃.

Another group of compounds according to this embodiment are compounds of formula (I-9h) which are compounds of formula (I-9) and any of the preferred embodiments of formula (I-9) wherein Q₁ is hydrogen, halogen, trifluoromethyl, fluoroisopropyl, cyclopropyl, cyanocyclopropyl, cyanoisopropyl, cyanoisopropoxy, trifluoroethoxy, difluoropropoxy, —N(R₄)₂, —N(R₄)COR₅, or —N(R₄)CON(R₄)₂, in each of which R₄ is independently either hydrogen or methyl and R₅ is methyl, ethyl or cyclopropyl, or Q₁ is (oxazolidin-2-one)-3-yl, 2-pyridyloxy, N-linked pyrazolyl which is unsubstituted or is mono-substituted by chloro, cyano or trifluoromethyl, or Q₁ is N-linked triazolyl or C-linked pyrimidinyl; preferably Q₁ is hydrogen, chlorine, bromine, trifluoromethyl, 1-fluoro-1-methyl-ethyl, cyclopropyl, 1-cyanocyclopropyl, 1-cyano-1-methyl-ethyl, 1-cyano-1-methyl-ethoxy, 2,2,2-trifluoroethoxy, 2,2-difluoropropoxy, —NH(CH₃), —N(CH₃)COCH₃, —N(CH₃)CO(cyclopropyl), —N(H)CONH(CH₃), —N(CH₃)CONH(CH₃), (oxazolidin-2-one)-3-yl, 2-pyridyloxy, pyrazol-1-yl, 3-chloro-pyrazol-1-yl, 3-cyano-pyrazol-1-yl, 3-trifluoromethyl-pyrazol-1-yl, 1,2,4-triazol-1-yl or pyrimidin-2-yl.

Another group of compounds according to this embodiment are compounds of formula (I-9i) which are compounds of formula (I-9) and any of the preferred embodiments of formula (I-9) wherein Q₁ is hydrogen, 1-cyanocyclopropyl, or 3-chloro-pyrazol-1-yl.

Another group of compounds according to this embodiment are compounds of formula (I-9j) which are compounds of formula (I-9) and any of the preferred embodiments of formula (I-9) wherein Q₁ is chlorine, bromine, trifluoromethyl, cyclopropyl, 1-cyanocyclopropyl, 1-cyano-1-methyl-ethyl, 2,2,2-trifluoroethoxy, —NH(CH₃), —N(CH₃)COCH₃, —N(CH₃)CO(cyclopropyl), —N(H)CONH(CH₃), or —N(CH₃)CONH(CH₃).

Another group of compounds according to this embodiment are compounds of formula (I-9k) which are compounds of formula (I-9) and any of the preferred embodiments of formula (I-9) wherein Q₁ is (oxazolidin-2-one)-3-yl, 2-pyridyloxy, pyrazol-1-yl, 3-chloro-pyrazol-1-yl, 3-cyano-pyrazol-1-yl, 3-trifluoromethyl-pyrazol-1-yl, 1,2,4-triazol-1-yl or pyrimidin-2-yl.

One further outstanding group of compounds according to this embodiment are compounds of formula (I-91) which are compounds of formula (I-9) wherein: A₁ is CH₂ or O;
R₂ is —CH₂CF₃, —CH₂CF₂CHF₂ or —CH₂CF₂CF₃;
Q is a radical selected from the group consisting of formula Qa1 and Qb1, wherein
A₂ is N; and
Q₁ is hydrogen, chlorine, bromine, trifluoromethyl, 1-fluoro-1-methyl-ethyl, cyclopropyl, 1-cyanocyclopropyl, 1-cyano-1-methyl-ethyl, 1-cyano-1-methyl-ethoxy, 2,2,2-trifluoroethoxy, 2,2-difluoropropoxy, —NH₂, —NH(CH₃), —N(CH₃)₂, —NHCOCH₃, —N(CH₃)COCH₃, —N(CH₃)COCH₂CH₃, —NHCO(cyclopropyl), —N(CH₃)CO(cyclopropyl), —N(H)CONH₂, —N(H)CONH(CH₃), —N(H)CON(CH₃)₂, —N(CH₃)CONH₂, —N(CH₃)CONH(CH₃), —N(CH₃)CON(CH₃)₂, (oxazolidin-2-one)-3-yl, 2-pyridyloxy, pyrazol-1-yl, 3-chloro-pyrazol-1-yl, 3-cyano-pyrazol-1-yl, 3-trifluoromethyl-pyrazol-1-yl, 1,2,4-triazol-1-yl or pyrimidin-2-yl.

One further outstanding group of compounds according to this embodiment are compounds of formula (I-9m) which are compounds of formula (I-9) wherein:
A₁ is CH₂ or O;
R₂ is —CH₂CF₃, —CH₂CF₂CHF₂ or —CH₂CF₂CF₃;
Q is a radical selected from the group consisting of formula Qa1 and Qb1, wherein
A₂ is N; and
Q₁ is hydrogen, chlorine, bromine, trifluoromethyl, 1-fluoro-1-methyl-ethyl, cyclopropyl, 1-cyanocyclopropyl, 1-cyano-1-methyl-ethyl, 1-cyano-1-methyl-ethoxy, 2,2,2-trifluoroethoxy, 2,2-difluoropropoxy, —NH(CH₃), —N(CH₃)COCH₃, —N(CH₃)CO(cyclopropyl), —N(H)CONH(CH₃), —N(CH₃)CONH(CH₃), (oxazolidin-2-one)-3-yl, 2-pyridyloxy, pyrazol-1-yl, 3-chloro-pyrazol-1-yl, 3-cyano-pyrazol-1-yl, 3-trifluoromethyl-pyrazol-1-yl, 1,2,4-triazol-1-yl or pyrimidin-2-yl when Q is Qa1; or
Q₁ is hydrogen, chlorine, bromine, trifluoromethyl, 1-fluoro-1-methyl-ethyl, cyclopropyl, 1-cyanocyclopropyl, 1-cyano-1-methyl-ethyl, 1-cyano-1-methyl-ethoxy, 2,2,2-trifluoroethoxy, 2,2-difluoropropoxy, —NH(CH₃), —N(CH₃)COCH₃, —N(CH₃)CO(cyclopropyl), —N(H)CONH(CH₃), —N(CH₃)CONH(CH₃), (oxazolidin-2-one)-3-yl, 2-pyridyloxy, pyrazol-1-yl, 3-chloro-pyrazol-1-yl, 3-cyano-pyrazol-1-yl, 3-trifluoromethyl-pyrazol-1-yl, 1,2,4-triazol-1-yl or pyrimidin-2-yl when Q is Qb1.

One further outstanding group of compounds according to this embodiment are compounds of formula (I-9n) which are compounds of formula (I-9) wherein:
A₁ is CH₂ or O;
R₂ is —CH₂CF₃, —CH₂CF₂CHF₂ or —CH₂CF₂CF₃; preferably —CH₂CF₃ or —CH₂CF₂CF₃, Q is a radical selected from the group consisting of formula Qa1 and Qb1, wherein
A₂ is N; and
Q₁ is hydrogen, chlorine, bromine, trifluoromethyl, 1-fluoro-1-methyl-ethyl, cyclopropyl, 1-cyanocyclopropyl, 1-cyano-1-methyl-ethyl, 1-cyano-1-methyl-ethoxy, 2,2,2-trifluoroethoxy, 2,2-difluoropropoxy, —NH(CH₃), —N(CH₃)COCH₃, 2-pyridyloxy, 3-chloro-pyrazol-1-yl, 1,2,4-triazol-1-yl or pyrimidin-2-yl.

One further outstanding group of compounds according to this embodiment are compounds of formula (I-9n-1) which are compounds of formula (I-9n) wherein:
Q₁ is hydrogen, trifluoromethyl, 1-fluoro-1-methyl-ethyl, cyclopropyl, 1-cyanocyclopropyl, 1-cyano-1-methyl-ethyl, 1-cyano-1-methyl-ethoxy, 2,2,2-trifluoroethoxy, 2,2-difluoropropoxy, —NH(CH₃), —N(CH₃)COCH₃, 2-pyridyloxy, 3-chloro-pyrazol-1-yl, 1,2,4-triazol-1-yl or pyrimidin-2-yl.

One further outstanding group of compounds according to this embodiment are compounds of formula (I-90) which are compounds of formula (I-9) wherein:
A₁ is CH₂ or O;
R₂ is —CH₂CF₃ or —CH₂CF₂CF₃,
Q is the radical Qa1, wherein
A₂ is N; and
Q₁ is hydrogen, chlorine, bromine, trifluoromethyl, 1-fluoro-1-methyl-ethyl, cyclopropyl, 1-cyanocyclopropyl, 1-cyano-1-methyl-ethyl, 1-cyano-1-methyl-ethoxy, 2,2,2-trifluoroethoxy, 2,2-difluoropropoxy, —NH(CH₃), —N(CH₃)COCH₃, 2-pyridyloxy, 3-chloro-pyrazol-1-yl, 1,2,4-triazol-1-yl or pyrimidin-2-yl.

One further outstanding group of compounds according to this embodiment are compounds of formula (I-9o-1) which are compounds of formula (I-90) wherein:
Q₁ is hydrogen, trifluoromethyl, 1-fluoro-1-methyl-ethyl, cyclopropyl, 1-cyanocyclopropyl, 1-cyano-1-methyl-ethyl, 1-cyano-1-methyl-ethoxy, 2,2,2-trifluoroethoxy, 2,2-difluoropropoxy, —NH(CH₃), —N(CH₃)COCH₃, 2-pyridyloxy, or 3-chloro-pyrazol-1-yl.

One further outstanding group of compounds according to this embodiment are compounds of formula (I-9p) which are compounds of formula (I-9) wherein:
A₁ is CH₂ or O;
R₂ is —CH₂CF₃ or —CH₂CF₂CF₃,
Q is the radical Qb1, wherein
A₂ is N; and
Q₁ is hydrogen, chlorine, bromine, trifluoromethyl, 1-fluoro-1-methyl-ethyl, cyclopropyl, 1-cyanocyclopropyl, 1-cyano-1-methyl-ethyl, 1-cyano-1-methyl-ethoxy, 2,2,2-trifluoroethoxy, 2,2-difluoropropoxy, —NH(CH₃), —N(CH₃)COCH₃, 2-pyridyloxy, 3-chloro-pyrazol-1-yl, 1,2,4-triazol-1-yl or pyrimidin-2-yl.

One further outstanding group of compounds according to this embodiment are compounds of formula (I-9p-1) which are compounds of formula (I-9p) wherein:
Q₁ is cyclopropyl, 1,2,4-triazol-1-yl or pyrimidin-2-yl.

Compounds according to the invention may possess any number of benefits including, inter alia, advantageous levels of biological activity for protecting plants against insects or superior properties for use as agrochemical active ingredients (for example, greater biological activity, an advantageous spectrum of activity, an increased safety profile, improved physico-chemical properties, or increased biodegradability or environmental profile). In particular, it has been surprisingly found that certain compounds of formula (I) may show an advantageous safety profile with respect to non-target arthropods, in particular pollinators such as honey bees, solitary bees, and bumble bees. Most particularly, Apis mellifera.

In another aspect the present invention provides a composition comprising an insecticidally, acaricidally, nematicidally or molluscicidally effective amount of a compound of formula (I), or an agrochemically acceptable salt, stereoisomer, enantiomer, tautomer or N-oxide thereof, as defined in any of the embodiments under compounds of formula (I-1), (I-2), (I-3), (I-4), (I-5), (I-6), (I-7), (I-8), and (I-9) (above), and, optionally, an auxiliary or diluent.

In a further aspect the present invention provides a method of combating and controlling insects, acarines, nematodes or molluscs which comprises applying to a pest, to a locus of a pest, or to a plant susceptible to attack by a pest an insecticidally, acaricidally, nematicidally or molluscicidally effective amount of a compound of formula (I), or an agrochemically acceptable salt, stereoisomer, enantiomer, tautomer or N-oxide thereof, as defined in any of the embodiments under compounds of formula (I-1), (I-2), (I-3), (I-4), (I-5), (I-6), (I-7), (I-8), and (I-9) (above) or a composition as defined above.

In a yet further aspect, the present invention provides a method for the protection of plant propagation material from the attack by insects, acarines, nematodes or molluscs, which comprises treating the propagation material or the site, where the propagation material is planted, with a composition as defined above.

The process according to the invention for preparing compounds of formula I is carried out in principle by methods known to those skilled in the art. More specifically, and as described in scheme 1 and 2, the subgroup of compounds of formula I, wherein X is SO (sulfoxide) and/or SO₂ (sulfone), may be obtained by means of an oxidation reaction of the corresponding sulfide compounds of formula I, wherein X is S, involving reagents such as, for example, m-chloroperoxybenzoic acid (mCPBA), hydrogen peroxide, oxone, sodium periodate, sodium hypochlorite or tert-butyl hypochlorite amongst other oxidants. The oxidation reaction is generally conducted in the presence of a solvent. Examples of the solvent to be used in the reaction include aliphatic halogenated hydrocarbons such as dichloromethane and chloroform; alcohols such as methanol and ethanol; acetic acid; water; and mixtures thereof. The amount of the oxidant to be used in the reaction is generally 1 to 3 moles, preferably 1 to 1.2 moles, relative to 1 mole of the sulfide compounds I to produce the sulfoxide compounds I, and preferably 2 to 2.2 moles of oxidant, relative to 1 mole of the sulfide compounds I to produce the sulfone compounds I. Such oxidation reactions are disclosed, for example, in WO 2013/018928.

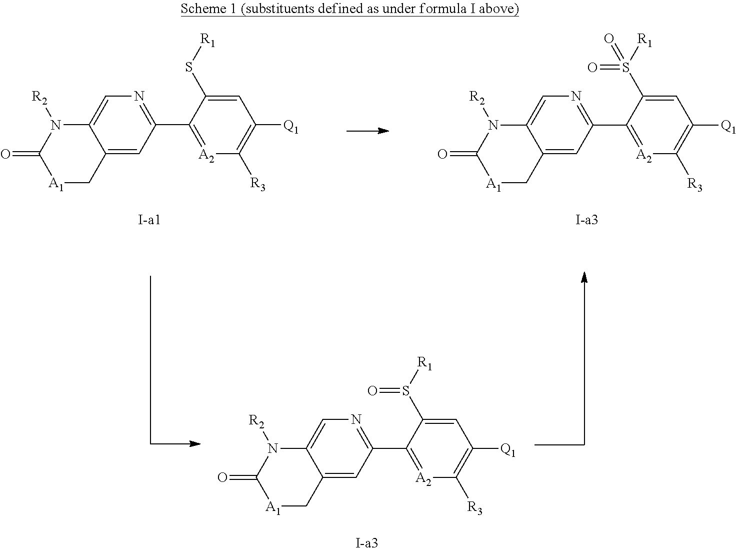

The chemistry described previously in scheme 1 to access compounds of formula I-a2 and I-a3 from compounds of formula I-a1 can be applied analogously (scheme 2) for the preparation of compounds of formula I-a5 and I-a6 from compounds of formula I-a4, wherein all substituent definitions mentioned previously remain valid.

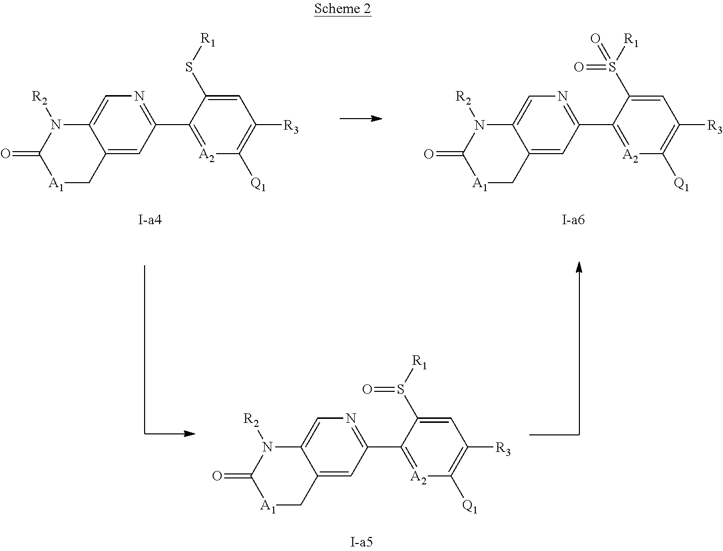

The subgroup of compounds of formula I, wherein R₂ is as defined in formula I and wherein Q is defined as Qa, in which Q₁, R₃, X, A₂ and R₁ are as defined in formula I and wherein A₁ is CH₂, may be defined as compounds of formula I-Qa-1 (scheme 3).

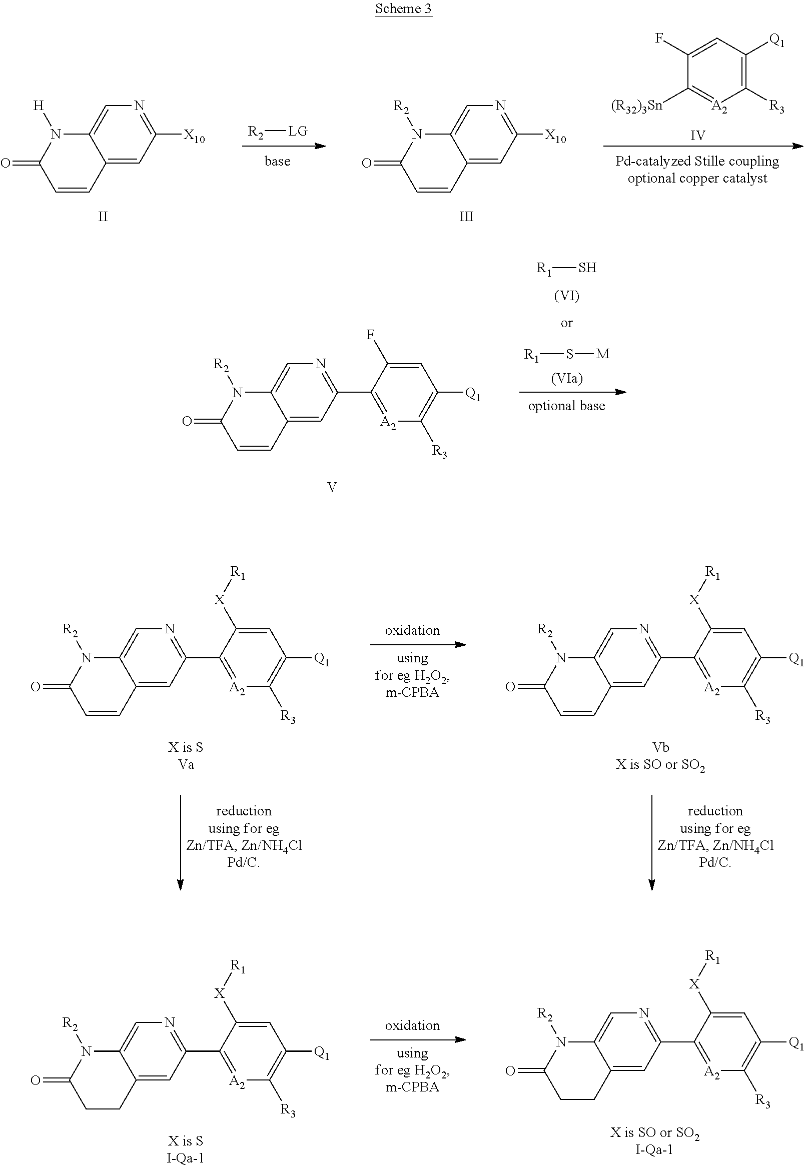

Compounds of formula I-Qa-1, wherein X is SO or SO₂, and in which R₁, R₂, Q₁, A₂ and R₃ are as defined in formula I, can be prepared from compounds of formula Vb, wherein X is SO or SO₂, and in which R₁, R₂, Q₁, A₂ and R₃ are as defined in formula I, by reduction reaction using reagents such as zinc powder and ammonium chloride, preferably an aqueous saturated ammonium chloride solution, optionally in the presence of an acid (preferably in catalytic amount) such as trifluoroacetic acid, hydrochloric acid amongst others, in ether solvents such as tetrahydrofuran or dioxane, at temperatures between 0° C. and refluxing conditions. Alternatively, such a reduction may also be achieved under conditions known to a person skilled in the art, for example by using molecular hydrogen (H₂), optionally under pressure, usually in the presence of a catalyst such as for example Raney-Nickel, or using transfer hydrogenation conditions (for example, ammonium formiate and 5-10% palladium on charcoal in tetrahydrofuran around room temperature). Compounds of formula Vb, wherein X is SO or SO₂, and in which R₁, R₂, Q₁, A₂ and R₃ are as defined in formula I, can be prepared from compounds of formula Va, wherein X is S, and in which R₁, R₂, Q₁, A₂ and R₃ are as defined in formula I, using oxidating reaction as described in scheme 1. Alternatively compounds of formula I-Qa-1 can be prepared from compounds of formula Va by involving the same chemistry as described above, but by changing the order of the steps (i.e. by running a reduction step on Va, wherein X is S, to form I-Qa-1, wherein X is S, followed by the oxidation step on I-Qa-1, wherein X is S to form I-Qa-1, wherein X is SO or SO₂.

Compounds of formula Va, wherein X is S, and in which R₁, R₂, Q₁, A₂ and R₃ are as defined in formula I, can be prepared by reacting compounds of formula V, wherein R₂, Q₁, A₂ and R₃ are as defined in formula I, with a reagent of the formula VI

R₁—SH (VI),

or a salt thereof, wherein R₁ is as defined in formula I, optionally in the presence of a suitable base, such as alkali metal carbonates, for example sodium carbonate and potassium carbonate, or alkali metal hydrides such as sodium hydride, or alkali metal hydroxides such as sodium hydroxide and potassium hydroxide, or sodium or potassium tert-butoxide, in an inert solvent at temperatures preferably between 25-120° C. Examples of solvent to be used include ethers such as tetrahydrofuran THF, ethylene glycol dimethyl ether, tert-butylmethyl ether, and 1,4-dioxane, aromatic hydrocarbons such as toluene and xylene, nitriles such as acetonitrile or polar aprotic solvents such as N,N-dimethylformamide, N,N-dimethylacetamide, N-methyl-2-pyrrolidone NMP or dimethyl sulfoxide. Examples of salts of the compound of formula VI include compounds of the formula VIa

R₁—S-M (VIa),

wherein R₁ is as defined above and wherein M is, for example, sodium or potassium. Such a process to prepare compounds of formula Va from compounds of formula V can be found, for example, in WO16/091731.

Alternatively, this reaction to form Va can be carried out in the presence of a palladium catalyst, such as tris(dibenzylideneacetone)dipalladium(0), in the presence of a phosphine ligand, such as Xanthphos, in an inert solvent, for example, xylene at temperatures between 100-160° C., preferably 140° C., as described in Tetrahedron 2005, 61, 5253-5259.

Compounds of formula V, wherein R₂, Q₁, A₂ and R₃ are as defined in formula I, can be prepared by a Stille reaction between compounds of formula (IV), wherein Q₁, A₂ and R₃ are as defined in formula I above, and wherein R₃₂ is C₁-C₁₀alkyl, preferably n-butyl or methyl, and compounds of formula III, wherein R₂ is as defined in formula I above and in which X₁₀ is a leaving group like, for example, chlorine, bromine or iodine (preferably chlorine or bromine), or an aryl- or alkylsulfonate such as trifluoromethanesulfonate. Such Stille reactions are usually carried out in the presence of a palladium catalyst, for example tetrakis(triphenylphosphine)palladium(0), palladium(II) acetate or bis(triphenylphosphine)palladium(II) dichloride, and in the presence of ligand such as phosphine ligand Xanthphos, XPhos amongst others in an inert solvent such as N,N-dimethylformamide, acetonitrile, toluene or dioxane, optionally in the presence of an additive, such as cesium fluoride, or lithium chloride, and optionally in the presence of a further catalyst, for example copper(I)iodide. Such Stille couplings are also well known to those skilled in the art, and have been described in for example J. Org. Chem., 2005, 70, 8601-8604, J. Org. Chem., 2009, 74, 5599-5602, and Angew. Chem. Int. Ed., 2004, 43, 1132-1136.

Compounds of formula III, wherein R₂ is as defined in formula I above and wherein X₁₀ is a leaving group like, for example, chlorine, bromine or iodine (preferably chlorine or bromine), or an aryl- or alkylsulfonate such as trifluoromethanesulfonate, can be prepared by reacting compounds of formula II, wherein X₁₀ is a halogen (or a pseudo-halogen leaving group, such as a triflate), with reagents of the formula R₂-LG, wherein R₂ is as defined in formula I, and in which LG is a halogen, preferably iodine, bromine or chlorine (or a pseudo-halogen leaving group, such as a (halo)alkyl or phenyl sulfonate ester, e.g. triflate), in the presence of a base, such as sodium hydride or an alkaline earth metal hydride, carbonate (e.g. sodium carbonate, potassium carbonate or cesium carbonate) or hydroxide, in an inert solvent such as tetrahydrofuran, dioxane, N,N-dimethylformamide DMF, N,N-dimethylacetamide or acetonitrile and the like, at temperatures between 0 and 120° C., by procedures well known to those skilled in the art, and described for example in WO 21/136722.

The subgroup of compounds of formula I, wherein R₂ is as defined in formula I and wherein Q is defined as Qb, in which Q₁, R₃, X, A₂ and R₁ are as defined in formula I and wherein A₁ is CH₂, may be defined as compounds of formula I-Qb-1 (scheme 4).

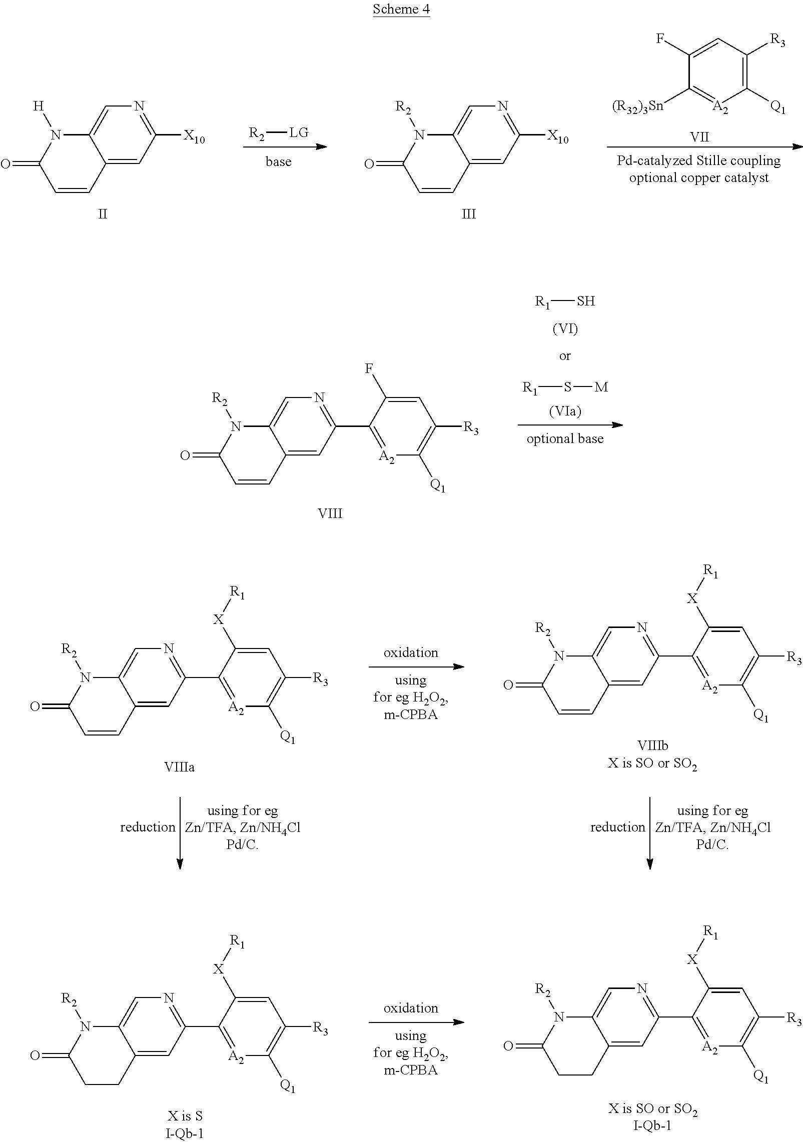

The chemistry described previously in scheme 3 to access compounds of formula I-Qa-1 from compounds of formula II can be applied analogously (scheme 4) for the preparation of compounds of formula I-Qb-1 from compounds of formula II, wherein all substituent definitions mentioned previously remain valid.

Compounds of formula VI, wherein R₁ is as defined in formula I, and compounds of formula VIa, wherein R₁ is as defined above and wherein M is, for example, sodium or potassium, are either known, commercially available or may be prepared by methods known to a person skilled in the art.

Compounds of formula IV and compounds of formula VII, wherein Q₁, A₂ and R₃ are as defined in formula I above, and wherein R₃₂ is C₁-C₁₀alkyl, preferably n-butyl or methyl; and reagents of the formula R₂-LG, wherein R₂ is as defined in formula I, and in which LG is a halogen, preferably iodine, bromine or chlorine (or a pseudo-halogen leaving group, such as a (halo)alkyl or phenyl sulfonate ester, e.g. triflate);
are either known, commercially available or may be prepared by methods known to a person skilled in the art.

Alternatively compounds of formula Va, wherein R₁, R₂, R₃, Q₁ and X are as defined in formula I above, and in which A₂ is N, can be prepared following scheme 5.

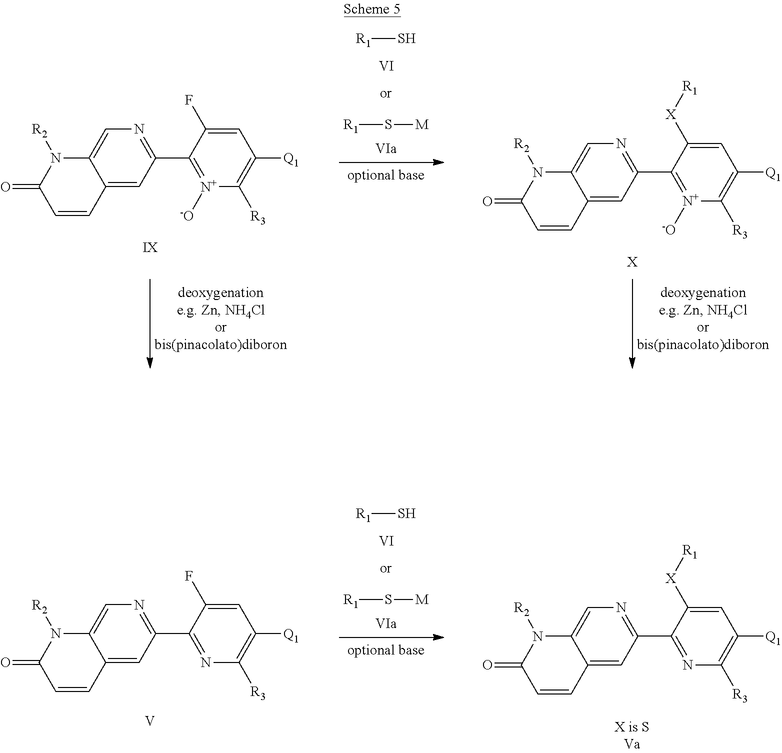

Compounds of formula Va, wherein R₁, R₂, R₃, and Q₁ are as defined in formula I above, and in which A₂ is N and X is S, can be prepared (scheme 5) by deoxygenation of compounds of formula X, wherein X is S, and in which R₁, R₂, Q₁, and R₃ are as defined in formula I, using reagents such as zinc powder and ammonium chloride, preferably an aqueous saturated ammonium chloride solution, in ether solvents such as tetrahydrofuran or dioxane, at temperatures between 0° C. and refluxing conditions. Alternatively, such a reduction may also be achieved under conditions known to a person skilled in the art, for example by involving iron powder in acetic acid, or using molecular hydrogen (H₂), optionally under pressure, usually in the presence of a catalyst such as for example Raney-Nickel, or using transfer hydrogenation conditions (for example, ammonium formiate and 5-10% palladium on charcoal in tetrahydrofuran around room temperature), or using bis(pinacolato)diboron (4,4,5,5-tetramethyl-2-(4,4,5,5-tetramethyl-1,3,2-dioxaborolan-2-yl)-1,3,2-dioxaborolane) in for example acetonitrile, or using phosphorus based reagents such as phosphorus trichloride, triethyl phosphite or triphenyl phosphine.

Compounds of formula X, wherein X is S, and in which R₁, R₂, Q₁, and R₃ are as defined in formula I, can be prepared from compounds of formula IX, wherein R₂, R₃ and Q₁ are as described in formula I above, by analogous procedure as described in scheme 3 for the preparation of compounds of formula Va from compounds of formula V. Both transformations IX to X and X to Va have been described for example in WO 21/136722.

Alternatively, compounds of formula Va, wherein X is S, may be prepared from compounds of formula IX, by involving the same chemistry as described above, but by changing the order of the steps (i.e. by running the sequence IX to V via deoxygenation/reduction, followed by reaction of V with VI or VIa to form Va, wherein all substituent definitions mentioned previously remain valid).

Alternatively compounds of formula VIII-a described in scheme 4 can be prepared by the chemistry described previously in scheme 5 to access compounds of formula Va from compounds of formula IX and can be applied analogously (scheme 6) for the preparation of compounds of formula VIII-a from compounds of formula XI, wherein all substituent definitions mentioned previously remain valid.

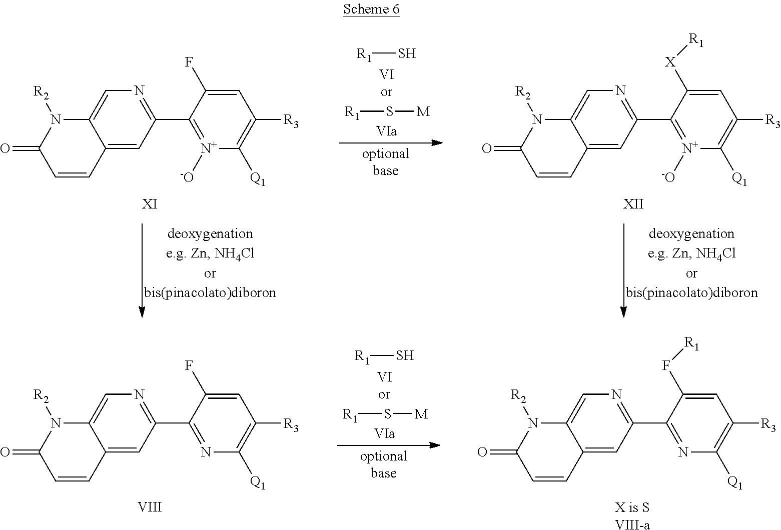

Compounds of formula IX, wherein R₂, Q₁ and R₃ are as defined in formula I,

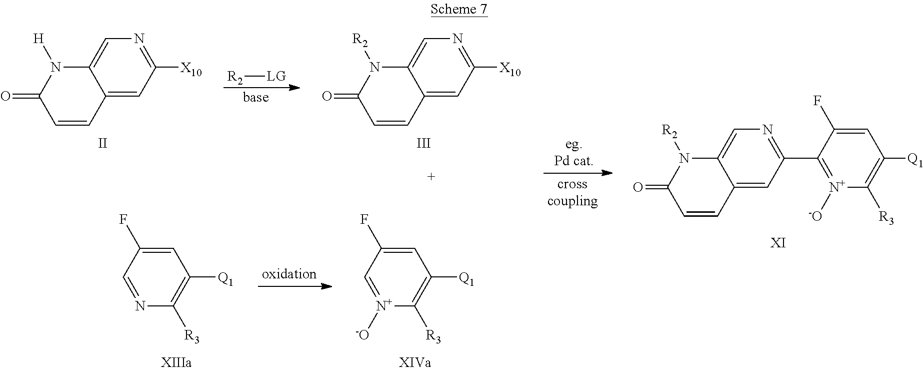

can be prepared (scheme 7) by a cross-coupling reaction between compounds of formula III, wherein R₂ is as defined in formula I above and wherein X₁₀ is a halogen (or a pseudo-halogen leaving group, such as a triflate), preferably bromine or chlorine, and compounds of formula XIVa, wherein Q₁ and R₃ are as defined in formula I, under metal catalysis (preferably palladium catalysis) conditions, for example involving [1,1′bis(diphenylphosphino)ferrocene]dichloropalladium (PdCl₂(dppf), optionally as a complex with dichloromethane (preferably a 1:1 complex), in presence of a base such as 2,2,6,6-tetramethylpiperidide zinc chloride lithium chloride (TMPZnCl·LiCl; commercial or prepared according to Org. Lett. 2009, 11, 1837-1840), preferably in form of a solution of the tetramethylpiperidinyl zinc chloride lithium chloride complex in tetrahydrofuran, in ether solvents such as tetrahydrofuran, dioxane or 1,2-dimethoxyethane, preferably tetrahydrofuran, at temperatures between 0° C. and refluxing conditions, preferably between room temperature and 80° C., preferably under inert atmosphere, and optionally microwave irradiation. Such cross-coupling conditions have been described in, for example, Org. Lett. 2012, 14, 862-865 or WO 21/136722.

Alternatively, this cross-coupling step may also be performed under Fagnou-type conditions (described by Fagnou et al. in, for example, Org. Lett. 2011, 13, 2310-13 and J. Am. Chem. Soc. 2009, 131, 3291-3306) involving palladium acetate and a phosphine ligand such as tri-tert-butylphosphonium tetrafluoroborate (PtBu₃-HBF₄), in the presence of a base such as potassium carbonate or cesium carbonate, in solvents such as tetrahydrofuran, dioxane, acetonitrile, N,N-dimethylformamide or toluene, at temperatures between 0° C. and 150° C., preferably between room temperature and 120° C., preferably under inert atmosphere, and optionally microwave irradiation.

Compounds of formula III, wherein R₂ is as defined in formula I above and wherein X₁₀ is a leaving group like, for example, chlorine, bromine or iodine (preferably chlorine or bromine), or an aryl- or alkylsulfonate such as trifluoromethanesulfonate, can be prepared by reacting compounds of formula II, wherein X₁₀ is a halogen (or a pseudo-halogen leaving group, such as a triflate), with reagents of the formula R₂-LG, wherein R₂ is as defined in formula I, and in which LG is a halogen, preferably iodine, bromine or chlorine (or a pseudo-halogen leaving group, such as a (halo)alkyl or phenyl sulfonate ester, e.g. triflate), in the presence of a base, such as sodium hydride or an alkaline earth metal hydride, carbonate (e.g. sodium carbonate, potassium carbonate or cesium carbonate) or hydroxide, in an inert solvent such as tetrahydrofuran, dioxane, N,N-dimethylformamide DMF, N,N-dimethylacetamide or acetonitrile and the like, at temperatures between 0 and 120° C., by procedures well known to those skilled in the art.

Compounds of formula XIVa, wherein Q₁ and R₃ are as defined in formula I, can be prepared by oxidation of compounds of formula XIIIa, wherein Q₁ and R₃ are as defined in formula I, under conditions known to those skilled in the art, involving for example, meta-chloro perbenzoic acid in an inert solvent such as ethyl acetate, chloroform or methylene chloride, at temperatures between 0° C. and 80° C., preferably 10 to 70° C. Alternatively, other suitable oxidizing agents may be used, such as for example methyltrioxorhenium and hydrogen peroxide (either aqueous or as a urea complex), hydrogen peroxide in acetic acid, or the H₂O₂/urea adduct in the presence of an acid anhydride, e.g. trifluoroacetic anhydride. Such oxidations are known from the literature, for example from J. Med. Chem. 1989, 32, 2561, WO 00/15615, WO 20/182577 or WO 21/136722.

Compounds of formula XIIIa, wherein Q₁ and R₃ are as defined in formula I are either known, commercially available or may be prepared by methods known to a person skilled in the art or by analogy to descriptions found for example in WO 20/182577.

The chemistry described previously in scheme 7 to access compounds of formula IX can be applied analogously (scheme 8) for the preparation of compounds of formula XI, wherein all substituent definitions mentioned previously remain valid.

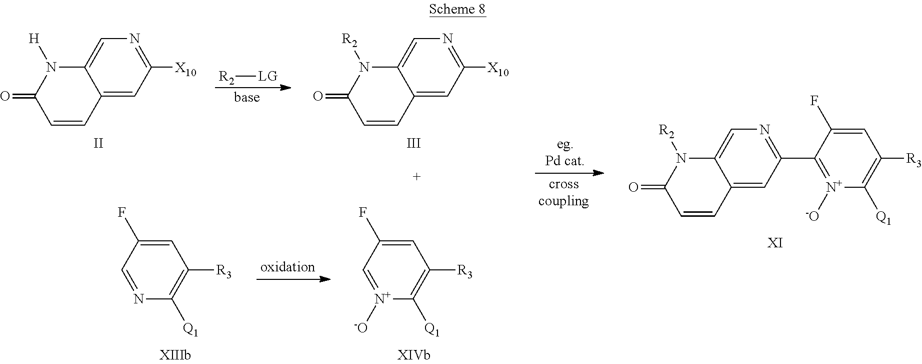

Compounds of formula XIIIb, wherein Q₁ and R₃ are as defined in formula I, are either known, commercially available, or may be prepared by methods known to a person skilled in the art. Compounds of formula II can be prepared following scheme 9.

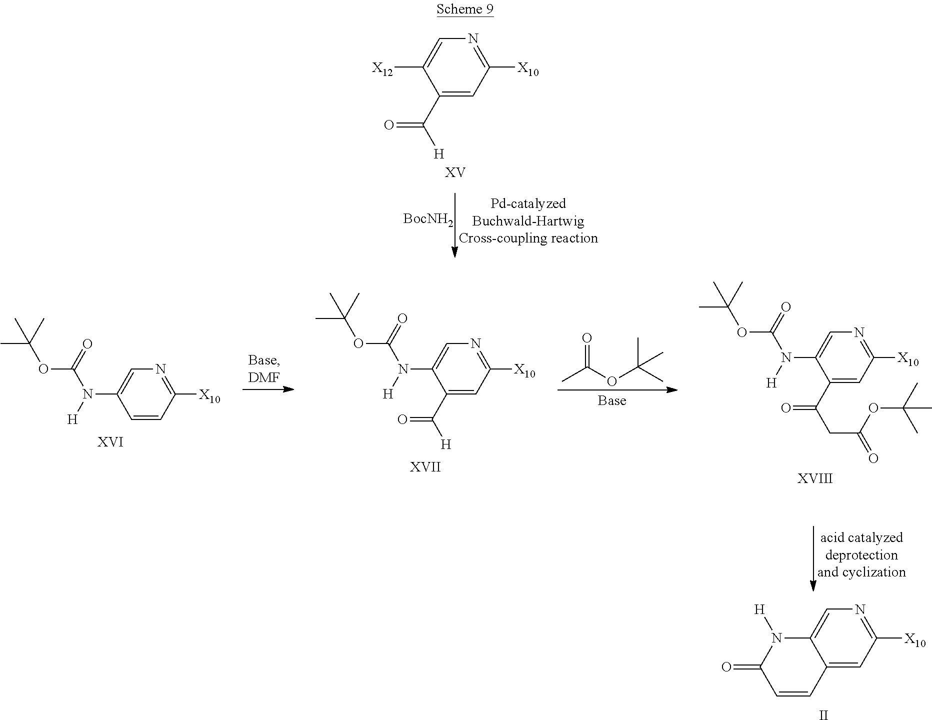

Compounds of formula II, wherein X₁₀ is a halogen (or a pseudo-halogen leaving group, such as a triflate), preferably bromine or chlorine, can be prepared (scheme 9) by acid catalyzed deprotection of BOC-functional groups (tert-butoxycarbonyl) and subsequent intramolecular cyclization of amine and carboxylic acid to form the carboxamide. Such reactions can be performed in the presence of acids such as trifluoroacetic acid, hydrochloric acid, sulfuric acid amongst others and optionally in the presence of solvents such as halogenated solvents like dichloromethane, dichloroethane, water amongst others and at temperature between room temperature and boiling point of the solvent or reagent. Compounds of formula XVIII, wherein X₁₀ is a halogen (or a pseudo-halogen leaving group, such as a triflate), preferably bromine or chlorine, can be prepared by the reaction of compounds of formula XVII, wherein X₁₀ is a halogen (or a pseudo-halogen leaving group, such as a triflate), preferably bromine or chlorine, and tert-butyl acetate in the presence of a suitable base such as n-BuLi, lithium diisopropylamide, Li-TMP amongst others and in the presence of solvent such as tetrahydrofuran, dioxane, dimethylformamide amongst other and at temperatures between −78° C. and boiling point of solvent. Compounds of formula XVII, wherein X₁₀ is a halogen (or a pseudo-halogen leaving group, such as a triflate), preferably bromine or chlorine, can be prepared by the metallation of compounds of formula XVI, wherein X₁₀ is a halogen (or a pseudo-halogen leaving group, such as a triflate), preferably bromine or chlorine, using base such as n-BuLi, lithium diisopropylamide, Li-TMP and subsequently reacting with DMF in the presence of solvent such as tetrahydrofuran, dioxane, dimethylformamide. Such reactions are known for example in J. Org Chem., 1990, 55, 4744. Alternatively compounds of formula XVII, can be prepared by palladium catalyzed selective Buchwald-Hartwig cross-coupling reaction between compounds of formula XV, wherein X₁₀ and X₁₂ are independently halogen (or a pseudo-halogen leaving group, such as a triflate), preferably bromine or chlorine and tert-butyl carbamate (BOCNH₂). Such reactions can be performed in the presence of metal catalyst (preferably palladium catalyst) such as [1,1′bis(diphenylphosphino)ferrocene]-dichloropalladium (PdCl₂(dppf), or Pd(OAc)₂ and in the presence of ligand such as tributylphosphine, dppf, Xantphos, XPhos and in presence of a base such as sodium tert-butoxide, potassium carbonate, cesium carbonate, sodium carbonate and in the presence of solvents such as tetrahydrofuran, dioxane or 1,2-dimethoxyethane, toluene, and at temperatures between 0° C. and refluxing conditions, preferably under inert atmosphere, and optionally microwave irradiation. Such reactions are well known in the literature and described for example in Chem. Rev. 2016, 116, 19, 12564-12649 and J. Org. Chem. 1999, 64, 15, 5575-5580.

Alternatively compounds of formula II, wherein X₁₀ is a halogen (or a pseudo-halogen leaving group, such as a triflate), preferably bromine or chlorine,

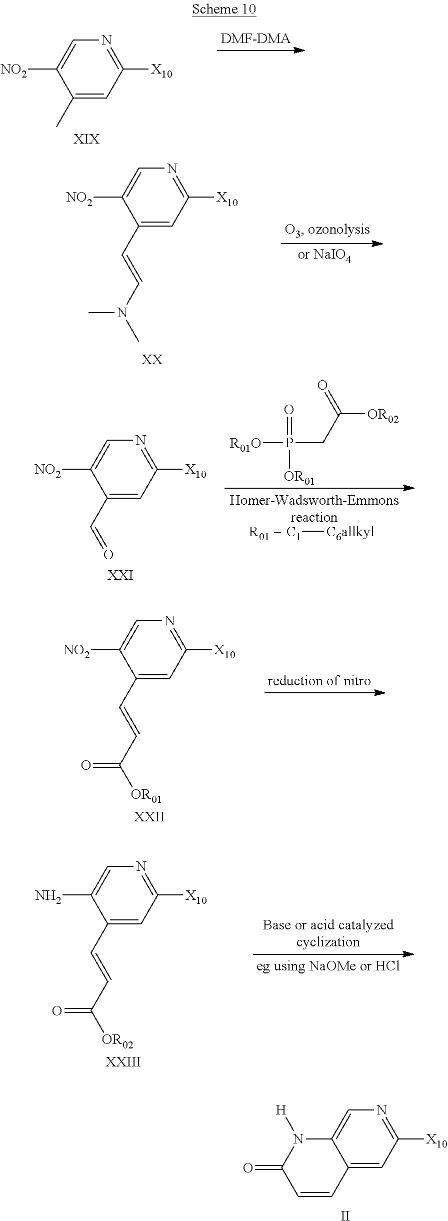

can be prepared following scheme 10 and analogous to procedure as described in WO 2000/049015.

Alternatively compounds of formula II, wherein X₁₀ is a halogen (or a pseudo-halogen leaving group, such as a triflate), preferably bromine or chlorine,

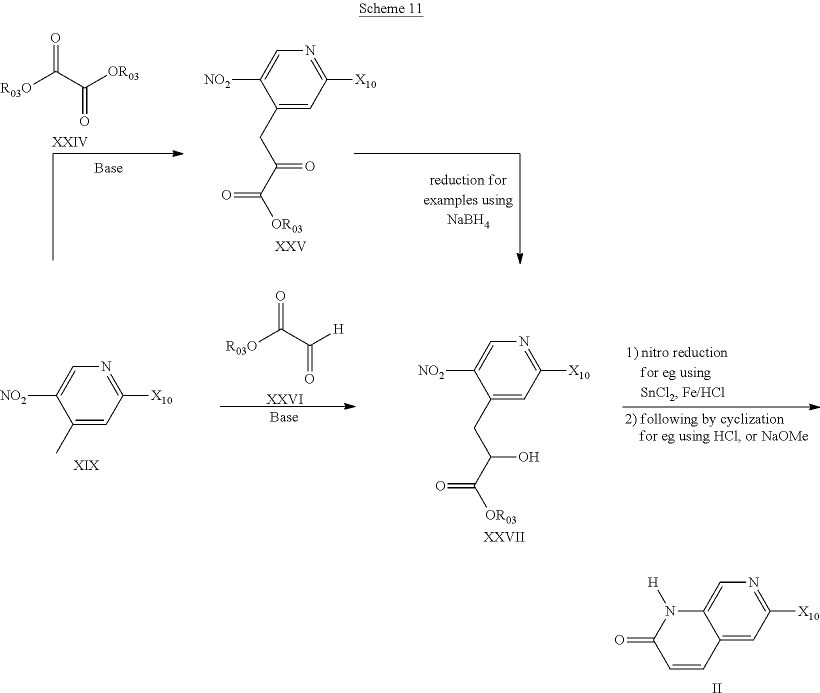

can be prepared (scheme 11) from compounds of formula XXVII, wherein X₁₀ is a halogen (or a pseudo-halogen leaving group, such as a triflate) preferably bromine or chlorine, and wherein R₀₃ is C₁-C₆alkyl, benzyl, or aryl an group, via nitro reduction and subsequent intramolecular cyclization in the presence of an acid catalyst such as acetic acid, hydrochloric acid HCl, sulfuric acid H₂SO₄, or trifluoroacetic acid TFA, optionally in the presence of a solvent (such as tetrahydrofuran or dioxane) or in the presence of a base, such as sodium methoxide.

Nitro reduction typically make use of reagents such as iron in the presence of ammonium chloride, iron in the presence of acetic acid, Sn/HCl, or tetrahydroxydiboron amongst others, and at temperature between 0° C. to boiling point of the reaction mixture. Such reactions are known in the literature and for example described for example in Synthesis, 2018, 50, 1765-1768, Org. Lett. 2014, 16, 19, 5192-5195, Organic Letters (2019), 21(9), 3465-3469 and Synthetic Communications, 2007, 37, 2777-2786.

Compounds of formula XXV may exist in different tautomeric forms, such as XXVa and/or XXVb:

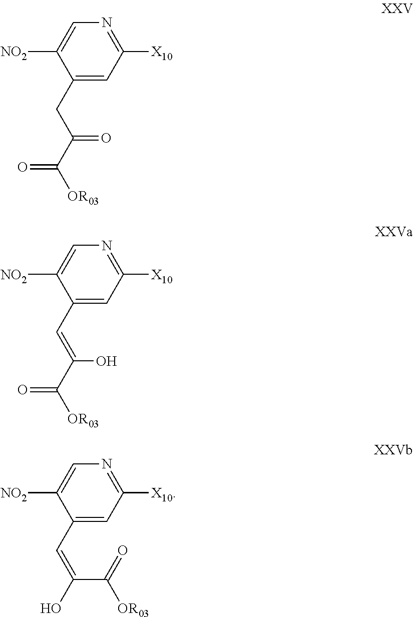

This invention covers all such isomers and tautomers and mixtures thereof in all proportions.

Compounds of formula XXVII, wherein X₁₀ is a halogen (or a pseudo-halogen leaving group, such as a triflate), preferably bromine or chlorine, and wherein R₀₃ is C₁-C₆alkyl, benzyl, or an aryl group, can be prepared by reacting compounds of formula XIX, wherein X₁₀ is a halogen (or a pseudo-halogen leaving group, such as a triflate), preferably bromine or chlorine, and compounds of formula XXVI, wherein R₀₃ is C₁-C₆alkyl, benzyl, or an aryl group in the presence of base such as potassium tert-butoxide, sodium methoxide, sodium ethoxide, sodium hydride, n-butyl lithium, 1,8-diazabicyclo(5.4.0)undec-7-ene (DBU), lithium diisopropylamide amongst other similar bases optionally in the presence of a solvent such as tetrahydrofuran, methanol, dioxane, ethanol, DMF and at temperature in between −78° C. to boiling point of the reaction mixture.

Alternatively compounds of formula XXVII, wherein X₁₀ is a halogen (or a pseudo-halogen leaving group, such as a triflate), preferably bromine or chlorine, and wherein R₀₃ is C₁-C₆alkyl, benzyl, or an aryl group, can be prepared in two steps from compounds of formula XIX. The first step involves reacting compounds of formula XIX and compounds of formula XXIV, wherein R₀₃ is C₁-C₆alkyl, benzyl, or an aryl group in the presence of base such as potassium tert-butoxide, sodium methoxide, sodium ethoxide, sodium hydride, n-butyl lithium, 1,8-diazabicyclo(5.4.0)undec-7-ene (DBU), lithium diisopropylamide amongst other similar bases, optionally in the presence of solvent such as tetrahydrofuran, methanol, dioxane, ethanol, DMF and at temperature in between −78° C. to the boiling point of the reaction mixture. And the second step involves the carbonyl reduction of compounds of formula XXV, wherein X₁₀ is a halogen (or a pseudo-halogen leaving group, such as a triflate), preferably bromine or chlorine, and in which R₀₃ is C₁-C₆alkyl, benzyl, or an aryl group, in the presence of reducing agent such as a boron based reducing agent for example sodium borohydride, borane, or an aluminum based reagent, for example diisobutylaluminium hydride, or lithium aluminum hydride, in the presence of solvent such as tetrahydrofuran, methanol, dioxane, or ethanol, optionally in mixtures with water, and at temperatures preferably between 0° and 30° C. Such two step procedures are known in the literature, for example as described in WO2005/044802.

The preparation of compounds of formula II, wherein X₁₀ is a halogen (or a pseudo-halogen leaving group, such as a triflate), preferably bromine or chlorine, along the path XIX to XXV to XXVII to II (scheme 11), has been described for example in WO 21/136722.

Compounds of formula XV, wherein X₁₀ and X₁₂ are independently halogen (or a pseudo-halogen leaving group, such as a triflate), preferably bromine or chlorine; and
compounds of formula XVI, wherein X₁₀ is a halogen (or a pseudo-halogen leaving group, such as a triflate), preferably bromine or chlorine; and compounds of formula XIX, wherein X₁₀ is a halogen (or a pseudo-halogen leaving group, such as a triflate), preferably bromine or chlorine; and
compounds of formula XXIV and XXVI, wherein R₀₃ is C₁-C₆alkyl, benzyl, or an aryl group; are either known, commercially available, or may be prepared by methods known to a person skilled in the art.

The subgroup of compounds of formula I, wherein R₂ is as defined in formula I and wherein Q is defined as Qa, in which Q₁, R₃, X, A₂ and R₁ are as defined in formula I and wherein A₁ is O, may be defined as compounds of formula I-Qa-2 (scheme 12).

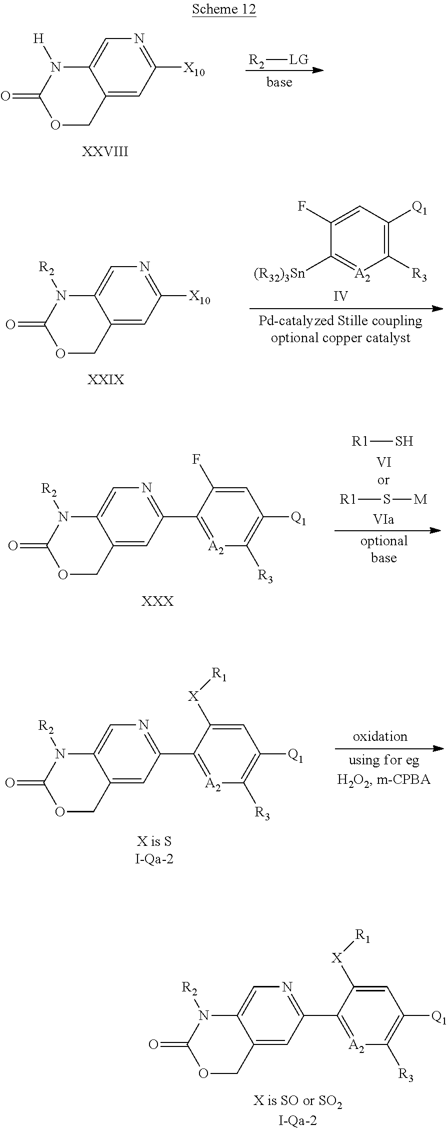

Compounds of formula I-Qa-2, wherein X, R₁, R₂, Q₁, A₂ and R₃ are as defined in formula I, can be prepared from compounds of formula XXVIII by following analogous procedures as described in scheme 3 for the preparation of compounds of formula I-Qa-1 from compounds of formula II (but omitting the reduction steps Va, respectively Vb to I-Qa-1), wherein all substituent definitions mentioned previously remain valid.

The subgroup of compounds of formula I, wherein R₂ is as defined in formula I and wherein Q is defined as Qb, in which Q₁, R₃, X, A₂ and R₁ are as defined in formula I and wherein A₁ is O, may be defined as compounds of formula I-Qb-2 (scheme 13).

The chemistry described previously in scheme 12 to access compounds of formula I-Qa-2 from compounds of formula XXVIII can be applied analogously (scheme 13) for the preparation of compounds of formula I-Qb-2 from compounds of formula XXVIII, wherein all substituent definitions mentioned previously remain valid.

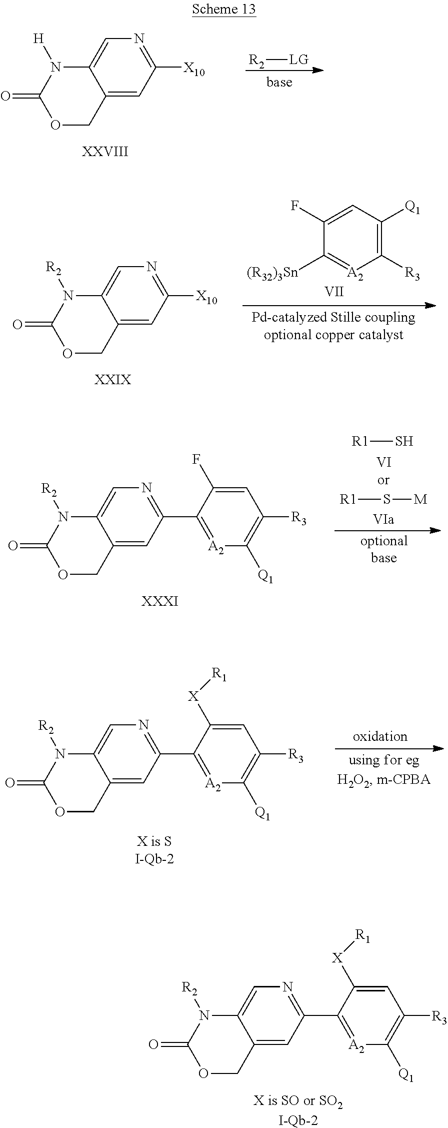

Alternatively compounds of formula I-Qa-2, wherein R₁, R₂, R₃, Q₁ and X are as defined in formula I above, and in which A₂ is N, can be prepared following scheme 14 analogously to procedures described in scheme 5 for the preparation of compounds of formula Va from compounds of formula IX.

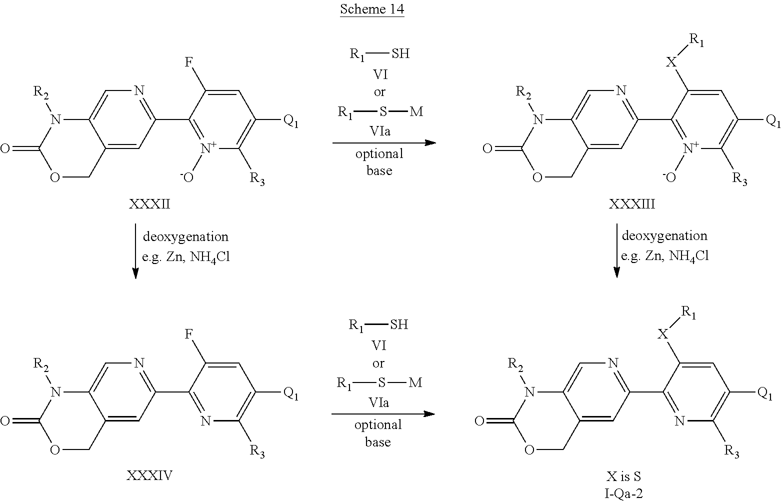

The chemistry described previously in scheme 14 to access compounds of formula I-Qa-2 from compounds of formula XXXII can be applied analogously (scheme 15) for the preparation of compounds of formula I-Qb-2 from compounds of formula XXXV, wherein all substituent definitions mentioned previously remain valid.

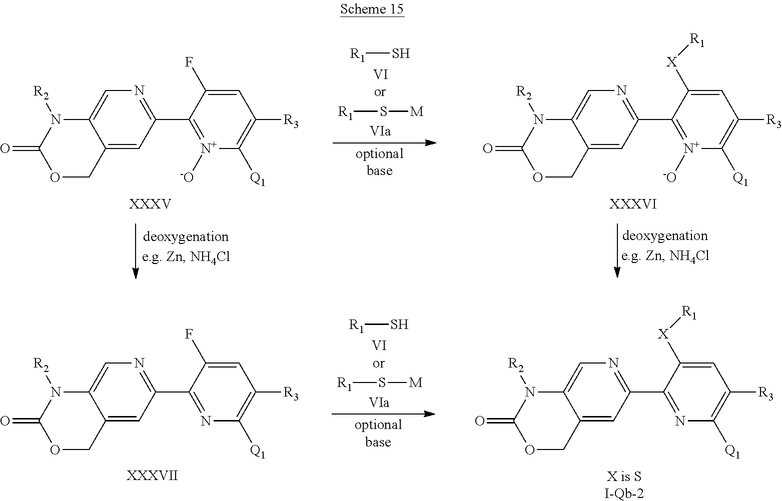

Compounds of formula XXXII, wherein R₂, Q₁ and R₃ are as defined in formula I,

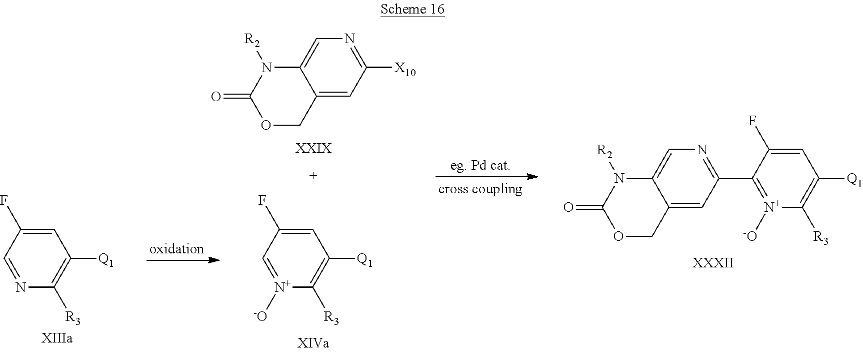

can be prepared (scheme 16) by a cross-coupling reaction between compounds of formula XXIX, wherein R₂ is as defined in formula I above and, wherein X₁₀ is a halogen (or a pseudo-halogen leaving group, such as a triflate), preferably bromine or chlorine, and compounds of formula XIVa, wherein Q₁ and R₃ are as defined in formula I, under metal catalysis (preferably palladium catalysis) conditions, for example involving [1,1′bis(diphenylphosphino)ferrocene]dichloropalladium (PdCl₂(dppf), optionally as a complex with dichloromethane (preferably a 1:1 complex), in presence of a base such as 2,2,6,6-tetramethylpiperidide zinc chloride lithium chloride (TMPZnCl·LiCl; commercial or prepared according to Org. Lett. 2009, 11, 1837-1840), preferably in form of a solution of the tetramethylpiperidinyl zinc chloride lithium chloride complex in tetrahydrofuran, in ether solvents such as tetrahydrofuran, dioxane or 1,2-dimethoxyethane, preferably tetrahydrofuran, at temperatures between 0° C. and refluxing conditions, preferably between room temperature and 80° C., preferably under inert atmosphere, and optionally microwave irradiation. Such cross-coupling conditions have been described in, for example, Org. Lett. 2012, 14, 862-865, and analogously in WO 21/136722. Alternatively, this cross-coupling step may also be performed under Fagnou-type conditions (described by Fagnou et al. in, for example, Org. Lett. 2011, 13, 2310-13 and J. Am. Chem. Soc. 2009, 131, 3291-3306) involving palladium acetate and a phosphine ligand such as tri-tert-butylphosphonium tetrafluoroborate (PtBu₃-HBF₄), in the presence of a base such as potassium carbonate or cesium carbonate, in solvents such as tetrahydrofuran, dioxane, acetonitrile, N,N-dimethylformamide or toluene, at temperatures between 0° C. and 150° C., preferably between room temperature and 120° C., preferably under inert atmosphere, and optionally microwave irradiation.

Compounds of formula XIVa, wherein Q₁ and R₃ are as defined in formula I, can be prepared by oxidation of compounds of formula XIIIa, wherein Q₁ and R₃ are as defined in formula I, under conditions known to those skilled in the art, involving for example, meta-chloro perbenzoic acid in an inert solvent such as ethyl acetate, chloroform or methylene chloride, at temperatures between 0° C. and 80° C., preferably 10 to 70° C. Alternatively, other suitable oxidizing agents may be used, such as for example methyltrioxorhenium and hydrogen peroxide (either aqueous or as a urea complex), hydrogen peroxide in acetic acid, or the H₂O₂/urea adduct in the presence of an acid anhydride, e.g. trifluoroacetic anhydride. Such oxidations are known from the literature, for example from J. Med. Chem. 1989, 32, 2561, WO 00/15615, WO20/182577 or WO 21/136722.

Compounds of formula XIIIa, wherein Q₁ and R₃ are as defined in formula I, is either known, commercially available or may be prepared by methods known to a person skilled in the art or by analogy to descriptions found for example in WO 20/182577.

The chemistry described previously in scheme 16 to access compounds of formula XXXII from compounds of formula XXIX can be applied analogously (scheme 17) for the preparation of compounds of formula XXXV from compounds of formula XXIX, wherein all substituent definitions mentioned previously remain valid.

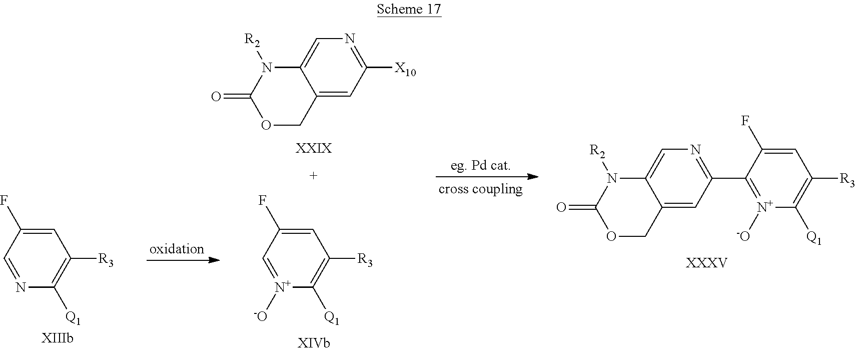

Compounds of formula XXIX, wherein R₂ is as defined in formula I above, and wherein X₁₀ is a halogen (or a pseudo-halogen leaving group, such as a triflate), preferably bromine or chlorine, can be prepared following scheme 18.

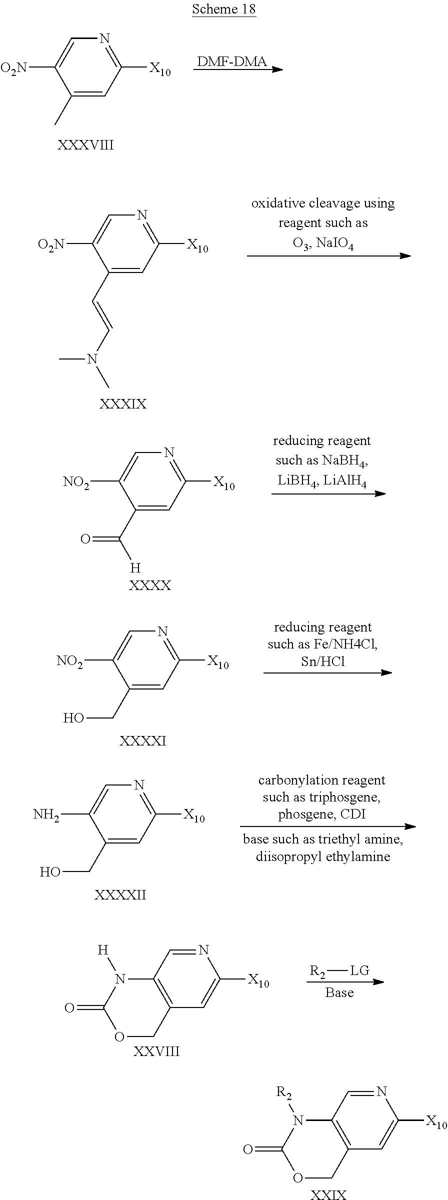

In scheme 18, compounds of formula XXIX, wherein R₂ is as defined in formula I above, and wherein X₁₀ is a halogen (or a pseudo-halogen leaving group, such as a triflate), preferably bromine or chlorine, can be prepared by reacting compounds of formula XXVIII, wherein X₁₀ is a halogen (or a pseudo-halogen leaving group, such as a triflate), with reagents of the formula R₂-LG, wherein R₂ is as defined in formula I, and in which LG is a halogen, preferably iodine, bromine or chlorine (or a pseudo-halogen leaving group, such as a (halo)alkyl or phenyl sulfonate ester, e.g. triflate), in the presence of a base, such as sodium hydride or an alkaline earth metal hydride, carbonate (e.g. sodium carbonate, potassium carbonate or cesium carbonate) or hydroxide, in an inert solvent such as tetrahydrofuran, dioxane, N,N-dimethylformamide DMF, N,N-dimethylacetamide or acetonitrile and the like, at temperatures between 0 and 120° C., by procedures well known to those skilled in the art.

Compounds of formula XXVIII, wherein X₁₀ is a halogen (or a pseudo-halogen leaving group, such as a triflate), can be prepared by reacting compounds of formula XXXXII, wherein X₁₀ is a halogen (or a pseudo-halogen leaving group, such as a triflate), with a carbonylating reagent (source of CO) such as phosgene, triphosgene (bis(trichloromethyl)carbonate), or carbonyl diimidazole CDI, in the presence of a base such as triethylamine, N,N-diisopropylethylamine, or potassium carbonate amongst others, in the presence of a solvent such as dioxane, tetrahydrofuran, or dichloromethane amongst others, and at temperature between −20° C. to the boiling point of the reaction mixture. Such reactions are known in the literature and for example described in Tetrahedron Letters 53 (2012) 4892-4895.

Compounds of formula XXXXII, wherein X₁₀ is a halogen (or a pseudo-halogen leaving group, such as a triflate), can be prepared from compounds of formula XXXXI, wherein X₁₀ is a halogen (or a pseudo-halogen leaving group, such as a triflate), via nitro reduction using reagents such as iron in the presence of ammonium chloride, Sn/HCl, or tetrahydroxydiboron amongst others, and at temperature between 0° C. to boiling point of the reaction mixture. The reaction can be carried out in the presence of a solvent such as methanol, ethanol, tetrahydrofuran, or dioxane (optionally in mixture with water). Such reactions are known in the literature and for example described for example in Synthesis, 2018, 50, 1765-1768, Org. Lett. 2014, 16, 19, 5192-5195, Organic Letters (2019), 21(9), 3465-3469 and Synthetic Communications, 2007, 37, 2777-2786.

Compounds of formula XXXXI, wherein X₁₀ is a halogen (or a pseudo-halogen leaving group, such as a triflate), can be prepared from compounds of formula XXXX, wherein X₁₀ is a halogen (or a pseudo-halogen leaving group, such as a triflate), via reduction of the aldehyde functionality and by using a reducing reagent. Examples of such reducing reagent include for example sodium borohydride, lithium borohydride and lithium aluminum hydride. The reaction can be carried out in the presence of a solvent such as methanol, ethanol, tetrahydrofuran, or dichloromethane, and at temperature between −78° C. to the boiling point of the reaction mixture. Such reactions are known in the literature and described for example in European Journal of Organic Chemistry (2009), (21), 3567-3572, Org. Lett. 2014, 16, 19, 5192-5195.

Compounds of formula XXXX, wherein X₁₀ is a halogen (or a pseudo-halogen leaving group, such as a triflate), can be prepared from compounds of formula XXXIX, wherein X₁₀ is a halogen (or a pseudo-halogen leaving group, such as a triflate), via oxidative cleavage of the enamine functionality by using reagents such as sodium periodate or ozone (see also scheme 10), in solvents such as tetrahydrofuran or dioxane (optionally in mixture with water), and for example at temperatures between 0° and 30° C.

Compounds of formula XXXIX, wherein X₁₀ is a halogen (or a pseudo-halogen leaving group, such as a triflate), can be prepared by reacting compounds of formula XXXVIII, wherein X₁₀ is a halogen (or a pseudo-halogen leaving group, such as a triflate), with N,N-dimethylformamide dimethyl acetal (DMF-DMA or 1,1-dimethoxy-N,N-dimethylmethylamine), optionally in the presence of a base catalyst or mediator such as 1,8-diazabicyclo[5.4.0]undec-7-ene, and optionally in the presence of a solvent such as N,N-dimethylformamide, tetrahydrofuran, or acetonitrile, and at temperatures between 0° C. to the boiling point of the reaction mixture, or the boiling point of N,N-dimethylformamide dimethyl acetal.

Such two-step conversion of compounds of formula XXXVIII to compounds of formula XXXX is known in the literature and described for example in Tetrahedron Letters, 1994, Vol. 335. No. 2, 219-222.

### Compounds of Formula XXIXa

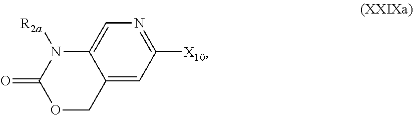 [0351] wherein
R₂ₐ is hydrogen or C₁-C₆haloalky (the group R₂ as defined under formula I above); and X₁₀ is a halogen or a pseudo-halogen leaving group, such as a triflate, are novel, especially developed for the preparation of the compounds of formula I according to the invention and therefore represent a further object of the invention. The preferences and preferred embodiments of the substituents of the compounds of formula I are also valid for the compounds of formula XXIXa. In one aspect of the present embodiment, R₂ₐ is preferably hydrogen; in another aspect of the present embodiment, R₂ₐ is preferably C₁-C₆haloalky (R₂ as defined under formula I above). Preferably, X₁₀ is bromo or chloro; even more preferably X₁₀ is bromo.

The subgroup of compounds of formula I, wherein A₁ and R₂ are as defined in formula I and wherein Q is defined as Qb, in which Q₁, R₃, X and R₁ are as defined in formula I and wherein A₂ is N, may be defined as compounds of formula I-Qb (scheme 19). Such compounds of formula I-Qb can be prepared following scheme 19. In the particular situation when Q₁ is an optionally substituted triazole linked via a nitrogen atom to the ring which contains the group A₂, then compounds of formula I-Qb, wherein X is S and in which A₁, R₁, R₂, Q₁ and R₃ are as defined in formula I,

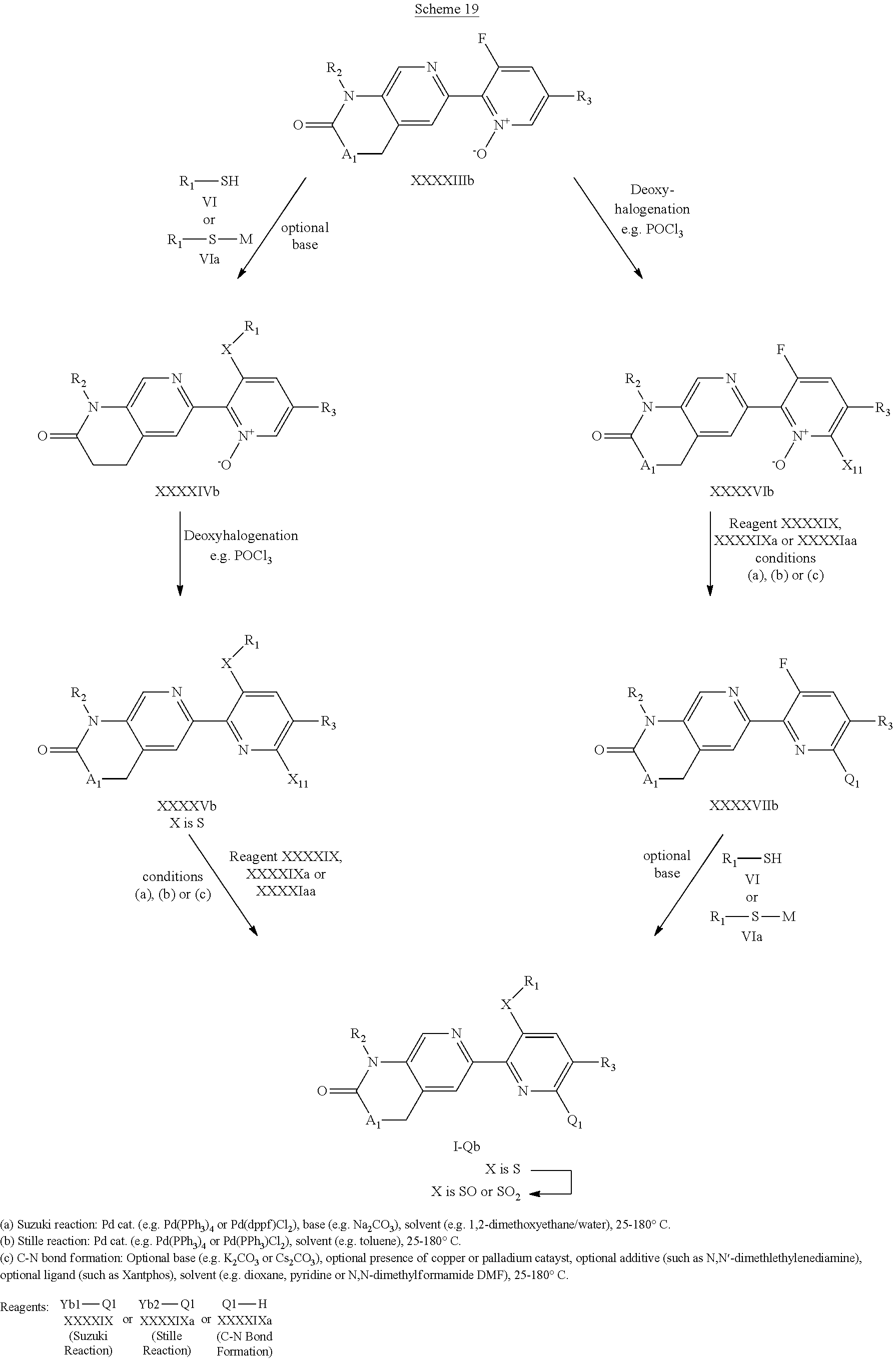

may be prepared (scheme 19) from compounds of formula XXXXVb, wherein X is S and in which A₁, R₁, R₂ and R₃ are as defined in formula I, and wherein X₁₁ is a leaving group like, for example, chlorine, bromine or iodine (preferably chlorine or bromine), or an aryl- or alkylsulfonate such as trifluoromethanesulfonate, by reaction (C—N bond formation) with an optionally substituted triazole Q₁-H (which contains an appropriate NH functionality) (XXXXIXaa), wherein Q₁ is N-linked triazolyl, in solvents such as alcohols (eg. methanol, ethanol, isopropanol, or higher boiling linear or branched alcohols), pyridine or acetic acid, optionally in the presence of an additional base, such as potassium carbonate K₂CO₃ or cesium carbonate Cs₂CO₃, optionally in the presence of a copper catalyst, for example copper(I) iodide, at temperatures between 30-180° C., optionally under microwave irradiation.

In the particular situation within scheme 19 when Q₁ is —N(R₄)COR₅, or —N(R₄)CON(R₄)₂, wherein R₄ and R₅ are as defined in formula I, then compounds of formula I-Qb, wherein X is S, may be prepared from compounds of formula XXXXVb, wherein X is S and in which A₁, R₁, R₂ and R₃ are as defined in formula I, and wherein X₁₁ is a leaving group like, for example, chlorine, bromine or iodine (preferably chlorine or bromine), or an aryl- or alkylsulfonate such as trifluoromethanesulfonate, by reaction (C—N bond formation) with a reagent Q₁-H (XXXXIXaa) equivalent to HN(R₄)COR₅, or HN(R₄)CON(R₄)₂, wherein R₄ and R₅ are as defined in formula I. Such a reaction is performed in the presence of a base, such as potassium carbonate, cesium carbonate, sodium hydroxide, in an inert solvent, such as toluene, dimethylformamide DMF, N-methyl pyrrolidine NMP, dimethyl sulfoxide DMSO, dioxane, tetrahydrofuran THF, and the like, optionally in the presence of a catalyst, for example palladium(II)acetate, bis(dibenzylideneacetone)palladium(0) (Pd(dba)₂) or tris(dibenzylideneacetone) dipalladium(0) (Pd₂(dba)₃, optionally in form of a chloroform adduct), or a palladium pre-catalyst such as for example tert-BuBrettPhos Pd G3 [(2-Di-tert-butylphosphino-3,6-dimethoxy-2′,4′,6′-triisopropyl-1,1′-biphenyl)-2-(2′-amino-1,1′-biphenyl)]palladium(II) methanesulfonate or BrettPhos Pd G3 [(2-di-cyclohexylphosphino-3,6-dimethoxy-2′,4′,6′-triisopropyl-1,1′-biphenyl)-2-(2′-amino-1,1′-biphenyl)]palladium(II) methanesulfonate, and optionally in the presence of a ligand, for example SPhos, t-BuBrettPhos or Xantphos, at temperatures between 60-120° C., optionally under microwave irradiation.

In the particular situation within scheme 19 when Q₁ is —N(R₄)₂, wherein R₄ is as defined in formula I, then compounds of formula I-Qb, wherein X is S, may be prepared from compounds of formula XXXXVb, wherein X is S and in which A₁, R₁, R₂ and R₃ are as defined in formula I, and wherein X₁₁ is a leaving group like, for example, chlorine, bromine or iodine (preferably chlorine or bromine), or an aryl- or alkylsulfonate such as trifluoromethanesulfonate, by reaction (C—N bond formation) with a reagent Q₁-H (XXXXIXaa) equivalent to HN(R₄)₂, or a salt thereof (such as a hydrohalide salt, preferably a hydrochloride or a hydrobromide salt, or a trifluoroacetic acid salt, or any other equivalent salt), wherein R₄ is as defined in formula I. Such a reaction is commonly performed in an inert solvent such as alcohols, amides, esters, ethers, nitriles and water, particularly preferred are methanol, ethanol, 2,2,2-trifluoroethanol, propanol, isopropanol, N,N-dimethylformamide, N,N-dimethylacetamide, dioxane, tetrahydrofuran, dimethoxyethane, acetonitrile, ethyl acetate, toluene, water or mixtures thereof, at temperatures between 0-150° C., optionally under microwave irradiation or pressurized conditions using an autoclave, optionally in the presence of a copper catalyst, such as copper powder, copper(I) iodide or copper sulfate (optionally in form of a hydrate), or mixtures thereof, optionally in presence a ligand, for example diamine ligands (e.g. N,N′-dimethylethylenediamine or trans-cyclohexyldiamine) or dibenzylideneacetone (dba), or 1,10-phenanthroline, and optionally in presence of a base such as potassium phosphate.

Reagents HN(R₄)₂, HN(R₄)COR₅, or HN(R₄)CON(R₄)₂, wherein R₄ and R₅ are as defined in formula I, are either known, commercially available or may be prepared by methods known to a person skilled in the art.

Alternatively, compounds of formula I-Qb, wherein X is S, may be prepared by a Suzuki reaction (scheme 19), which involves for example, reacting compounds of formula XXXXVb, wherein X is S and in which A₁, R₁, R₂ and R₃ are as defined in formula I, and wherein X₁₁ is a leaving group like, for example, chlorine, bromine or iodine (preferably chlorine or bromine), or an aryl- or alkylsulfonate such as trifluoromethanesulfonate, with compounds of formula XXXXIX, wherein Q₁ is as defined in formula I, and wherein Y_{b1} can be a boron-derived functional group, such as for example B(OH)₂ or B(OR_{b1})₂ wherein R_{b1} can be a C₁-C₄alkyl group or the two groups OR_{b1} can form together with the boron atom a five membered ring, as for example a pinacol boronic ester. The reaction may be catalyzed by a palladium based catalyst, for example tetrakis(triphenyl-phosphine)palladium(0), (1,1′bis(diphenylphosphino) ferrocene)dichloro-palladium-dichloromethane (1:1 complex) or chloro(2-dicyclohexylphosphino-2′,4′,6′-triisopropyl-1,1′-biphenyl)[2-(2′-amino-1,1′-biphenyl)]palladium(II) (XPhos palladacycle), in presence of a base, like sodium carbonate, tripotassium phosphate or cesium fluoride, in a solvent or a solvent mixture, like, for example dioxane, acetonitrile, N,N-dimethylformamide, a mixture of 1,2-dimethoxyethane and water or of dioxane/water, or of toluene/water, preferably under inert atmosphere. The reaction temperature can preferentially range from room temperature to the boiling point of the reaction mixture, or the reaction may be performed under microwave irradiation. Such Suzuki reactions are well known to those skilled in the art and have been reviewed, for example, in J. Orgmet. Chem. 576, 1999, 147-168.

Alternatively compounds of formula I-Qb, wherein X is S, may be prepared by a Stille reaction between compounds of formula XXXXIXa, wherein Q₁ is as defined above, and wherein Y_{b2} is a trialkyl tin derivative, preferably tri-n-butyl tin or tri-methyl-tin, and compounds of formula XXXXVb, wherein X is S and in which A₁, R₁, R₂ and R₃ are as defined in formula I, and wherein X₁₁ is a leaving group like, for example, chlorine, bromine or iodine (preferably chlorine or bromine), or an aryl- or alkylsulfonate such as trifluoromethanesulfonate. Such Stille reactions are usually carried out in the presence of a palladium catalyst, for example tetrakis(triphenylphosphine)palladium(0), or bis(triphenylphosphine) palladium(II) dichloride, in an inert solvent such as N,N-dimethylformamide, acetonitrile, toluene or dioxane, optionally in the presence of an additive, such as cesium fluoride, or lithium chloride, and optionally in the presence of a further catalyst, for example copper(I)iodide. Such Stille couplings are also well known to those skilled in the art, and have been described in for example J. Org. Chem., 2005, 70, 8601-8604, J. Org. Chem., 2009, 74, 5599-5602, and Angew. Chem. Int. Ed., 2004, 43, 1132-1136.

When Q₁ is a five-membered aromatic ring system linked via a nitrogen atom to the ring which contains the substituent A₂, then compounds of formula I-Qb, wherein X is S, may be prepared from compounds of formula XXXXVb, wherein X is S and in which A₁, R₁, R₂ and R₃ are as defined in formula I, and wherein X₁₁ is a leaving group like, for example, chlorine, bromine or iodine (preferably chlorine or bromine), or an aryl- or alkylsulfonate such as trifluoromethanesulfonate, by reaction with a heterocycle Q₁-H (which contains an appropriate NH functionality) (XXXXIXaa), wherein Q₁ is as defined above, in the presence of a base, such as potassium carbonate K₂CO₃ or cesium carbonate Cs₂CO₃, optionally in the presence of a copper catalyst, for example copper(I) iodide, with or without an additive such as L-proline, N,N′-dimethylcyclohexane-1,2-diamine or N,N′-dimethyl-ethylene-diamine, in an inert solvent such as N-methylpyrrolidone NMP or N,N-dimethylformamide DMF at temperatures between 30-150° C., optionally under microwave irradiation.

A large number of compounds of the formula (XXXXIX), (XXXXIXa) and (XXXXIXaa) are commercially available or can be prepared by those skilled in the art.

Alternatively, compounds of formula I-Qb, wherein X is SO or SO₂, may be prepared from compounds of formula XXXXVb, wherein X is SO or SO₂ and in which A₁, R₁, R₂ and R₃ are as defined in formula I, and wherein X₁₁ is a leaving group like, for example, chlorine, bromine or iodine (preferably chlorine or bromine), or an aryl- or alkylsulfonate such as trifluoromethanesulfonate, by involving the same chemistry as described above, but by changing the order of the steps (i.e. by running an oxidation step on XXXXVb, wherein X is S, to form XXXXVb, wherein X is SO or SO₂, followed by the sequence XXXXVb (X is SO or SO₂) to I-Qb (X is SO or SO₂) via Suzuki, Stille or C—N bond formation).

Oxidation of compounds of formula XXXXVb, wherein X is S and in which A₁, R₁, R₂, and R₃ are as defined in formula I, and wherein X₁₁ is a leaving group like, for example, chlorine, bromine or iodine (preferably chlorine or bromine), or an aryl- or alkylsulfonate such as trifluoromethanesulfonate, with a suitable oxidizing agent, into compounds of formula XXXXVb, wherein X is SO or SO₂ may be achieved under conditions already described above.

Compounds of formula XXXXVb, wherein X is S and in which A₁, R₁, R₂ and R₃ are as defined in formula I, and wherein X₁₁ is a leaving group like, for example, chlorine, bromine or iodine (preferably chlorine or bromine), or an aryl- or alkylsulfonate such as trifluoromethanesulfonate, may be prepared (scheme 19) by reacting compounds of formula XXXXIVb, wherein X is S and in which A₁, R₁, R₂, and R₃ are as defined in formula I, with a halogenating agent such as phosphorus oxychloride POCl₃ or phosphorus oxybromide, neat or in an appropriate solvent, such as chloroform or toluene, optionally in the presence of a base, such as triethylamine or pyridine, at temperatures between room temperature and refluxing conditions. Such deoxyhalogenation have been described in, for example, WO16/116338.

Compounds of formula XXXXIVb, wherein X is S and in which A₁, R₁, R₂ and R₃ are as defined in formula I, may be prepared by reacting compounds of formula XXXXIIIb, wherein A₁, R₂ and R₃ are as defined in formula I, with reagents of formula VI or VIa, wherein R₁ is as defined in formula I, under conditions already described above (see text scheme 3).

Alternatively, compounds of formula I-Qb, wherein X is S, SO or SO₂, may be prepared (scheme 19) from compounds of formula XXXXIIIb, by involving the same chemistry as described above, but by changing the order of the steps (i.e. by running the sequence XXXXIIIb to XXXXVIb, XXXXVIb to XXXXVIIb which was described previously, and XXXXVIIb to I-Qb, followed by oxidation, and wherein all substituent definitions mentioned previously remain valid).

Compounds of formula XXXXIIIb, wherein A₁, R₂ and R₃ are as defined in formula I, can be prepared from compounds of formula XIIIb, respectively XIVb, in which Q₁ is hydrogen and R₃ is as defined in formula I, by following procedures analogous to those described in schemes 8 and 17.

The subgroup of compounds of formula I, wherein A₁ and R₂ are as defined in formula I and wherein Q is defined as Qa, in which Q₁, R₃, X and R₁ are as defined in formula I and wherein A₂ is N, may be defined as compounds of formula I-Qa (scheme 20). Such compounds of formula I-Qa can be prepared following scheme 20. In the particular situation when R₃ is C₁-C₄alkyl, then compounds of formula I-Qa, wherein X is S and in which A₁, R₁, R₂, Q₁ and R₃ are as defined in formula I,

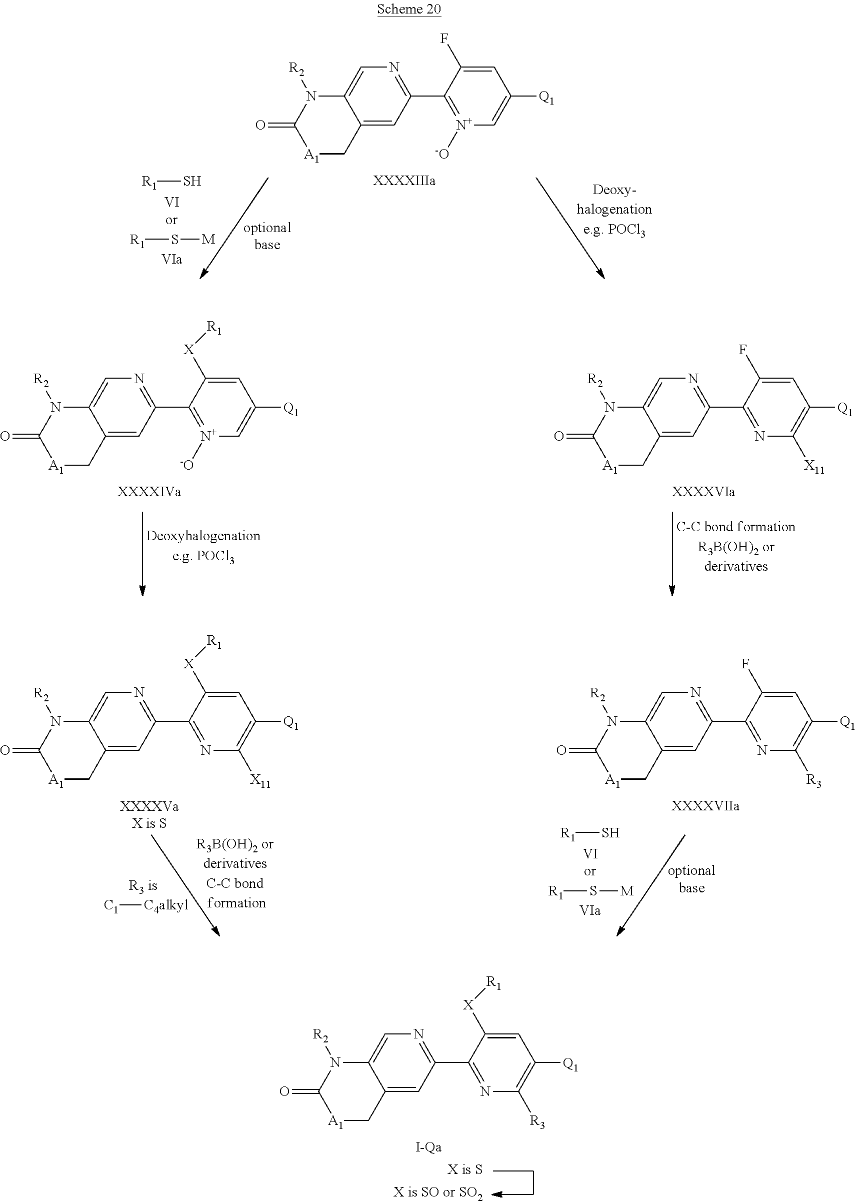

may be prepared (scheme 20) from compounds of formula XXXXVa, wherein X is S and in which A₁, R₁, Q₁ and R₂ are as defined in formula I, and wherein X₁₁ is a leaving group like, for example, chlorine, bromine or iodine (preferably chlorine or bromine), or an aryl- or alkylsulfonate such as trifluoromethanesulfonate, by means of a C—C bond formation reaction typically under palladium-catalyzed (alternatively nickel-catalyzed) cross-coupling conditions. Such Suzuki-Miyaura cross-coupling reactions between compounds of formula XXXXVa and C₁-C₄alkyl boronic acids of the formula R₃B(OH)₂, wherein R₃ is C₁-C₄alkyl, or the corresponding C₁-C₄alkyl boronate ester derivatives, or the corresponding 6-membered tri(C₁-C₄alkyl) boroxine derivatives of the formula (R₃BO)₃, wherein R₃ is C₁-C₄alkyl, are well known to a person skilled in the art. In the particular situation where R₃ is methyl, compounds of formula XXXXVa can be reacted, for example, with trimethylboroxine (also known as 2,4,6-trimethyl-1,3,5,2,4,6-trioxatriborinane) in the presence of palladium catalyst, such as tetrakis(triphenylphosphine)-palladium(0) or [1,1′-bis(diphenylphosphino) ferrocene]palladium(II) dichloride dichloromethane complex, and a base, such as sodium or potassium carbonate, in a solvent, such as N,N-dimethylformamide, dioxane or dioxane-water mixtures, at temperatures between room temperature and 160° C., optionally under microwave heating conditions, and preferably under inert atmosphere. Such conditions are described, for example, in Tetrahedron Letters (2000), 41(32), 6237-6240.

Compounds of formula XXXXVa, wherein X is S and in which A₁, R₁, R₂ and Q₁ are as defined in formula I, and wherein X₁₁ is a leaving group like, for example, chlorine, bromine or iodine (preferably chlorine or bromine), or an aryl- or alkylsulfonate such as trifluoromethanesulfonate, may be prepared from compounds of formula XXXXIIIa (via compounds of formula XXXXIVa), wherein Q₁ is as defined in formula I, in a sequence and under conditions already described above (see text scheme 19), and wherein all substituent definitions mentioned previously remain valid).

Alternatively, compounds of formula I-Qa, wherein X is SO or SO₂, may be prepared from compounds of formula XXXXVa, wherein X is SO or SO₂ and in which A₁, R₁, R₂ and Q₁ are as defined in formula I, and wherein X₁₁ is a leaving group like, for example, chlorine, bromine or iodine (preferably chlorine or bromine), or an aryl- or alkylsulfonate such as trifluoromethanesulfonate, by involving the same chemistry as described above, but by changing the order of the steps (i.e. by running an oxidation step on XXXXVa, wherein X is S, to form XXXXVa, wherein X is SO or SO₂, followed by the sequence XXXXVa (X is SO or SO₂) to I-Qa (X is SO or SO₂) via C—C bond formation with R₃B(OH)₂, or equivalent).

Oxidation of compounds of formula XXXXVa, wherein X is S and in which A₁, R₁, R₂ and Q₁ are as defined in formula I, and wherein X₁₁ is a leaving group like, for example, chlorine, bromine or iodine (preferably chlorine or bromine), or an aryl- or alkylsulfonate such as trifluoromethanesulfonate, with a suitable oxidizing agent, into compounds of formula XXXXVa, wherein X is SO or SO₂ may be achieved under conditions already described above.

Alternatively, compounds of formula I-Qa, wherein X is S, SO or SO₂, may be prepared (scheme 20) from compounds of formula XXXXIIIa, by involving the same chemistry as just described above, but by changing the order of the steps (i.e. by running the sequence XXXXIIIa to XXXXVIa, XXXXVIa to XXXXVIIa which was described previously, and XXXXVIIa to I-Qa, followed by oxidation, and wherein all substituent definitions mentioned previously remain valid).

In the particular situation when R₃ is hydrogen, then compounds of formula I-Qa, wherein X is S, SO or SO₂, and in which A₁, R₁, R₂ and Q₁ are as defined in formula I, may alternatively be prepared (scheme 20) from compounds of formula XXXXVa, wherein X is S, SO or SO₂, and in which A₁, R₁, R₂ and Q₁ are as defined in formula I, and wherein X₁₁ is a leaving group like, for example, chlorine, bromine or iodine (preferably chlorine or bromine), or an aryl- or alkylsulfonate such as trifluoro-methanesulfonate, by means of a reductive dehalogenation. Such a hydrodehalogenation can be achieved, for example, using zinc dust and acetic acid or trifluoroacetic acid, or mixtures thereof, at temperatures between 0° C. and 120° C., preferably between 50° C. and reflux temperature, as described, for example, in Journal of the Chemical Society, Perkin Transactions 1: Organic and Bio-Organic Chemistry (1972-1999), (10), 2501-6, 1983 or in US20100076027.

C₁-C₄alkyl boronic acids of the formula R₃B(OH)₂, wherein R₃ is C₁-C₄alkyl, or the corresponding C₁-C₄alkyl boronate ester derivatives, or the corresponding 6-membered tri(C₁-C₄alkyl) boroxine derivatives of the formula (R₃BO)₃, wherein R₃ is C₁-C₄alkyl, are either known, commercially available or may be prepared by methods known to a person skilled in the art.

Compounds of formula XXXXIIIa, wherein A₁, R₂ and Q₁ are as defined in formula I, can be prepared from compounds of formula XIIIa, respectively XIVa, wherein Q₁ is as defined in formula I and in which R₃ is hydrogen, by following procedures analogous to those described in schemes 7 and 16.

The reactants can be reacted in the presence of a base. Examples of suitable bases are alkali metal or alkaline earth metal hydroxides, alkali metal or alkaline earth metal hydrides, alkali metal or alkaline earth metal amides, alkali metal or alkaline earth metal alkoxides, alkali metal or alkaline earth metal acetates, alkali metal or alkaline earth metal carbonates, alkali metal or alkaline earth metal dialkylamides or alkali metal or alkaline earth metal alkylsilylamides, alkylamines, alkylenediamines, free or N-alkylated saturated or unsaturated cycloalkylamines, basic heterocycles, ammonium hydroxides and carbocyclic amines. Examples which may be mentioned are sodium hydroxide, sodium hydride, sodium amide, sodium methoxide, sodium acetate, sodium carbonate, potassium tert-butoxide, potassium hydroxide, potassium carbonate, potassium hydride, lithium diisopropylamide, potassium bis(trimethylsilyl)amide, calcium hydride, triethylamine, diisopropylethylamine, triethylenediamine, cyclohexylamine, N-cyclohexyl-N,N-dimethylamine, N,N-diethylaniline, pyridine, 4-(N,N-dimethylamino)pyridine, quinuclidine, N-methylmorpholine, benzyltrimethylammonium hydroxide and 1,8-diazabicyclo[5.4.0]undec-7-ene (DBU).

The reactants can be reacted with each other as such, i.e. without adding a solvent or diluent. In most cases, however, it is advantageous to add an inert solvent or diluent or a mixture of these. If the reaction is carried out in the presence of a base, bases which are employed in excess, such as triethylamine, pyridine, N-methylmorpholine or N,N-diethylaniline, may also act as solvents or diluents.

The reactions are advantageously carried out in a temperature range from approximately −80° C. to approximately +140° C., preferably from approximately −30° C. to approximately +100° C., in many cases in the range between ambient temperature and approximately +80° C.

A compound of formula I can be converted in a manner known per se into another compound of formula I by replacing one or more substituents of the starting compound of formula I in the customary manner by (an)other substituent(s) according to the invention, and by post modification of compounds of with reactions such as oxidation, alkylation, reduction, acylation and other methods known by those skilled in the art.

Depending on the choice of the reaction conditions and starting materials which are suitable in each case, it is possible, for example, in one reaction step only to replace one substituent by another substituent according to the invention, or a plurality of substituents can be replaced by other substituents according to the invention in the same reaction step.

Salts of compounds of formula I can be prepared in a manner known per se. Thus, for example, acid addition salts of compounds of formula I are obtained by treatment with a suitable acid or a suitable ion exchanger reagent and salts with bases are obtained by treatment with a suitable base or with a suitable ion exchanger reagent.

Salts of compounds of formula I can be converted in the customary manner into the free compounds I, acid addition salts, for example, by treatment with a suitable basic compound or with a suitable ion exchanger reagent and salts with bases, for example, by treatment with a suitable acid or with a suitable ion exchanger reagent.

Salts of compounds of formula I can be converted in a manner known per se into other salts of compounds of formula I, acid addition salts, for example, into other acid addition salts, for example by treatment of a salt of inorganic acid such as hydrochloride with a suitable metal salt such as a sodium, barium or silver salt, of an acid, for example with silver acetate, in a suitable solvent in which an inorganic salt which forms, for example silver chloride, is insoluble and thus precipitates from the reaction mixture.

Depending on the procedure or the reaction conditions, the compounds of formula I, which have salt-forming properties can be obtained in free form or in the form of salts.

The compounds of formula I and, where appropriate, the tautomers thereof, in each case in free form or in salt form, can be present in the form of one of the isomers which are possible or as a mixture of these, for example in the form of pure isomers, such as antipodes and/or diastereomers, or as isomer mixtures, such as enantiomer mixtures, for example racemates, diastereomer mixtures or racemate mixtures, depending on the number, absolute and relative configuration of asymmetric carbon atoms which occur in the molecule and/or depending on the configuration of non-aromatic double bonds which occur in the molecule; the invention relates to the pure isomers and also to all isomer mixtures which are possible and is to be understood in each case in this sense hereinabove and hereinbelow, even when stereochemical details are not mentioned specifically in each case.

Diastereomer mixtures or racemate mixtures of compounds of formula I, in free form or in salt form, which can be obtained depending on which starting materials and procedures have been chosen can be separated in a known manner into the pure diasteromers or racemates on the basis of the physicochemical differences of the components, for example by fractional crystallization, distillation and/or chromatography.

Enantiomer mixtures, such as racemates, which can be obtained in a similar manner can be resolved into the optical antipodes by known methods, for example by recrystallization from an optically active solvent, by chromatography on chiral adsorbents, for example high-performance liquid chromatography (HPLC) on acetyl celulose, with the aid of suitable microorganisms, by cleavage with specific, immobilized enzymes, via the formation of inclusion compounds, for example using chiral crown ethers, where only one enantiomer is complexed, or by conversion into diastereomeric salts, for example by reacting a basic end-product racemate with an optically active acid, such as a carboxylic acid, for example camphor, tartaric or malic acid, or sulfonic acid, for example camphorsulfonic acid, and separating the diastereomer mixture which can be obtained in this manner, for example by fractional crystallization based on their differing solubilities, to give the diastereomers, from which the desired enantiomer can be set free by the action of suitable agents, for example basic agents.

Pure diastereomers or enantiomers can be obtained according to the invention not only by separating suitable isomer mixtures, but also by generally known methods of diastereoselective or enantioselective synthesis, for example by carrying out the process according to the invention with starting materials of a suitable stereochemistry.

N-oxides can be prepared by reacting a compound of the formula I with a suitable oxidizing agent, for example the H₂O₂/urea adduct in the presence of an acid anhydride, e.g. trifluoroacetic anhydride. Such oxidations are known from the literature, for example from J. Med. Chem., 32 (12), 2561-73, 1989 or WO 2000/15615.

It is advantageous to isolate or synthesize in each case the biologically more effective isomer, for example enantiomer or diastereomer, or isomer mixture, for example enantiomer mixture or diastereomer mixture, if the individual components have a different biological activity.

The compounds of formula I and, where appropriate, the tautomers thereof, in each case in free form or in salt form, can, if appropriate, also be obtained in the form of hydrates and/or include other solvents, for example those which may have been used for the crystallization of compounds which are present in solid form.

The compounds according to the following Tables A-1 to A-60, Tables B-1 to B-60, Tables C-1 to C-60 and Tables D-1 to D-60 below can be prepared according to the methods described above. The examples which follow are intended to illustrate the invention and show preferred compounds of formula I.

The tables A-1 to A-60 below illustrate specific compounds of the invention.

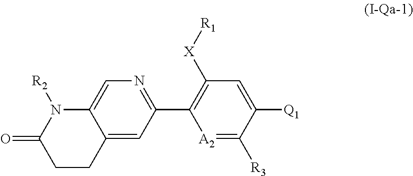

TABLE Y
Substituent definitions of Q₁

| Index | Q₁ |
|---|---|
| 1 | H |
| 2 | —N(CH₃)COCH₂CH₃ |
| 3 | Cl |
| 4 | Br |
| 5 | —N(CH₃)COcycloC3 |
| 6 | CF₃ |
| 7 | —NH₂ |
| 8 | —NH(CH₃) |
| 9 | —N(CH₃)₂ |
| 10 | —NHCOCH₃ |
| 11 | —N(CH₃)COCH₃ |
| 12 | 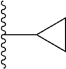 [SMILES: *C1CC1] |
| 13 | 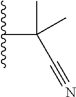 [SMILES: *C(C)(C)C#N] |
| 14 | 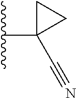 [SMILES: *C1(C#N)CC1] |
| 15 | —OCH₂CF₃ |
| 16 | 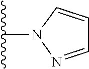 [SMILES: *n1cccn1] |
| 17 | 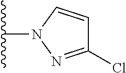 [SMILES: *n1ccc(Cl)n1] |
| 18 | 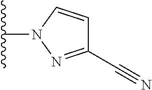 [SMILES: *n1ccc(C#N)n1] |
| 19 | 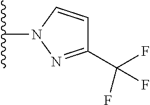 [SMILES: *n1ccc(C(F)(F)F)n1] |
| 20 | 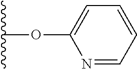 [SMILES: *Oc1ccccn1] |
| 21 | 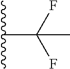 [SMILES: *C(C)(F)F] |
| 22 | 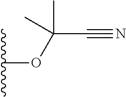 [SMILES: *OC(C)(C)C#N] |
| 23 | 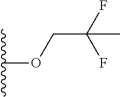 [SMILES: *OCC(C)(F)F] |

In the table Y and in tables A, “cycloC3” represents cyclopropyl.

Table A-1 provides 23 compounds A-1.001 to A-1.023 of formula I-Qa-1 wherein R₂ is CH₂CF₂CF₃, A₂ is N, R₃ is H, X is S, R₁ is ethyl and Q₁ is as defined in table Y.

For example, compound A-22.017 is

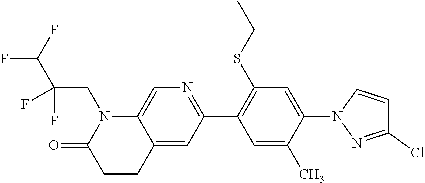 [SMILES: CCSc1cc(-n2ccc(Cl)n2)c(C)cc1-c1cc2c(cn1)N(CC(F)(F)C(F)F)C(=O)CC2]

Table A-2 provides 23 compounds A-2.001 to A-2.023 of formula I-Qa-1 wherein R₂ is CH₂CF₂CF₃, A₂ is N, R₃ is H, X is SO, R₁ is ethyl and Q₁ is as defined in table Y.

Table A-3 provides 23 compounds A-3.001 to A-3.023 of formula I-Qa-1 wherein R₂ is CH₂CF₂CF₃, A₂ is N, R₃ is H, X is SO₂, R₁ is ethyl and Q₁ is as defined in table Y.

Table A-4 provides 23 compounds A-4.001 to A-4.023 of formula I-Qa-1 wherein R₂ is CH₂CF₂CF₃, A₂ is N, R₃ is Me, X is S, R₁ is ethyl and Q₁ is as defined in table Y.

Table A-5 provides 23 compounds A-5.001 to A-5.023 of formula I-Qa-1 wherein R₂ is CH₂CF₂CF₃, A₂ is N, R₃ is Me, X is SO, R₁ is ethyl and Q₁ is as defined in table Y.

Table A-6 provides 23 compounds A-6.001 to A-6.023 of formula I-Qa-1 wherein R₂ is CH₂CF₂CF₃, A₂ is N, R₃ is Me, X is SO₂, R₁ is ethyl and Q₁ is as defined in table Y.

Table A-7 provides 23 compounds A-7.001 to A-7.023 of formula I-Qa-1 wherein R₂ is CH₂CF₂CF₃, A₂ is CH, R₃ is H, X is S, R₁ is ethyl and Q₁ is as defined in table Y.

Table A-8 provides 23 compounds A-8.001 to A-8.023 of formula I-Qa-1 wherein R₂ is CH₂CF₂CF₃, A₂ is CH, R₃ is H, X is SO, R₁ is ethyl and Q₁ is as defined in table Y.

Table A-9 provides 23 compounds A-9.001 to A-9.023 of formula I-Qa-1 wherein R₂ is CH₂CF₂CF₃, A₂ is CH, R₃ is H, X is SO₂, R₁ is ethyl and Q₁ is as defined in table Y.

Table A-10 provides 23 compounds A-10.001 to A-10.023 of formula I-Qa-1 wherein R₂ is CH₂CF₂CF₃, A₂ is CH, R₃ is Me, X is S, R₁ is ethyl and Q₁ is as defined in table Y.

Table A-11 provides 23 compounds A-11.001 to A-11.023 of formula I-Qa-1 wherein R₂ is CH₂CF₂CF₃, A₂ is CH, R₃ is Me, X is SO, R₁ is ethyl and Q₁ is as defined in table Y.

Table A-12 provides 23 compounds A-12.001 to A-12.023 of formula I-Qa-1 wherein R₂ is CH₂CF₂CF₃, A₂ is CH, R₃ is Me, X is SO₂, R₁ is ethyl and Q₁ is as defined in table Y.

Table A-13 provides 23 compounds A-13.001 to A-13.023 of formula I-Qa-1 wherein R₂ is CH₂CF₂CHF₂, A₂ is N, R₃ is H, X is S, R₁ is ethyl and Q₁ is as defined in table Y.

Table A-14 provides 23 compounds A-14.001 to A-14.023 of formula I-Qa-1 wherein R₂ is CH₂CF₂CHF₂, A₂ is N, R₃ is H, X is SO, R₁ is ethyl and Q₁ is as defined in table Y.

Table A-15 provides 23 compounds A-15.001 to A-15.023 of formula I-Qa-1 wherein R₂ is CH₂CF₂CHF₂, A₂ is N, R₃ is H, X is SO₂, R₁ is ethyl and Q₁ is as defined in table Y.

Table A-16 provides 23 compounds A-16.001 to A-16.023 of formula I-Qa-1 wherein R₂ is CH₂CF₂CHF₂, A₂ is N, R₃ is Me, X is S, R₁ is ethyl and Q₁ is as defined in table Y.

Table A-17 provides 23 compounds A-17.001 to A-17.023 of formula I-Qa-1 wherein R₂ is CH₂CF₂CHF₂, A₂ is N, R₃ is Me, X is SO, R₁ is ethyl and Q₁ is as defined in table Y.

Table A-18 provides 23 compounds A-18.001 to A-18.023 of formula I-Qa-1 wherein R₂ is CH₂CF₂CHF₂, A₂ is N, R₃ is Me, X is SO₂, R₁ is ethyl and Q₁ is as defined in table Y.

Table A-19 provides 23 compounds A-19.001 to A-19.023 of formula I-Qa-1 wherein R₂ is CH₂CF₂CHF₂, A₂ is CH, R₃ is H, X is S, R₁ is ethyl and Q₁ is as defined in table Y.

Table A-20 provides 23 compounds A-20.001 to A-20.023 of formula I-Qa-1 wherein R₂ is CH₂CF₂CHF₂, A₂ is CH, R₃ is H, X is SO, R₁ is ethyl and Q₁ is as defined in table Y.

Table A-21 provides 23 compounds A-21.001 to A-21.023 of formula I-Qa-1 wherein R₂ is CH₂CF₂CHF₂, A₂ is CH, R₃ is H, X is SO₂, R₁ is ethyl and Q₁ is as defined in table Y.

Table A-22 provides 23 compounds A-22.001 to A-22.023 of formula I-Qa-1 wherein R₂ is CH₂CF₂CHF₂, A₂ is CH, R₃ is Me, X is S, R₁ is ethyl and Q₁ is as defined in table Y.

Table A-23 provides 23 compounds A-23.001 to A-23.023 of formula I-Qa-1 wherein R₂ is CH₂CF₂CHF₂, A₂ is CH, R₃ is Me, X is SO, R₁ is ethyl and Q₁ is as defined in table Y.

Table A-24 provides 23 compounds A-24.001 to A-24.023 of formula I-Qa-1 wherein R₂ is CH₂CF₂CHF₂, A₂ is CH, R₃ is Me, X is SO₂, R₁ is ethyl and Q₁ is as defined in table Y.

Table A-25 provides 23 compounds A-25.001 to A-25.023 of formula I-Qa-1 wherein R₂ is CH₂CF₃, A₂ is N, R₃ is H, X is S, R₁ is ethyl and Q₁ is as defined in table Y.

Table A-26 provides 23 compounds A-26.001 to A-26.023 of formula I-Qa-1 wherein R₂ is CH₂CF₃, A₂ is N, R₃ is H, X is SO, R₁ is ethyl and Q₁ is as defined in table Y.

Table A-27 provides 23 compounds A-27.001 to A-27.023 of formula I-Qa-1 wherein R₂ is CH₂CF₃, A₂ is N, R₃ is H, X is SO₂, R₁ is ethyl and Q₁ is as defined in table Y.

Table A-28 provides 23 compounds A-28.001 to A-28.023 of formula I-Qa-1 wherein R₂ is CH₂CF₃, A₂ is N, R₃ is Me, X is S, R₁ is ethyl and Q₁ is as defined in table Y.

Table A-29 provides 23 compounds A-29.001 to A-29.023 of formula I-Qa-1 wherein R₂ is CH₂CF₃, A₂ is N, R₃ is Me, X is SO, R₁ is ethyl and Q₁ is as defined in table Y.

Table A-30 provides 23 compounds A-30.001 to A-30.023 of formula I-Qa-1 wherein R₂ is CH₂CF₃, A₂ is N, R₃ is Me, X is SO₂, R₁ is ethyl and Q₁ is as defined in table Y.

Table A-31 provides 23 compounds A-31.001 to A-31.023 of formula I-Qa-1 wherein R₂ is CH₂CF₃, A₂ is CH, R₃ is H, X is S, R₁ is ethyl and Q₁ is as defined in table Y.

Table A-32 provides 23 compounds A-32.001 to A-32.023 of formula I-Qa-1 wherein R₂ is CH₂CF₃, A₂ is CH, R₃ is H, X is SO, R₁ is ethyl and Q₁ is as defined in table Y.

Table A-33 provides 23 compounds A-33.001 to A-33.023 of formula I-Qa-1 wherein R₂ is CH₂CF₃, A₂ is CH, R₃ is H, X is SO₂, R₁ is ethyl and Q₁ is as defined in table Y.

Table A-34 provides 23 compounds A-34.001 to A-34.023 of formula I-Qa-1 wherein R₂ is CH₂CF₃, A₂ is CH, R₃ is Me, X is S, R₁ is ethyl and Q₁ is as defined in table Y.

Table A-35 provides 23 compounds A-35.001 to A-35.023 of formula I-Qa-1 wherein R₂ is CH₂CF₃, A₂ is CH, R₃ is Me, X is SO, R₁ is ethyl and Q₁ is as defined in table Y.

Table A-36 provides 23 compounds A-36.001 to A-36.023 of formula I-Qa-1 wherein R₂ is CH₂CF₃, A₂ is CH, R₃ is Me, X is SO₂, R₁ is ethyl and Q₁ is as defined in table Y.

Table A-37 provides 23 compounds A-37.001 to A-37.023 of formula I-Qa-1 wherein R₂ is CH₂CHF₂, A₂ is N, R₃ is H, X is S, R₁ is ethyl and Q₁ is as defined in table Y.

Table A-38 provides 23 compounds A-38.001 to A-38.023 of formula I-Qa-1 wherein R₂ is CH₂CHF₂, A₂ is N, R₃ is H, X is SO, R₁ is ethyl and Q₁ is as defined in table Y.

Table A-39 provides 23 compounds A-39.001 to A-39.023 of formula I-Qa-1 wherein R₂ is CH₂CHF₂, A₂ is N, R₃ is H, X is SO₂, R₁ is ethyl and Q₁ is as defined in table Y.

Table A-40 provides 23 compounds A-40.001 to A-40.023 of formula I-Qa-1 wherein R₂ is CH₂CHF₂, A₂ is N, R₃ is Me, X is S, R₁ is ethyl and Q₁ is as defined in table Y.

Table A-41 provides 23 compounds A-41.001 to A-41.023 of formula I-Qa-1 wherein R₂ is CH₂CHF₂, A₂ is N, R₃ is Me, X is SO, R₁ is ethyl and Q₁ is as defined in table Y.

Table A-42 provides 23 compounds A-42.001 to A-42.023 of formula I-Qa-1 wherein R₂ is CH₂CHF₂, A₂ is N, R₃ is Me, X is SO₂, R₁ is ethyl and Q₁ is as defined in table Y.

Table A-43 provides 23 compounds A-43.001 to A-43.023 of formula I-Qa-1 wherein R₂ is CH₂CHF₂, A₂ is CH, R₃ is H, X is S, R₁ is ethyl and Q₁ is as defined in table Y.

Table A-44 provides 23 compounds A-44.001 to A-44.023 of formula I-Qa-1 wherein R₂ is CH₂CHF₂, A₂ is CH, R₃ is H, X is SO, R₁ is ethyl and Q₁ is as defined in table Y.

Table A-45 provides 23 compounds A-45.001 to A-45.023 of formula I-Qa-1 wherein R₂ is CH₂CHF₂, A₂ is CH, R₃ is H, X is SO₂, R₁ is ethyl and Q₁ is as defined in table Y.

Table A-46 provides 23 compounds A-46.001 to A-46.023 of formula I-Qa-1 wherein R₂ is CH₂CHF₂, A₂ is CH, R₃ is Me, X is S, R₁ is ethyl and Q₁ is as defined in table Y.

Table A-47 provides 23 compounds A-47.001 to A-47.023 of formula I-Qa-1 wherein R₂ is CH₂CHF₂, A₂ is CH, R₃ is Me, X is SO, R₁ is ethyl and Q₁ is as defined in table Y.

Table A-48 provides 23 compounds A-48.001 to A-48.023 of formula I-Qa-1 wherein R₂ is CH₂CHF₂, A₂ is CH, R₃ is Me, X is SO₂, R₁ is ethyl and Q₁ is as defined in table Y.

Table A-49 provides 23 compounds A-49.001 to A-49.023 of formula I-Qa-1 wherein R₂ is CH₂CF₂CHFCF₃, A₂ is N, R₃ is H, X is S, R₁ is ethyl and Q₁ is as defined in table Y.

Table A-50 provides 23 compounds A-50.001 to A-50.023 of formula I-Qa-1 wherein R₂ is CH₂CF₂CHFCF₃, A₂ is N, R₃ is H, X is SO, R₁ is ethyl and Q₁ is as defined in table Y.

Table A-51 provides 23 compounds A-51.001 to A-51.023 of formula I-Qa-1 wherein R₂ is CH₂CF₂CHFCF₃, A₂ is N, R₃ is H, X is SO₂, R₁ is ethyl and Q₁ is as defined in table Y.

Table A-52 provides 23 compounds A-52.001 to A-52.023 of formula I-Qa-1 wherein R₂ is CH₂CF₂CHFCF₃, A₂ is N, R₃ is Me, X is S, R₁ is ethyl and Q₁ is as defined in table Y.

Table A-53 provides 23 compounds A-53.001 to A-53.023 of formula I-Qa-1 wherein R₂ is CH₂CF₂CHFCF₃, A₂ is N, R₃ is Me, X is SO, R₁ is ethyl and Q₁ is as defined in table Y.

Table A-54 provides 23 compounds A-54.001 to A-54.023 of formula I-Qa-1 wherein R₂ is CH₂CF₂CHFCF₃, A₂ is N, R₃ is Me, X is SO₂, R₁ is ethyl and Q₁ is as defined in table Y.

Table A-55 provides 23 compounds A-55.001 to A-55.023 of formula I-Qa-1 wherein R₂ is CH₂CF₂CHFCF₃, A₂ is CH, R₃ is H, X is S, R₁ is ethyl and Q₁ is as defined in table Y.

Table A-56 provides 23 compounds A-56.001 to A-56.023 of formula I-Qa-1 wherein R₂ is CH₂CF₂CHFCF₃, A₂ is CH, R₃ is H, X is SO, R₁ is ethyl and Q₁ is as defined in table Y.

Table A-57 provides 23 compounds A-57.001 to A-57.023 of formula I-Qa-1 wherein R₂ is CH₂CF₂CHFCF₃, A₂ is CH, R₃ is H, X is SO₂, R₁ is ethyl and Q₁ is as defined in table Y.

Table A-58 provides 23 compounds A-58.001 to A-58.023 of formula I-Qa-1 wherein R₂ is CH₂CF₂CHFCF₃, A₂ is CH, R₃ is Me, X is S, R₁ is ethyl and Q₁ is as defined in table Y.

Table A-59 provides 23 compounds A-59.001 to A-59.023 of formula I-Qa-1 wherein R₂ is CH₂CF₂CHFCF₃, A₂ is CH, R₃ is Me, X is SO, R₁ is ethyl and Q₁ is as defined in table Y.

Table A-60 provides 23 compounds A-60.001 to A-60.023 of formula I-Qa-1 wherein R₂ is CH₂CF₂CHFCF₃, A₂ is CH, R₃ is Me, X is SO₂, R₁ is ethyl and Q₁ is as defined in table Y.

The tables B-1 to B-60 below illustrate further specific compounds of the invention.

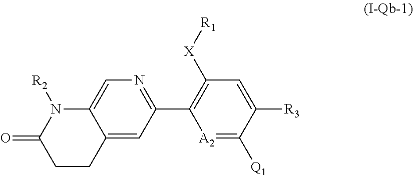

TABLE Z
Substituent definitions of Q₁

| Index | Q₁ |
|---|---|
| 1 | H |
| 2 | Cl |
| 3 | Br |
| 4 | —N(CH₃)COCH₂CH₃ |
| 5 | —NH₂ |
| 6 | —NH(CH₃) |
| 7 | —N(CH₃)₂ |
| 8 | —NHCOCH₃ |
| 9 | —N(CH₃)COCH₃ |
| 10 | —NHCOcycloC3 |
| 11 | —N(CH₃)COcycloC3 |
| 12 | 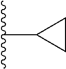 [SMILES: *C1CC1] |
| 13 | —N(H)CONH₂ |
| 14 | —N(H)CONH(CH₃) |
| 15 | —N(H)CON(CH₃)₂ |
| 16 | —N(CH₃)CONH₂ |
| 17 | —N(CH₃)CONH(CH₃) |
| 18 | —N(CH₃)CON(CH₃)₂ |
| 19 | 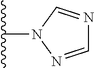 [SMILES: *n1cncn1] |
| 20 | 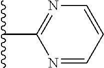 [SMILES: *c1ncccn1] |
| 21 | 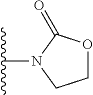 [SMILES: *N1CCOC1=O] |

In the table Z and in tables B, “cycloC3” represents cyclopropyl.

Table B-1 provides 21 compounds B-1.001 to B-1.021 of formula I-Qb-1 wherein R₂ is CH₂CF₂CF₃, A₂ is N, R₃ is H, X is S, R₁ is ethyl and Q₁ is as defined in table Z.

Table B-2 provides 21 compounds B-2.001 to B-2.021 of formula I-Qb-1 wherein R₂ is CH₂CF₂CF₃, A₂ is N, R₃ is H, X is SO, R₁ is ethyl and Q₁ is as defined in table Z.

Table B-3 provides 21 compounds B-3.001 to B-3.021 of formula I-Qb-1 wherein R₂ is CH₂CF₂CF₃, A₂ is N, R₃ is H, X is SO₂, R₁ is ethyl and Q₁ is as defined in table Z.

Table B-4 provides 21 compounds B-4.001 to B-4.021 of formula I-Qb-1 wherein R₂ is CH₂CF₂CF₃, A₂ is N, R₃ is Me, X is S, R₁ is ethyl and Q₁ is as defined in table Z.

Table B-5 provides 21 compounds B-5.001 to B-5.021 of formula I-Qb-1 wherein R₂ is CH₂CF₂CF₃, A₂ is N, R₃ is Me, X is SO, R₁ is ethyl and Q₁ is as defined in table Z.

Table B-6 provides 21 compounds B-6.001 to B-6.021 of formula I-Qb-1 wherein R₂ is CH₂CF₂CF₃, A₂ is N, R₃ is Me, X is SO₂, R₁ is ethyl and Q₁ is as defined in table Z.

Table B-7 provides 21 compounds B-7.001 to B-7.021 of formula I-Qb-1 wherein R₂ is CH₂CF₂CF₃, A₂ is CH, R₃ is H, X is S, R₁ is ethyl and Q₁ is as defined in table Z.

Table B-8 provides 21 compounds B-8.001 to B-8.021 of formula I-Qb-1 wherein R₂ is CH₂CF₂CF₃, A₂ is CH, R₃ is H, X is SO, R₁ is ethyl and Q₁ is as defined in table Z.

Table B-9 provides 21 compounds B-9.001 to B-9.021 of formula I-Qb-1 wherein R₂ is CH₂CF₂CF₃, A₂ is CH, R₃ is H, X is SO₂, R₁ is ethyl and Q₁ is as defined in table Z.

Table B-10 provides 21 compounds B-10.001 to B-10.021 of formula I-Qb-1 wherein R₂ is CH₂CF₂CF₃, A₂ is CH, R₃ is Me, X is S, R₁ is ethyl and Q₁ is as defined in table Z.

Table B-11 provides 21 compounds B-11.001 to B-11.021 of formula I-Qb-1 wherein R₂ is CH₂CF₂CF₃, A₂ is CH, R₃ is Me, X is SO, R₁ is ethyl and Q₁ is as defined in table Z.

Table B-12 provides 21 compounds B-12.001 to B-12.021 of formula I-Qb-1 wherein R₂ is CH₂CF₂CF₃, A₂ is CH, R₃ is Me, X is SO₂, R₁ is ethyl and Q₁ is as defined in table Z.

Table B-13 provides 21 compounds B-13.001 to B-13.021 of formula I-Qb-1 wherein R₂ is CH₂CF₂CHF₂, A₂ is N, R₃ is H, X is S, R₁ is ethyl and Q₁ is as defined in table Z.

Table B-14 provides 21 compounds B-14.001 to B-14.021 of formula I-Qb-1 wherein R₂ is CH₂CF₂CHF₂, A₂ is N, R₃ is H, X is SO, R₁ is ethyl and Q₁ is as defined in table Z.

Table B-15 provides 21 compounds B-15.001 to B-15.021 of formula I-Qb-1 wherein R₂ is CH₂CF₂CHF₂, A₂ is N, R₃ is H, X is SO₂, R₁ is ethyl and Q₁ is as defined in table Z.

Table B-16 provides 21 compounds B-16.001 to B-16.021 of formula I-Qb-1 wherein R₂ is CH₂CF₂CHF₂, A₂ is N, R₃ is Me, X is S, R₁ is ethyl and Q₁ is as defined in table Z.

Table B-17 provides 21 compounds B-17.001 to B-17.021 of formula I-Qb-1 wherein R₂ is CH₂CF₂CHF₂, A₂ is N, R₃ is Me, X is SO, R₁ is ethyl and Q₁ is as defined in table Z.

Table B-18 provides 21 compounds B-18.001 to B-18.021 of formula I-Qb-1 wherein R₂ is CH₂CF₂CHF₂, A₂ is N, R₃ is Me, X is SO₂, R₁ is ethyl and Q₁ is as defined in table Z.

Table B-19 provides 21 compounds B-19.001 to B-19.021 of formula I-Qb-1 wherein R₂ is CH₂CF₂CHF₂, A₂ is CH, R₃ is H, X is S, R₁ is ethyl and Q₁ is as defined in table Z.

Table B-20 provides 21 compounds B-20.001 to B-20.021 of formula I-Qb-1 wherein R₂ is CH₂CF₂CHF₂, A₂ is CH, R₃ is H, X is SO, R₁ is ethyl and Q₁ is as defined in table Z.

Table B-21 provides 21 compounds B-21.001 to B-21.021 of formula I-Qb-1 wherein R₂ is CH₂CF₂CHF₂, A₂ is CH, R₃ is H, X is SO₂, R₁ is ethyl and Q₁ is as defined in table Z.

Table B-22 provides 21 compounds B-22.001 to B-22.021 of formula I-Qb-1 wherein R₂ is CH₂CF₂CHF₂, A₂ is CH, R₃ is Me, X is S, R₁ is ethyl and Q₁ is as defined in table Z.

Table B-23 provides 21 compounds B-23.001 to B-23.021 of formula I-Qb-1 wherein R₂ is CH₂CF₂CHF₂, A₂ is CH, R₃ is Me, X is SO, R₁ is ethyl and Q₁ is as defined in table Z.

Table B-24 provides 21 compounds B-24.001 to B-24.021 of formula I-Qb-1 wherein R₂ is CH₂CF₂CHF₂, A₂ is CH, R₃ is Me, X is SO₂, R₁ is ethyl and Q₁ is as defined in table Z.

Table B-25 provides 21 compounds B-25.001 to B-25.021 of formula I-Qb-1 wherein R₂ is CH₂CF₃, A₂ is N, R₃ is H, X is S, R₁ is ethyl and Q₁ is as defined in table Z.

Table B-26 provides 21 compounds B-26.001 to B-26.021 of formula I-Qb-1 wherein R₂ is CH₂CF₃, A₂ is N, R₃ is H, X is SO, R₁ is ethyl and Q₁ is as defined in table Z.

Table B-27 provides 21 compounds B-27.001 to B-27.021 of formula I-Qb-1 wherein R₂ is CH₂CF₃, A₂ is N, R₃ is H, X is SO₂, R₁ is ethyl and Q₁ is as defined in table Z.

Table B-28 provides 21 compounds B-28.001 to B-28.021 of formula I-Qb-1 wherein R₂ is CH₂CF₃, A₂ is N, R₃ is Me, X is S, R₁ is ethyl and Q₁ is as defined in table Z.

Table B-29 provides 21 compounds B-29.001 to B-29.021 of formula I-Qb-1 wherein R₂ is CH₂CF₃, A₂ is N, R₃ is Me, X is SO, R₁ is ethyl and Q₁ is as defined in table Z.

Table B-30 provides 21 compounds B-30.001 to B-30.021 of formula I-Qb-1 wherein R₂ is CH₂CF₃, A₂ is N, R₃ is Me, X is SO₂, R₁ is ethyl and Q₁ is as defined in table Z.

Table B-31 provides 21 compounds B-31.001 to B-31.021 of formula I-Qb-1 wherein R₂ is CH₂CF₃, A₂ is CH, R₃ is H, X is S, R₁ is ethyl and Q₁ is as defined in table Z.

Table B-32 provides 21 compounds B-32.001 to B-32.021 of formula I-Qb-1 wherein R₂ is CH₂CF₃, A₂ is CH, R₃ is H, X is SO, R₁ is ethyl and Q₁ is as defined in table Z.

Table B-33 provides 21 compounds B-33.001 to B-33.021 of formula I-Qb-1 wherein R₂ is CH₂CF₃, A₂ is CH, R₃ is H, X is SO₂, R₁ is ethyl and Q₁ is as defined in table Z.

Table B-34 provides 21 compounds B-34.001 to B-34.021 of formula I-Qb-1 wherein R₂ is CH₂CF₃, A₂ is CH, R₃ is Me, X is S, R₁ is ethyl and Q₁ is as defined in table Z.

Table B-35 provides 21 compounds B-35.001 to B-35.021 of formula I-Qb-1 wherein R₂ is CH₂CF₃, A₂ is CH, R₃ is Me, X is SO, R₁ is ethyl and Q₁ is as defined in table Z.

Table B-36 provides 21 compounds B-36.001 to B-36.021 of formula I-Qb-1 wherein R₂ is CH₂CF₃, A₂ is CH, R₃ is Me, X is SO₂, R₁ is ethyl and Q₁ is as defined in table Z.

Table B-37 provides 21 compounds B-37.001 to B-37.021 of formula I-Qb-1 wherein R₂ is CH₂CHF₂, A₂ is N, R₃ is H, X is S, R₁ is ethyl and Q₁ is as defined in table Z.

Table B-38 provides 21 compounds B-38.001 to B-38.021 of formula I-Qb-1 wherein R₂ is CH₂CHF₂, A₂ is N, R₃ is H, X is SO, R₁ is ethyl and Q₁ is as defined in table Z.

Table B-39 provides 21 compounds B-39.001 to B-39.021 of formula I-Qb-1 wherein R₂ is CH₂CHF₂, A₂ is N, R₃ is H, X is SO₂, R₁ is ethyl and Q₁ is as defined in table Z.

Table B-40 provides 21 compounds B-40.001 to B-40.021 of formula I-Qb-1 wherein R₂ is CH₂CHF₂, A₂ is N, R₃ is Me, X is S, R₁ is ethyl and Q₁ is as defined in table Z.

Table B-41 provides 21 compounds B-41.001 to B-41.021 of formula I-Qb-1 wherein R₂ is CH₂CHF₂, A₂ is N, R₃ is Me, X is SO, R₁ is ethyl and Q₁ is as defined in table Z.

Table B-42 provides 21 compounds B-42.001 to B-42.021 of formula I-Qb-1 wherein R₂ is CH₂CHF₂, A₂ is N, R₃ is Me, X is SO₂, R₁ is ethyl and Q₁ is as defined in table Z.

Table B-43 provides 21 compounds B-43.001 to B-43.021 of formula I-Qb-1 wherein R₂ is CH₂CHF₂, A₂ is CH, R₃ is H, X is S, R₁ is ethyl and Q₁ is as defined in table Z.

Table B-44 provides 21 compounds B-44.001 to B-44.021 of formula I-Qb-1 wherein R₂ is CH₂CHF₂, A₂ is CH, R₃ is H, X is SO, R₁ is ethyl and Q₁ is as defined in table Z.

Table B-45 provides 21 compounds B-45.001 to B-45.021 of formula I-Qb-1 wherein R₂ is CH₂CHF₂, A₂ is CH, R₃ is H, X is SO₂, R₁ is ethyl and Q₁ is as defined in table Z.

Table B-46 provides 21 compounds B-46.001 to B-46.021 of formula I-Qb-1 wherein R₂ is CH₂CHF₂, A₂ is CH, R₃ is Me, X is S, R₁ is ethyl and Q₁ is as defined in table Z.

Table B-47 provides 21 compounds B-47.001 to B-47.021 of formula I-Qb-1 wherein R₂ is CH₂CHF₂, A₂ is CH, R₃ is Me, X is SO, R₁ is ethyl and Q₁ is as defined in table Z.

Table B-48 provides 21 compounds B-48.001 to B-48.021 of formula I-Qb-1 wherein R₂ is CH₂CHF₂, A₂ is CH, R₃ is Me, X is SO₂, R₁ is ethyl and Q₁ is as defined in table Z.

Table B-49 provides 21 compounds B-49.001 to B-49.021 of formula I-Qb-1 wherein R₂ is CH₂CF₂CHFCF₃, A₂ is N, R₃ is H, X is S, R₁ is ethyl and Q₁ is as defined in table Z.

Table B-50 provides 21 compounds B-50.001 to B-50.021 of formula I-Qb-1 wherein R₂ is CH₂CF₂CHFCF₃, A₂ is N, R₃ is H, X is SO, R₁ is ethyl and Q₁ is as defined in table Z.

Table B-51 provides 21 compounds B-51.001 to B-51.021 of formula I-Qb-1 wherein R₂ is CH₂CF₂CHFCF₃, A₂ is N, R₃ is H, X is SO₂, R₁ is ethyl and Q₁ is as defined in table Z.

Table B-52 provides 21 compounds B-52.001 to B-52.021 of formula I-Qb-1 wherein R₂ is CH₂CF₂CHFCF₃, A₂ is N, R₃ is Me, X is S, R₁ is ethyl and Q₁ is as defined in table Z.

Table B-53 provides 21 compounds B-53.001 to B-53.021 of formula I-Qb-1 wherein R₂ is CH₂CF₂CHFCF₃, A₂ is N, R₃ is Me, X is SO, R₁ is ethyl and Q₁ is as defined in table Z.

Table B-54 provides 21 compounds B-54.001 to B-54.021 of formula I-Qb-1 wherein R₂ is CH₂CF₂CHFCF₃, A₂ is N, R₃ is Me, X is SO₂, R₁ is ethyl and Q₁ is as defined in table Z.

Table B-55 provides 21 compounds B-55.001 to B-55.021 of formula I-Qb-1 wherein R₂ is CH₂CF₂CHFCF₃, A₂ is CH, R₃ is H, X is S, R₁ is ethyl and Q₁ is as defined in table Z.

Table B-56 provides 21 compounds B-56.001 to B-56.021 of formula I-Qb-1 wherein R₂ is CH₂CF₂CHFCF₃, A₂ is CH, R₃ is H, X is SO, R₁ is ethyl and Q₁ is as defined in table Z.

Table B-57 provides 21 compounds B-57.001 to B-57.021 of formula I-Qb-1 wherein R₂ is CH₂CF₂CHFCF₃, A₂ is CH, R₃ is H, X is SO₂, R₁ is ethyl and Q₁ is as defined in table Z.

Table B-58 provides 21 compounds B-58.001 to B-58.021 of formula I-Qb-1 wherein R₂ is CH₂CF₂CHFCF₃, A₂ is CH, R₃ is Me, X is S, R₁ is ethyl and Q₁ is as defined in table Z.

Table B-59 provides 21 compounds B-59.001 to B-59.021 of formula I-Qb-1 wherein R₂ is CH₂CF₂CHFCF₃, A₂ is CH, R₃ is Me, X is SO, R₁ is ethyl and Q₁ is as defined in table Z.

Table B-60 provides 21 compounds B-60.001 to B-60.021 of formula I-Qb-1 wherein R₂ is CH₂CF₂CHFCF₃, A₂ is CH, R₃ is Me, X is SO₂, R₁ is ethyl and Q₁ is as defined in table Z.

The tables C-1 to C-60 below illustrate further specific compounds of the invention.

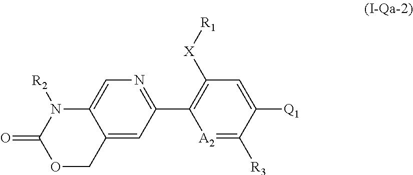

In the table Y and in tables C, “cycloC3” represents cyclopropyl.

Table C-1 provides 23 compounds C-1.001 to C-1.023 of formula I-Qa-2 wherein R₂ is CH₂CF₂CF₃, A₂ is N, R₃ is H, X is S, R₁ is ethyl and Q₁ is as defined in table Y.

Table C-2 provides 23 compounds C-2.001 to C-2.023 of formula I-Qa-2 wherein R₂ is CH₂CF₂CF₃, A₂ is N, R₃ is H, X is SO, R₁ is ethyl and Q₁ is as defined in table Y.

Table C-3 provides 23 compounds C-3.001 to C-3.023 of formula I-Qa-2 wherein R₂ is CH₂CF₂CF₃, A₂ is N, R₃ is H, X is SO₂, R₁ is ethyl and Q₁ is as defined in table Y.

Table C-4 provides 23 compounds C-4.001 to C-4.023 of formula I-Qa-2 wherein R₂ is CH₂CF₂CF₃, A₂ is N, R₃ is Me, X is S, R₁ is ethyl and Q₁ is as defined in table Y.

Table C-5 provides 23 compounds C-5.001 to C-5.023 of formula I-Qa-2 wherein R₂ is CH₂CF₂CF₃, A₂ is N, R₃ is Me, X is SO, R₁ is ethyl and Q₁ is as defined in table Y.

Table C-6 provides 23 compounds C-6.001 to C-6.023 of formula I-Qa-2 wherein R₂ is CH₂CF₂CF₃, A₂ is N, R₃ is Me, X is SO₂, R₁ is ethyl and Q₁ is as defined in table Y.

Table C-7 provides 23 compounds C-7.001 to C-7.023 of formula I-Qa-2 wherein R₂ is CH₂CF₂CF₃, A₂ is CH, R₃ is H, X is S, R₁ is ethyl and Q₁ is as defined in table Y.

Table C-8 provides 23 compounds C-8.001 to C-8.023 of formula I-Qa-2 wherein R₂ is CH₂CF₂CF₃, A₂ is CH, R₃ is H, X is SO, R₁ is ethyl and Q₁ is as defined in table Y.

Table C-9 provides 23 compounds C-9.001 to C-9.023 of formula I-Qa-2 wherein R₂ is CH₂CF₂CF₃, A₂ is CH, R₃ is H, X is SO₂, R₁ is ethyl and Q₁ is as defined in table Y.

Table C-10 provides 23 compounds C-10.001 to C-10.023 of formula I-Qa-2 wherein R₂ is CH₂CF₂CF₃, A₂ is CH, R₃ is Me, X is S, R₁ is ethyl and Q₁ is as defined in table Y.

Table C-11 provides 23 compounds C-11.001 to C-11.023 of formula I-Qa-2 wherein R₂ is CH₂CF₂CF₃, A₂ is CH, R₃ is Me, X is SO, R₁ is ethyl and Q₁ is as defined in table Y.

Table C-12 provides 23 compounds C-12.001 to C-12.023 of formula I-Qa-2 wherein R₂ is CH₂CF₂CF₃, A₂ is CH, R₃ is Me, X is SO₂, R₁ is ethyl and Q₁ is as defined in table Y.

Table C-13 provides 23 compounds C-13.001 to C-13.023 of formula I-Qa-2 wherein R₂ is CH₂CF₂CHF₂, A₂ is N, R₃ is H, X is S, R₁ is ethyl and Q₁ is as defined in table Y.

Table C-14 provides 23 compounds C-14.001 to C-14.023 of formula I-Qa-2 wherein R₂ is CH₂CF₂CHF₂, A₂ is N, R₃ is H, X is SO, R₁ is ethyl and Q₁ is as defined in table Y.

Table C-15 provides 23 compounds C-15.001 to C-15.023 of formula I-Qa-2 wherein R₂ is CH₂CF₂CHF₂, A₂ is N, R₃ is H, X is SO₂, R₁ is ethyl and Q₁ is as defined in table Y.

Table C-16 provides 23 compounds C-16.001 to C-16.023 of formula I-Qa-2 wherein R₂ is CH₂CF₂CHF₂, A₂ is N, R₃ is Me, X is S, R₁ is ethyl and Q₁ is as defined in table Y.

Table C-17 provides 23 compounds C-17.001 to C-17.023 of formula I-Qa-2 wherein R₂ is CH₂CF₂CHF₂, A₂ is N, R₃ is Me, X is SO, R₁ is ethyl and Q₁ is as defined in table Y.

Table C-18 provides 23 compounds C-18.001 to C-18.023 of formula I-Qa-2 wherein R₂ is CH₂CF₂CHF₂, A₂ is N, R₃ is Me, X is SO₂, R₁ is ethyl and Q₁ is as defined in table Y.

Table C-19 provides 23 compounds C-19.001 to C-19.023 of formula I-Qa-2 wherein R₂ is CH₂CF₂CHF₂, A₂ is CH, R₃ is H, X is S, R₁ is ethyl and Q₁ is as defined in table Y.

Table C-20 provides 23 compounds C-20.001 to C-20.023 of formula I-Qa-2 wherein R₂ is CH₂CF₂CHF₂, A₂ is CH, R₃ is H, X is SO, R₁ is ethyl and Q₁ is as defined in table Y.

Table C-21 provides 23 compounds C-21.001 to C-21.023 of formula I-Qa-2 wherein R₂ is CH₂CF₂CHF₂, A₂ is CH, R₃ is H, X is SO₂, R₁ is ethyl and Q₁ is as defined in table Y.

Table C-22 provides 23 compounds C-22.001 to C-22.023 of formula I-Qa-2 wherein R₂ is CH₂CF₂CHF₂, A₂ is CH, R₃ is Me, X is S, R₁ is ethyl and Q₁ is as defined in table Y.

Table C-23 provides 23 compounds C-23.001 to C-23.023 of formula I-Qa-2 wherein R₂ is CH₂CF₂CHF₂, A₂ is CH, R₃ is Me, X is SO, R₁ is ethyl and Q₁ is as defined in table Y.

Table C-24 provides 23 compounds C-24.001 to C-24.023 of formula I-Qa-2 wherein R₂ is CH₂CF₂CHF₂, A₂ is CH, R₃ is Me, X is SO₂, R₁ is ethyl and Q₁ is as defined in table Y.

Table C-25 provides 23 compounds C-25.001 to C-25.023 of formula I-Qa-2 wherein R₂ is CH₂CF₃, A₂ is N, R₃ is H, X is S, R₁ is ethyl and Q₁ is as defined in table Y.

Table C-26 provides 23 compounds C-26.001 to C-26.023 of formula I-Qa-2 wherein R₂ is CH₂CF₃, A₂ is N, R₃ is H, X is SO, R₁ is ethyl and Q₁ is as defined in table Y.

Table C-27 provides 23 compounds C-27.001 to C-27.023 of formula I-Qa-2 wherein R₂ is CH₂CF₃, A₂ is N, R₃ is H, X is SO₂, R₁ is ethyl and Q₁ is as defined in table Y.

Table C-28 provides 23 compounds C-28.001 to C-28.023 of formula I-Qa-2 wherein R₂ is CH₂CF₃, A₂ is N, R₃ is Me, X is S, R₁ is ethyl and Q₁ is as defined in table Y.

Table C-29 provides 23 compounds C-29.001 to C-29.023 of formula I-Qa-2 wherein R₂ is CH₂CF₃, A₂ is N, R₃ is Me, X is SO, R₁ is ethyl and Q₁ is as defined in table Y.

Table C-30 provides 23 compounds C-30.001 to C-30.023 of formula I-Qa-2 wherein R₂ is CH₂CF₃, A₂ is N, R₃ is Me, X is SO₂, R₁ is ethyl and Q₁ is as defined in table Y.

Table C-31 provides 23 compounds C-31.001 to C-31.023 of formula I-Qa-2 wherein R₂ is CH₂CF₃, A₂ is CH, R₃ is H, X is S, R₁ is ethyl and Q₁ is as defined in table Y.

Table C-32 provides 23 compounds C-32.001 to C-32.023 of formula I-Qa-2 wherein R₂ is CH₂CF₃, A₂ is CH, R₃ is H, X is SO, R₁ is ethyl and Q₁ is as defined in table Y.

Table C-33 provides 23 compounds C-33.001 to C-33.023 of formula I-Qa-2 wherein R₂ is CH₂CF₃, A₂ is CH, R₃ is H, X is SO₂, R₁ is ethyl and Q₁ is as defined in table Y.

Table C-34 provides 23 compounds C-34.001 to C-34.023 of formula I-Qa-2 wherein R₂ is CH₂CF₃, A₂ is CH, R₃ is Me, X is S, R₁ is ethyl and Q₁ is as defined in table Y.

Table C-35 provides 23 compounds C-35.001 to C-35.023 of formula I-Qa-2 wherein R₂ is CH₂CF₃, A₂ is CH, R₃ is Me, X is SO, R₁ is ethyl and Q₁ is as defined in table Y.

Table C-36 provides 23 compounds C-36.001 to C-36.023 of formula I-Qa-2 wherein R₂ is CH₂CF₃, A₂ is CH, R₃ is Me, X is SO₂, R₁ is ethyl and Q₁ is as defined in table Y.

Table C-37 provides 23 compounds C-37.001 to C-37.023 of formula I-Qa-2 wherein R₂ is CH₂CHF₂, A₂ is N, R₃ is H, X is S, R₁ is ethyl and Q₁ is as defined in table Y.

Table C-38 provides 23 compounds C-38.001 to C-38.023 of formula I-Qa-2 wherein R₂ is CH₂CHF₂, A₂ is N, R₃ is H, X is SO, R₁ is ethyl and Q₁ is as defined in table Y.

Table C-39 provides 23 compounds C-39.001 to C-39.023 of formula I-Qa-2 wherein R₂ is CH₂CHF₂, A₂ is N, R₃ is H, X is SO₂, R₁ is ethyl and Q₁ is as defined in table Y.

Table C-40 provides 23 compounds C-40.001 to C-40.023 of formula I-Qa-2 wherein R₂ is CH₂CHF₂, A₂ is N, R₃ is Me, X is S, R₁ is ethyl and Q₁ is as defined in table Y.

Table C-41 provides 23 compounds C-41.001 to C-41.023 of formula I-Qa-2 wherein R₂ is CH₂CHF₂, A₂ is N, R₃ is Me, X is SO, R₁ is ethyl and Q₁ is as defined in table Y.

Table C-42 provides 23 compounds C-42.001 to C-42.023 of formula I-Qa-2 wherein R₂ is CH₂CHF₂, A₂ is N, R₃ is Me, X is SO₂, R₁ is ethyl and Q₁ is as defined in table Y.

Table C-43 provides 23 compounds C-43.001 to C-43.023 of formula I-Qa-2 wherein R₂ is CH₂CHF₂, A₂ is CH, R₃ is H, X is S, R₁ is ethyl and Q₁ is as defined in table Y.

Table C-44 provides 23 compounds C-44.001 to C-44.023 of formula I-Qa-2 wherein R₂ is CH₂CHF₂, A₂ is CH, R₃ is H, X is SO, R₁ is ethyl and Q₁ is as defined in table Y.

Table C-45 provides 23 compounds C-45.001 to C-45.023 of formula I-Qa-2 wherein R₂ is CH₂CHF₂, A₂ is CH, R₃ is H, X is SO₂, R₁ is ethyl and Q₁ is as defined in table Y.

Table C-46 provides 23 compounds C-46.001 to C-46.023 of formula I-Qa-2 wherein R₂ is CH₂CHF₂, A₂ is CH, R₃ is Me, X is S, R₁ is ethyl and Q₁ is as defined in table Y.

Table C-47 provides 23 compounds C-47.001 to C-47.023 of formula I-Qa-2 wherein R₂ is CH₂CHF₂, A₂ is CH, R₃ is Me, X is SO, R₁ is ethyl and Q₁ is as defined in table Y.

Table C-48 provides 23 compounds C-48.001 to C-48.023 of formula I-Qa-2 wherein R₂ is CH₂CHF₂, A₂ is CH, R₃ is Me, X is SO₂, R₁ is ethyl and Q₁ is as defined in table Y.

Table C-49 provides 23 compounds C-49.001 to C-49.023 of formula I-Qa-2 wherein R₂ is CH₂CF₂CHFCF₃, A₂ is N, R₃ is H, X is S, R₁ is ethyl and Q₁ is as defined in table Y.

Table C-50 provides 23 compounds C-50.001 to C-50.023 of formula I-Qa-2 wherein R₂ is CH₂CF₂CHFCF₃, A₂ is N, R₃ is H, X is SO, R₁ is ethyl and Q₁ is as defined in table Y.

Table C-51 provides 23 compounds C-51.001 to C-51.023 of formula I-Qa-2 wherein R₂ is CH₂CF₂CHFCF₃, A₂ is N, R₃ is H, X is SO₂, R₁ is ethyl and Q₁ is as defined in table Y.

Table C-52 provides 23 compounds C-52.001 to C-52.023 of formula I-Qa-2 wherein R₂ is CH₂CF₂CHFCF₃, A₂ is N, R₃ is Me, X is S, R₁ is ethyl and Q₁ is as defined in table Y.

Table C-53 provides 23 compounds C-53.001 to C-53.023 of formula I-Qa-2 wherein R₂ is CH₂CF₂CHFCF₃, A₂ is N, R₃ is Me, X is SO, R₁ is ethyl and Q₁ is as defined in table Y.

Table C-54 provides 23 compounds C-54.001 to C-54.023 of formula I-Qa-2 wherein R₂ is CH₂CF₂CHFCF₃, A₂ is N, R₃ is Me, X is SO₂, R₁ is ethyl and Q₁ is as defined in table Y.

Table C-55 provides 23 compounds C-55.001 to C-55.023 of formula I-Qa-2 wherein R₂ is CH₂CF₂CHFCF₃, A₂ is CH, R₃ is H, X is S, R₁ is ethyl and Q₁ is as defined in table Y.

Table C-56 provides 23 compounds C-56.001 to C-56.023 of formula I-Qa-2 wherein R₂ is CH₂CF₂CHFCF₃, A₂ is CH, R₃ is H, X is SO, R₁ is ethyl and Q₁ is as defined in table Y.

Table C-57 provides 23 compounds C-57.001 to C-57.023 of formula I-Qa-2 wherein R₂ is CH₂CF₂CHFCF₃, A₂ is CH, R₃ is H, X is SO₂, R₁ is ethyl and Q₁ is as defined in table Y.

Table C-58 provides 23 compounds C-58.001 to C-58.023 of formula I-Qa-2 wherein R₂ is CH₂CF₂CHFCF₃, A₂ is CH, R₃ is Me, X is S, R₁ is ethyl and Q₁ is as defined in table Y.

Table C-59 provides 23 compounds C-59.001 to C-59.023 of formula I-Qa-2 wherein R₂ is CH₂CF₂CHFCF₃, A₂ is CH, R₃ is Me, X is SO, R₁ is ethyl and Q₁ is as defined in table Y.

Table C-60 provides 23 compounds C-60.001 to C-60.023 of formula I-Qa-2 wherein R₂ is CH₂CF₂CHFCF₃, A₂ is CH, R₃ is Me, X is S02, R₁ is ethyl and Q₁ is as defined in table Y.

The tables D-1 to D-60 below illustrate further specific compounds of the invention.

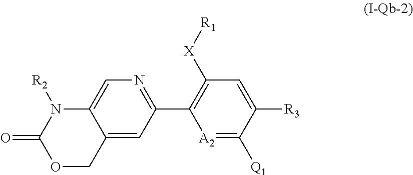

In the table Z and in tables D, “cycloC3” represents cyclopropyl.

Table D-1 provides 21 compounds D-1.001 to D-1.021 of formula I-Qb-2 wherein R₂ is CH₂CF₂CF₃, A₂ is N, R₃ is H, X is S, R₁ is ethyl and Q₁ is as defined in table Z.

Table D-2 provides 21 compounds D-2.001 to D-2.021 of formula I-Qb-2 wherein R₂ is CH₂CF₂CF₃, A₂ is N, R₃ is H, X is SO, R₁ is ethyl and Q₁ is as defined in table Z.

Table D-3 provides 21 compounds D-3.001 to D-3.021 of formula I-Qb-2 wherein R₂ is CH₂CF₂CF₃, A₂ is N, R₃ is H, X is SO₂, R₁ is ethyl and Q₁ is as defined in table Z.

Table D-4 provides 21 compounds D-4.001 to D-4.021 of formula I-Qb-2 wherein R₂ is CH₂CF₂CF₃, A₂ is N, R₃ is Me, X is S, R₁ is ethyl and Q₁ is as defined in table Z.

Table D-5 provides 21 compounds D-5.001 to D-5.021 of formula I-Qb-2 wherein R₂ is CH₂CF₂CF₃, A₂ is N, R₃ is Me, X is SO, R₁ is ethyl and Q₁ is as defined in table Z.

Table D-6 provides 21 compounds D-6.001 to D-6.021 of formula I-Qb-2 wherein R₂ is CH₂CF₂CF₃, A₂ is N, R₃ is Me, X is SO₂, R₁ is ethyl and Q₁ is as defined in table Z.

Table D-7 provides 21 compounds D-7.001 to D-7.021 of formula I-Qb-2 wherein R₂ is CH₂CF₂CF₃, A₂ is CH, R₃ is H, X is S, R₁ is ethyl and Q₁ is as defined in table Z.

Table D-8 provides 21 compounds D-8.001 to D-8.021 of formula I-Qb-2 wherein R₂ is CH₂CF₂CF₃, A₂ is CH, R₃ is H, X is SO, R₁ is ethyl and Q₁ is as defined in table Z.

Table D-9 provides 21 compounds D-9.001 to D-9.021 of formula I-Qb-2 wherein R₂ is CH₂CF₂CF₃, A₂ is CH, R₃ is H, X is SO₂, R₁ is ethyl and Q₁ is as defined in table Z.

Table D-10 provides 21 compounds D-10.001 to D-10.021 of formula I-Qb-2 wherein R₂ is CH₂CF₂CF₃, A₂ is CH, R₃ is Me, X is S, R₁ is ethyl and Q₁ is as defined in table Z.

Table D-11 provides 21 compounds D-11.001 to D-11.021 of formula I-Qb-2 wherein R₂ is CH₂CF₂CF₃, A₂ is CH, R₃ is Me, X is SO, R₁ is ethyl and Q₁ is as defined in table Z.

Table D-12 provides 21 compounds D-12.001 to D-12.021 of formula I-Qb-2 wherein R₂ is CH₂CF₂CF₃, A₂ is CH, R₃ is Me, X is SO₂, R₁ is ethyl and Q₁ is as defined in table Z.

Table D-13 provides 21 compounds D-13.001 to D-13.021 of formula I-Qb-2 wherein R₂ is CH₂CF₂CHF₂, A₂ is N, R₃ is H, X is S, R₁ is ethyl and Q₁ is as defined in table Z.

Table D-14 provides 21 compounds D-14.001 to D-14.021 of formula I-Qb-2 wherein R₂ is CH₂CF₂CHF₂, A₂ is N, R₃ is H, X is SO, R₁ is ethyl and Q₁ is as defined in table Z.

Table D-15 provides 21 compounds D-15.001 to D-15.021 of formula I-Qb-2 wherein R₂ is CH₂CF₂CHF₂, A₂ is N, R₃ is H, X is SO₂, R₁ is ethyl and Q₁ is as defined in table Z.

Table D-16 provides 21 compounds D-16.001 to D-16.021 of formula I-Qb-2 wherein R₂ is CH₂CF₂CHF₂, A₂ is N, R₃ is Me, X is S, R₁ is ethyl and Q₁ is as defined in table Z.

Table D-17 provides 21 compounds D-17.001 to D-17.021 of formula I-Qb-2 wherein R₂ is CH₂CF₂CHF₂, A₂ is N, R₃ is Me, X is SO, R₁ is ethyl and Q₁ is as defined in table Z.

Table D-18 provides 21 compounds D-18.001 to D-18.021 of formula I-Qb-2 wherein R₂ is CH₂CF₂CHF₂, A₂ is N, R₃ is Me, X is SO₂, R₁ is ethyl and Q₁ is as defined in table Z.

Table D-19 provides 21 compounds D-19.001 to D-19.021 of formula I-Qb-2 wherein R₂ is CH₂CF₂CHF₂, A₂ is CH, R₃ is H, X is S, R₁ is ethyl and Q₁ is as defined in table Z.

Table D-20 provides 21 compounds D-20.001 to D-20.021 of formula I-Qb-2 wherein R₂ is CH₂CF₂CHF₂, A₂ is CH, R₃ is H, X is SO, R₁ is ethyl and Q₁ is as defined in table Z.

Table D-21 provides 21 compounds D-21.001 to D-21.021 of formula I-Qb-2 wherein R₂ is CH₂CF₂CHF₂, A₂ is CH, R₃ is H, X is SO₂, R₁ is ethyl and Q₁ is as defined in table Z.

Table D-22 provides 21 compounds D-22.001 to D-22.021 of formula I-Qb-2 wherein R₂ is CH₂CF₂CHF₂, A₂ is CH, R₃ is Me, X is S, R₁ is ethyl and Q₁ is as defined in table Z.

Table D-23 provides 21 compounds D-23.001 to D-23.021 of formula I-Qb-2 wherein R₂ is CH₂CF₂CHF₂, A₂ is CH, R₃ is Me, X is SO, R₁ is ethyl and Q₁ is as defined in table Z.

Table D-24 provides 21 compounds D-24.001 to D-24.021 of formula I-Qb-2 wherein R₂ is CH₂CF₂CHF₂, A₂ is CH, R₃ is Me, X is SO₂, R₁ is ethyl and Q₁ is as defined in table Z.

Table D-25 provides 21 compounds D-25.001 to D-25.021 of formula I-Qb-2 wherein R₂ is CH₂CF₃, A₂ is N, R₃ is H, X is S, R₁ is ethyl and Q₁ is as defined in table Z.

Table D-26 provides 21 compounds D-26.001 to D-26.021 of formula I-Qb-2 wherein R₂ is CH₂CF₃, A₂ is N, R₃ is H, X is SO, R₁ is ethyl and Q₁ is as defined in table Z.

Table D-27 provides 21 compounds D-27.001 to D-27.021 of formula I-Qb-2 wherein R₂ is CH₂CF₃, A₂ is N, R₃ is H, X is SO₂, R₁ is ethyl and Q₁ is as defined in table Z.

Table D-28 provides 21 compounds D-28.001 to D-28.021 of formula I-Qb-2 wherein R₂ is CH₂CF₃, A₂ is N, R₃ is Me, X is S, R₁ is ethyl and Q₁ is as defined in table Z.

Table D-29 provides 21 compounds D-29.001 to D-29.021 of formula I-Qb-2 wherein R₂ is CH₂CF₃, A₂ is N, R₃ is Me, X is SO, R₁ is ethyl and Q₁ is as defined in table Z.

Table D-30 provides 21 compounds D-30.001 to D-30.021 of formula I-Qb-2 wherein R₂ is CH₂CF₃, A₂ is N, R₃ is Me, X is SO₂, R₁ is ethyl and Q₁ is as defined in table Z.

Table D-31 provides 21 compounds D-31.001 to D-31.021 of formula I-Qb-2 wherein R₂ is CH₂CF₃, A₂ is CH, R₃ is H, X is S, R₁ is ethyl and Q₁ is as defined in table Z.

Table D-32 provides 21 compounds D-32.001 to D-32.021 of formula I-Qb-2 wherein R₂ is CH₂CF₃, A₂ is CH, R₃ is H, X is SO, R₁ is ethyl and Q₁ is as defined in table Z.

Table D-33 provides 21 compounds D-33.001 to D-33.021 of formula I-Qb-2 wherein R₂ is CH₂CF₃, A₂ is CH, R₃ is H, X is SO₂, R₁ is ethyl and Q₁ is as defined in table Z.

Table D-34 provides 21 compounds D-34.001 to D-34.021 of formula I-Qb-2 wherein R₂ is CH₂CF₃, A₂ is CH, R₃ is Me, X is S, R₁ is ethyl and Q₁ is as defined in table Z.

Table D-35 provides 21 compounds D-35.001 to D-35.021 of formula I-Qb-2 wherein R₂ is CH₂CF₃, A₂ is CH, R₃ is Me, X is SO, R₁ is ethyl and Q₁ is as defined in table Z.

Table D-36 provides 21 compounds D-36.001 to D-36.021 of formula I-Qb-2 wherein R₂ is CH₂CF₃, A₂ is CH, R₃ is Me, X is SO₂, R₁ is ethyl and Q₁ is as defined in table Z.

Table D-37 provides 21 compounds D-37.001 to D-37.021 of formula I-Qb-2 wherein R₂ is CH₂CHF₂, A₂ is N, R₃ is H, X is S, R₁ is ethyl and Q₁ is as defined in table Z.

Table D-38 provides 21 compounds D-38.001 to D-38.021 of formula I-Qb-2 wherein R₂ is CH₂CHF₂, A₂ is N, R₃ is H, X is SO, R₁ is ethyl and Q₁ is as defined in table Z.

Table D-39 provides 21 compounds D-39.001 to D-39.021 of formula I-Qb-2 wherein R₂ is CH₂CHF₂, A₂ is N, R₃ is H, X is SO₂, R₁ is ethyl and Q₁ is as defined in table Z.

Table D-40 provides 21 compounds D-40.001 to D-40.021 of formula I-Qb-2 wherein R₂ is CH₂CHF₂, A₂ is N, R₃ is Me, X is S, R₁ is ethyl and Q₁ is as defined in table Z.

Table D-41 provides 21 compounds D-41.001 to D-41.021 of formula I-Qb-2 wherein R₂ is CH₂CHF₂, A₂ is N, R₃ is Me, X is SO, R₁ is ethyl and Q₁ is as defined in table Z.

Table D-42 provides 21 compounds D-42.001 to D-42.021 of formula I-Qb-2 wherein R₂ is CH₂CHF₂, A₂ is N, R₃ is Me, X is SO₂, R₁ is ethyl and Q₁ is as defined in table Z.

Table D-43 provides 21 compounds D-43.001 to D-43.021 of formula I-Qb-2 wherein R₂ is CH₂CHF₂, A₂ is CH, R₃ is H, X is S, R₁ is ethyl and Q₁ is as defined in table Z.

Table D-44 provides 21 compounds D-44.001 to D-44.021 of formula I-Qb-2 wherein R₂ is CH₂CHF₂, A₂ is CH, R₃ is H, X is SO, R₁ is ethyl and Q₁ is as defined in table Z.

Table D-45 provides 21 compounds D-45.001 to D-45.021 of formula I-Qb-2 wherein R₂ is CH₂CHF₂, A₂ is CH, R₃ is H, X is SO₂, R₁ is ethyl and Q₁ is as defined in table Z.

Table D-46 provides 21 compounds D-46.001 to D-46.021 of formula I-Qb-2 wherein R₂ is CH₂CHF₂, A₂ is CH, R₃ is Me, X is S, R₁ is ethyl and Q₁ is as defined in table Z.

Table D-47 provides 21 compounds D-47.001 to D-47.021 of formula I-Qb-2 wherein R₂ is CH₂CHF₂, A₂ is CH, R₃ is Me, X is SO, R₁ is ethyl and Q₁ is as defined in table Z.

Table D-48 provides 21 compounds D-48.001 to D-48.021 of formula I-Qb-2 wherein R₂ is CH₂CHF₂, A₂ is CH, R₃ is Me, X is SO₂, R₁ is ethyl and Q₁ is as defined in table Z.

Table D-49 provides 21 compounds D-49.001 to D-49.021 of formula I-Qb-2 wherein R₂ is CH₂CF₂CHFCF₃, A₂ is N, R₃ is H, X is S, R₁ is ethyl and Q₁ is as defined in table Z.

Table D-50 provides 21 compounds D-50.001 to D-50.021 of formula I-Qb-2 wherein R₂ is CH₂CF₂CHFCF₃, A₂ is N, R₃ is H, X is SO, R₁ is ethyl and Q₁ is as defined in table Z.

Table D-51 provides 21 compounds D-51.001 to D-51.021 of formula I-Qb-2 wherein R₂ is CH₂CF₂CHFCF₃, A₂ is N, R₃ is H, X is SO₂, R₁ is ethyl and Q₁ is as defined in table Z.

Table D-52 provides 21 compounds D-52.001 to D-52.021 of formula I-Qb-2 wherein R₂ is CH₂CF₂CHFCF₃, A₂ is N, R₃ is Me, X is S, R₁ is ethyl and Q₁ is as defined in table Z.

Table D-53 provides 21 compounds D-53.001 to D-53.021 of formula I-Qb-2 wherein R₂ is CH₂CF₂CHFCF₃, A₂ is N, R₃ is Me, X is SO, R₁ is ethyl and Q₁ is as defined in table Z.

Table D-54 provides 21 compounds D-54.001 to D-54.021 of formula I-Qb-2 wherein R₂ is CH₂CF₂CHFCF₃, A₂ is N, R₃ is Me, X is SO₂, R₁ is ethyl and Q₁ is as defined in table Z.

Table D-55 provides 21 compounds D-55.001 to D-55.021 of formula I-Qb-2 wherein R₂ is CH₂CF₂CHFCF₃, A₂ is CH, R₃ is H, X is S, R₁ is ethyl and Q₁ is as defined in table Z.

Table D-56 provides 21 compounds D-56.001 to D-56.021 of formula I-Qb-2 wherein R₂ is CH₂CF₂CHFCF₃, A₂ is CH, R₃ is H, X is SO, R₁ is ethyl and Q₁ is as defined in table Z.

Table D-57 provides 21 compounds D-57.001 to D-57.021 of formula I-Qb-2 wherein R₂ is CH₂CF₂CHFCF₃, A₂ is CH, R₃ is H, X is SO₂, R₁ is ethyl and Q₁ is as defined in table Z.

Table D-58 provides 21 compounds D-58.001 to D-58.021 of formula I-Qb-2 wherein R₂ is CH₂CF₂CHFCF₃, A₂ is CH, R₃ is Me, X is S, R₁ is ethyl and Q₁ is as defined in table Z.

Table D-59 provides 21 compounds D-59.001 to D-59.021 of formula I-Qb-2 wherein R₂ is CH₂CF₂CHFCF₃, A₂ is CH, R₃ is Me, X is SO, R₁ is ethyl and Q₁ is as defined in table Z.

Table D-60 provides 21 compounds D-60.001 to D-60.021 of formula I-Qb-2 wherein R₂ is CH₂CF₂CHFCF₃, A₂ is CH, R₃ is Me, X is SO₂, R₁ is ethyl and Q₁ is as defined in table Z.

The compounds of formula I according to the invention are preventively and/or curatively valuable active ingredients in the field of pest control, even at low rates of application, which have a very favorable biocidal spectrum and are well tolerated by warm-blooded species, fish and plants. The active ingredients according to the invention act against all or individual developmental stages of normally sensitive, but also resistant, animal pests, such as insects or representatives of the order Acarina. The insecticidal or acaricidal activity of the active ingredients according to the invention can manifest itself directly, i. e. in destruction of the pests, which takes place either immediately or only after some time has elapsed, for example during ecdysis, or indirectly, for example in a reduced oviposition and/or hatching rate, a good activity corresponding to a destruction rate (mortality) of at least 50 to 60%.

Examples of the above-mentioned animal pests are:
from the order Acarina, for example,
Acalitus spp, Aculus spp, Acaricalus spp, Aceria spp, Acarus siro, Amblyomma spp., Argas spp., Boophilus spp., Brevipalpus spp., Bryobia spp, Calipitrimerus spp., Chorioptes spp., Dermanyssus gallinae, Dermatophagoides spp, Eotetranychus spp, Eriophyes spp., Hemitarsonemus spp, Hyalomma spp., Ixodes spp., Olygonychus spp, Ornithodoros spp., Polyphagotarsone latus, Panonychus spp., Phyllocoptruta oleivora, Phytonemus spp, Polyphagotarsonemus spp, Psoroptes spp., Rhipicephalus spp., Rhizoglyphus spp., Sarcoptes spp., Steneotarsonemus spp, Tarsonemus spp. and Tetranychus spp.;
from the order Anoplura, for example,
Haematopinus spp., Linognathus spp., Pediculus spp., Pemphigus spp. and Phylloxera spp.;
from the order Coleoptera, for example,
Agriotes spp., Amphimallon majale, Anomala orientalis, Anthonomus spp., Aphodius spp, Astylus atromaculatus, Ataenius spp, Atomaria linearis, Chaetocnema tibialis, Cerotoma spp, Conoderus spp, Cosmopolites spp., Cotinis nitida, Curculio spp., Cyclocephala spp, Dermestes spp., Diabrotica spp., Diloboderus abderus, Epilachna spp., Eremnus spp., Heteronychus arator, Hypothenemus hampei, Lagria vilosa, Leptinotarsa decemLineata, Lissorhoptrus spp., Liogenys spp, Maecolaspis spp, Maladera castanea, Megascelis spp, Melighetes aeneus, Melolontha spp., Myochrous armatus, Orycaephilus spp., Otiorhynchus spp., Phyllophaga spp, Phlyctinus spp., Popillia spp., Psylliodes spp., Rhyssomatus aubtilis, Rhizopertha spp., Scarabeidae, Sitophilus spp., Sitotroga spp., Somaticus spp, Sphenophorus spp, Sternechus subsignatus, Tenebrio spp., Tribolium spp. and Trogoderma spp.;
from the order Diptera, for example,
Aedes spp., Anopheles spp, Antherigona soccata, Bactrocea oleae, Bibio hortulanus, Bradysia spp, Calliphora erythrocephala, Ceratitis spp., Chrysomyia spp., Culex spp., Cuterebra spp., Dacus spp., Delia spp, Drosophila melanogaster, Fannia spp., Gastrophilus spp., Geomyza tripunctata, Glossina spp., Hypoderma spp., Hyppobosca spp., Liriomyza spp., Lucilia spp., Melanagromyza spp., Musca spp., Oestrus spp., Orseolia spp., Oscinella frit, Pegomyia hyoscyami, Phorbia spp., Rhagoletis spp, Rivelia quadrifasciata, Scatella spp, Sciara spp., Stomoxys spp., Tabanus spp., Tannia spp. and Tipula spp.;
from the order Hemiptera, for example,
Acanthocoris scabrator, Acrosternum spp, Adelphocoris lineolatus, Amblypelta nitida, Bathycoelia thalassina, Blissus spp, Cimex spp., Clavigralla tomentosicollis, Creontiades spp, Distantiella theobroma, Dichelops furcatus, Dysdercus spp., Edessa spp, Euschistus spp., Eurydema pulchrum, Eurygaster spp., Halyomorpha halys, Horcias nobilellus, Leptocorisa spp., Lygus spp, Margarodes spp, Murgantia histrionic, Neomegalotomus spp, Nesidiocoris tenuis, Nezara spp., Nysius simulans, Oebalus insularis, Piesma spp., Piezodorus spp, Rhodnius spp., Sahlbergella singularis, Scaptocoris castanea, Scotinophara spp., Thyanta spp, Triatoma spp., Vatiga illudens; Acyrthosium pisum, Adalges spp, Agalliana ensigera, Agonoscena targionii, Aleurodicus spp, Aleurocanthus spp, Aleurolobus barodensis, Aleurothrixus floccosus, Aleyrodes brassicae, Amarasca biguttula, Amritodus atkinsoni, Aonidiella spp., Aphididae, Aphis spp., Aspidiotus spp., Aulacorthum solani, Bactericera cockerelli, Bemisia spp, Brachycaudus spp, Brevicoryne brassicae, Cacopsylla spp, Cavariella aegopodii Scop., Ceroplaster spp., Chrysomphalus aonidium, Chrysomphalus dictyospermi, Cicadella spp, Cofana spectra, Cryptomyzus spp, Cicadulina spp, Coccus hesperidum, Dalbulus maidis, Dialeurodes spp, Diaphorina citri, Diuraphis noxia, Dysaphis spp, Empoasca spp., Eriosoma larigerum, Erythroneura spp., Gascardia spp., Glycaspis brimblecombei, Hyadaphis pseudobrassicae, Hyalopterus spp, Hyperomyzus pallidus, Idioscopus clypealis, Jacobiasca lybica, Laodelphax spp., Lecanium corni, Lepidosaphes spp., Lopaphis erysimi, Lyogenys maidis, Macrosiphum spp., Mahanarva spp, Metcalfa pruinosa, Metopolophium dirhodum, Myndus crudus, Myzus spp., Neotoxoptera sp, Nephotettix spp., Nilaparvata spp., Nippolachnus piri Mats, Odonaspis ruthae, Oregma lanigera Zehnter, Parabemisia myricae, Paratrioza cockerelli, Parlatoria spp., Pemphigus spp., Peregrinus maidis, Perkinsiella spp, Phorodon humuli, Phylloxera spp, Planococcus spp., Pseudaulacaspis spp., Pseudococcus spp., Pseudatomoscelis seriatus, Psylla spp., Pulvinaria aethiopica, Quadraspidiotus spp., Quesada gigas, Recilia dorsalis, Rhopalosiphum spp., Saissetia spp., Scaphoideus spp., Schizaphis spp., Sitobion spp., Sogatella furcifera, Spissistilus festinus, Tarophagus Proserpina, Toxoptera spp, Trialeurodes spp, Tridiscus sporoboli, Trionymus spp, Trioza erytreae, Unaspis citri, Zygina flammigera, Zyginidia scutellaris,
from the order Hymenoptera, for example,
Acromyrmex, Arge spp, Atta spp., Cephus spp., Diprion spp., Diprionidae, Gilpinia polytoma, Hoplo-campa spp., Lasius spp., Monomorium pharaonis, Neodiprion spp., Pogonomyrmex spp, Slenopsis invicta, Solenopsis spp. and Vespa spp.;
from the order Isoptera, for example,
Coptotermes spp, Corniternes cumulans, Incisitermes spp, Macrotermes spp, Mastotermes spp, Microtermes spp, Reticulitermes spp.; Solenopsis geminate
from the order Lepidoptera, for example,
Acleris spp., Adoxophyes spp., Aegeria spp., Agrotis spp., Alabama argillaceae, Amylois spp., Anticarsia gemmatalis, Archips spp., Argyresthia spp, Argyrotaenia spp., Autographa spp., Bucculatrix thurberiella, Busseola fusca, Cadra cautella, Carposina nipponensis, Chilo spp., Choristoneura spp., Chrysoteuchia topiaria, Clysia ambiguella, Cnaphalocrocis spp., Cnephasia spp., Cochylis spp., Coleophora spp., Colias lesbia, Cosmophila flava, Crambus spp, Crocidolomia binotalis, Cryptophlebia leucotreta, Cydalima perspectalis, Cydia spp., Diaphania perspectalis, Diatraea spp., Diparopsis castanea, Earias spp., Eldana saccharina, Ephestia spp., Epinotia spp, Estigmene acrea, Etiella zinckinella, Eucosma spp., Eupoecilia ambiguella, Euproctis spp., Euxoa spp., Feltia jaculiferia, Grapholita spp., Hedya nubiferana, Heliothis spp., Hellula undalis, Herpetogramma spp, Hyphantria cunea, Keiferia lycopersicella, Lasmopalpus lignosellus, Leucoptera scitella, Lithocollethis spp., Lobesia botrana, Loxostege bifidalis, Lymantria spp., Lyonetia spp., Malacosoma spp., Mamestra brassicae, Manduca sexta, Mythimna spp, Noctua spp, Operophtera spp., Orniodes indica, Ostrinia nubilalis, Pammene spp., Pandemis spp., Panolis flammea, Papaipema nebris, Pectinophora gossypiela, Perileucoptera coffeella, Pseudaletia unipuncta, Phthorimaea operculella, Pieris rapae, Pieris spp., Plutella xylostella, Prays spp., Pseudoplusia spp, Rachiplusia nu, Richia albicosta, Scirpophaga spp., Sesamia spp., Sparganothis spp., Spodoptera spp., Sylepta derogate, Synanthedon spp., Thaumetopoea spp., Tortrix spp., Trichoplusia ni, Tuta absoluta, and Yponomeuta spp.;
from the order Mallophaga, for example,
Damalinea spp. and Trichodectes spp.;
from the order Orthoptera, for example,
Blatta spp., Blattella spp., Gryllotalpa spp., Leucophaea maderae, Locusta spp., Neocurtilla hexadactyla, Periplaneta spp. , Scapteriscus spp, and Schistocerca spp.;
from the order Psocoptera, for example,
Liposcelis spp.;
from the order Siphonaptera, for example,
Ceratophyllus spp., Ctenocephalides spp. and Xenopsylla cheopis;
from the order Thysanoptera, for example,
Calliothrips phaseoli, Frankliniella spp., Heliothrips spp, Hercinothrips spp., Parthenothrips spp, Scirtothrips aurantii, Sericothrips variabilis, Taeniothrips spp., Thrips spp;
from the order Thysanura, for example, Lepisma saccharina.

The active ingredients according to the invention can be used for controlling, i. e. containing or destroying, pests of the abovementioned type which occur in particular on plants, especially on useful plants and ornamentals in agriculture, in horticulture and in forests, or on organs, such as fruits, flowers, foliage, stalks, tubers or roots, of such plants, and in some cases even plant organs which are formed at a later point in time remain protected against these pests.

Suitable target crops are, in particular, cereals, such as wheat, barley, rye, oats, rice, maize or sorghum; beet, such as sugar or fodder beet; fruit, for example pomaceous fruit, stone fruit or soft fruit, such as apples, pears, plums, peaches, almonds, cherries or berries, for example strawberries, raspberries or blackberries; leguminous crops, such as beans, lentils, peas or soya; oil crops, such as oilseed rape, mustard, poppies, olives, sunflowers, coconut, castor, cocoa or ground nuts; cucurbits, such as pumpkins, cucumbers or melons; fibre plants, such as cotton, flax, hemp or jute; citrus fruit, such as oranges, lemons, grapefruit or tangerines; vegetables, such as spinach, lettuce, asparagus, cabbages, carrots, onions, tomatoes, potatoes or bell peppers; Lauraceae, such as avocado, Cinnamonium or camphor; and also tobacco, nuts, coffee, eggplants, sugarcane, tea, pepper, grapevines, hops, the plantain family and latex plants.

The compositions and/or methods of the present invention may be also used on any ornamental and/or vegetable crops, including flowers, shrubs, broad-leaved trees and evergreens.

For example the invention may be used on any of the following ornamental species: Ageratum spp., Alonsoa spp., Anemone spp., Anisodontea capsenisis, Anthemis spp., Antirrhinum spp., Aster spp., Begonia spp. (e.g. B. elatior, B. semperflorens, B. tubereux), Bougainvillea spp., Brachycome spp., Brassica spp. (ornamental), Calceolaria spp., Capsicum annuum, Catharanthus roseus, Canna spp., Centaurea spp., Chrysanthemum spp., Cineraria spp. (C. maritime), Coreopsis spp., Crassula coccinea, Cuphea ignea, Dahlia spp., Delphinium spp., Dicentra spectabilis, Dorotheantus spp., Eustoma grandiflorum, Forsythia spp., Fuchsia spp., Geranium gnaphalium, Gerbera spp., Gomphrena globosa, Heliotropium spp., Helianthus spp., Hibiscus spp., Hortensia spp., Hydrangea spp., Hypoestes phyllostachya, Impatiens spp. (I. Walleriana), Iresines spp., Kalanchoe spp., Lantana camara, Lavatera trimestris, Leonotis leonurus, Lilium spp., Mesembryanthemum spp., Mimulus spp., Monarda spp., Nemesia spp., Tagetes spp., Dianthus spp. (carnation), Canna spp., Oxalis spp., Bellis spp., Pelargonium spp. (P. peltatum, P. Zonale), Viola spp. (pansy), Petunia spp., Phlox spp., Plecthranthus spp., Poinsettia spp., Parthenocissus spp. (P. quinquefolia, P. tricuspidata), Primula spp., Ranunculus spp., Rhododendron spp., Rosa spp. (rose), Rudbeckia spp., Saintpaulia spp., Salvia spp., Scaevola aemola, Schizanthus wisetonensis, Sedum spp., Solanum spp., Surfinia spp., Tagetes spp., Nicotinia spp., Verbena spp., Zinnia spp. and other bedding plants.

For example the invention may be used on any of the following vegetable species: Allium spp. (A. sativum, A. cepa, A. oschaninii, A. Porrum, A. ascalonicum, A. fistulosum), Anthriscus cerefolium, Apium graveolus, Asparagus officinalis, Beta vulgarus, Brassica spp. (B. Oleracea, B. Pekinensis, B. rapa), Capsicum annuum, Cicer arietinum, Cichorium endivia, Cichorum spp. (C. intybus, C. endivia), Citrillus lanatus, Cucumis spp. (C. sativus, C. melo), Cucurbita spp. (C. pepo, C. maxima), Cyanara spp. (C. scolymus, C. cardunculus), Daucus carota, Foeniculum vulgare, Hypericum spp., Lactuca sativa, Lycopersicon spp. (L. esculentum, L. lycopersicum), Mentha spp., Ocimum basilicum, Petroselinum crispum, Phaseolus spp. (P. vulgaris, P. coccineus), Pisum sativum, Raphanus sativus, Rheum rhaponticum, Rosemarinus spp., Salvia spp., Scorzonera hispanica, Solanum melongena, Spinacea oleracea, Valerianella spp. (V. locusta, V. eriocarpa) and Vicia faba.

Preferred ornamental species include African violet, Begonia, Dahlia, Gerbera, Hydrangea, Verbena, Rosa, Kalanchoe, Poinsettia, Aster, Centaurea, Coreopsis, Delphinium, Monarda, Phlox, Rudbeckia, Sedum, Petunia, Viola, Impatiens, Geranium, Chrysanthemum, Ranunculus, Fuchsia, Salvia, Hortensia, rosemary, sage, St. Johnswort, mint, sweet pepper, tomato and cucumber.

The active ingredients according to the invention are especially suitable for controlling Aphis craccivora, Diabrotica balteata, Heliothis virescens, Myzus persicae, Plutella xylostella and Spodoptera littoralis in cotton, vegetable, maize, rice and soya crops. The active ingredients according to the invention are further especially suitable for controlling Mamestra (preferably in vegetables), Cydia pomonella (preferably in apples), Empoasca (preferably in vegetables, vineyards), Leptinotarsa (preferably in potatos) and Chilo supressalis (preferably in rice).

The active ingredients according to the invention are especially suitable for controlling Aphis craccivora, Diabrotica balteata, Heliothis virescens, Myzus persicae, Plutella xylostella and Spodoptera littoralis in cotton, vegetable, maize, rice and soya crops. The active ingredients according to the invention are further especially suitable for controlling Mamestra (preferably in vegetables), Cydia pomonella (preferably in apples), Empoasca (preferably in vegetables, vineyards), Leptinotarsa (preferably in potatos) and Chilo supressalis (preferably in rice).

In a further aspect, the invention may also relate to a method of controlling damage to plant and parts thereof by plant parasitic nematodes (Endoparasitic-, Semiendoparasitic- and Ectoparasitic nematodes), especially plant parasitic nematodes such as root knot nematodes, Meloidogyne hapla, Meloidogyne incognita, Meloidogyne javanica, Meloidogyne arenaria and other Meloidogyne species; cyst-forming nematodes, Globodera rostochiensis and other Globodera species; Heterodera avenae, Heterodera glycines, Heterodera schachtii, Heterodera trifolii, and other Heterodera species; Seed gall nematodes, Anguina species; Stem and foliar nematodes, Aphelenchoides species; Sting nematodes, Belonolaimus longicaudatus and other Belonolaimus species; Pine nematodes, Bursaphelenchus xylophilus and other Bursaphelenchus species; Ring nematodes, Criconema species, Criconemella species, Criconemoides species, Mesocriconema species; Stem and bulb nematodes, Ditylenchus destructor, Ditylenchus dipsaci and other Ditylenchus species; Awl nematodes, Dolichodorus species; Spiral nematodes, Heliocotylenchus multicinctus and other Helicotylenchus species; Sheath and sheathoid nematodes, Hemicycliophora species and Hemicriconemoides species; Hirshmanniella species; Lance nematodes, Hoploaimus species; false rootknot nematodes, Nacobbus species; Needle nematodes, Longidorus elongatus and other Longidorus species; Pin nematodes, Pratylenchus species; Lesion nematodes, Pratylenchus neglectus, Pratylenchus penetrans, Pratylenchus curvitatus, Pratylenchus goodeyi and other Pratylenchus species; Burrowing nematodes, Radopholus similis and other Radopholus species; Reniform nematodes, Rotylenchus robustus, Rotylenchus reniformis and other Rotylenchus species; Scutellonema species; Stubby root nematodes, Trichodorus primitivus and other Trichodorus species, Paratrichodorus species; Stunt nematodes, Tylenchorhynchus claytoni, Tylenchorhynchus dubius and other Tylenchorhynchus species; Citrus nematodes, Tylenchulus species; Dagger nematodes, Xiphinema species; and other plant parasitic nematode species, such as Subanguina spp., Hypsoperine spp., Macroposthonia spp., Melinius spp., Punctodera spp., and Quinisulcius spp.

The compounds of the invention may also have activity against the molluscs. Examples of which include, for example, Ampullariidae; Arion (A. ater, A. circumscriptus, A. hortensis, A. rufus); Bradybaenidae (Bradybaena fruticum); Cepaea (C. hortensis, C. Nemoralis); ochlodina; Deroceras (D. agrestis, D. empiricorum, D. laeve, D. reticulatum); Discus (D. rotundatus); Euomphalia; Galba (G. trunculata); Helicelia (H. itala, H. obvia); Helicidae Helicigona arbustorum); Helicodiscus; Helix (H. aperta); Limax (L. cinereoniger, L. flavus, L. marginatus, L. maximus, L. tenellus); Lymnaea; Milax (M. gagates, M. marginatus, M. sowerbyi); Opeas; Pomacea (P. canaticulata); Vallonia and Zanitoides.

The term “crops” is to be understood as including also crop plants which have been so transformed by the use of recombinant DNA techniques that they are capable of synthesising one or more selectively acting toxins, such as are known, for example, from toxin-producing bacteria, especially those of the genus Bacillus.

Toxins that can be expressed by such transgenic plants include, for example, insecticidal proteins, for example insecticidal proteins from Bacillus cereus or Bacillus popilliae; or insecticidal proteins from Bacillus thuringiensis, such as 6-endotoxins, e.g. Cry1Ab, Cry1Ac, Cry1F, Cry1 Fa2, Cry2Ab, Cry3A, Cry3Bb1 or Cry9C, or vegetative insecticidal proteins (Vip), e.g. Vip1, Vip2, Vip3 or Vip3A; or insecticidal proteins of bacteria colonising nematodes, for example Photorhabdus spp. or Xenorhabdus spp., such as Photorhabdus luminescens, Xenorhabdus nematophilus; toxins produced by animals, such as scorpion toxins, arachnid toxins, wasp toxins and other insect-specific neurotoxins; toxins produced by fungi, such as Streptomycetes toxins, plant lectins, such as pea lectins, barley lectins or snowdrop lectins; agglutinins; proteinase inhibitors, such as trypsin inhibitors, serine protease inhibitors, patatin, cystatin, papain inhibitors; ribosome-inactivating proteins (RIP), such as ricin, maize-RIP, abrin, luffin, saporin or bryodin; steroid metabolism enzymes, such as 3-hydroxysteroidoxidase, ecdysteroid-UDP-glycosyl-transferase, cholesterol oxidases, ecdysone inhibitors, HMG-COA-reductase, ion channel blockers, such as blockers of sodium or calcium channels, juvenile hormone esterase, diuretic hormone receptors, stilbene synthase, bibenzyl synthase, chitinases and glucanases.

In the context of the present invention there are to be understood by 6-endotoxins, for example Cry1Ab, Cry1Ac, Cry1F, Cry1Fa2, Cry2Ab, Cry3A, Cry3Bb1 or Cry9C, or vegetative insecticidal proteins (Vip), for example Vip1, Vip2, Vip3 or Vip3A, expressly also hybrid toxins, truncated toxins and modified toxins. Hybrid toxins are produced recombinantly by a new combination of different domains of those proteins (see, for example, WO 02/15701). Truncated toxins, for example a truncated Cry1Ab, are known. In the case of modified toxins, one or more amino acids of the naturally occurring toxin are replaced. In such amino acid replacements, preferably non-naturally present protease recognition sequences are inserted into the toxin, such as, for example, in the case of Cry3A055, a cathepsin-G-recognition sequence is inserted into a Cry3A toxin (see WO 03/018810). Examples of such toxins or transgenic plants capable of synthesising such toxins are disclosed, for example, in EP-A-0 374 753, WO 93/07278, WO 95/34656, EP-A-0 427 529, EP-A-451 878 and WO 03/052073.

The processes for the preparation of such transgenic plants are generally known to the person skilled in the art and are described, for example, in the publications mentioned above. Cryl-type deoxyribonucleic acids and their preparation are known, for example, from WO 95/34656, EP-A-0 367 474, EP-A-0 401 979 and WO 90/13651.

The toxin contained in the transgenic plants imparts to the plants tolerance to harmful insects. Such insects can occur in any taxonomic group of insects, but are especially commonly found in the beetles (Coleoptera), two-winged insects (Diptera) and moths (Lepidoptera).

Transgenic plants containing one or more genes that code for an insecticidal resistance and express one or more toxins are known and some of them are commercially available. Examples of such plants are: YieldGard® (maize variety that expresses a Cry1Ab toxin); YieldGard Rootworm® (maize variety that expresses a Cry3Bb1 toxin); YieldGard Plus® (maize variety that expresses a Cry1Ab and a Cry3Bb1 toxin); Starlink® (maize variety that expresses a Cry9C toxin); Herculex I® (maize variety that expresses a Cryl Fa2 toxin and the enzyme phosphinothricine N-acetyltransferase (PAT) to achieve tolerance to the herbicide glufosinate ammonium); NuCOTN 33B® (cotton variety that expresses a Cry1Ac toxin); Bollgard I® (cotton variety that expresses a Cry1Ac toxin); Bollgard II® (cotton variety that expresses a Cry1Ac and a Cry2Ab toxin); VipCot® (cotton variety that expresses a Vip3A and a Cry1Ab toxin); NewLeaf® (potato variety that expresses a Cry3A toxin); NatureGard®, Agrisure® GT Advantage (GA21 glyphosate-tolerant trait), Agrisure® CB Advantage (Bt11 corn borer (CB) trait) and Protecta®.

Further examples of such transgenic crops are:
1. Bt11 Maize from Syngenta Seeds SAS, Chemin de I'Hobit 27, F-31 790 St. Sauveur, France, registration number C/FR/96/05/10. Genetically modified Zea mays which has been rendered resistant to attack by the European corn borer (Ostrinia nubilalis and Sesamia nonagrioides) by transgenic expression of a truncated Cry1Ab toxin. Bt11 maize also transgenically expresses the enzyme PAT to achieve tolerance to the herbicide glufosinate ammonium.
2. Bt176 Maize from Syngenta Seeds SAS, Chemin de I'Hobit 27, F-31 790 St. Sauveur, France, registration number C/FR/96/05/10. Genetically modified Zea mays which has been rendered resistant to attack by the European corn borer (Ostrinia nubilalis and Sesamia nonagrioides) by transgenic expression of a Cry1Ab toxin. Bt176 maize also transgenically expresses the enzyme PAT to achieve tolerance to the herbicide glufosinate ammonium.
3. MIR604 Maize from Syngenta Seeds SAS, Chemin de I'Hobit 27, F-31 790 St. Sauveur, France, registration number C/FR/96/05/10. Maize which has been rendered insect-resistant by transgenic expression of a modified Cry3A toxin. This toxin is Cry3A055 modified by insertion of a cathepsin-G-protease recognition sequence. The preparation of such transgenic maize plants is described in WO 03/018810.
4. MON 863 Maize from Monsanto Europe S.A. 270-272 Avenue de Tervuren, B-1150 Brussels, Belgium, registration number C/DE/02/9. MON 863 expresses a Cry3Bb1 toxin and has resistance to certain Coleoptera insects.
5. IPC 531 Cotton from Monsanto Europe S.A. 270-272 Avenue de Tervuren, B-1150 Brussels, Belgium, registration number C/ES/96/02.
6. 1507 Maize from Pioneer Overseas Corporation, Avenue Tedesco, 7 B-1160 Brussels, Belgium, registration number C/NL/00/10. Genetically modified maize for the expression of the protein Cryl F for achieving resistance to certain Lepidoptera insects and of the PAT protein for achieving tolerance to the herbicide glufosinate ammonium.
7. NK603×MON 810 Maize from Monsanto Europe S.A. 270-272 Avenue de Tervuren, B-1150 Brussels, Belgium, registration number C/GB/02/M3/03. Consists of conventionally bred hybrid maize varieties by crossing the genetically modified varieties NK603 and MON 810. NK603×MON 810 Maize transgenically expresses the protein CP4 EPSPS, obtained from Agrobacterium sp. strain CP4, which imparts tolerance to the herbicide Roundup® (contains glyphosate), and also a Cry1Ab toxin obtained from Bacillus thuringiensis subsp. kurstaki which brings about tolerance to certain Lepidoptera, include the European corn borer.

Transgenic crops of insect-resistant plants are also described in BATS (Zentrum für Biosicherheit und Nachhaltigkeit, Zentrum BATS, Clarastrasse 13, 4058 Basel, Switzerland) Report 2003, (http://bats.ch).

The term “crops” is to be understood as including also crop plants which have been so transformed by the use of recombinant DNA techniques that they are capable of synthesising antipathogenic substances having a selective action, such as, for example, the so-called “pathogenesis-related proteins” (PRPs, see e.g. EP-A-0 392 225). Examples of such antipathogenic substances and transgenic plants capable of synthesising such antipathogenic substances are known, for example, from EP-A-0 392 225, WO 95/33818 and EP-A-0 353 191. The methods of producing such transgenic plants are generally known to the person skilled in the art and are described, for example, in the publications mentioned above.

Crops may also be modified for enhanced resistance to fungal (for example Fusarium, Anthracnose, or Phytophthora), bacterial (for example Pseudomonas) or viral (for example potato leafroll virus, tomato spotted wilt virus, cucumber mosaic virus) pathogens.

Crops also include those that have enhanced resistance to nematodes, such as the soybean cyst nematode.

Crops that are tolerance to abiotic stress include those that have enhanced tolerance to drought, high salt, high temperature, chill, frost, or light radiation, for example through expression of NF-YB or other proteins known in the art.

Antipathogenic substances which can be expressed by such transgenic plants include, for example, ion channel blockers, such as blockers for sodium and calcium channels, for example the viral KP1, KP4 or KP6 toxins; stilbene synthases; bibenzyl synthases; chitinases; glucanases; the so-called “pathogenesis-related proteins” (PRPs; see e.g. EP-A-0 392 225); antipathogenic substances produced by microorganisms, for example peptide antibiotics or heterocyclic antibiotics (see e.g. WO 95/33818) or protein or polypeptide factors involved in plant pathogen defence (so-called “plant disease resistance genes”, as described in WO 03/000906).

Further areas of use of the compositions according to the invention are the protection of stored goods and store rooms and the protection of raw materials, such as wood, textiles, floor coverings or buildings, and also in the hygiene sector, especially the protection of humans, domestic animals and productive livestock against pests of the mentioned type.

The present invention also provides a method for controlling pests (such as mosquitoes and other disease vectors; see also http://www.who.int/malaria/vector_control/irs/en/). In one embodiment, the method for controlling pests comprises applying the compositions of the invention to the target pests, to their locus or to a surface or substrate by brushing, rolling, spraying, spreading or dipping. By way of example, an IRS (indoor residual spraying) application of a surface such as a wall, ceiling or floor surface is contemplated by the method of the invention. In another embodiment, it is contemplated to apply such compositions to a substrate such as non-woven or a fabric material in the form of (or which can be used in the manufacture of) netting, clothing, bedding, curtains and tents.

In one embodiment, the method for controlling such pests comprises applying a pesticidally effective amount of the compositions of the invention to the target pests, to their locus, or to a surface or substrate so as to provide effective residual pesticidal activity on the surface or substrate. Such application may be made by brushing, rolling, spraying, spreading or dipping the pesticidal composition of the invention. By way of example, an IRS application of a surface such as a wall, ceiling or floor surface is contemplated by the method of the invention so as to provide effective residual pesticidal activity on the surface. In another embodiment, it is contemplated to apply such compositions for residual control of pests on a substrate such as a fabric material in the form of (or which can be used in the manufacture of) netting, clothing, bedding, curtains and tents.

Substrates including non-woven, fabrics or netting to be treated may be made of natural fibres such as cotton, raffia, jute, flax, sisal, hessian, or wool, or synthetic fibres such as polyamide, polyester, polypropylene, polyacrylonitrile or the like. The polyesters are particularly suitable. The methods of textile treatment are known, e.g. WO 2008/151984, WO 2003/034823, U.S. Pat. No. 5,631,072, WO 2005/64072, WO2006/128870, EP 1724392, WO 2005113886 or WO 2007/090739.

Further areas of use of the compositions according to the invention are the field of tree injection/trunk treatment for all ornamental trees as well all sort of fruit and nut trees.

In the field of tree injection/trunk treatment, the compounds according to the present invention are especially suitable against wood-boring insects from the order Lepidoptera as mentioned above and from the order Coleoptera, especially against woodborers listed in the following tables A and B:

TABLE A
Examples of exotic woodborers of economic importance.

| Family | Species | Host or Crop Infested |
|---|---|---|
| Buprestidae | Agrilus planipennis | Ash |
| Cerambycidae | Anoplura glabripennis | Hardwoods |
| Scolytidae | Xylosandrus crassiusculus | Hardwoods |
| | X. mutilatus | Hardwoods |
| | Tomicus piniperda | Conifers |

TABLE B
Examples of native woodborers of economic importance.

| Family | Species | Host or Crop Infested |
|---|---|---|
| Buprestidae | Agrilus anxius | Birch |
| | Agrilus politus | Willow, Maple |
| | Agrilus sayi | Bayberry, Sweetfern |
| | Agrilus vittaticolllis | Apple, Pear, Cranberry, |
| | | Serviceberry, Hawthorn |
| | Chrysobothris femorata | Apple, Apricot, Beech, Boxelder, |
| | | Cherry, Chestnut, Currant, Elm, |
| | | Hawthorn, Hackberry, Hickory, |
| | | Horsechestnut, Linden, Maple, |
| | | Mountain-ash, Oak, Pecan, Pear, |
| | | Peach, Persimmon, Plum, Poplar, |
| | | Quince, Redbud, Serviceberry, Sycamore, Walnut, |
| | | Willow |
| | Texania campestris | Basswood, Beech, Maple, Oak, |
| | | Sycamore, Willow, Yellow-poplar |
| Cerambycidae | Goes pulverulentus | Beech, Elm, Nuttall, Willow, Black |
| | | oak, Cherrybark oak, Water oak, |
| | | Sycamore |
| | Goes tigrinus | Oak |
| | Neoclytus acuminatus | Ash, Hickory, Oak, Walnut, Birch, |
| | | Beech, Maple, Eastern |
| | | hophornbeam, Dogwood, |
| | | Persimmon, Redbud, Holly, |
| | | Hackberry, Black locust, |
| | | Honeylocust, Yellow-poplar, |
| | | Chestnut, Osage-orange, Sassafras, |
| | | Lilac, Mountain-mahogany, Pear, |
| | | Cherry, Plum, Peach, Apple, Elm, |
| | | Basswood, Sweetgum |
| | Neoptychodes trilineatus | Fig, Alder, Mulberry, Willow, Netleaf |
| | | hackberry |
| | Oberea ocellata | Sumac, Apple, Peach, Plum, Pear, |
| | | Currant, Blackberry |
| | Oberea tripunctata | Dogwood, Viburnum, Elm, |
| | | Sourwood, Blueberry, |
| | | Rhododendron, Azalea, Laurel, |
| | | Poplar, Willow, Mulberry |
| | Oncideres cingulata | Hickory, Pecan, Persimmon, Elm, |
| | | Sourwood, Basswood, Honeylocust, |
| | | Dogwood, Eucalyptus, Oak, |
| | | Hackberry, Maple, Fruit trees |
| | Saperda calcarata | Poplar |
| | Strophiona nitens | Chestnut, Oak, Hickory, Walnut, |
| | | Beech, Maple |
| Scolytidae | Corthylus columbianus | Maple, Oak, Yellow-poplar, Beech, |
| | | Boxelder, Sycamore, Birch, |
| | | Basswood, Chestnut, Elm |
| | Dendroctonus frontalis | Pine |
| | Dryocoetes betulae | Birch, Sweetgum, Wild cherry, |
| | | Beech, Pear |
| | Monarthrum fasciatum | Oak, Maple, Birch, Chestnut, |
| | | Sweetgum, Blackgum, Poplar, |
| | | Hickory, Mimosa, Apple, Peach, Pine |
| | Phloeotribus liminaris | Peach, Cherry, Plum, Black cherry, |
| | | Elm, Mulberry, Mountain-ash |
| | Pseudopityophthorus pruinosus | Oak, American beech, Black cherry, |
| | | Chickasaw plum, Chestnut, Maple, |
| | | Hickory, Hornbeam, Hophornbeam |
| Sesiidae | Paranthrene simulans | Oak, American chestnut |
| | Sannina uroceriformis | Persimmon |
| | Synanthedon exitiosa | Peach, Plum, Nectarine, Cherry, |
| | | Apricot, Almond, Black cherry |
| | Synanthedon pictipes | Peach, Plum, Cherry, Beach, Black |
| | | Cherry |
| | Synanthedon rubrofascia | Tupelo |
| | Synanthedon scitula | Dogwood, Pecan, Hickory, Oak, |
| | | Chestnut, Beech, Birch, Black cherry, |
| | | Elm, Mountain-ash, Viburnum, |
| | | Willow, Apple, Loquat, Ninebark, |
| | | Bayberry |
| | Vitacea polistiformis | Grape |

The present invention may be also used to control any insect pests that may be present in turfgrass, including for example beetles, caterpillars, fire ants, ground pearls, millipedes, sow bugs, mites, mole crickets, scales, mealybugs ticks, spittlebugs, southern chinch bugs and white grubs. The present invention may be used to control insect pests at various stages of their life cycle, including eggs, larvae, nymphs and adults.

In particular, the present invention may be used to control insect pests that feed on the roots of turfgrass including white grubs (such as Cyclocephala spp. (e.g. masked chafer, C. lurida), Rhizotrogus spp. (e.g. European chafer, R. majalis), Cotinus spp. (e.g. Green June beetle, C. nitida), Popillia spp. (e.g. Japanese beetle, P. japonica), Phyllophaga spp. (e.g. May/June beetle), Ataenius spp. (e.g. Black turfgrass ataenius, A. spretulus), Maladera spp. (e.g. Asiatic garden beetle, M. castanea) and Tomarus spp.), ground pearls (Margarodes spp.), mole crickets (tawny, southern, and short-winged; Scapteriscus spp., Gryllotalpa africana) and leatherjackets (European crane fly, Tipula spp.).

The present invention may also be used to control insect pests of turfgrass that are thatch dwelling, including armyworms (such as fall armyworm Spodoptera frugiperda, and common armyworm Pseudaletia unipuncta), cutworms, billbugs (Sphenophorus spp., such as S. venatus verstitus and S. parvulus), and sod webworms (such as Crambus spp. and the tropical sod webworm, Herpetogramma phaeopteralis).

The present invention may also be used to control insect pests of turfgrass that live above the ground and feed on the turfgrass leaves, including chinch bugs (such as southern chinch bugs, Blissus insularis), Bermudagrass mite (Eriophyes cynodoniensis), rhodesgrass mealybug (Antonina graminis), two-lined spittlebug (Propsapia bicincta), leafhoppers, cutworms (Noctuidae family), and greenbugs.

The present invention may also be used to control other pests of turfgrass such as red imported fire ants (Solenopsis invicta) that create ant mounds in turf.

In the hygiene sector, the compositions according to the invention are active against ectoparasites such as hard ticks, soft ticks, mange mites, harvest mites, flies (biting and licking), parasitic fly larvae, lice, hair lice, bird lice and fleas.

Examples of such parasites are:

Of the order Anoplurida: Haematopinus spp., Linognathus spp., Pediculus spp. and Phtirus spp., Solenopotes spp.

Of the order Mallophagida: Trimenopon spp., Menopon spp., Trinoton spp., Bovicola spp., Werneckiella spp., Lepikentron spp., Damalina spp., Trichodectes spp. and Felicola spp.

Of the order Diptera and the suborders Nematocerina and Brachycerina, for example Aedes spp., Anopheles spp., Culex spp., Simulium spp., Eusimulium spp., Phlebotomus spp., Lutzomyia spp., Culicoides spp., Chrysops spp., Hybomitra spp., Atylotus spp., Tabanus spp., Haematopota spp., Philipomyia spp., Braula spp., Musca spp., Hydrotaea spp., Stomoxys spp., Haematobia spp., Morellia spp., Fannia spp., Glossina spp., Calliphora spp., Lucilia spp., Chrysomyia spp., Wohlfahrtia spp., Sarcophaga spp., Oestrus spp., Hypoderma spp., Gasterophilus spp., Hippobosca spp., Lipoptena spp. and Melophagus spp.

Of the order Siphonapterida, for example Pulex spp., Ctenocephalides spp., Xenopsylla spp., Ceratophyllus spp.

Of the order Heteropterida, for example Cimex spp., Triatoma spp., Rhodnius spp., Panstrongylus spp.

Of the order Blattarida, for example Blatta orientalis, Periplaneta americana, Blattelagermanica and Supella spp.

Of the subclass Acaria (Acarida) and the orders Meta- and Meso-stigmata, for example Argas spp., Ornithodorus spp., Otobius spp., Ixodes spp., Amblyomma spp., Boophilus spp., Dermacentor spp., Haemophysalis spp., Hyalomma spp., Rhipicephalus spp., Dermanyssus spp., Raillietia spp., Pneumonyssus spp., Sternostoma spp. and Varroa spp.

Of the orders Actinedida (Prostigmata) and Acaridida (Astigmata), for example Acarapis spp., Cheyletiella spp., Ornithocheyletia spp., Myobia spp., Psorergatesspp., Demodex spp., Trombicula spp., Listrophorus spp., Acarus spp., Tyrophagus spp., Caloglyphus spp., Hypodectes spp., Pterolichus spp., Psoroptes spp., Chorioptes spp., Otodectes spp., Sarcoptes spp., Notoedres spp., Knemidocoptes spp., Cytodites spp. and Laminosioptes spp.

The compositions according to the invention are also suitable for protecting against insect infestation in the case of materials such as wood, textiles, plastics, adhesives, glues, paints, paper and card, leather, floor coverings and buildings.

The compositions according to the invention can be used, for example, against the following pests: beetles such as Hylotrupes bajulus, Chlorophorus pilosis, Anobium punctatum, Xestobium rufovillosum, Ptilinuspecticornis, Dendrobium pertinex, Ernobius mollis, Priobium carpini, Lyctus brunneus, Lyctus africanus, Lyctus planicollis, Lyctus linearis, Lyctus pubescens, Trogoxylon aequale, Minthesrugicollis, Xyleborus spec., Tryptodendron spec., Apate monachus, Bostrychus capucins, Heterobostrychus brunneus, Sinoxylon spec. and Dinoderus minutus, and also hymenopterans such as Sirexjuvencus, Urocerus gigas, Urocerus gigas taignus and Urocerus augur, and termites such as Kalotermes flavicollis, Cryptotermes brevis, Heterotermes indicola, Reticulitermes flavipes, Reticulitermes santonensis, Reticulitermes lucifugus, Mastotermes darwiniensis, Zootermopsis nevadensis and Coptotermes formosanus, and bristletails such as Lepisma saccharina.

The compounds according to the invention can be used as pesticidal agents in unmodified form, but they are generally formulated into compositions in various ways using formulation adjuvants, such as carriers, solvents and surface-active substances. The formulations can be in various physical forms, e.g. in the form of dusting powders, gels, wettable powders, water-dispersible granules, water-dispersible tablets, effervescent pellets, emulsifiable concentrates, microemulsifiable concentrates, oil-in-water emulsions, oil-flowables, aqueous dispersions, oily dispersions, suspo-emulsions, capsule suspensions, emulsifiable granules, soluble liquids, water-soluble concentrates (with water or a water-miscible organic solvent as carrier), impregnated polymer films or in other forms known e.g. from the Manual on Development and Use of FAO and WHO Specifications for Pesticides, United Nations, First Edition, Second Revision (2010). Such formulations can either be used directly or diluted prior to use. The dilutions can be made, for example, with water, liquid fertilisers, micronutrients, biological organisms, oil or solvents.

The formulations can be prepared e.g. by mixing the active ingredient with the formulation adjuvants in order to obtain compositions in the form of finely divided solids, granules, solutions, dispersions or emulsions. The active ingredients can also be formulated with other adjuvants, such as finely divided solids, mineral oils, oils of vegetable or animal origin, modified oils of vegetable or animal origin, organic solvents, water, surface-active substances or combinations thereof.

The active ingredients can also be contained in very fine microcapsules. Microcapsules contain the active ingredients in a porous carrier. This enables the active ingredients to be released into the environment in controlled amounts (e.g. slow-release). Microcapsules usually have a diameter of from 0.1 to 500 microns. They contain active ingredients in an amount of about from 25 to 95% by weight of the capsule weight. The active ingredients can be in the form of a monolithic solid, in the form of fine particles in solid or liquid dispersion or in the form of a suitable solution. The encapsulating membranes can comprise, for example, natural or synthetic rubbers, cellulose, styrene/butadiene copolymers, polyacrylonitrile, polyacrylate, polyesters, polyamides, polyureas, polyurethane or chemically modified polymers and starch xanthates or other polymers that are known to the person skilled in the art. Alternatively, very fine microcapsules can be formed in which the active ingredient is contained in the form of finely divided particles in a solid matrix of base substance, but the microcapsules are not themselves encapsulated.

The formulation adjuvants that are suitable for the preparation of the compositions according to the invention are known per se. As liquid carriers there may be used: water, toluene, xylene, petroleum ether, vegetable oils, acetone, methyl ethyl ketone, cyclohexanone, acid anhydrides, acetonitrile, acetophenone, amyl acetate, 2-butanone, butylene carbonate, chlorobenzene, cyclohexane, cyclohexanol, alkyl esters of acetic acid, diacetone alcohol, 1,2-dichloropropane, diethanolamine, p-diethylbenzene, diethylene glycol, diethylene glycol abietate, diethylene glycol butyl ether, diethylene glycol ethyl ether, diethylene glycol methyl ether, N,N-dimethylformamide, dimethyl sulfoxide, 1,4-dioxane, dipropylene glycol, dipropylene glycol methyl ether, dipropylene glycol dibenzoate, diproxitol, alkylpyrrolidone, ethyl acetate, 2-ethylhexanol, ethylene carbonate, 1,1,1-trichloroethane, 2-heptanone, alpha-pinene, d-limonene, ethyl lactate, ethylene glycol, ethylene glycol butyl ether, ethylene glycol methyl ether, gamma-butyrolactone, glycerol, glycerol acetate, glycerol diacetate, glycerol triacetate, hexadecane, hexylene glycol, isoamyl acetate, isobornyl acetate, isooctane, isophorone, isopropylbenzene, isopropyl myristate, lactic acid, laurylamine, mesityl oxide, methoxy-propanol, methyl isoamyl ketone, methyl isobutyl ketone, methyl laurate, methyl octanoate, methyl oleate, methylene chloride, m-xylene, n-hexane, n-octylamine, octadecanoic acid, octylamine acetate, oleic acid, oleylamine, o-xylene, phenol, polyethylene glycol, propionic acid, propyl lactate, propylene carbonate, propylene glycol, propylene glycol methyl ether, p-xylene, toluene, triethyl phosphate, triethylene glycol, xylenesulfonic acid, paraffin, mineral oil, trichloroethylene, perchloroethylene, ethyl acetate, amyl acetate, butyl acetate, propylene glycol methyl ether, diethylene glycol methyl ether, methanol, ethanol, isopropanol, and alcohols of higher molecular weight, such as amyl alcohol, tetrahydrofurfuryl alcohol, hexanol, octanol, ethylene glycol, propylene glycol, glycerol, N-methyl-2-pyrrolidone and the like.

Suitable solid carriers are, for example, talc, titanium dioxide, pyrophyllite clay, silica, attapulgite clay, kieselguhr, limestone, calcium carbonate, bentonite, calcium montmorillonite, cottonseed husks, wheat flour, soybean flour, pumice, wood flour, ground walnut shells, lignin and similar substances.

A large number of surface-active substances can advantageously be used in both solid and liquid formulations, especially in those formulations which can be diluted with a carrier prior to use. Surface-active substances may be anionic, cationic, non-ionic or polymeric and they can be used as emulsifiers, wetting agents or suspending agents or for other purposes. Typical surface-active substances include, for example, salts of alkyl sulfates, such as diethanolammonium lauryl sulfate; salts of alkylarylsulfonates, such as calcium dodecylbenzenesulfonate; alkylphenol/alkylene oxide addition products, such as nonylphenol ethoxylate; alcohol/alkylene oxide addition products, such as tridecylalcohol ethoxylate; soaps, such as sodium stearate; salts of alkylnaphthalenesulfonates, such as sodium dibutylnaphthalenesulfonate; dialkyl esters of sulfosuccinate salts, such as sodium di(2-ethylhexyl)sulfosuccinate; sorbitol esters, such as sorbitol oleate; quaternary amines, such as lauryltrimethylammonium chloride, polyethylene glycol esters of fatty acids, such as polyethylene glycol stearate; block copolymers of ethylene oxide and propylene oxide; and salts of mono- and di-alkylphosphate esters; and also further substances described e.g. in McCutcheon's Detergents and Emulsifiers Annual, MC Publishing Corp., Ridgewood New Jersey (1981).

Further adjuvants that can be used in pesticidal formulations include crystallisation inhibitors, viscosity modifiers, suspending agents, dyes, anti-oxidants, foaming agents, light absorbers, mixing auxiliaries, antifoams, complexing agents, neutralising or pH-modifying substances and buffers, corrosion inhibitors, fragrances, wetting agents, take-up enhancers, micronutrients, plasticisers, glidants, lubricants, dispersants, thickeners, antifreezes, microbicides, and liquid and solid fertilisers.

The compositions according to the invention can include an additive comprising an oil of vegetable or animal origin, a mineral oil, alkyl esters of such oils or mixtures of such oils and oil derivatives. The amount of oil additive in the composition according to the invention is generally from 0.01 to 10%, based on the mixture to be applied. For example, the oil additive can be added to a spray tank in the desired concentration after a spray mixture has been prepared. Preferred oil additives comprise mineral oils or an oil of vegetable origin, for example rapeseed oil, olive oil or sunflower oil, emulsified vegetable oil, alkyl esters of oils of vegetable origin, for example the methyl derivatives, or an oil of animal origin, such as fish oil or beef tallow. Preferred oil additives comprise alkyl esters of C₈-C₂₂ fatty acids, especially the methyl derivatives of C₁₂-C₁₈ fatty acids, for example the methyl esters of lauric acid, palmitic acid and oleic acid (methyl laurate, methyl palmitate and methyl oleate, respectively). Many oil derivatives are known from the Compendium of Herbicide Adjuvants, 10^{th} Edition, Southern Illinois University, 2010.

The inventive compositions generally comprise from 0.1 to 99% by weight, especially from 0.1 to 95% by weight, of compounds of the present invention and from 1 to 99.9% by weight of a formulation adjuvant which preferably includes from 0 to 25% by weight of a surface-active substance. Whereas commercial products may preferably be formulated as concentrates, the end user will normally employ dilute formulations.

The rates of application vary within wide limits and depend on the nature of the soil, the method of application, the crop plant, the pest to be controlled, the prevailing climatic conditions, and other factors governed by the method of application, the time of application and the target crop. As a general guideline compounds may be applied at a rate of from 1 to 2000 I/ha, especially from 10 to 1000 I/ha.

Preferred formulations can have the following compositions (weight %):

### Emulsifiable Concentrates:

active ingredient: 1 to 95%, preferably 60 to 90%
surface-active agent: 1 to 30%, preferably 5 to 20%
liquid carrier: 1 to 80%, preferably 1 to 35%

### Dusts:

active ingredient: 0.1 to 10%, preferably 0.1 to 5%
solid carrier: 99.9 to 90%, preferably 99.9 to 99%

### Suspension Concentrates:

active ingredient: 5 to 75%, preferably 10 to 50%
water: 94 to 24%, preferably 88 to 30%
surface-active agent: 1 to 40%, preferably 2 to 30%

### Wettable Powders:

active ingredient: 0.5 to 90%, preferably 1 to 80%
surface-active agent: 0.5 to 20%, preferably 1 to 15%
solid carrier: 5 to 95%, preferably 15 to 90%

### Granules:

active ingredient: 0.1 to 30%, preferably 0.1 to 15%
solid carrier: 99.5 to 70%, preferably 97 to 85%

The following Examples further illustrate, but do not limit, the invention.

| Wettable powders | a) | b) | c) |
|---|---|---|---|
| active ingredients | 25% | 50% | 75% |
| sodium lignosulfonate | 5% | 5% | — |
| sodium lauryl sulfate | 3% | — | 5% |
| sodium diisobutylnaphthalenesulfonate | — | 6% | 10% |
| phenol polyethylene glycol ether | — | 2% | — |
| (7-8 mol of ethylene oxide) | | | |
| highly dispersed silicic acid | 5% | 10% | 10% |
| Kaolin | 62% | 27% | — |

The combination is thoroughly mixed with the adjuvants and the mixture is thoroughly ground in a suitable mill, affording wettable powders that can be diluted with water to give suspensions of the desired concentration.

| Powders for dry seed treatment | a) | b) | c) |
|---|---|---|---|
| active ingredients | 25% | 50% | 75% |
| light mineral oil | 5% | 5% | 5% |
| highly dispersed silicic acid | 5% | 5% | — |
| Kaolin | 65% | 40% | — |
| Talcum | — | | 20% |

The combination is thoroughly mixed with the adjuvants and the mixture is thoroughly ground in a suitable mill, affording powders that can be used directly for seed treatment.

| Emulsifiable concentrate | |
|---|---|
| active ingredients | 10% |
| octylphenol polyethylene glycol ether | 3% |
| (4-5 mol of ethylene oxide) | |
| calcium dodecylbenzenesulfonate | 3% |
| castor oil polyglycol ether (35 mol of ethylene oxide) | 4% |
| Cyclohexanone | 30% |
| xylene mixture | 50% |

Emulsions of any required dilution, which can be used in plant protection, can be obtained from this concentrate by dilution with water.

| Dusts | a) | b) | c) |
|---|---|---|---|
| Active ingredients | 5% | 6% | 4% |
| Talcum | 95% | — | — |
| Kaolin | — | 94% | — |
| mineral filler | — | — | 96% |

Ready-for-use dusts are obtained by mixing the combination with the carrier and grinding the mixture in a suitable mill. Such powders can also be used for dry dressings for seed.

| Extruder granules | |
|---|---|
| Active ingredients | 15% |
| sodium lignosulfonate | 2% |
| carboxymethylcellulose | 1% |
| Kaolin | 82% |

The combination is mixed and ground with the adjuvants, and the mixture is moistened with water. The mixture is extruded and then dried in a stream of air.

| Coated granules | |
|---|---|
| Active ingredients | 8% |
| polyethylene glycol (mol. wt. 200) | 3% |
| Kaolin | 89% |

The finely ground combination is uniformly applied, in a mixer, to the kaolin moistened with polyethylene glycol. Non-dusty coated granules are obtained in this manner.

### Suspension Concentrate

| | |
|---|---|
| active ingredients | 40% |
| propylene glycol | 10% |
| nonylphenol polyethylene glycol ether (15 mol of ethylene oxide) | 6% |
| Sodium lignosulfonate | 10% |
| carboxymethylcellulose | 1% |
| silicone oil (in the form of a 75% emulsion in water) | 1% |
| Water | 32% |

The finely ground combination is intimately mixed with the adjuvants, giving a suspension concentrate from which suspensions of any desired dilution can be obtained by dilution with water. Using such dilutions, living plants as well as plant propagation material can be treated and protected against infestation by microorganisms, by spraying, pouring or immersion.

### Flowable Concentrate for Seed Treatment

| | |
|---|---|
| active ingredients | 40% |
| propylene glycol | 5% |
| copolymer butanol PO/EO | 2% |
| Tristyrenephenole with 10-20 moles EO | 2% |
| 1,2-benzisothiazolin-3-one | 0.5% |
| (in the form of a 20% solution in water) | |
| monoazo-pigment calcium salt | 5% |
| Silicone oil (in the form of a 75% emulsion in water) | 0.2% |
| Water | 45.3% |

The finely ground combination is intimately mixed with the adjuvants, giving a suspension concentrate from which suspensions of any desired dilution can be obtained by dilution with water. Using such dilutions, living plants as well as plant propagation material can be treated and protected against infestation by microorganisms, by spraying, pouring or immersion.

### Slow Release Capsule Suspension

28 parts of the combination are mixed with 2 parts of an aromatic solvent and 7 parts of toluene diisocyanate/polymethylene-polyphenylisocyanate-mixture (8:1). This mixture is emulsified in a mixture of 1.2 parts of polyvinylalcohol, 0.05 parts of a defoamer and 51.6 parts of water until the desired particle size is achieved. To this emulsion a mixture of 2.8 parts 1,6-diaminohexane in 5.3 parts of water is added. The mixture is agitated until the polymerization reaction is completed. The obtained capsule suspension is stabilized by adding 0.25 parts of a thickener and 3 parts of a dispersing agent. The capsule suspension formulation contains 28% of the active ingredients. The medium capsule diameter is 8-15 microns. The resulting formulation is applied to seeds as an aqueous suspension in an apparatus suitable for that purpose.

Formulation types include an emulsion concentrate (EC), a suspension concentrate (SC), a suspo-emulsion (SE), a capsule suspension (CS), a water dispersible granule (WG), an emulsifiable granule (EG), an emulsion, water in oil (EO), an emulsion, oil in water (EW), a micro-emulsion (ME), an oil dispersion (OD), an oil miscible flowable (OF), an oil miscible liquid (OL), a soluble concentrate (SL), an ultra-low volume suspension (SU), an ultra-low volume liquid (UL), a technical concentrate (TK), a dispersible concentrate (DC), a wettable powder (WP), a soluble granule (SG) or any technically feasible formulation in combination with agriculturally acceptable adjuvants.

### PREPARATORY EXAMPLES

“Mp” means melting point in ° C. Free radicals represent methyl groups. ¹H NMR measurements were recorded on a Brucker 400 MHz spectrometer, chemical shifts are given in ppm relevant to a TMS standard. Spectra measured in deuterated solvents as indicated. Either one of the LCMS methods below was used to characterize the compounds. The characteristic LCMS values obtained for each compound were the retention time (“Rt”, recorded in minutes) and the measured molecular ion (M+H)⁺ or (M−H)⁻.

LCMS Methods:

Method 1:

Spectra were recorded on a Mass Spectrometer from Waters (SQD Single quadrupole mass spectrometer) equipped with an electrospray source (Polarity: positive or negative ions, Full Scan, Capillary: 3.00 kV, Cone range: 41 V, Source Temperature: 150° C., Desolvation Temperature: 500° C., Cone Gas Flow: 50 L/Hr, Desolvation Gas Flow: 1000 L/Hr, Mass range: 110 to 800 Da) and a H-Class UPLC from Waters: quaternary pump, heated column compartment and diode-array detector. Column: Acquity UPLC HSS T3 C18, 1.8 μm, 30×2.1 mm, Temp: 40° C., DAD Wavelength range (nm): 200 to 400, Solvent Gradient: A=water+5% Acetonitrile+0.1% HCOOH, B=Acetonitrile+0.05% HCOOH: gradient: 0 min 10% B; 0.-0.2 min 10-50% B; 0.2-0.7 min 50-100% B; 0.7-1.3 min 100% B; 1.3-1.4 min 100-10% B; 1.4-1.6 min 10% B; Flow (mL/min) 0.6.

### Method 2:

Spectra were recorded on a Mass Spectrometer from Agilent Technologies (6410 Triple Quadrupole mass spectrometer) equipped with an electrospray source (Polarity: positive or negative ions, MS2 Scan, Capillary: 4.00 kV, Fragmentor: 100 V, Desolvatation Temperature: 350° C., Gas Flow: 11 L/min, Nebulizer Gas: 45 psi, Mass range: 110 to 1000 Da) and a 1200 Series HPLC from Agilent: quaternary pump, heated column compartment and diode-array detector. Column: KINETEX EVO C18, 2.6 μm, 50×4.6 mm, Temp: 40° C., DAD Wavelength range (nm): 210 to 400, Solvent Gradient: A=water+5% Acetonitrile+0.1% HCOOH, B=Acetonitrile+0.1% HCOOH: gradient: 0 min 10% B, 90% A; 0.9-1.8 min 100% B; 1.8-2.2 min 100-10% B; 2.2-2.5 min 10% B; Flow (mL/min) 1.8.

### Method 3:

Spectra were recorded on a Mass Spectrometer from Waters (Acquity QDa Mass Spectrometer) equipped with an electrospray source (Polarity: Positive and Negative Polarity Switch), Capillary: 0.8 kV, Cone range: 25 V, Extractor: V (No extractor voltage for QDa detector) Source Temperature: 120° C., Desolvation Temperature: 600° C., Cone Gas Flow: 50 L/h, Desolvation Gas Flow: 1000 L/h, Mass range: 110 to 850 Da) and an Acquity UPLC from Waters: Quaternary solvent manager, heated column compartment, diode-array detector. Column: Waters UPLC HSS T3, 1.8 μm, 30×2.1 mm, Temp: 40° C., PDA Wavelength range (nm): 230 to 400, Solvent Gradient: A=Water with 0.1% formic acid: Acetonitrile: 95:5 v/v, B=Acetonitrile with 0.05% formic acid, Gradient: 0 min-1.0 min, 10% B-90% A; 1.0 min-4.50 min 10%-100% B; 4.51 min-5.30 min, 100% B, 0% A; 5.31 min-5.50 min 100%-10% B; 5.51 min-6.00 min, 10% B, 90% A; Flow (ml/min) 0.6.

### Method 4:

Spectra were recorded on a Mass Spectrometer from Agilent Technologies (6410 Triple Quadrupole mass spectrometer) equipped with an electrospray source (Polarity: positive or negative ions, MS2 Scan, Capillary: 7.00 kV, Fragmentor: 120 V, Desolvatation Temperature: 350° C., Gas Flow: 11 L/min, Nebulizer Gas: 40 psi, Mass range: 110 to 1000 Da) and a 1200 Series HPLC from Agilent: quaternary pump, heated column compartment and diode-array detector. Column: KINETEX EVO C18, 2.6 μm, 50×4.6 mm, Temp: 40° C., Detector VWD Wavelength: 254 nm, Solvent Gradient: A=water+5% Acetonitrile+0.1% HCOOH, B=Acetonitrile+0.1% HCOOH: gradient: 0 min 10% B, 90% A; 0.9-1.8 min 100% B; 1.8-2.2 min 100-10% B; 2.2-2.5 min 10% B; Flow (mL/min) 1.8.

### Example P1: Preparation of 6-(3-ethylsulfonyl-2-pyridyl)-1-(2,2,3,3,3-pentafluoropropyl)-3,4-dihydro-1,7-naphthyridin-2-one (compound P1)

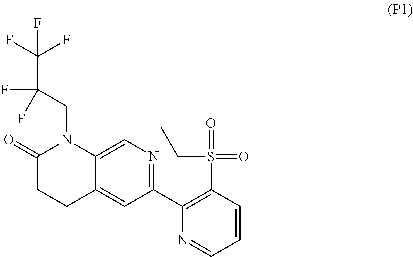 [SMILES: CCS(=O)(=O)c1cccnc1-c1cc2c(cn1)N(CC(F)(F)C(F)(F)F)C(=O)CC2]

### Step 1: Preparation of ethyl 3-(2-chloro-5-nitro-4-pyridyl)-2-oxo-propanoate (intermediate I-1)

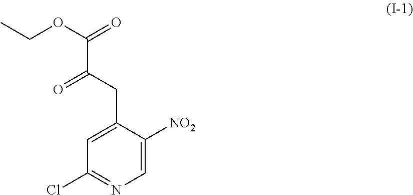 [SMILES: CCOC(=O)C(=O)Cc1cc(Cl)ncc1[N+](=O)[O-]]

To a solution of 2-chloro-4-methyl-5-nitropyridine (CAS 23056-33-9, 1.00 g, 5.79 mmol) in diethyl oxalate (7.59 mL, 54.5 mmol) under nitrogen atmosphere was added 1,8-diazabicyclo[5.4.0]undec-7-ene (1.15 mL, 7.53 mmol) at room temperature over a period of 10 minutes. The reaction mixture was stirred at room temperature overnight. The reaction mixture was then poured into ice cold water (10 mL), acidified with 2N hydrochloric acid (3 mL) and stirred for 5 minutes. The solvent was decanted off and the resulting residue was stirred in ice cold methanol for 20 minutes. The precipitate was filtered and dried in vacuo to afford pure ethyl 3-(2-chloro-5-nitro-4-pyridyl)-2-oxo-propanoate. LCMS (Method 1): Rt=0.96 min, m/z=271/273 (M−H)⁻.

### Step 2: Preparation of ethyl 3-(2-chloro-5-nitro-4-pyridyl)-2-hydroxy-propanoate (intermediate I-2)

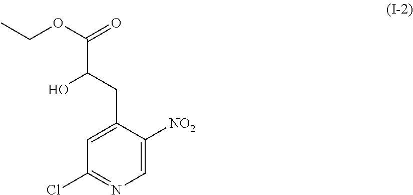 [SMILES: CCOC(=O)C(O)Cc1cc(Cl)ncc1[N+](=O)[O-]]

To an ice cooled solution of ethyl 3-(2-chloro-5-nitro-4-pyridyl)-2-oxo-propanoate (intermediate I-1 prepared as described above, 0.500 g, 1.80 mmol) in tetrahydrofuran (4 mL) and water (1 mL) was added portionwise sodium borohydride (70 mg, 1.8 mmol) at 0° C. The reaction mixture was stirred for 15 minutes at 0° C. Ice cold water was added to the reaction mixture and it was quenched at 0° C. with saturated aqueous ammonium chloride. The resulting suspension was extracted three times with ethyl acetate. The combined organic layers were dried over sodium sulfate, filtered and concentrated in vacuo to afford ethyl 3-(2-chloro-5-nitro-4-pyridyl)-2-hydroxy-propanoate which was used without further purification. LCMS (Method 1): Rt=0.88 min, m/z=275/277 (M+H)⁺.

### Step 3: Preparation of 6-chloro-1H-1,7-naphthyridin-2-one (intermediate I-3)

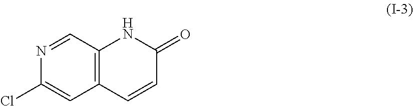

To a solution of ethyl 3-(2-chloro-5-nitro-4-pyridyl)-2-hydroxy-propanoate (intermediate I-2 prepared as described above, 0.400 g, 0.970 mmol) in acetic acid (6 mL) was added iron (0.220 g, 3.90 mmol) at room temperature. The reaction mixture was heated up to 70° C. for 30 minutes. To this reaction mixture were then added 1,4-dioxane (4 mL) and 6N hydrochloric acid (3 mL) at 70° C. The temperature was increased to 90° C. and kept stirring for 4 hours. After cooling down to room temperature, the reaction mixture was filtered over celite and washed with ethyl acetate. The filtrate was concentrated under reduced pressure. The obtained residue was diluted with water (20 mL), cooled to 0° C. and neutralized with saturated aqueous sodium bicarbonate (25 mL). The suspension was filtered and dried under reduced pressure. The crude residue was taken in ethanol:1,2-dichloroethane (1:1, 20 mL) and heated to 70° C. for 30 minutes. The resulting hot solution was filtered over celite and concentrated in vacuo to afford 6-chloro-1H-1,7-naphthyridin-2-one as a brown solid. LCMS (Method 1): Rt=0.36 min, m/z=181/183 (M+H)⁺. ¹H NMR (400 MHz, DMSO-d6) δ ppm 6.82 (d, 1H) 7.83 (s, 1H) 7.92 (d, 1H) 8.44 (s, 1H) 11.99-12.23 (m, 1H).

### Step 4: Preparation of 6-chloro-1-(2,2,3,3,3-pentafluoropropyl)-1,7-naphthyridin-2-one (intermediate I-4)

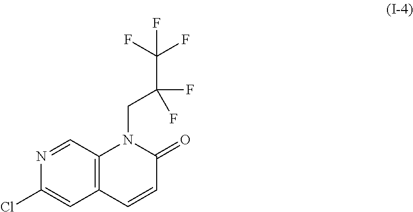 [SMILES: O=c1ccc2cc(Cl)ncc2n1CC(F)(F)C(F)(F)F]

To a solution of 6-chloro-1H-1,7-naphthyridin-2-one (intermediate I-3 prepared as described above, 2.50 g, 13.8 mmol) in tetrahydrofuran (50 mL) were added at room temperature potassium carbonate (4.98 g, 36.0 mmol) and 2,2,3,3,3-pentafluoropropyl trifluoromethanesulfonate (3.31 mL, 19.4 mmol). The reaction mixture was stirred at 70° C. for 9 hours and then at 50° C. for overnight. After cooling down to room temperature, it was poured into water and extracted with ethyl acetate. The combined organic layers were washed with brine, dried over sodium sulfate, filtered and concentrated in vacuo. The crude was purified by combiflash (silica gel, ethyl acetate in cyclohexane) to afford 6-chloro-1-(2,2,3,3,3-pentafluoropropyl)-1,7-naphthyridin-2-one. LCMS (Method 1): Rt=0.99 min, m/z=313/315 (M+H)⁺. ¹H NMR (400 MHz, chloroform-d) δ ppm 5.03 (br s, 2H) 6.97 (d, 1H) 7.51 (s, 1H) 7.67 (d, 1H) 8.57 (s, 1H).

### Step 5: Preparation of 6-(3-fluoro-2-pyridyl)-1-(2,2,3,3,3-pentafluoropropyl)-1,7-naphthyridin-2-one (intermediate I-5)

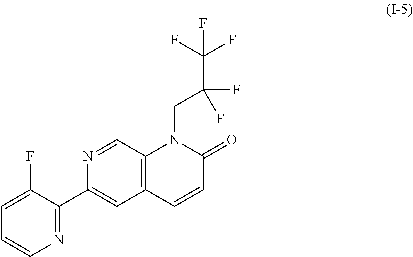 [SMILES: O=c1ccc2cc(-c3ncccc3F)ncc2n1CC(F)(F)C(F)(F)F]

To a solution of 6-chloro-1-(2,2,3,3,3-pentafluoropropyl)-1,7-naphthyridin-2-one (intermediate I-4 prepared as described above, 0.800 g, 2.56 mmol) in 1,4-dioxane (15 mL) was added 2-dicyclohexylphosphino-2′,4′,6′-triisopropylbiphenyl (XPhos) (0.30 g, 0.61 mmol). The reaction mixture was degassed under nitrogen atmosphere for 15 minutes, before adding palladium(II) acetate (0.059 g, 0.26 mmol). The reaction mixture was again degassed for additional 15 minutes, before adding tributyl-(3-fluoro-2-pyridyl)stannane (1.51 g, 3.84 mmol). The reaction mass was then heated up to 90° C. and stirred overnight. After cooling down to room temperature, it was poured into water and extracted with ethyl acetate. The combined organic layers were washed with brine, dried over sodium sulfate, filtered and concentrated in vacuo. The crude was purified by combiflash (silica gel, ethyl acetate in cyclohexane) to afford 6-(3-fluoro-2-pyridyl)-1-(2,2,3,3,3-pentafluoropropyl)-1,7-naphthyridin-2-one. LCMS (Method 1): Rt=0.95 min, m/z=374 (M+H)⁺.

### Step 6: Preparation of 6-(3-ethylsulfanyl-2-pyridyl)-1-(2,2,3,3,3-pentafluoropropyl)-1,7-naphthyridin-2-one (intermediate I-6)

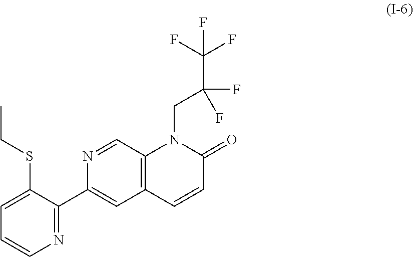 [SMILES: CCSc1cccnc1-c1cc2ccc(=O)n(CC(F)(F)C(F)(F)F)c2cn1]

To a solution of 6-(3-fluoro-2-pyridyl)-1-(2,2,3,3,3-pentafluoropropyl)-1,7-naphthyridin-2-one (intermediate I-5 prepared as described above, 0.200 g, 0.536 mmol) in dry N,N-dimethylformamide (5 mL) was added at room temperature under nitrogen atmosphere ethylsulfanylsodium (0.110 g, 1.18 mmol). The reaction mixture was stirred at room temperature for 90 minutes. Water was then added and it was extracted with ethyl acetate. The combined organic layers were washed with brine, dried over sodium sulfate, filtered and concentrated in vacuo. The crude was purified by combiflash (silica gel, ethyl acetate in cyclohexane) to afford 6-(3-ethylsulfanyl-2-pyridyl)-1-(2,2,3,3,3-pentafluoropropyl)-1,7-naphthyridin-2-one. LCMS (Method 1): Rt=1.07 min, m/z=416 (M+H)⁺.

### Step 7: Preparation of 6-(3-ethylsulfonyl-2-pyridyl)-1-(2,2,3,3,3-pentafluoropropyl)-1,7-naphthyridin-2-one (intermediate I-7)

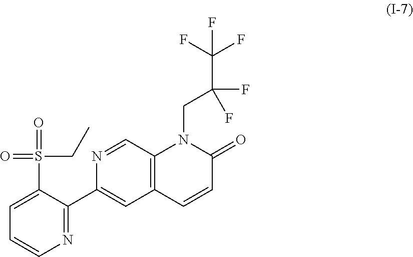 [SMILES: CCS(=O)(=O)c1cccnc1-c1cc2ccc(=O)n(CC(F)(F)C(F)(F)F)c2cn1]

To a solution of 6-(3-ethylsulfanyl-2-pyridyl)-1-(2,2,3,3,3-pentafluoropropyl)-1,7-naphthyridin-2-one (intermediate I-6 prepared as described above, 0.065 g, 0.16 mmol) in dichloromethane (5 mL) was added 3-chloroperbenzoic acid (0.085 g, 0.34 mmol) at 0° C. The reaction mixture was stirred at room temperature for 2 hours. The reaction mixture was then quenched with saturated aqueous potassium carbonate and water, and extracted with ethyl acetate. The combined organic layers were washed with brine, dried over sodium sulfate, filtered and concentrated in vacuo. The crude was purified by combiflash (silica gel, ethyl acetate in cyclohexane) to afford 6-(3-ethylsulfonyl-2-pyridyl)-1-(2,2,3,3,3-pentafluoropropyl)-1,7-naphthyridin-2-one as an off-white solid. LCMS (Method 1): Rt=0.97 min, m/z=448 (M+H)⁺.

### Step 8: Preparation of 6-(3-ethylsulfonyl-2-pyridyl)-1-(2,2,3,3,3-pentafluoropropyl)-3,4-dihydro-1,7-naphthyridin-2-one (compound P1)

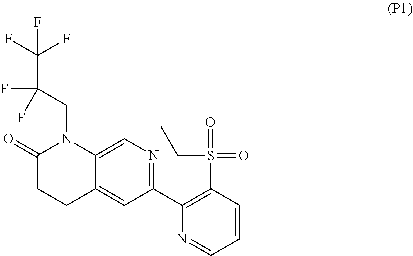 [SMILES: CCS(=O)(=O)c1cccnc1-c1cc2c(cn1)N(CC(F)(F)C(F)(F)F)C(=O)CC2]

To a 0° C. cooled solution of 6-(3-ethylsulfonyl-2-pyridyl)-1-(2,2,3,3,3-pentafluoropropyl)-1,7-naphthyridin-2-one (intermediate I-7 prepared as described above, 0.025 g, 0.055 mmol) in tetrahydrofuran (0.5 mL) was added a solution of saturated aqueous ammonium chloride (0.25 mL) followed by zinc (0.036 g, 0.558 mmol) and catalytic amount of trifluoroacetic acid (0.000638 g, 0.0055 mmol). The reaction mixture was allowed to come to at room temperature and stirred for 16 hours. The reaction mixture was quenched with saturated aqueous ammonium chloride (5 mL), and the mixture was extracted with ethyl acetate (3×10 mL). The combined organic layers were dried over sodium sulfate, filtered and concentrated in vacuo. The crude was purified by combiflash (silica gel, ethyl acetate in cyclohexane) to afford pure 6-(3-ethylsulfonyl-2-pyridyl)-1-(2,2,3,3,3-pentafluoropropyl)-3,4-dihydro-1,7-naphthyridin-2-one as a faint yellow solid. LCMS (Method 1): Rt=0.97 min, m/z=450 (M+H)⁺. ¹H NMR (400 MHz, chloroform-d) δ ppm 1.40 (t, 3H) 2.83 (dd, 2H) 3.05-3.12 (m, 2H) 3.94 (q, 2H) 4.80 (br s, 2H) 7.57 (dd, 1H) 7.74 (s, 1H) 8.39 (s, 1H) 8.50 (dd, 1H) 8.88 (dd, 1H).

### Example P2: Preparation of 1-[5-ethylsulfonyl-6-[2-oxo-1-(2,2,2-trifluoroethyl)-3,4-dihydro-1,7-naphthyridin-6-yl]-3-pyridyl]cyclopropanecarbonitrile (compound P2)

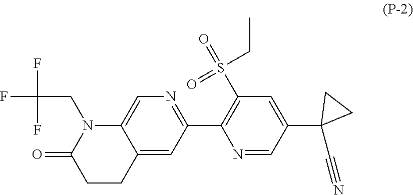 [SMILES: CCS(=O)(=O)c1cc(C2(C#N)CC2)cnc1-c1cc2c(cn1)N(CC(F)(F)F)C(=O)CC2]

### Step 1: Preparation of ethyl 3-(2-chloro-5-nitro-4-pyridyl)-2-oxo-propanoate (intermediate I-8)

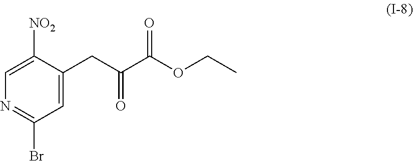 [SMILES: CCOC(=O)C(=O)Cc1cc(Br)ncc1[N+](=O)[O-]]

To a solution of 2-bromo-4-methyl-5-nitro-pyridine (5.00 g, 23.0 mmol) in diethyl oxalate (30.2 mL, 217 mmol) under nitrogen atmosphere was added 1,8-diazabicyclo[5.4.0]undec-7-ene (4.03 mL, 26.5 mmol) at room temperature over a period of 10 minutes. The reaction mixture was stirred at room temperature overnight. The reaction mixture was then poured into ice cold water (50 mL), acidified with 2N hydrochloric acid (20 mL) and stirred for 15 minutes. The solvent was decanted off and the resulting residue was stirred in ice cold ethanol (50 mL) for 20 minutes. The precipitate was filtered through a Buchner funnel and dried in vacuo to afford pure ethyl 3-(2-bromo-5-nitro-4-pyridyl)-2-oxo-propanoate as a solid. LCMS (Method 1): Rt=0.96 min, m/z=317/319 (M+H)⁺.

### Step 2: Preparation of ethyl 3-(2-bromo-5-nitro-4-pyridyl)-2-hydroxy-propanoate (intermediate I-9)

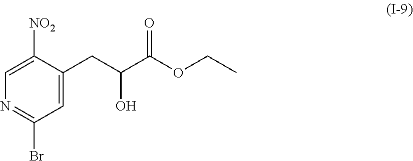 [SMILES: CCOC(=O)C(O)Cc1cc(Br)ncc1[N+](=O)[O-]]

To a 0° C. cooled solution of ethyl 3-(2-bromo-5-nitro-4-pyridyl)-2-oxo-propanoate (intermediate I-8 prepared as described above, 3.00 g, 9.46 mmol) in tetrahydrofuran (60 mL) was added portion wise sodium borohydride (0.438 g, 11.4 mmol). The reaction mass was slowly allowed to come to 5° C. over a period of 30 minutes. The reaction mass was quenched with ice cold water (50 mL), neutralised with saturated aqueous saturated aqueous ammonium chloride (50 mL), and the resulting suspension was extracted with ethyl acetate (3×100 mL). The combined organic layers were dried over sodium sulfate, filtered and concentrated in vacuo. The crude was purified by combiflash (silica gel, 15% ethyl acetate in cyclohexane) to afford pure ethyl 3-(2-bromo-5-nitro-4-pyridyl)-2-hydroxy-propanoate as a faint yellow thick oil. LCMS (Method 1): Rt=0.90 min, m/z=319/321 (M+H)⁺.

### Step 3: Preparation of 6-bromo-1H-1,7-naphthyridin-2-one (intermediate I-10)

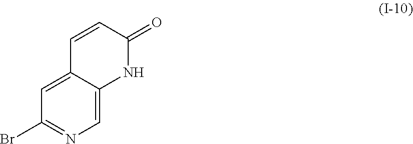 [SMILES: O=c1ccc2cc(Br)ncc2[nH]1]

(1-10)

To a solution of ethyl 3-(2-bromo-5-nitro-4-pyridyl)-2-hydroxy-propanoate (intermediate I-9 prepared as described above, 0.500 g, 1.57 mmol) in acetic acid (5 mL) was added iron (0.351 g, 6.27 mmol) at room temperature. The reaction mixture was heated up to 70° C. for 60 minutes. To this reaction mixture were then added 1,4-dioxane (10 mL) and 5N hydrochloric acid (10 mL) at 70° C. The temperature was increased to 90° C. and kept stirring for 4 hours. The progress of the reaction was monitored by LCMS. LCMS shows partial conversion, additional 5N hydrochloric acid (5 mL) was added and stirred at 90° C. for another 12 hours. After cooling down to room temperature, the reaction mixture was filtered over celite and washed with ethyl acetate (2×10 mL). The filtrate was concentrated under reduced pressure. The obtained residue was diluted with water (10 mL), cooled to 0° C. and neutralized with a solution of saturated aqueous sodium bicarbonate (35 mL). The resulting suspension was extracted with ethyl acetate (7×30 mL). The combined organic layers were dried over sodium sulfate, filtered and concentrated in vacuo. The crude compound was purified by washing with n-pentane (3×5 mL), dried in vacuo to afford 6-bromo-1H-1,7-naphthyridin-2-one as an off-white solid. LCMS (Method 1): Rt=0.68 min, m/z=225/227 (M+H)⁺.

### Step 4: Preparation of 6-bromo-1-(2,2,2-trifluoroethyl)-1,7-naphthyridin-2-one (intermediate I-11)

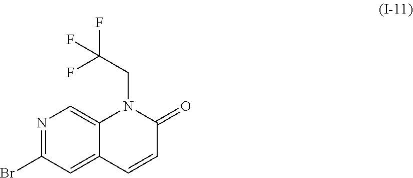 [SMILES: O=c1ccc2cc(Br)ncc2n1CC(F)(F)F]

To a solution of 6-bromo-1H-1,7-naphthyridin-2-one (intermediate I-10 prepared as described above, 6.00 g, 25.06 mmol) in tetrahydrofuran (60 mL) was added potassium carbonate (12.12 g, 87.71 mmol) followed by addition of 2,2,2-trifluoroethyl trifluoromethanesulfonate (11.2 mL, 75.18 mmol) at room temperature. The reaction mass was stirred at 75° C. for 5 hours. After cooling down to room temperature, the reaction mass was concentrated in vacuo, then quenched with ice cooled water and extracted with ethyl acetate (2×150 mL). The combined organic layers were washed with brine, dried over sodium sulfate, filtered and concentrated in vacuo. The crude was purified by combiflash (silica gel, ethyl acetate in cyclohexane) to afford 6-bromo-1-(2,2,2-trifluoroethyl)-1,7-naphthyridin-2-one. LCMS (Method 1): Rt=1.01 min, m/z=307/309 (M+H)⁺. ¹H NMR (400 MHz, chloroform-d) δ ppm 5.00 (br d, 2H) 6.95 (d, 1H) 7.62-7.68 (m, 2H) 8.58 (s, 1H).

### Step 5: Preparation of 1-[5-fluoro-1-oxido-6-[2-oxo-1-(2,2,2-trifluoroethyl)-1,7-naphthyridin-6-yl]pyridin-1-ium-3-yl]cyclopropanecarbonitrile (intermediate I-12)

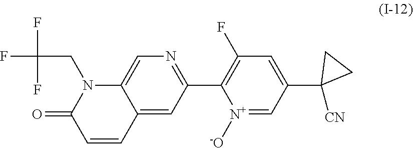

A solution of 2,2,6,6-tetramethylpiperidinylzinc chloride lithium chloride complex in tetrahydrofuran (14.6 mL, 14.65 mmol, 1 mol/l) was added dropwise at 10° C. to a degassed solution of 1-(5-fluoro-1-oxido-pyridin-1-ium-3-yl)cyclopropanecarbonitrile (CAS 2489316-32-5, prepared as described in WO2020182577) (2.61 g, 14.65 mmol) in tetrahydrofuran (30 mL) under nitrogen and stirred for 15 minutes. A degassed solution of 6-bromo-1-(2,2,2-trifluoroethyl)-1,7-naphthyridin-2-one (intermediate 1-11 prepared as described above, 3.0 g, 9.76 mmol) in tetrahydrofuran (30 mL) was added to the reaction mixture at 10° C. After complete addition, Pd(dppf)Cl₂ (0.47 g, 0.63 mmol) was added and the reaction mass was heated at 60° C. for 15 hours. The reaction mass was quenched with a saturated aqueous sodium bicarbonate solution (60 mL) and extracted with ethyl acetate (3×60 mL). The combined organic layers were washed with water followed by brine, dried over sodium sulphate, filtered and concentrated in vacuo. The crude was purified by combiflash (silica gel, 0-10% methanol in ethyl acetate) to afford 1-[5-fluoro-1-oxido-6-[2-oxo-1-(2,2,2-trifluoroethyl)-1,7-naphthyridin-6-yl]pyridin-1-ium-3-yl]cyclopropanecarbonitrile along with unreacted 1-(5-fluoro-1-oxido-pyridin-1-ium-3-yl)cyclopropanecarbonitrile. This material was used as such in the next step. LCMS (Method 1): Rt=0.93 min, m/z=405 (M+H)⁺.

### Step 6: Preparation of 1-[5-ethylsulfanyl-1-oxido-6-[2-oxo-1-(2,2,2-trifluoroethyl)-1,7-naphthyridin-6-yl]pyridin-1-ium-3-yl]cyclopropanecarbonitrile (intermediate I-13)

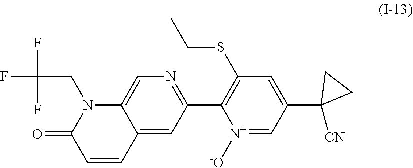

To a 0° C. cooled solution of 1-[5-fluoro-1-oxido-6-[2-oxo-1-(2,2,2-trifluoroethyl)-1,7-naphthyridin-6-yl]pyridin-1-ium-3-yl]cyclopropanecarbonitrile (intermediate I-12 prepared as described above, 2.469 g, 6.107 mmol) in N,N-dimethylformamide (15 mL) was added sodium ethanethiolate (0.770 g, 9.16 mmol) under nitrogen atmosphere. The reaction mixture stirred at room temperature for 2 hours. The reaction mass was quenched with ice cold water (100 mL) and extracted with ethyl acetate (3×). The combined organic layers were washed with water followed by brine, dried over sodium sulphate, filtered and concentrated in vacuo. The crude was purified by combiflash (silica gel, 0-10% methanol in cyclohexane) to afford 1-[5-ethylsulfanyl-1-oxido-6-[2-oxo-1-(2,2,2-trifluoroethyl)-1,7-naphthyridin-6-yl]pyridin-1-ium-3-yl]cyclopropanecarbonitrile with unreacted 1-(5-fluoro-1-oxido-pyridin-1-ium-3-yl)cyclopropanecarbonitrile. LCMS (Method 1): Rt=0.98 min, m/z=447 (M+H)⁺.

### Step 7: Preparation of 1-[5-ethylsulfanyl-6-[2-oxo-1-(2,2,2-trifluoroethyl)-1,7-naphthyridin-6-yl]-3-pyridyl]cyclopropanecarbonitrile (intermediate I-14)

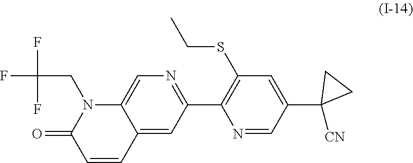 [SMILES: CCSc1cc(C2(C#N)CC2)cnc1-c1cc2ccc(=O)n(CC(F)(F)F)c2cn1]

To the solution of 1-[5-ethylsulfanyl-1-oxido-6-[2-oxo-1-(2,2,2-trifluoroethyl)-1,7-naphthyridin-6-yl]pyridin-1-ium-3-yl]cyclopropanecarbonitrile (intermediate I-13 prepared as described above, 1.769 g, 3.96 mmol) in dry acetonitrile (35.4 mL) was added 4,4,5,5-tetramethyl-2-(4,4,5,5-tetramethyl-1,3,2-dioxaborolan-2-yl)-1,3,2-dioxaborolane (1.509 g, 5.944 mmol) and the reaction mixture was stirred at 70° C. for 3 hrs, followed by at 70° C. for 12 hours. The reaction mass was diluted with water (100 mL) and extracted with ethyl acetate (3×100 mL). The combined organic layers were washed with brine (50 mL), dried over anhydrous sodium sulphate, filtered and concentrated in vacuo. The crude was purified by combiflash (silica gel, 0-40% ethyl acetate in cyclohexane) to afford 1 1-[5-ethylsulfanyl-6-[2-oxo-1-(2,2,2-trifluoroethyl)-1,7-naphthyridin-6-yl]-3-pyridyl]cyclopropanecarbonitrile as an off white solid. LCMS (Method 1): Rt=1.09 min, m/z=431 (M+H)⁺. ¹H NMR (400 MHz, chloroform-d) δ ppm 1.38 (t, 3H) 1.52-1.58 (m, 2H) 1.85-1.90 (m, 2H) 2.98 (q, 2H) 5.08 (br d, 2H) 6.95 (d, 1H) 7.71 (d, 1H) 7.81 (d, 1H) 8.24-8.30 (m, 2H) 8.94 (s, 1H).

### Step 8: Preparation of 1-[5-ethylsulfanyl-6-[2-oxo-1-(2,2,2-trifluoroethyl)-3,4-dihydro-1,7-naphthyridin-6-yl]-3-pyridyl]cyclopropanecarbonitrile (compound P6)

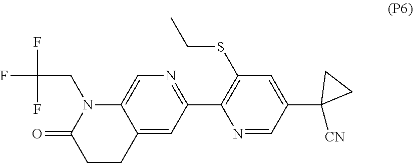 [SMILES: CCSc1cc(C2(C#N)CC2)cnc1-c1cc2c(cn1)N(CC(F)(F)F)C(=O)CC2]

To a 0° C. cooled solution of 1-[5-ethylsulfanyl-6-[2-oxo-1-(2,2,2-trifluoroethyl)-1,7-naphthyridin-6-yl]-3-pyridyl]cyclopropanecarbonitrile (intermediate I-14 prepared as described above, 0.680 g, 1.580 mmol) in tetrahydrofuran (13.6 mL) was added a solution of saturated aqueous ammonium chloride (6.8 mL) followed by addition of catalytic amount of trifluoroacetic acid (0.018 g, 0.158 mmol,) and zinc (0.516 g, 7.90 mmol). The reaction mixture was stirred at room temperature for 2.5 hours. The reaction mass was quenched with saturated aqueous ammonium chloride solution (50 mL), extracted with ethyl acetate (3×50 mL). The combined organic layers were dried over sodium sulfate, filtered and concentrated in vacuo. The crude was purified by combiflash (silica gel, 0-50% ethyl acetate in cyclohexane) to afford pure 1-[5-ethylsulfanyl-6-[2-oxo-1-(2,2,2-trifluoroethyl)-3,4-dihydro-1,7-naphthyridin-6-yl]-3-pyridyl]cyclopropanecarbonitrile as a white solid. LCMS (Method 3): Rt=1.10 min, m/z=433 (M+H)⁺. ¹H NMR (400 MHz, chloroform-d) δ ppm 1.38 (t, 3H) 1.51-1.56 (m, 2H) 1.84-1.89 (m, 2H) 2.80 (dd, 2H) 2.97 (q, 2H) 3.05-3.11 (m, 2H) 4.73 (q, 2H) 7.68 (d, 1H) 7.95 (s, 1H) 8.25 (d, 1H) 8.52 (s, 1H).

### Step 9: Preparation of 1-[5-ethylsulfonyl-6-[2-oxo-1-(2,2,2-trifluoroethyl)-3,4-dihydro-1,7-naphthyridin-6-yl]-3-pyridyl]cyclopropanecarbonitrile (compound P2)

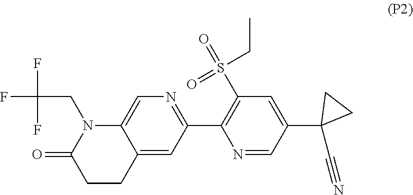 [SMILES: CCS(=O)(=O)c1cc(C2(C#N)CC2)cnc1-c1cc2c(cn1)N(CC(F)(F)F)C(=O)CC2]

To a 0° C. cooled solution of 1-[5-ethylsulfanyl-6-[2-oxo-1-(2,2,2-trifluoroethyl)-3,4-dihydro-1,7-naphthyridin-6-yl]-3-pyridyl]cyclopropanecarbonitrile (compound P6 prepared as described above, 0.370 g, 0.8555 mmol) in acetonitrile (7.4 mL) was added 3-chlorobenzenecarboperoxoic acid (0.464 g, 1.882 mmol, 70 mass %). The reaction mass was stirred at 0-10° C. for 2 hours. The reaction mass was quenched with 2N aqueous sodium hydroxide solution (10 mL) and water (20 mL), extracted with ethyl acetate (3×30 mL), The combined organic layers were washed with brine (30 mL), dried over sodium sulfate, filtered and concentrated in vacuo. The crude was purified by combiflash (silica gel, 0-80% ethyl acetate in cyclohexane) to afford pure 1-[5-ethylsulfonyl-6-[2-oxo-1-(2,2,2-trifluoroethyl)-3,4-dihydro-1,7-naphthyridin-6-yl]-3-pyridyl]cyclopropanecarbonitrile as a white solid. LCMS (Method 1): Rt=1.03 min, m/z=465 (M+H)⁺. ¹H NMR (400 MHz, chloroform-d) δ ppm 1.41 (t, 3H) 1.60-1.63 (m, 2H) 1.94-1.99 (m, 2H) 2.83 (dd, 2H) 3.07-3.13 (m, 2H) 3.98 (q, 2H) 4.74 (q, 2H) 7.75 (s, 1H) 8.23 (d, 1H) 8.40 (s, 1H) 8.94 (d, 1H).

### Example P3: Preparation of 1-[5-ethylsulfonyl-6-[2-oxo-1-(2,2,3,3,3-pentafluoropropyl)-4H-pyrido[3,4-d][1,3]oxazin-6-yl]-3-pyridyl]cyclopropanecarbonitrile (compound P3)

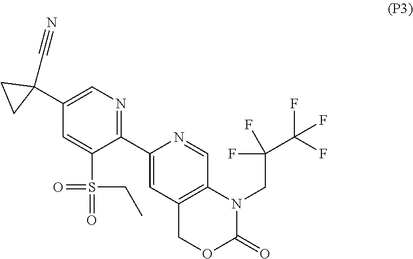 [SMILES: CCS(=O)(=O)c1cc(C2(C#N)CC2)cnc1-c1cc2c(cn1)N(CC(F)(F)C(F)(F)F)C(=O)OC2]

### Step 1: Preparation of (E)-2-(2-bromo-5-nitro-4-pyridyl)-N,N-dimethyl-ethenamine (intermediate I-16)

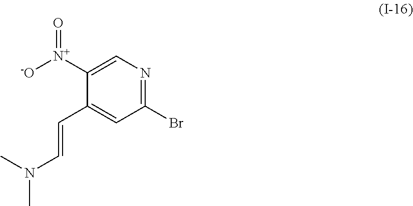

To a solution of 2-bromo-4-methyl-5-nitro-pyridine (10.0 g, 43.8 mmol) in N,N-dimethylformamide (153 mL) was added 1,1-dimethoxy-N,N-dimethylmethylamine (DMF-DMA, 24 mL, 175 mmol) followed by 1,8-diazabicyclo[5.4.0]undec-7-ene (0.668 ml, 4.38 mmol) dropwise through syringe and the reaction mass was stirred at room temperature for overnight. The reaction mixture was concentrated in vacuo to afford (E)-2-(2-bromo-5-nitro-4-pyridyl)-N,N-dimethyl-ethenamine. This material was used as such in the next step. LCMS (Method 2): Rt=1.42 min, m/z=272/274 (M+H)⁺.

### Step 2: Preparation of 2-bromo-5-nitro-pyridine-4-carbaldehyde (intermediate I-17)

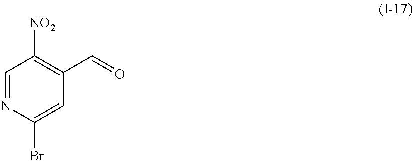 [SMILES: O=Cc1cc(Br)ncc1[N+](=O)[O-]]

To a solution of (E)-2-(2-bromo-5-nitro-4-pyridyl)-N,N-dimethyl-ethenamine (intermediate I-16 prepared as described above, 1.0 g, 3.3 mmol) in tetrahydrofuran (10 mL) and water (10 mL) was added sodium periodate (2.6 g, 12 mmol). The reaction mixture was stirred at room temperature for overnight. Upon completion, the reaction mixture was quenched with water, and the mixture was extracted with ethyl acetate (3×150 mL). The combined organic layers were dried over sodium sulfate, filtered and concentrated in vacuo to afford 2-bromo-5-nitro-pyridine-4-carbaldehyde. This material was used as such in the next step. ¹H NMR (400 MHz, chloroform-d) δ ppm 8.04 (s, 1H) 9.23 (s, 1H) 10.30 (s, 1H).

### Step 3: Preparation of (2-bromo-5-nitro-4-pyridyl)methanol (intermediate I-18)

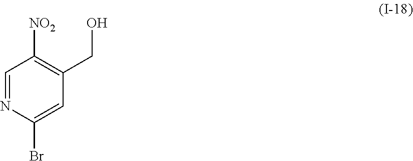 [SMILES: O=[N+]([O-])c1cnc(Br)cc1CO]

To a 0° C. cooled solution of 2-bromo-5-nitro-pyridine-4-carbaldehyde (intermediate I-17 prepared as described above, 0.500 g, 2.056 mmol) in methanol (5 mL) was added sodium borohydride (0.086 g, 2.056 mmol) portionwise and continued stirring for 5 minutes under nitrogen. The reaction mass was quenched with water (30 mL) and extracted with ethyl acetate (2×30 mL). The combined organic layers were dried over sodium sulfate, filtered and concentrated in vacuo. The crude was purified by combiflash (silica gel, ethyl acetate in cyclohexane) to afford pure (2-bromo-5-nitro-4-pyridyl)methanol. LCMS (Method 1): Rt=0.76 min, m/z=233/235 (M+H)⁺.

### Step 4: Preparation of (5-amino-2-bromo-4-pyridyl)methanol (intermediate I-19)

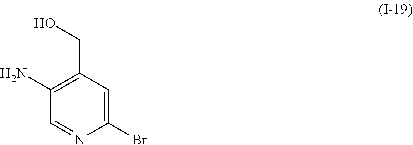

To a solution of (2-bromo-5-nitro-4-pyridyl)methanol (intermediate I-18 prepared as described above, 0.100 g, 0.4291 mmol) in tetrahydrofuran (0.9 mL), ethanol (0.9 mL) and water (0.3 mL) was added iron (0.126 g, 2.1457 mmol) portion wise followed by ammonium chloride (0.035 gm, 0.64372 mmol.) The reaction mixture was stirred at 95° C. for 4 hours. The reaction mixture was cooled to room temperature and then diluted with ethanol and filtered through celite, the filtrate was then concentrated in vacuo. The obtained residue was dissolved in ethyl acetate and washed with water followed by brine. The organic layer was dried over sodium sulfate, filtered and concentrated in vacuo to afford (5-amino-2-bromo-4-pyridyl)methanol. This material was used as such in the next step. LCMS (Method 1): Rt=0.17 min, m/z=203/205 (M+H)⁺.

### Step 5: Preparation of 6-bromo-1,4-dihydropyrido[3,4-d][1,3]oxazin-2-one (intermediate I-20)

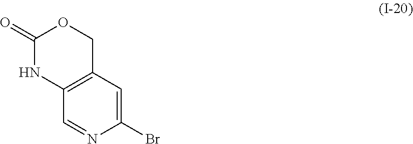 [SMILES: O=C1Nc2cnc(Br)cc2CO1]

To a 0° C. cooled solution of (5-amino-2-bromo-4-pyridyl)methanol (intermediate I-19 prepared as described above, 1.2 g, 5.3 mmol) in anhydrous 1,4-dioxane (12 mL) was added triethylamine (1.5 mL, 11 mmol) followed by bis(trichloromethyl) carbonate (0.81 g, 2.7 mmol) in one portion under nitrogen. The reaction mixture was stirred at room temperature for overnight. The reaction mass was quenched with ice cold water and extracted with ethyl acetate (3×10 mL). The combined organic layers were dried over sodium sulfate and concentrated in vacuo to afford 6-bromo-1,4-dihydropyrido[3,4-d][1,3]oxazin-2-one as an off white solid. LCMS (Method 1): Rt=0.22 min, m/z=229/231 (M+H)⁺. ¹H NMR (400 MHz, DMSO-d6) δ ppm 5.34-5.37 (m, 2H) 7.54 (s, 1H) 7.92 (s, 1H) 10.53 (s, 1H).

### Step 6: Preparation of 6-bromo-1-(2,2,3,3,3-pentafluoropropyl)-4H-pyrido[3,4-d][1,3]oxazin-2-one (intermediate I-21)

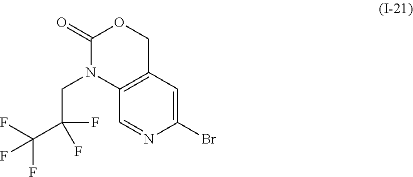 [SMILES: O=C1OCc2cc(Br)ncc2N1CC(F)(F)C(F)(F)F]

To a 0° C. cooled solution of 6-bromo-1,4-dihydropyrido[3,4-d][1,3]oxazin-2-one (intermediate I-20 prepared as described above, 2.02 g, 8.38 mmol) in acetonitrile (20.2 mL) was added dipotassium; carbonate (1.74 g, 12.6 mmol) under nitrogen. 2,2,3,3,3-pentafluoropropyl trifluoromethanesulfonate (4.87 g, 16.8 mmol) was added dropwise to the reaction mass. The reaction mass was stirred at room temperature for overnight. The reaction mass was quenched with ice cold water and extracted with ethyl acetate (3×50 mL). The combined organic layers were dried over sodium sulfate and concentrated in vacuo. The crude was purified by combiflash (silica gel, ethyl acetate in cyclohexane) to afford pure 6-bromo-1-(2,2,3,3,3-pentafluoropropyl)-4H-pyrido[3,4-d][1,3]oxazin-2-one) as a pure cream colored solid. LCMS (Method 1): Rt=1.06 min, m/z=361/363 (M+H)⁺. ¹H NMR (400 MHz, chloroform-d) δ ppm 4.64-4.85 (m, 2H) 5.27 (s, 2H) 7.36 (s, 1H) 8.18 (s, 1H).

### Step 7: Preparation of 1-[5-fluoro-1-oxido-6-[2-oxo-1-(2,2,3,3,3-pentafluoropropyl)-4H-pyrido[3,4-d][1,3]oxazin-6-yl]pyridin-1-ium-3-yl]cyclopropanecarbonitrile (intermediate I-22)

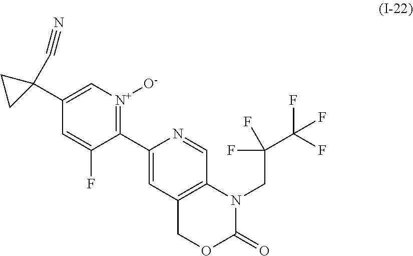 [SMILES: N#CC1(c2cc(F)c(-c3cc4c(cn3)N(CC(F)(F)C(F)(F)F)C(=O)OC4)[n+]([O-])c2)CC1]

A solution of 2,2,6,6-tetramethylpiperidinylzinc chloride lithium chloride complex in tetrahydrofuran (7.6 mL, 7.6 mmol, 1 mol/l) was added dropwise at 0° C. to a degassed solution of 1-(5-fluoro-1-oxido-pyridin-1-ium-3-yl)cyclopropanecarbonitrile (CAS 2489316-32-5, prepared as described in WO2020182577) (1.3 g, 7.6 mmol) in tetrahydrofuran (13 mL) under nitrogen and stirred for 15 minutes. A degassed solution of 6-bromo-1-(2,2,3,3,3-pentafluoropropyl)-4H-pyrido[3,4-d][1,3]oxazin-2-one (intermediate I-21 prepared as described above, 2.1 g, 5.8 mmol) in tetrahydrofuran (21 mL) was added to the reaction mixture at 10° C. After complete addition, Pd(dppf)Cl2 (0.26 g, 0.35 mmol) was added and the reaction mass was heated at 60° C. for 16 hours. The reaction mass was quenched with a saturated aqueous sodium bicarbonate solution (30 mL) and extracted with ethyl acetate (3×). The combined organic layers were washed with water followed by brine, dried over sodium sulphate, filtered and concentrated in vacuo. The crude was purified by combiflash (silica gel, 65% ethyl acetate in cyclohexane) to afford 1-[5-fluoro-1-oxido-6-[2-oxo-1-(2,2,3,3,3-pentafluoropropyl)-4H-pyrido[3,4-d][1,3]oxazin-6-yl]pyridin-1-ium-3-yl]cyclopropanecarbonitrile along with unreacted 1-(5-fluoro-1-oxido-pyridin-1-ium-3-yl)cyclopropanecarbonitrile. This material was used as such in the next step. LCMS (Method 1): Rt=0.99 min, m/z=459 (M+H)⁺.

Similarly, 6-(3-fluoro-1-oxido-pyridin-1-ium-2-yl)-1-(2,2,3,3,3-pentafluoropropyl)-4H-pyrido[3,4-d][1,3]oxazin-2-one (intermediate I-37) can be prepared from 3-fluoropyridine N-oxide (CAS 695-37-4) and 6-bromo-1-(2,2,3,3,3-pentafluoropropyl)-4H-pyrido[3,4-d][1,3]oxazin-2-one (intermediate I-21). LCMS (Method 1): m/z=394 (M+H)⁺, Rt 0.92 min.

### Step 8: Preparation of 1-[5-ethylsulfanyl-1-oxido-6-[2-oxo-1-(2,2,3,3,3-pentafluoropropyl)-4H-pyrido[3,4-d][1,3]oxazin-6-yl]pyridin-1-ium-3-yl]cyclopropanecarbonitrile (intermediate I-23)

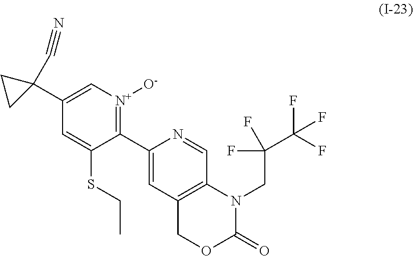 [SMILES: CCSc1cc(C2(C#N)CC2)c[n+]([O-])c1-c1cc2c(cn1)N(CC(F)(F)C(F)(F)F)C(=O)OC2]

To a 0° C. cooled solution of 1-[5-fluoro-1-oxido-6-[2-oxo-1-(2,2,3,3,3-pentafluoropropyl)-4H-pyrido[3,4-d][1,3]oxazin-6-yl]pyridin-1-ium-3-yl]cyclopropanecarbonitrile (intermediate I-22 prepared as described above, 0.360 g, 0.785 mmol) in N-Methyl-2-Pyrrolidone (7.2 mL) was added sodium ethanethiolate (0.123 g, 1.178 mmol) under nitrogen atmosphere. The reaction mixture stirred at room temperature for 5 hours. The reaction was monitored by LCMS. LCMS shows partial conversion, additional sodium ethanethiolate (0.123 g, 1.178 mmol) was added to the reaction mass and stirred at room temperature for another 16 hours. Upon completion, the reaction mass was quenched with ice cold water (10 mL) and extracted with ethyl acetate (3×). The combined organic layers were washed with water followed by brine, dried over sodium sulphate, filtered and concentrated in vacuo. The crude was purified by combiflash (silica gel, 4% methanol in cyclohexane) to afford pure 1-[5-ethylsulfanyl-1-oxido-6-[2-oxo-1-(2,2,3,3,3-pentafluoropropyl)-4H-pyrido[3,4-d][1,3]oxazin-6-yl]pyridin-1-ium-3-yl]cyclopropanecarbonitrile) as a brown thick oil. LCMS (Method 1): Rt=1.01 min. m/z=501 (M+H)⁺. Similarly, 6-(3-ethylsulfanyl-1-oxido-pyridin-1-ium-2-yl)-1-(2,2,3,3,3-pentafluoropropyl)-4H-pyrido[3,4-d][1,3]oxazin-2-one (intermediate I-38) can be prepared from 6-(3-fluoro-1-oxido-pyridin-1-ium-2-yl)-1-(2,2,3,3,3-pentafluoropropyl)-4H-pyrido[3,4-d][1,3]oxazin-2-one (intermediate I-37 prepared as described above). LCMS (Method 1): m/z=436 (M+H)⁺, Rt 0.96 min.

### Step 9: Preparation of 1-[5-ethylsulfanyl-6-[2-oxo-1-(2,2,3,3,3-pentafluoropropyl)-4H-pyrido[3,4-d][1,3]oxazin-6-yl]-3-pyridyl]cyclopropanecarbonitrile (compound P4)

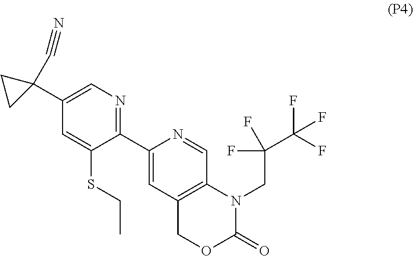 [SMILES: CCSc1cc(C2(C#N)CC2)cnc1-c1cc2c(cn1)N(CC(F)(F)C(F)(F)F)C(=O)OC2]

To 0° C. cooled solution of 1-[5-ethylsulfanyl-1-oxido-6-[2-oxo-1-(2,2,3,3,3-pentafluoropropyl)-4H-pyrido[3,4-d][1,3]oxazin-6-yl]pyridin-1-ium-3-yl]cyclopropanecarbonitrile (intermediate I-23 prepared as described above, 0.250 g, 0.499 mmol) in tetrahydrofuran (5 mL) was added a solution of saturated aqueous ammonium chloride (2.5 mL) followed by addition of zinc (0.0980 g, 1.499 mmol). The reaction mixture was stirred at room temperature for 22 hours. The reaction mass was quenched with water (30 mL) and extracted with ethyl acetate (3×30 mL). The combined organic layers were dried over anhydrous sodium sulphate, filtered and concentrated in vacuo. The crude was purified by combiflash (silica gel, 55% ethyl acetate in cyclohexane) to afford pure 1-[5-ethylsulfanyl-6-[2-oxo-1-(2,2,3,3,3-pentafluoropropyl)-4H-pyrido[3,4-d][1,3]oxazin-6-yl]-3-pyridyl]cyclopropanecarbonitrile as a white solid. LCMS (Method 1): Rt=1.13 min, m/z=485 (M+H)⁺. ¹H NMR (400 MHz, chloroform-d) δ ppm 1.36-1.42 (m, 3H) 1.51-1.57 (m, 2H) 1.82-1.90 (m, 2H) 2.92-3.01 (m, 2H) 4.74-4.88 (m, 2H) 5.38 (s, 2H) 7.70 (d, 1H) 7.99 (s, 1H) 8.22-8.26 (m, 1H) 8.51 (s, 1H).

### Step 10: Preparation of 1-[5-ethylsulfonyl-6-[2-oxo-1-(2,2,3,3,3-pentafluoropropyl)-4H-pyrido[3,4-d][1,3]oxazin-6-yl]-3-pyridyl]cyclopropanecarbonitrile (compound P3)

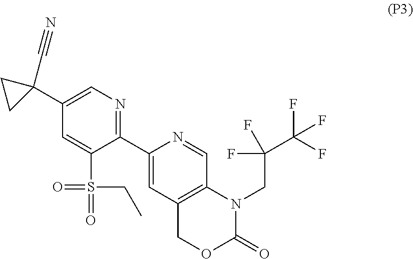 [SMILES: CCS(=O)(=O)c1cc(C2(C#N)CC2)cnc1-c1cc2c(cn1)N(CC(F)(F)C(F)(F)F)C(=O)OC2]

To a 0° C. cooled solution of 1-[5-ethylsulfanyl-6-[2-oxo-1-(2,2,3,3,3-pentafluoropropyl)-4H-pyrido[3,4-d][1,3]oxazin-6-yl]-3-pyridyl]cyclopropanecarbonitrile (compound P4 prepared as described above, 0.220 g, 0.454 mmol) in benzotrifluoride (5 mL) was added 3-chlorobenzenecarboperoxoic acid (0.246 g, 0.999 mmol, 70 mass %). The reaction mass was stirred at room temperature for 2 hours. The reaction mass was quenched with 2N aqueous sodium hydroxide solution (20 mL) and water (10 mL), extracted with ethyl acetate (2×50 mL). The combined organic layers were washed with brine (20 mL), dried over sodium sulfate, filtered and concentrated in vacuo. The crude was purified by combiflash (silica gel, 60% ethyl acetate in cyclohexane) to afford pure 1-[5-ethylsulfonyl-6-[2-oxo-1-(2,2,3,3,3-pentafluoropropyl)-4H-pyrido[3,4-d][1,3]oxazin-6-yl]-3-pyridyl]cyclopropanecarbonitrile as a white solid. LCMS (Method 2): Rt=1.37 min, m/z=517 (M+H)⁺. ¹H NMR (400 MHz, chloroform-d) δ ppm 1.41 (t, 3H) 1.55-1.68 (m, 2H) 1.94-2.01 (m, 2H) 3.95 (q, 2H) 4.75-4.91 (m, 2H) 5.39 (s, 2H) 7.74 (s, 1H) 8.23 (d, 1H) 8.39 (s, 1H) 8.95 (d, 1H).

### Example P13: Preparation of 6-[3-ethylsulfonyl-6-(1,2,4-triazol-1-yl)-2-pyridyl]-1-(2,2,3,3,3-pentafluoropropyl)-3,4-dihydro-1,7-naphthyridin-2-one (compound P13)

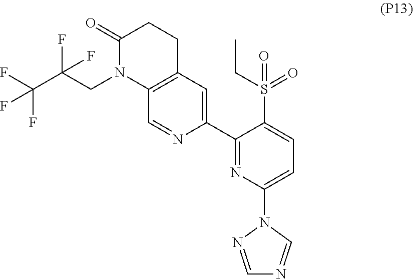 [SMILES: CCS(=O)(=O)c1ccc(-n2cncn2)nc1-c1cc2c(cn1)N(CC(F)(F)C(F)(F)F)C(=O)CC2]

### Step 1: Preparation of 6-(6-chloro-3-ethylsulfanyl-2-pyridyl)-1-(2,2,3,3,3-pentafluoropropyl)-3,4-dihydro-1,7-naphthyridin-2-one (intermediate I-33)

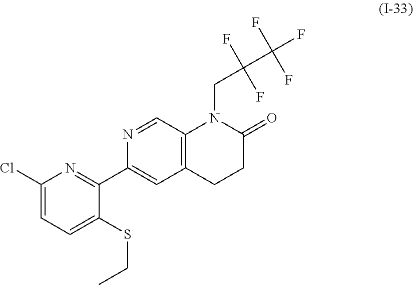 [SMILES: CCSc1ccc(Cl)nc1-c1cc2c(cn1)N(CC(F)(F)C(F)(F)F)C(=O)CC2]

To a solution of 6-(6-chloro-3-ethylsulfanyl-2-pyridyl)-1-(2,2,3,3,3-pentafluoropropyl)-1,7-naphthyridin-2-one (prepared as described in WO21/136722) (3.3 g, 5.1 mmol, 70 mass %) in tetrahydrofuran (66 mL) at 0° C. was added a solution of saturated aqueous ammonium chloride (33 mL) followed by addition of a catalytic amount of trifluoroacetic acid (0.059 g, 0.51 mmol) and zinc (2.4 g, 36 mmol). The reaction mixture was stirred at room temperature for 2 hours, then quenched with a saturated aqueous ammonium chloride solution (10 mL) and the product extracted with ethyl acetate (3×50 mL). The combined organic layers were dried over sodium sulfate, filtered and concentrated in vacuo. The crude was purified by combiflash (ethyl acetate in cyclohexane) to afford 6-(6-chloro-3-ethylsulfanyl-2-pyridyl)-1-(2,2,3,3,3-pentafluoropropyl)-3,4-dihydro-1,7-naphthyridin-2-one (intermediate I-33) as a white solid. LCMS (Method 1): Rt=1.19 min, m/z=452/454 (M+H)⁺.

### Step 2: Preparation of 6-(6-chloro-3-ethylsulfonyl-2-pyridyl)-1-(2,2,3,3,3-pentafluoropropyl)-3,4-dihydro-1,7-naphthyridin-2-one (intermediate I-34)

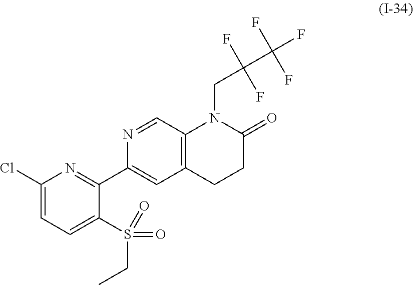 [SMILES: CCS(=O)(=O)c1ccc(Cl)nc1-c1cc2c(cn1)N(CC(F)(F)C(F)(F)F)C(=O)CC2]

To a solution of 6-(6-chloro-3-ethylsulfanyl-2-pyridyl)-1-(2,2,3,3,3-pentafluoropropyl)-3,4-dihydro-1,7-naphthyridin-2-one (intermediate I-33 prepared as described above) (540 mg, 1.20 mmol) in trifluoromethylbenzene (5.4 mL) at 0° C. was added 3-chlorobenzenecarboperoxoic acid (648.2 mg, 2.63 mmol, 70 mass %) portionwise. The reaction mixture was stirred at room temperature for 1.5 hours, then poured into an aqueous saturated sodium bicarbonate solution (80 ml) and the product extracted with ethyl acetate (3×80 mL). The combined organic layers were dried over sodium sulfate, filtered and concentrated in vacuo. The crude product was purified by combiflash (silica gel, 30% ethyl acetate in cyclohexane) to afford 6-(6-chloro-3-ethylsulfonyl-2-pyridyl)-1-(2,2,3,3,3-pentafluoropropyl)-3,4-dihydro-1,7-naphthyridin-2-one (intermediate I-34). LCMS (Method 1): Rt=1.15 min, m/z=484/486 (M+H)⁺.

### Step 3: Preparation of 6-[3-ethylsulfonyl-6-(1,2,4-triazol-1-yl)-2-pyridyl]-1-(2,2,3,3,3-pentafluoropropyl)-3,4-dihydro-1,7-naphthyridin-2-one (compound P13)

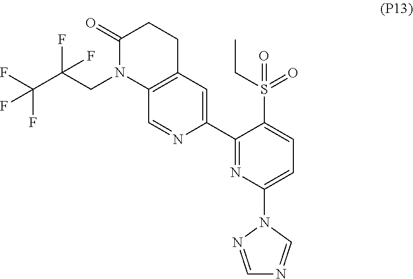 [SMILES: CCS(=O)(=O)c1ccc(-n2cncn2)nc1-c1cc2c(cn1)N(CC(F)(F)C(F)(F)F)C(=O)CC2]

To a solution of 6-(6-chloro-3-ethylsulfonyl-2-pyridyl)-1-(2,2,3,3,3-pentafluoropropyl)-3,4-dihydro-1,7-naphthyridin-2-one (intermediate I-34 prepared as described above) (100 mg, 0.207 mmol) in acetonitrile (1 mL) were added 1H-1,2,4-triazole (21.4 mg, 0.31 mmol) and potassium carbonate (42.85 mg, 0.31 mmol). The reaction mixture was stirred at 90° C. for 2 hours, then diluted with water. The precipitated product was filtered off, washed with n-pentane and dried to afford 6-[3-ethylsulfonyl-6-(1,2,4-triazol-1-yl)-2-pyridyl]-1-(2,2,3,3,3-pentafluoropropyl)-3,4-dihydro-1,7-naphthyridin-2-one (compound P13) as a solid. LCMS (Method 1): Rt=1.04 min, m/z=517 (M+H)⁺.

### Example P17: Preparation of 6-(6-cyclopropyl-3-ethylsulfonyl-2-pyridyl)-1-(2,2,3,3,3-pentafluoropropyl)-4H-pyrido[3,4-d][1,3]oxazin-2-one (compound P17)

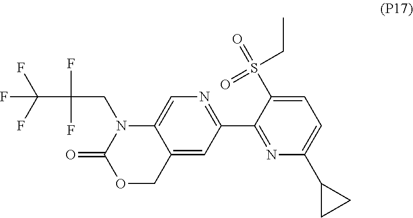 [SMILES: CCS(=O)(=O)c1ccc(C2CC2)nc1-c1cc2c(cn1)N(CC(F)(F)C(F)(F)F)C(=O)OC2]

### Step 1: Preparation of 6-(6-chloro-3-ethylsulfanyl-2-pyridyl)-1-(2,2,3,3,3-pentafluoropropyl)-4H-pyrido[3,4-d][1,3]oxazin-2-one (intermediate I-35)

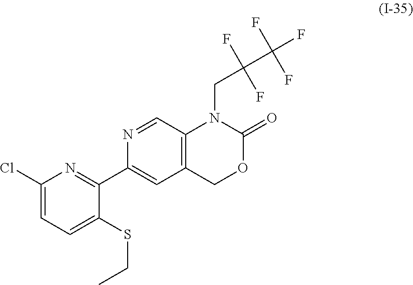 [SMILES: CCSc1ccc(Cl)nc1-c1cc2c(cn1)N(CC(F)(F)C(F)(F)F)C(=O)OC2]

6-(3-ethylsulfanyl-1-oxido-pyridin-1-ium-2-yl)-1-(2,2,3,3,3-pentafluoropropyl)-4H-pyrido[3,4-d][1,3]oxazin-2-one (intermediate I-38 prepared as described above) (2.3 g, 5.3 mmol) was dissolved in phosphoryl chloride (23 mL). The solution was stirred for 3 hours at room temperature. The reaction mixture was slowly poured on ice water, neutralized with aqueous saturated sodium bicarbonate and the product extracted with ethyl acetate (3×20 ml). The combined organic layers were dried over anhydrous sodium sulfate, filtered and concentrated under reduced pressure. Purification of the crude material by combiflash (silica gel, 30-35% ethyl acetate in cyclohexane) afforded 6-(6-chloro-3-ethylsulfanyl-2-pyridyl)-1-(2,2,3,3,3-pentafluoropropyl)-4H-pyrido[3,4-d][1,3]oxazin-2-one (intermediate 1-35). LCMS (Method 1): Rt=1.20 min, m/z=454/456 (M+H)⁺.

### Step 2: Preparation of 6-(6-chloro-3-ethylsulfonyl-2-pyridyl)-1-(2,2,3,3,3-pentafluoropropyl)-4H-pyrido[3,4-d][1,3]oxazin-2-one (intermediate I-36)

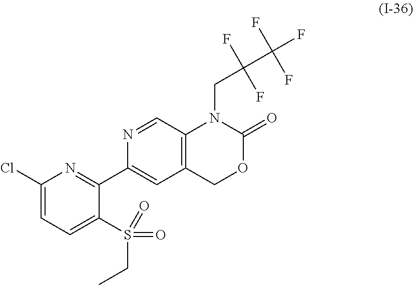 [SMILES: CCS(=O)(=O)c1ccc(Cl)nc1-c1cc2c(cn1)N(CC(F)(F)C(F)(F)F)C(=O)OC2]

To a solution of 6-(6-chloro-3-ethylsulfanyl-2-pyridyl)-1-(2,2,3,3,3-pentafluoropropyl)-4H-pyrido[3,4-d][1,3]oxazin-2-one (intermediate I-35 prepared as described above) (800 mg, 1.76 mmol) in trifluoromethylbenzene (8 mL) at 0° C. was added 3-chlorobenzenecarboperoxoic acid (956 mg, 3.88 mmol, 70 mass %) portionwise. The reaction mixture was stirred at room temperature for 1.5 hours, then poured into an aqueous saturated sodium bicarbonate solution (50 ml) and the product extracted with ethyl acetate (3×20 mL). The combined organic layers were dried over sodium sulfate, filtered and concentrated in vacuo. The crude product was purified by combiflash (silica gel, 30% ethyl acetate in cyclohexane) to afford 6-(6-chloro-3-ethylsulfonyl-2-pyridyl)-1-(2,2,3,3,3-pentafluoropropyl)-4H-pyrido[3,4-d][1,3]oxazin-2-one (intermediate I-36). LCMS (Method 1): Rt=1.12 min, m/z=486/488 (M+H)⁺.

### Step 3: Preparation of 6-(6-cyclopropyl-3-ethylsulfonyl-2-pyridyl)-1-(2,2,3,3,3-pentafluoropropyl)-4H-pyrido[3,4-d][1,3]oxazin-2-one (compound P17)

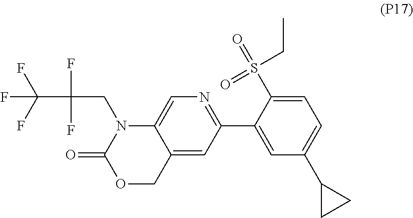 [SMILES: CCS(=O)(=O)c1ccc(C2CC2)cc1-c1cc2c(cn1)N(CC(F)(F)C(F)(F)F)C(=O)OC2]

To a solution of 6-(6-chloro-3-ethylsulfonyl-2-pyridyl)-1-(2,2,3,3,3-pentafluoropropyl)-4H-pyrido[3,4-d][1,3]oxazin-2-one (intermediate I-36 prepared as described above) (250 mg, 0.51 mmol) in toluene (5 mL) and water (0.75 mL) were added potassium carbonate (213 mg, 1.54 mmol) and cyclopropylboronic acid (140 mg, 1.54 mmol). The mixture was degassed with nitrogen for 10 minutes, then [1,1′-bis(diphenyl-phosphino)ferrocene]dichloropalladium(II) dichloromethane complex (22 mg, 0.026 mmol) was added and the mixture further degassed with nitrogen for 5 minutes. The reaction mixture was heated in the microwave at 110° C. for 1.5 hours, diluted with water (10 mL) and the product extracted three times with ethyl acetate. The combined organic layers were washed with brine, dried over anhydrous sodium sulfate, filtered and concentrated under reduced pressure. Purification of the crude material by combiflash (silica gel, 0-10% methanol in ethyl acetate) afforded of 6-(6-cyclopropyl-3-ethylsulfonyl-2-pyridyl)-1-(2,2,3,3,3-pentafluoropropyl)-4H-pyrido[3,4-d][1,3]oxazin-2-one (compound P17) as a solid. LCMS (Method 3): Rt=1.14 min. m/z=492 (M+H)⁺.

TABLE P
Examples of compounds of formula (I)

| | | | LCMS | | | |
|---|---|---|---|---|---|---|
| | | | Rₜ | [M + H]⁺ | | Mp |
| No | IUPAC name | Structures | (min) | (measured) | Method | (° C.) |
| P1 | 6-(3-ethylsulfonyl-2-pyridyl)-1- (2,2,3,3,3-pentafluoropropyl)-3,4- dihydro-1,7-naphthyridin-2-one | 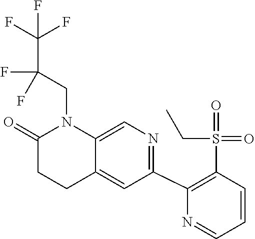 [SMILES: CCS(=O)(=O)c1cccnc1-c1cc2c(cn1)N(CC(F)(F)C(F)(F)F)C(=O)CC2] | 0.97 | 450 | 1 | 172-174 |
| P2 | 1-[5-ethylsulfonyl-6-[2-oxo-1- (2,2,2-trifluoroethyl)-3,4-dihydro- 1,7-naphthyridin-6-yl]-3- pyridyl]cyclopropanecarbonitrile | 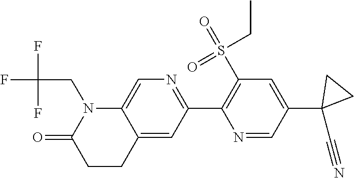 [SMILES: CCS(=O)(=O)c1cc(C2(C#N)CC2)cnc1-c1cc2c(cn1)N(CC(F)(F)F)C(=O)CC2] | 1.03 | 465 | 1 | 208-210 |
| P3 | 1-[5-ethylsulfonyl-6-[2-oxo-1- (2,2,3,3,3-pentafluoropropyl)-4H- pyrido[3,4-d][1,3]oxazin-6-yl]-3- pyridyl]cyclopropanecarbonitrile | 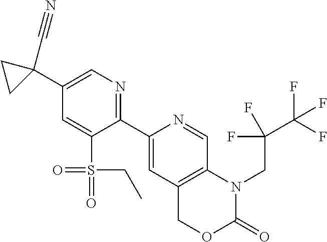 [SMILES: CCS(=O)(=O)c1cc(C2(C#N)CC2)cnc1-c1cc2c(cn1)N(CC(F)(F)C(F)(F)F)C(=O)OC2] | 1.37 | 517 | 2 | 208-210 |
| P4 | 1-[5-ethylsulfanyl-6-[2-oxo-1- (2,2,3,3,3-pentafluoropropyl)-4H- pyrido[3,4-d][1,3]oxazin-6-yl]-3- pyridyl]cyclopropanecarbonitrile | 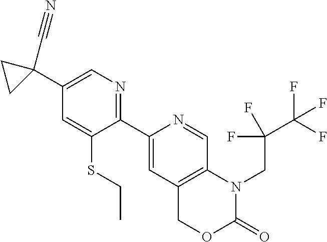 [SMILES: CCSc1cc(C2(C#N)CC2)cnc1-c1cc2c(cn1)N(CC(F)(F)C(F)(F)F)C(=O)OC2] | 1.13 | 485 | 1 | 146-148 |
| P5 | 6-[5-(3-chloropyrazol-1-yl)-3- ethylsulfonyl-2-pyridyl]-1- (2,2,3,3,3-pentafluoropropyl)-3,4- dihydro-1,7-naphthyridin-2-one | 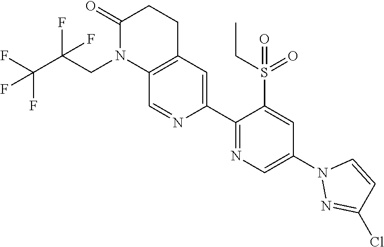 [SMILES: CCS(=O)(=O)c1cc(-n2ccc(Cl)n2)cnc1-c1cc2c(cn1)N(CC(F)(F)C(F)(F)F)C(=O)CC2] | 1.17 | 550/552 | 3 | 246-247 |
| P6 | 1-[5-ethylsulfanyl-6-[2-oxo-1- (2,2,2-trifluoroethyl)-3,4-dihydro- 1,7-naphthyridin-6-yl]-3- pyridyl]cyclopropanecarbonitrile | 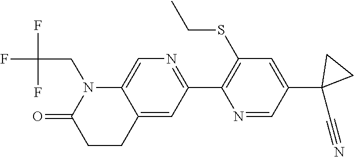 [SMILES: CCSc1cc(C2(C#N)CC2)cnc1-c1cc2c(cn1)N(CC(F)(F)F)C(=O)CC2] | 1.10 | 433 | 3 | 180-181 |
| P7 | N-[5-ethylsulfanyl-6-[2-oxo-1- (2,2,3,3,3-pentafluoropropyl)-3,4- dihydro-1,7-naphthyridin-6-yl]-3- pyridyl]-N-methyl-acetamide | 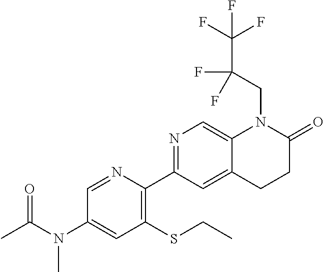 [SMILES: CCSc1cc(N(C)C(C)=O)cnc1-c1cc2c(cn1)N(CC(F)(F)C(F)(F)F)C(=O)CC2] | 1.05 | 489 | 3 | 162-164 |
| P8 | N-[5-ethylsulfonyl-6-[2-oxo-1- (2,2,3,3,3-pentafluoropropyl)-3,4- dihydro-1,7-naphthyridin-6-yl]-3- pyridyl]-N-methyl-acetamide | 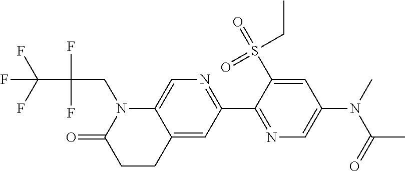 [SMILES: CCS(=O)(=O)c1cc(N(C)C(C)=O)cnc1-c1cc2c(cn1)N(CC(F)(F)C(F)(F)F)C(=O)CC2] | 1.01 | 521 | 3 | 214-216 |
| P9 | 6-[3-ethylsulfonyl-5- (trifluoromethyl)-2-pyridyl]-1- (2,2,3,3,3-pentafluoropropyl)-3,4- dihydro-1,7-naphthyridin-2-one | 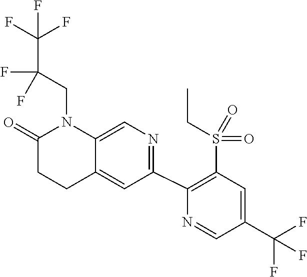 [SMILES: CCS(=O)(=O)c1cc(C(F)(F)F)cnc1-c1cc2c(cn1)N(CC(F)(F)C(F)(F)F)C(=O)CC2] | 1.10 | 518 | 1 | 197-198 |
| P10 | 6-[3-ethylsulfonyl-5- (trifluoromethyl)-2-pyridyl]-1- (2,2,3,3,3-pentafluoropropyl)-4H- pyrido[3,4-d][1,3]oxazin-2-one | 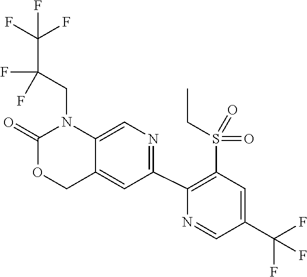 [SMILES: CCS(=O)(=O)c1cc(C(F)(F)F)cnc1-c1cc2c(cn1)N(CC(F)(F)C(F)(F)F)C(=O)OC2] | 1.48 | 520 | 4 | 194-196 |
| P11 | 2-[5-ethylsulfonyl-6-[2-oxo-1- (2,2,3,3,3-pentafluoropropyl)-3,4- dihydro-1,7-naphthyridin-6-yl]-3- pyridyl]-2-methyl-propanenitrile | 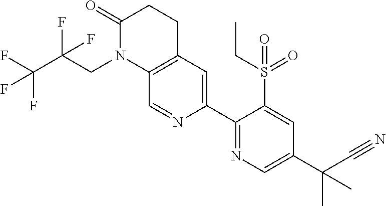 [SMILES: CCS(=O)(=O)c1cc(C(C)(C)C#N)cnc1-c1cc2c(cn1)N(CC(F)(F)C(F)(F)F)C(=O)CC2] | 1.11 | 517 | 3 | 206-208 |
| P12 | 1-[5-ethylsulfonyl-6-[2-oxo-1- (2,2,3,3,3-pentafluoropropyl)-3,4- dihydro-1,7-naphthyridin-6-yl]-3- pyridyl]cyclopropanecarbonitrile | 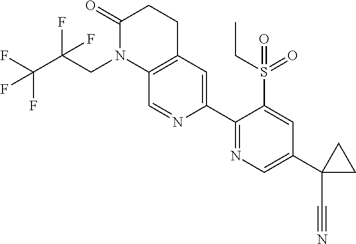 [SMILES: CCS(=O)(=O)c1cc(C2(C#N)CC2)cnc1-c1cc2c(cn1)N(CC(F)(F)C(F)(F)F)C(=O)CC2] | 1.08 | 515 | 3 | 188-190 |
| P13 | 6-[3-ethylsulfonyl-6-(1,2,4-triazol- 1-yl)-2-pyridyl]-1-(2,2,3,3,3- pentafluoropropyl)-3,4-dihydro- 1,7-naphthyridin-2-one | 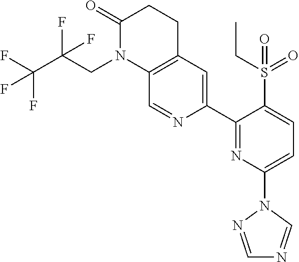 [SMILES: CCS(=O)(=O)c1ccc(-n2cncn2)nc1-c1cc2c(cn1)N(CC(F)(F)C(F)(F)F)C(=O)CC2] | 1.04 | 517 | 1 | 268-270 |
| P14 | 6-(6-cyclopropyl-3-ethylsulfonyl- 2-pyridyl)-1-(2,2,3,3,3- pentafluoropropyl)-3,4-dihydro- 1,7-naphthyridin-2-one | 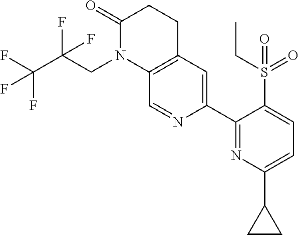 [SMILES: CCS(=O)(=O)c1ccc(C2CC2)nc1-c1cc2c(cn1)N(CC(F)(F)C(F)(F)F)C(=O)CC2] | 1.13 | 490 | 1 | 178-180 |
| P15 | 2-[5-ethylsulfonyl-6-[2-oxo-1- (2,2,3,3,3-pentafluoropropyl)-4H- pyrido[3,4-d][1,3]oxazin-6-yl]-3- pyridyl]-2-methyl-propanenitrile | 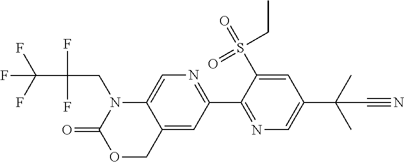 [SMILES: CCS(=O)(=O)c1cc(C(C)(C)C#N)cnc1-c1cc2c(cn1)N(CC(F)(F)C(F)(F)F)C(=O)OC2] | 1.15 | 519 | 3 | 200-202 |
| P16 | 6-[3-ethylsulfonyl-6-(1,2,4-triazol- 1-yl)-2-pyridyl]-1-(2,2,3,3,3- pentafluoropropyl)-4H-pyrido[3,4- d][1,3]oxazin-2-one | 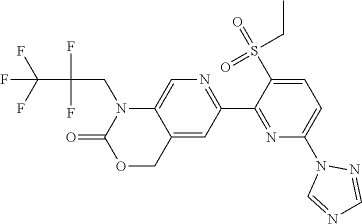 [SMILES: CCS(=O)(=O)c1ccc(-n2cncn2)nc1-c1cc2c(cn1)N(CC(F)(F)C(F)(F)F)C(=O)OC2] | 1.06 | 519 | 3 | 262-264 |
| P17 | 6-(6-cyclopropyl-3-ethylsulfonyl-2- pyridyl)-1-(2,2,3,3,3- pentafluoropropyl)-4H-pyrido[3,4- d][1,3]oxazin-2-one | 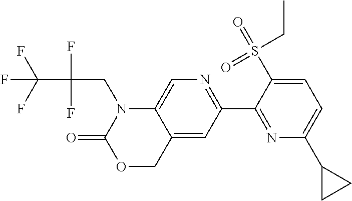 [SMILES: CCS(=O)(=O)c1ccc(C2CC2)nc1-c1cc2c(cn1)N(CC(F)(F)C(F)(F)F)C(=O)OC2] | 1.14 | 492 | 3 | 222-225 |
| P18 | N-[5-ethylsulfonyl-6-[2-oxo-1- (2,2,3,3,3-pentafluoropropyl)-4H- pyrido[3,4-d][1,3]oxazin-6-yl]-3- pyridyl]-N-methyl-acetamide | 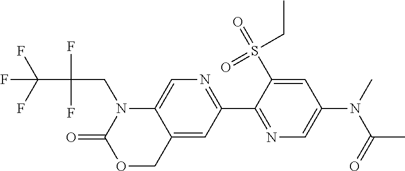 [SMILES: CCS(=O)(=O)c1cc(N(C)C(C)=O)cnc1-c1cc2c(cn1)N(CC(F)(F)C(F)(F)F)C(=O)OC2] | 1.06 | 523 | 3 | 250-252 |
| P19 | 6-(5-cyclopropyl-3-ethylsulfonyl-2- pyridyl)-1-(2,2,3,3,3- pentafluoropropyl)-3,4-dihydro- 1,7-naphthyridin-2-one | 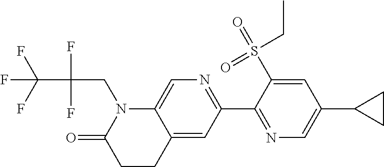 [SMILES: CCS(=O)(=O)c1cc(C2CC2)cnc1-c1cc2c(cn1)N(CC(F)(F)C(F)(F)F)C(=O)CC2] | 1.08 | 490 | 1 | 205-206 |
| P20 | 2-[[5-ethylsulfonyl-6-[2-oxo-1- (2,2,3,3,3-pentafluoropropyl)-3,4- dihydro-1,7-naphthyridin-6-yl]-3- pyridyl]oxy]-2-methyl- propanenitrile | 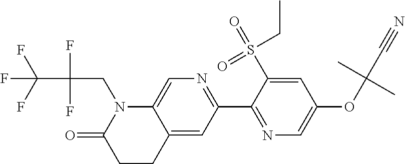 [SMILES: CCS(=O)(=O)c1cc(OC(C)(C)C#N)cnc1-c1cc2c(cn1)N(CC(F)(F)C(F)(F)F)C(=O)CC2] | 1.13 | 533 | 3 | 177-178 |
| P21 | 6-[3-ethylsulfonyl-5-(2-pyridyloxy)- 2-pyridyl]-1-(2,2,3,3,3- pentafluoropropyl)-3,4-dihydro- 1,7-naphthyridin-2-one | 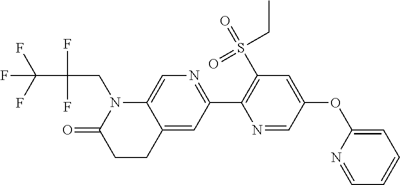 [SMILES: CCS(=O)(=O)c1cc(Oc2ccccn2)cnc1-c1cc2c(cn1)N(CC(F)(F)C(F)(F)F)C(=O)CC2] | 1.12 | 543 | 3 | 194-196 |
| P22 | 6-(3-ethylsulfonyl-6-pyrimidin- 2-yl-2-pyridyl)-1-(2,2,3,3,3- pentafluoropropyl)-3,4-dihydro- 1,7-naphthyridin-2-one | 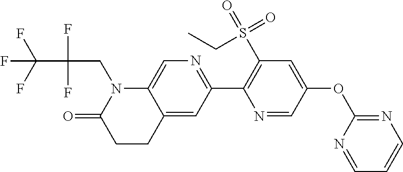 [SMILES: CCS(=O)(=O)c1cc(Oc2ncccn2)cnc1-c1cc2c(cn1)N(CC(F)(F)C(F)(F)F)C(=O)CC2] | 1.02 | 528 | 1 | 226-228 |
| P23 | 6-[3-ethylsulfonyl-5-(1-fluoro-1- methyl-ethyl)-2-pyridyl]-1- (2,2,3,3,3-pentafluoropropyl)-3,4- dihydro-1,7-naphthyridin-2-one | 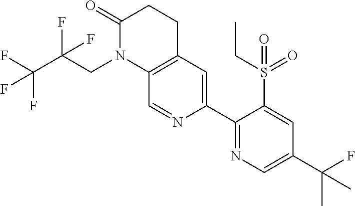 [SMILES: CCS(=O)(=O)c1cc(C(C)(C)F)cnc1-c1cc2c(cn1)N(CC(F)(F)C(F)(F)F)C(=O)CC2] | 1.48 | 510 | 4 | 198-200 |
| P24 | 6-[5-(2,2-difluoropropoxy)-3- ethylsulfonyl-2-pyridyl]-1- (2,2,3,3,3-pentafluoropropyl)-3,4- dihydro-1,7-naphthyridin-2-one | 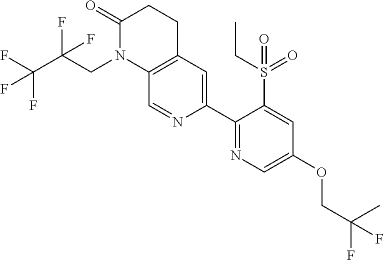 [SMILES: CCS(=O)(=O)c1cc(OCC(C)(F)F)cnc1-c1cc2c(cn1)N(CC(F)(F)C(F)(F)F)C(=O)CC2] | 1.50 | 544 | 4 | 162-164 |
| P25 | 6-[5-(2,2-difluoropropoxy)-3- ethylsulfanyl-2-pyridyl]-1- (2,2,3,3,3-pentafluoropropyl)-3,4- dihydro-1,7-naphthyridin-2-one | 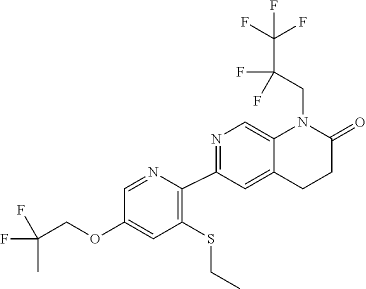 [SMILES: CCSc1cc(OCC(C)(F)F)cnc1-c1cc2c(cn1)N(CC(F)(F)C(F)(F)F)C(=O)CC2] | 1.53 | 512 | 4 | 122-124 |
| P26 | 6-[3-ethylsulfanyl-5- (trifluoromethyl)-2-pyridyl]-1- (2,2,3,3,3-pentafluoropropyl)-4H- pyrido[3,4-d][1,3]oxazin-2-one | 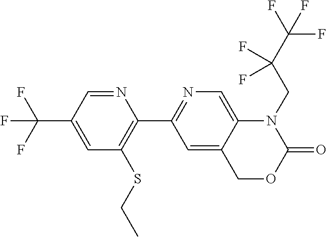 [SMILES: CCSc1cc(C(F)(F)F)cnc1-c1cc2c(cn1)N(CC(F)(F)C(F)(F)F)C(=O)OC2] | 1.27 | 488 | 3 | 168-170 |
| P27 | 6-[5-(2,2-difluoropropoxy)-3- ethylsulfanyl-2-pyridyl]-1- (2,2,3,3,3-pentafluoropropyl)-4H- pyrido[3,4-d][1,3]oxazin-2-one | 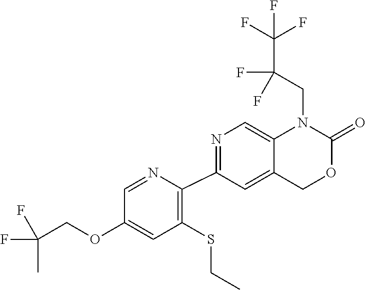 [SMILES: CCSc1cc(OCC(C)(F)F)cnc1-c1cc2c(cn1)N(CC(F)(F)C(F)(F)F)C(=O)OC2] | 1.17 | 514 | 3 | 158-160 |
| P28 | 6-[3-ethylsulfanyl-5-(methylamino)- 2-pyridyl]-1-(2,2,3,3,3- pentafluoropropyl)-4H-pyrido[3,4- d][1,3]oxazin-2-one | 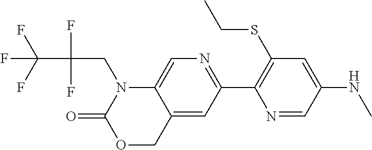 [SMILES: CCSc1cc(NC)cnc1-c1cc2c(cn1)N(CC(F)(F)C(F)(F)F)C(=O)OC2] | 1.09 | 449 | 3 | 212-214 |
| P29 | N-[5-ethylsulfanyl-6-[2-oxo-1- (2,2,3,3,3-pentafluoropropyl)-4H- pyrido[3,4-d][1,3]oxazin-6-yl]-3- pyridyl]-N-methyl-acetamide | 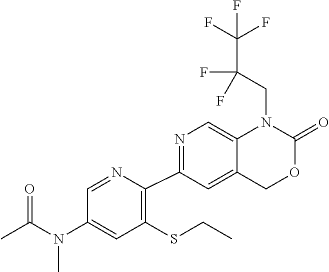 [SMILES: CCSc1cc(N(C)C(C)=O)cnc1-c1cc2c(cn1)N(CC(F)(F)C(F)(F)F)C(=O)OC2] | 1.07 | 491 | 3 | 154-156 |
| P30 | 6-[3-ethylsulfonyl-5-(1-fluoro-1- methyl-ethyl)-2-pyridyl]-1- (2,2,3,3,3-pentafluoropropyl)-4H- pyrido[3,4-d][1,3]oxazin-2-one | 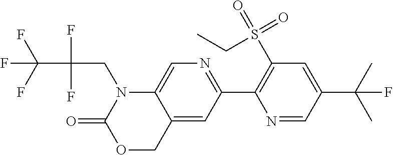 [SMILES: CCS(=O)(=O)c1cc(C(C)(C)F)cnc1-c1cc2c(cn1)N(CC(F)(F)C(F)(F)F)C(=O)OC2] | 1.13 | 512 | 3 | 192-194 |
| P31 | 6-[5-(2,2-difluoropropoxy)-3- ethylsulfonyl-2-pyridyl]-1- (2,2,3,3,3-pentafluoropropyl)-4H- pyrido[3,4-d][1,3]oxazin-2-one | 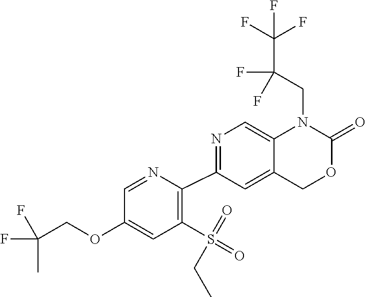 [SMILES: CCS(=O)(=O)c1cc(OCC(C)(F)F)cnc1-c1cc2c(cn1)N(CC(F)(F)C(F)(F)F)C(=O)OC2] | 1.13 | 546 | 3 | 198-200 |
| P32 | 6-[5-(2,2-difluoropropoxy)-3- ethylsulfinyl-2-pyridyl]-1-(2,2,3,3,3- pentafluoropropyl)-4H-pyrido[3,4- d][1,3]oxazin-2-one | 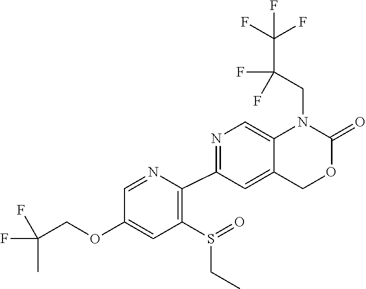 [SMILES: CCS(=O)c1cc(OCC(C)(F)F)cnc1-c1cc2c(cn1)N(CC(F)(F)C(F)(F)F)C(=O)OC2] | 1.21 | 530 | 3 | 158-160 |
| P33 | 2-[5-ethylsulfanyl-6-[2-oxo-1- (2,2,3,3,3-pentafluoropropyl)-4H- pyrido[3,4-d][1,3]oxazin-6-yl]-3- pyridyl]-2-methyl-propanenitrile | 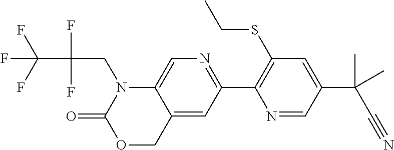 [SMILES: CCSc1cc(C(C)(C)C#N)cnc1-c1cc2c(cn1)N(CC(F)(F)C(F)(F)F)C(=O)OC2] | 1.22 | 487 | 1 | 174-176 |
| P34 | 1-[5-ethylsulfonyl-6-[2-oxo-1- (2,2,2-trifluoroethyl)-4H-pyrido[3,4- d][1,3]oxazin-6-yl]-3- pyridyl]cyclopropanecarbonitrile | 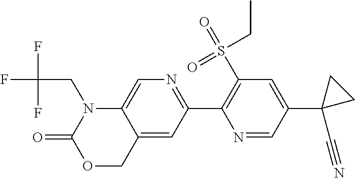 [SMILES: CCS(=O)(=O)c1cc(C2(C#N)CC2)cnc1-c1cc2c(cn1)N(CC(F)(F)F)C(=O)OC2] | 1.05 | 467 | 3 | 218-220 |
| P35 | 1-[5-ethylsulfanyl-6-[2-oxo-1- (2,2,2-trifluoroethyl)-4H-pyrido[3,4- d][1,3]oxazin-6-yl]-3- pyridyl]cyclopropanecarbonitrile | 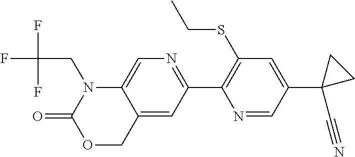 [SMILES: CCSc1cc(C2(C#N)CC2)cnc1-c1cc2c(cn1)N(CC(F)(F)F)C(=O)OC2] | 1.11 | 435 | 3 | 186-188 |

TABLE I
Examples of intermediates

| | | | LCMS | | | |
|---|---|---|---|---|---|---|
| | | | Rₜ | [M + H]⁺ | | Mp |
| No. | IUPAC name | Structures | (min) | (measured) | Method | (° C.) |
| I-24 | (3-chloropyrazol-1-yl)-5-fluoro- 1-oxido-pyridin-1-ium | 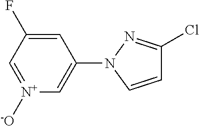 | 0.67 | 214/216 | 1 | — |
| I-25 | 3-fluoro-1-oxido-5- (trifluoromethyl)pyridin-1-ium | 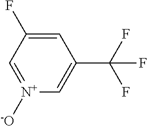 | 0.52 | 182 | 3 | — |
| I-26 | 2-(5-fluoro-1-oxido-pyridin-1- ium-3-yl)oxy-2-methyl- propanenitrile | 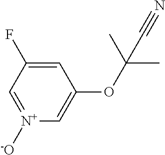 | 0.21 | 197 | 3 | — |
| I-27 | 2-[(5-fluoro-3-pyridyl)oxy]-2- methyl-propanenitrile | 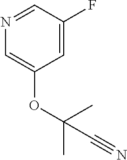 | 0.98 | 181 | 3 | — |
| I-28 | 3-fluoro-5-(1-fluoro-1-methyl- ethyl)-1-oxido-pyridin-1-ium | 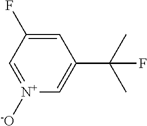 | 0.49 | 174 | 4 | — |
| I-29 | 3-fluoro-5-(1-fluoro-1-methyl- ethyl)pyridine | 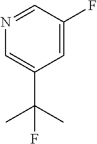 | 1.20 | 158 | 4 | — |
| I-30 | 3-(2,2-difluoropropoxy)-5- fluoro-1-oxido-pyridin-1-ium | 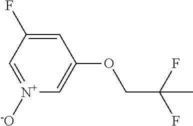 | 0.37 | 208 | 1 | — |
| I-31 | 3-(2,2-difluoropropoxy)-5- fluoro-pyridine | 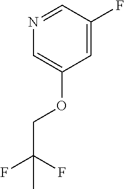 | 1.10 | 192 | 3 | — |
| I-32 | 6-bromo-1-(2,2,2- trifluoroethyl)-4H-pyrido[3,4- d][1,3]oxazin-2-one | 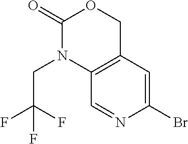 [SMILES: O=C1OCc2cc(Br)ncc2N1CC(F)(F)F] | 1.04 | 311/313 | 3 | — |
| I-33 | 6-(6-chloro-3-ethylsulfanyl-2- pyridyl)-1-(2,2,3,3,3- pentafluoropropyl)-3,4-dihydro- 1,7-naphthyridin-2-one | 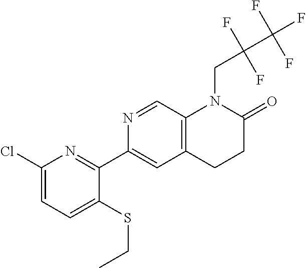 [SMILES: CCSc1ccc(Cl)nc1-c1cc2c(cn1)N(CC(F)(F)C(F)(F)F)C(=O)CC2] | 1.19 | 452/454 | 1 | — |
| I-34 | 6-(6-chloro-3-ethylsulfonyl-2- pyridyl)-1-(2,2,3,3,3- pentafluoropropyl)-3,4-dihydro- 1,7-naphthyridin-2-one | 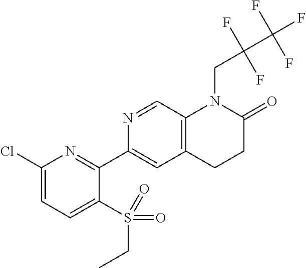 [SMILES: CCS(=O)(=O)c1ccc(Cl)nc1-c1cc2c(cn1)N(CC(F)(F)C(F)(F)F)C(=O)CC2] | 1.15 | 484/486 | 1 | — |
| I-35 | 6-(6-chloro-3-ethylsulfanyl-2- pyridyl)-1-(2,2,3,3,3- pentafluoropropyl)-4H- pyrido[3,4-d][1,3]oxazin-2-one | 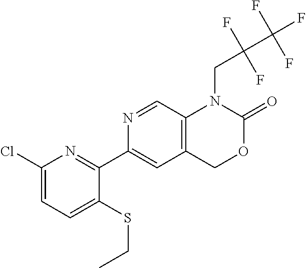 [SMILES: CCSc1ccc(Cl)nc1-c1cc2c(cn1)N(CC(F)(F)C(F)(F)F)C(=O)OC2] | 1.20 | 454/456 | 1 | — |
| I-36 | 6-(6-chloro-3-ethylsulfonyl-2- pyridyl)-1-(2,2,3,3,3- pentafluoropropyl)-4H- pyrido[3,4-d][1,3]oxazin-2-one | 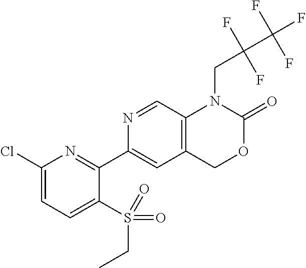 [SMILES: CCS(=O)(=O)c1ccc(Cl)nc1-c1cc2c(cn1)N(CC(F)(F)C(F)(F)F)C(=O)OC2] | 1.12 | 486/488 | 1 | — |
| I-37 | 6-(3-fluoro-1-oxido-pyridin-1- ium-2-yl)-1-(2,2,3,3,3- pentafluoropropyl)-4H- pyrido[3,4-d][1,3]oxazin-2-one | 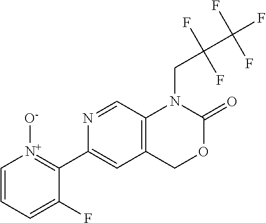 [SMILES: O=C1OCc2cc(-c3c(F)ccc[n+]3[O-])ncc2N1CC(F)(F)C(F)(F)F] | 0.92 | 394 | 1 | — |
| I-38 | 6-(3-ethylsulfanyl-1-oxido- pyridin-1-ium-2-yl)-1- (2,2,3,3,3-pentafluoropropyl)- 4H-pyrido[3,4-d][1,3]oxazin-2- one | 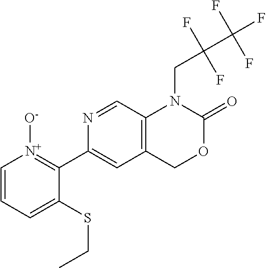 [SMILES: CCSc1ccc[n+]([O-])c1-c1cc2c(cn1)N(CC(F)(F)C(F)(F)F)C(=O)OC2] | 0.96 | 436 | 1 | — |

The activity of the compositions according to the invention can be broadened considerably, and adapted to prevailing circumstances, by adding other insecticidally, acaricidally and/or fungicidally active ingredients. The mixtures of the compounds of formula I with other insecticidally, acaricidally and/or fungicidally active ingredients may also have further surprising advantages which can also be described, in a wider sense, as synergistic activity. For example, better tolerance by plants, reduced phytotoxicity, insects can be controlled in their different development stages or better behaviour during their production, for example during grinding or mixing, during their storage or during their use. Suitable additions to active ingredients here are, for example, representatives of the following classes of active ingredients; organophosphorus compounds, nitrophenol derivatives, thioureas, juvenile hormones, formamidines, benzophenone derivatives, ureas, pyrrole derivatives, carbamates, pyrethroids, chlorinated hydrocarbons, acylureas, pyridylmethyleneamino derivatives, macrolides, neonicotinoids and Bacillus thuringiensis preparations.

The following mixtures of the compounds of formula I with active ingredients are preferred (the abbreviation “TX” means “one compound selected from the group consisting of the compounds described in Tables A-1 to A-60, Tables B-1 to B-60, Tables C-1 to C-60, Tables D-1 to D-60 and Table P of the present invention”):
an adjuvant selected from the group of substances consisting of petroleum oils (alternative name) (628)+TX;
abamectin+TX, acequinocyl+TX, acetamiprid+TX, acetoprole+TX, acrinathrin+TX, acynonapyr+TX, afidopyropen+TX, afoxolaner+TX, alanycarb+TX, allethrin+TX, alpha-cypermethrin+TX, alphamethrin+TX, amidoflumet+TX, aminocarb+TX, azocyclotin+TX, bensultap+TX, benzoximate+TX, benzpyrimoxan+TX, betacyfluthrin+TX, beta-cypermethrin+TX, bifenazate+TX, bifenthrin+TX, binapacryl+TX, bioallethrin+TX, S-bioallethrin+TX, bioresmethrin+TX, bistrifluron+TX, broflanilide+TX, brofluthrinate+TX, bromophos-ethyl+TX, buprofezine+TX, butocarboxim+TX, cadusafos+TX, carbaryl+TX, carbosulfan+TX, cartap+TX, CAS number: 1632218-00-8+TX, CAS number: 1808115-49-2+TX, CAS number: 2032403-97-5+TX, CAS number: 2044701-44-0+TX, CAS number: 2128706-05-6+TX, CAS number: 2095470-94-1+TX, CAS number: 2377084-09-6+TX, CAS number: 1445683-71-5+TX, CAS number: 2408220-94-8+TX, CAS number: 2408220-91-5+TX, CAS number: 1365070-72-9+TX, CAS number: 2171099-09-3+TX, CAS number: 2396747-83-2+TX, CAS number: 2133042-31-4+TX, CAS number: 2133042-44-9+TX, CAS number: 1445684-82-1+TX, CAS number: 1445684-82-1+TX, CAS number: 1922957-45-6+TX, CAS number: 1922957-46-7+TX, CAS number: 1922957-47-8+TX, CAS number: 1922957-48-9+TX, CAS number: 2415706-16-8+TX, CAS number: 1594624-87-9+TX, CAS number: 1594637-65-6+TX, CAS number: 1594626-19-3+TX, CAS number: 1990457-52-7+TX, CAS number: 1990457-55-0+TX, CAS number: 1990457-57-2+TX, CAS number: 1990457-77-6+TX, CAS number: 1990457-66-3+TX, CAS number: 1990457-85-6+TX, CAS number: 2220132-55-6+TX, CAS number: 1255091-74-7+TX, CAS number: 1305319-70-3+TX, CAS number: 1442448-92-1+TX, chlorantraniliprole+TX, chlordane+TX, chlorfenapyr+TX, chloroprallethrin+TX, chromafenozide+TX, clenpirin+TX, cloethocarb+TX, clothianidin+TX, 2-chlorophenyl N-methylcarbamate (CPMC)+TX, cyanofenphos+TX, cyantraniliprole+TX, cyclaniliprole+TX, cyclobutrifluram+TX, cycloprothrin+TX, cycloxaprid+TX, cyenopyrafen+TX, cyetpyrafen (or etpyrafen)+TX, cyflumetofen+TX, cyfluthrin+TX, cyhalodiamide+TX, cyhalothrin+TX, cypermethrin+TX, cyphenothrin+TX, cyproflanilide+TX, cyromazine+TX, deltamethrin+TX, diafenthiuron+TX, dialifos+TX, dibrom+TX, dicloromezotiaz+TX, diflovidazine+TX, diflubenzuron+TX, dimpropyridaz+TX, dinactin+TX, dinocap+TX, dinotefuran+TX, dioxabenzofos+TX, emamectin (or emamectin benzoate)+TX, empenthrin+TX, epsilon−momfluorothrin+TX, epsilon-metofluthrin+TX, esfenvalerate+TX, ethion+TX, ethiprole+TX, etofenprox+TX, etoxazole+TX, famphur+TX, fenazaquin+TX, fenfluthrin+TX, fenmezoditiaz+TX, fenitrothion+TX, fenobucarb+TX, fenothiocarb+TX, fenoxycarb+TX, fenpropathrin+TX, fenpyroximate+TX, fensulfothion+TX, fenthion+TX, fentinacetate+TX, fenvalerate+TX, fipronil+TX, flometoquin+TX, flonicamid+TX, fluacrypyrim+TX, fluazaindolizine+TX, fluazuron+TX, flubendiamide+TX, flubenzimine+TX, fluchlordiniliprole+TX, flucitrinate+TX, flucycloxuron+TX, flucythrinate+TX, fluensulfone+TX, flufenerim+TX, flufenprox+TX, flufiprole+TX, fluhexafon+TX, flumethrin+TX, fluopyram+TX, flupentiofenox+TX, flupyradifurone+TX, flupyrimin+TX, fluralaner+TX, fluvalinate+TX, fluxametamide+TX, fosthiazate+TX, gamma-cyhalothrin+TX, Gossyplure™+TX, guadipyr+TX, halofenozide+TX, halfenprox+TX, heptafluthrin+TX, hexythiazox+TX, hydramethylnon+TX, imicyafos+TX, imidacloprid+TX, imiprothrin+TX, indazapyroxamet+TX, indoxacarb+TX, iodomethane+TX, iprodione+TX, isocycloseram+TX, isothioate+TX, ivermectin+TX, kappa-bifenthrin+TX, kappa-tefluthrin+TX, lambda-Cyhalothrin+TX, lepimectin+TX, lotilaner+TX, lufenuron+TX, metaflumizone+TX, metaldehyde+TX, metam+TX, methomyl+TX, methoxyfenozide+TX, metofluthrin+TX, metolcarb+TX, mexacarbate+TX, milbemectin+TX, momfluorothrin+TX, niclosamide+TX, nicofluprole+TX; nitenpyram+TX, nithiazine+TX, omethoate+TX, oxamyl+TX, oxazosulfyl+TX, parathion-ethyl+TX, permethrin+TX, phenothrin+TX, phosphocarb+TX, piperonylbutoxide+TX, pirimicarb+TX, pirimiphos-ethyl+TX, pirimiphos-methyl+TX, Polyhedrosis virus+TX, prallethrin+TX, profenofos+TX, profluthrin+TX, propargite+TX, propetamphos+TX, propoxur+TX, prothiophos+TX, protrifenbute+TX, pyflubumide+TX, pymetrozine+TX, pyraclofos+TX, pyrafluprole+TX, pyridaben+TX, pyridalyl+TX, pyrifluquinazon+TX, pyrimidifen+TX, pyriminostrobin+TX, pyriprole+TX, pyriproxyfen+TX, resmethrin+TX, sarolaner+TX, selamectin+TX, silafluofen+TX, spinetoram+TX, spinosad+TX, spirodiclofen+TX, spiromesifen+TX, spiropidion+TX, spirotetramat+TX, spidoxamat+TX, sulfoxaflor+TX, tebufenozide+TX, tebufenpyrad+TX, tebupirimiphos+TX, tefluthrin+TX, temephos+TX, tetrachlorantraniliprole+TX, tetradiphon+TX, tetramethrin+TX, tetramethylfluthrin+TX, tetranactin+TX, tetraniliprole+TX, theta-cypermethrin+TX, thiacloprid+TX, thiamethoxam+TX, thiocyclam+TX, thiodicarb+TX, thiofanox+TX, thiometon+TX, thiosultap+TX, tigolaner+TX, tioxazafen+TX, tolfenpyrad+TX, toxaphene+TX, tralomethrin+TX, transfluthrin+TX, triazamate+TX, triazophos+TX, trichlorfon+TX, trichloronate+TX, trichlorphon+TX, trifluenfuronate+TX, triflumezopyrim+TX, tyclopyrazoflor+TX, zeta-cypermethrin+TX, Extract of seaweed and fermentation product derived from melasse+TX, Extract of seaweed and fermentation product derived from melasse comprising urea+TX, amino acids+TX, potassium and molybdenum and EDTA-chelated manganese+TX, Extract of seaweed and fermented plant products+TX, Extract of seaweed and fermented plant products comprising phytohormones+TX, vitamins+TX, EDTA-chelated copper+TX, zinc+TX, and iron+TX, azadirachtin+TX, Bacillus aizawai+TX, Bacillus chitinosporus AQ746 (NRRL Accession No B-21 618)+TX, Bacillus firmus+TX, Bacillus kurstaki+TX, Bacillus mycoides AQ726 (NRRL Accession No. B-21664)+TX, Bacillus pumilus (NRRL Accession No B-30087)+TX, Bacillus pumilus AQ717 (NRRL Accession No. B-21662)+TX, Bacillus sp. AQ178 (ATCC Accession No. 53522)+TX, Bacillus sp. AQ175 (ATCC Accession No. 55608)+TX, Bacillus sp. AQ177 (ATCC Accession No. 55609)+TX, Bacillus subtilis unspecified+TX, Bacillus subtilis AQ153 (ATCC Accession No. 55614)+TX, Bacillus subtilis AQ30002 (NRRL Accession No. B-50421)+TX, Bacillus subtilis AQ30004 (NRRL Accession No. B-50455)+TX, Bacillus subtilis AQ713 (NRRL Accession No. B-21661)+TX, Bacillus subtilis AQ743 (NRRL Accession No. B-21665)+TX, Bacillus thuringiensis AQ52 (NRRL Accession No. B-21619)+TX, Bacillus thuringiensis BD #32 (NRRL Accession No B-21530)+TX, Bacillus thuringiensis subspec. kurstaki BMP 123+TX, Beauveria bassiana+TX, D-limonene+TX, Granulovirus+TX, Harpin+TX, Helicoverpa armigera Nucleopolyhedrovirus+TX, Helicoverpa zea Nucleopolyhedrovirus+TX, Heliothis virescens Nucleopolyhedrovirus+TX, Heliothis punctigera Nucleopolyhedrovirus+TX, Metarhizium spp.+TX, Muscodor albus 620 (NRRL Accession No. 30547)+TX, Muscodor roseus A3-5 (NRRL Accession No. 30548)+TX, Neem tree based products+TX, Paecilomyces fumosoroseus+TX, Paecilomyces lilacinus+TX, Pasteuria nishizawae+TX, Pasteuria penetrans+TX, Pasteuria ramosa+TX, Pasteuria thornei+TX, Pasteuria usgae+TX, P-cymene+TX, Plutella xylostella Granulosis virus+TX, Plutella xylostella Nucleopolyhedrovirus+TX, Polyhedrosis virus+TX, pyrethrum+TX, QRD 420 (a terpenoid blend)+TX, QRD 452 (a terpenoid blend)+TX, QRD 460 (a terpenoid blend)+TX, Quillaja saponaria+TX, Rhodococcus globerulus AQ719 (NRRL Accession No B-21663)+TX, Spodoptera frugiperda Nucleopolyhedrovirus+TX, Streptomyces galbus (NRRL Accession No. 30232)+TX, Streptomyces sp. (NRRL Accession No. B-30145)+TX, Terpenoid blend+TX, and Verticillium spp.+TX;
an algicide selected from the group of substances consisting of bethoxazin [CCN]+TX, copper dioctanoate (IUPAC name) (170)+TX, copper sulfate (172)+TX, cybutryne [CCN]+TX, dichlone (1052)+TX, dichlorophen (232)+TX, endothal (295)+TX, fentin (347)+TX, hydrated lime [CCN]+TX, nabam (566)+TX, quinoclamine (714)+TX, quinonamid (1379)+TX, simazine (730)+TX, triphenyltin acetate (IUPAC name) (347) and triphenyltin hydroxide (IUPAC name) (347)+TX;
an anthelmintic selected from the group of substances consisting of abamectin (1)+TX, crufomate (1011)+TX, cyclobutrifluram+TX, doramectin (alternative name) [CCN]+TX, emamectin (291)+TX, emamectin benzoate (291)+TX, eprinomectin (alternative name) [CCN]+TX, ivermectin (alternative name) [CCN]+TX, milbemycin oxime (alternative name) [CCN]+TX, moxidectin (alternative name) [CCN]+TX, piperazine [CCN]+TX, selamectin (alternative name) [CCN]+TX, spinosad (737) and thiophanate (1435)+TX;
an avicide selected from the group of substances consisting of chloralose (127)+TX, endrin (1122)+TX, fenthion (346)+TX, pyridin-4-amine (IUPAC name) (23) and strychnine (745)+TX; a bactericide selected from the group of substances consisting of 1-hydroxy-1H-pyridine-2-thione (IUPAC name) (1222)+TX, 4-(quinoxalin-2-ylamino)benzenesulfonamide (IUPAC name) (748)+TX, 8-hydroxyquinoline sulfate (446)+TX, bronopol (97)+TX, copper dioctanoate (IUPAC name) (170)+TX, copper hydroxide (IUPAC name) (169)+TX, cresol [CCN]+TX, dichlorophen (232)+TX, dipyrithione (1105)+TX, dodicin (1112)+TX, fenaminosulf (1144)+TX, formaldehyde (404)+TX, hydrargaphen (alternative name) [CCN]+TX, kasugamycin (483)+TX, kasugamycin hydrochloride hydrate (483)+TX, nickel bis(dimethyldithiocarbamate) (IUPAC name) (1308)+TX, nitrapyrin (580)+TX, octhilinone (590)+TX, oxolinic acid (606)+TX, oxytetracycline (611)+TX, potassium hydroxyquinoline sulfate (446)+TX, probenazole (658)+TX, streptomycin (744)+TX, streptomycin sesquisulfate (744)+TX, tecloftalam (766)+TX, and thiomersal (alternative name) [CCN]+TX;
a biological agent selected from the group of substances consisting of Adoxophyes orana GV (alternative name) (12)+TX, Agrobacterium radiobacter (alternative name) (13)+TX, Amblyseius spp. (alternative name) (19)+TX, Anagrapha falcifera NPV (alternative name) (28)+TX, Anagrus atomus (alternative name) (29)+TX, Aphelinus abdominalis (alternative name) (33)+TX, Aphidius colemani (alternative name) (34)+TX, Aphidoletes aphidimyza (alternative name) (35)+TX, Autographa californica NPV (alternative name) (38)+TX, Bacillus firmus (alternative name) (48)+TX, Bacillus sphaericus Neide (scientific name) (49)+TX, Bacillus thuringiensis Berliner (scientific name) (51)+TX, Bacillus thuringiensis subsp. aizawai (scientific name) (51)+TX, Bacillus thuringiensis subsp. israelensis (scientific name) (51)+TX, Bacillus thuringiensis subsp. japonensis (scientific name) (51)+TX, Bacillus thuringiensis subsp. kurstaki (scientific name) (51)+TX, Bacillus thuringiensis subsp. tenebrionis (scientific name) (51)+TX, Beauveria bassiana (alternative name) (53)+TX, Beauveria brongniartii (alternative name) (54)+TX, Chrysoperla carnea (alternative name) (151)+TX, Cryptolaemus montrouzieri (alternative name) (178)+TX, Cydia pomonella GV (alternative name) (191)+TX, Dacnusa sibirica (alternative name) (212)+TX, Diglyphus isaea (alternative name) (254)+TX, Encarsia formosa (scientific name) (293)+TX, Eretmocerus eremicus (alternative name) (300)+TX, Helicoverpa zea NPV (alternative name) (431)+TX, Heterorhabditis bacteriophora and H. megidis (alternative name) (433)+TX, Hippodamia convergens (alternative name) (442)+TX, Leptomastix dactylopii (alternative name) (488)+TX, Macrolophus caliginosus (alternative name) (491)+TX, Mamestra brassicae NPV (alternative name) (494)+TX, Metaphycus helvolus (alternative name) (522)+TX, Metarhizium anisopliae var. acridum (scientific name) (523)+TX, Metarhizium anisopliae var. anisopliae (scientific name) (523)+TX, Neodiprion sertifer NPV and N. lecontei NPV (alternative name) (575)+TX, Orius spp. (alternative name) (596)+TX, Paecilomyces fumosoroseus (alternative name) (613)+TX, Phytoseiulus persimilis (alternative name) (644)+TX, Spodoptera exigua multicapsid nuclear polyhedrosis virus (scientific name) (741)+TX, Steinernema bibionis (alternative name) (742)+TX, Steinernema carpocapsae (alternative name) (742)+TX, Steinernema feltiae (alternative name) (742)+TX, Steinernema glaseri (alternative name) (742)+TX, Steinernema riobrave (alternative name) (742)+TX, Steinernema riobravis (alternative name) (742)+TX, Steinernema scapterisci (alternative name) (742)+TX, Steinernema spp. (alternative name) (742)+TX, Trichogramma spp. (alternative name) (826)+TX, Typhlodromus occidentalis (alternative name) (844) and Verticillium lecanii (alternative name) (848)+TX;
a soil sterilant selected from the group of substances consisting of iodomethane (IUPAC name) (542) and methyl bromide (537)+TX;
a chemosterilant selected from the group of substances consisting of apholate [CCN]+TX, bisazir (alternative name) [CCN]+TX, busulfan (alternative name) [CCN]+TX, diflubenzuron (250)+TX, dimatif (alternative name) [CCN]+TX, hemel [CCN]+TX, hempa [CCN]+TX, metepa [CCN]+TX, methiotepa [CCN]+TX, methyl apholate [CCN]+TX, morzid [CCN]+TX, penfluron (alternative name) [CCN]+TX, tepa [CCN]+TX, thiohempa (alternative name) [CCN]+TX, thiotepa (alternative name) [CCN]+TX, tretamine (alternative name) [CCN] and uredepa (alternative name) [CCN]+TX;
an insect pheromone selected from the group of substances consisting of (E)-dec-5-en-1-yl acetate with (E)-dec-5-en-1-ol (IUPAC name) (222)+TX, (E)-tridec-4-en-1-yl acetate (IUPAC name) (829)+TX, (E)-6-methylhept-2-en-4-ol (IUPAC name) (541)+TX, (E,Z)-tetradeca-4,10-dien-1-yl acetate (IUPAC name) (779)+TX, (Z)-dodec-7-en-1-yl acetate (IUPAC name) (285)+TX, (Z)-hexadec-11-enal (IUPAC name) (436)+TX, (Z)-hexadec-11-en-1-yl acetate (IUPAC name) (437)+TX, (Z)-hexadec-13-en-11-yn-1-yl acetate (IUPAC name) (438)+TX, (Z)-icos-13-en-10-one (IUPAC name) (448)+TX, (Z)-tetradec-7-en-1-al (IUPAC name) (782)+TX, (Z)-tetradec-9-en-1-ol (IUPAC name) (783)+TX, (Z)-tetradec-9-en-1-yl acetate (IUPAC name) (784)+TX, (7E,9Z)-dodeca-7,9-dien-1-yl acetate (IUPAC name) (283)+TX, (9Z,11E)-tetradeca-9,11-dien-1-yl acetate (IUPAC name) (780)+TX, (9Z,12E)-tetradeca-9,12-dien-1-yl acetate (IUPAC name) (781)+TX, 14-methyloctadec-1-ene (IUPAC name) (545)+TX, 4-methylnonan-5-ol with 4-methylnonan-5-one (IUPAC name) (544)+TX, alpha-multistriatin (alternative name) [CCN]+TX, brevicomin (alternative name) [CCN]+TX, codlelure (alternative name) [CCN]+TX, codlemone (alternative name) (167)+TX, cuelure (alternative name) (179)+TX, disparlure (277)+TX, dodec-8-en-1-yl acetate (IUPAC name) (286)+TX, dodec-9-en-1-yl acetate (IUPAC name) (287)+TX, dodeca-8+TX, 10-dien-1-yl acetate (IUPAC name) (284)+TX, dominicalure (alternative name) [CCN]+TX, ethyl 4-methyloctanoate (IUPAC name) (317)+TX, eugenol (alternative name) [CCN]+TX, frontalin (alternative name) [CCN]+TX, gossyplure (alternative name) (420)+TX, grandlure (421)+TX, grandlure I (alternative name) (421)+TX, grandlure II (alternative name) (421)+TX, grandlure III (alternative name) (421)+TX, grandlure IV (alternative name) (421)+TX, hexalure [CCN]+TX, ipsdienol (alternative name) [CCN]+TX, ipsenol (alternative name) [CCN]+TX, japonilure (alternative name) (481)+TX, lineatin (alternative name) [CCN]+TX, litlure (alternative name) [CCN]+TX, looplure (alternative name) [CCN]+TX, medlure [CCN]+TX, megatomoic acid (alternative name) [CCN]+TX, methyl eugenol (alternative name) (540)+TX, muscalure (563)+TX, octadeca-2,13-dien-1-yl acetate (IUPAC name) (588)+TX, octadeca-3,13-dien-1-yl acetate (IUPAC name) (589)+TX, orfralure (alternative name) [CCN]+TX, oryctalure (alternative name) (317)+TX, ostramone (alternative name) [CCN]+TX, siglure [CCN]+TX, sordidin (alternative name) (736)+TX, sulcatol (alternative name) [CCN]+TX, tetradec-11-en-1-yl acetate (IUPAC name) (785)+TX, trimedlure (839)+TX, trimedlure A (alternative name) (839)+TX, trimedlure B₁ (alternative name) (839)+TX, trimedlure B₂ (alternative name) (839)+TX, trimedlure C (alternative name) (839) and trunc-call (alternative name) [CCN]+TX;
an insect repellent selected from the group of substances consisting of 2-(octylthio)ethanol (IUPAC name) (591)+TX, butopyronoxyl (933)+TX, butoxy(polypropylene glycol) (936)+TX, dibutyl adipate (IUPAC name) (1046)+TX, dibutyl phthalate (1047)+TX, dibutyl succinate (IUPAC name) (1048)+TX, diethyltoluamide [CCN]+TX, dimethyl carbate [CCN]+TX, dimethyl phthalate [CCN]+TX, ethyl hexanediol (1137)+TX, hexamide [CCN]+TX, methoquin-butyl (1276)+TX, methylneodecanamide [CCN]+TX, oxamate [CCN] and picaridin [CCN]+TX;
a molluscicide selected from the group of substances consisting of bis(tributyltin) oxide (IUPAC name) (913)+TX, bromoacetamide [CCN]+TX, calcium arsenate [CCN]+TX, cloethocarb (999)+TX, copper acetoarsenite [CCN]+TX, copper sulfate (172)+TX, fentin (347)+TX, ferric phosphate (IUPAC name) (352)+TX, metaldehyde (518)+TX, methiocarb (530)+TX, niclosamide (576)+TX, niclosamide-olamine (576)+TX, pentachlorophenol (623)+TX, sodium pentachlorophenoxide (623)+TX, tazimcarb (1412)+TX, thiodicarb (799)+TX, tributyltin oxide (913)+TX, trifenmorph (1454)+TX, trimethacarb (840)+TX, triphenyltin acetate (IUPAC name) (347) and triphenyltin hydroxide (IUPAC name) (347)+TX, pyriprole [394730-71-3]+TX; a nematicide selected from the group of substances consisting of AKD-3088 (compound code)+TX, 1,2-dibromo-3-chloropropane (IUPAC/Chemical Abstracts name) (1045)+TX, 1,2-dichloropropane (IUPAC/Chemical Abstracts name) (1062)+TX, 1,2-dichloropropane with 1,3-dichloropropene (IUPAC name) (1063)+TX, 1,3-dichloropropene (233)+TX, 3,4-dichlorotetrahydrothiophene 1,1-dioxide (IUPAC/Chemical Abstracts name) (1065)+TX, 3-(4-chlorophenyl)-5-methylrhodanine (IUPAC name) (980)+TX, 5-methyl-6-thioxo-1,3,5-thiadiazinan-3-ylacetic acid (IUPAC name) (1286)+TX, 6-isopentenylaminopurine (alternative name) (210)+TX, abamectin (1)+TX, acetoprole [CCN]+TX, alanycarb (15)+TX, aldicarb (16)+TX, aldoxycarb (863)+TX, AZ 60541 (compound code)+TX, benclothiaz [CCN]+TX, benomyl (62)+TX, butylpyridaben (alternative name)+TX, cadusafos (109)+TX, carbofuran (118)+TX, carbon disulfide (945)+TX, carbosulfan (119)+TX, chloropicrin (141)+TX, chlorpyrifos (145)+TX, cloethocarb (999)+TX, cyclobutrifluram+TX, cytokinins (alternative name) (210)+TX, dazomet (216)+TX, DBCP (1045)+TX, DCIP (218)+TX, diamidafos (1044)+TX, dichlofenthion (1051)+TX, dicliphos (alternative name)+TX, dimethoate (262)+TX, doramectin (alternative name) [CCN]+TX, emamectin (291)+TX, emamectin benzoate (291)+TX, eprinomectin (alternative name) [CCN]+TX, ethoprophos (312)+TX, ethylene dibromide (316)+TX, fenamiphos (326)+TX, fenpyrad (alternative name)+TX, fensulfothion (1158)+TX, fosthiazate (408)+TX, fosthietan (1196)+TX, furfural (alternative name) [CCN]+TX, GY-81 (development code) (423)+TX, heterophos [CCN]+TX, iodomethane (IUPAC name) (542)+TX, isamidofos (1230)+TX, isazofos (1231)+TX, ivermectin (alternative name) [CCN]+TX, kinetin (alternative name) (210)+TX, mecarphon (1258)+TX, metam (519)+TX, metam-potassium (alternative name) (519)+TX, metam-sodium (519)+TX, methyl bromide (537)+TX, methyl isothiocyanate (543)+TX, milbemycin oxime (alternative name) [CCN]+TX, moxidectin (alternative name) [CCN]+TX, Myrothecium verrucaria composition (alternative name) (565)+TX, NC-184 (compound code)+TX, oxamyl (602)+TX, phorate (636)+TX, phosphamidon (639)+TX, phosphocarb [CCN]+TX, sebufos (alternative name)+TX, selamectin (alternative name) [CCN]+TX, spinosad (737)+TX, terbam (alternative name)+TX, terbufos (773)+TX, tetrachlorothiophene (IUPAC/Chemical Abstracts name) (1422)+TX, thiafenox (alternative name)+TX, thionazin (1434)+TX, triazophos (820)+TX, triazuron (alternative name)+TX, xylenols [CCN]+TX, YI-5302 (compound code) and zeatin (alternative name) (210)+TX, fluensulfone [318290-98-1]+TX, fluopyram+TX;
a nitrification inhibitor selected from the group of substances consisting of potassium ethylxanthate [CCN] and nitrapyrin (580)+TX;
a plant activator selected from the group of substances consisting of acibenzolar (6)+TX, acibenzolar-S-methyl (6)+TX, probenazole (658) and Reynoutria sachalinensis extract (alternative name) (720)+TX;
a rodenticide selected from the group of substances consisting of 2-isovalerylindan-1,3-dione (IUPAC name) (1246)+TX, 4-(quinoxalin-2-ylamino)benzenesulfonamide (IUPAC name) (748)+TX, alpha-chlorohydrin [CCN]+TX, aluminium phosphide (640)+TX, antu (880)+TX, arsenous oxide (882)+TX, barium carbonate (891)+TX, bisthiosemi (912)+TX, brodifacoum (89)+TX, bromadiolone (including alpha-bromadiolone)+TX, bromethalin (92)+TX, calcium cyanide (444)+TX, chloralose (127)+TX, chlorophacinone (140)+TX, cholecalciferol (alternative name) (850)+TX, coumachlor (1004)+TX, coumafuryl (1005)+TX, coumatetralyl (175)+TX, crimidine (1009)+TX, difenacoum (246)+TX, difethialone (249)+TX, diphacinone (273)+TX, ergocalciferol (301)+TX, flocoumafen (357)+TX, fluoroacetamide (379)+TX, flupropadine (1183)+TX, flupropadine hydrochloride (1183)+TX, gamma-HCH (430)+TX, HCH (430)+TX, hydrogen cyanide (444)+TX, iodomethane (IUPAC name) (542)+TX, lindane (430)+TX, magnesium phosphide (IUPAC name) (640)+TX, methyl bromide (537)+TX, norbormide (1318)+TX, phosacetim (1336)+TX, phosphine (IUPAC name) (640)+TX, phosphorus [CCN]+TX, pindone (1341)+TX, potassium arsenite [CCN]+TX, pyrinuron (1371)+TX, scilliroside (1390)+TX, sodium arsenite [CCN]+TX, sodium cyanide (444)+TX, sodium fluoroacetate (735)+TX, strychnine (745)+TX, thallium sulfate [CCN]+TX, warfarin (851) and zinc phosphide (640)+TX; a synergist selected from the group of substances consisting of 2-(2-butoxyethoxy)ethyl piperonylate (IUPAC name) (934)+TX, 5-(1,3-benzodioxol-5-yl)-3-hexylcyclohex-2-enone (IUPAC name) (903)+TX, farnesol with nerolidol (alternative name) (324)+TX, MB-599 (development code) (498)+TX, MGK 264 (development code) (296)+TX, piperonyl butoxide (649)+TX, piprotal (1343)+TX, propyl isomer (1358)+TX, S421 (development code) (724)+TX, sesamex (1393)+TX, sesasmolin (1394) and sulfoxide (1406)+TX;
an animal repellent selected from the group of substances consisting of anthraquinone (32)+TX, chloralose (127)+TX, copper naphthenate [CCN]+TX, copper oxychloride (171)+TX, diazinon (227)+TX, dicyclopentadiene (chemical name) (1069)+TX, guazatine (422)+TX, guazatine acetates (422)+TX, methiocarb (530)+TX, pyridin-4-amine (IUPAC name) (23)+TX, thiram (804)+TX, trimethacarb (840)+TX, zinc naphthenate [CCN] and ziram (856)+TX;
a virucide selected from the group of substances consisting of imanin (alternative name) [CCN] and ribavirin (alternative name) [CCN]+TX;
a wound protectant selected from the group of substances consisting of mercuric oxide (512)+TX, octhilinone (590) and thiophanate-methyl (802)+TX;
a biologically active substance selected from 1,1-bis(4-chloro-phenyl)-2-ethoxyethanol+TX, 2,4-dichlorophenyl benzenesulfonate+TX, 2-fluoro-N-methyl-N-1-naphthylacetamide+TX, 4-chlorophenyl phenyl sulfone+TX, acetoprole+TX, aldoxycarb+TX, amidithion+TX, amidothioate+TX, amiton+TX, amiton hydrogen oxalate+TX, amitraz+TX, aramite+TX, arsenous oxide+TX, azobenzene+TX, azothoate+TX, benomyl+TX, benoxa-fos+TX, benzyl benzoate+TX, bixafen+TX, brofenvalerate+TX, bromo-cyclen+TX, bromophos+TX, bromopropylate+TX, buprofezin+TX, butocarboxim+TX, butoxycarboxim+TX, butylpyridaben+TX, calcium polysulfide+TX, camphechlor+TX, carbanolate+TX, carbophenothion+TX, cymiazole+TX, chino-methionat+TX, chlorbenside+TX, chlordimeform+TX, chlordimeform hydrochloride+TX, chlorfenethol+TX, chlorfenson+TX, chlorfensulfide+TX, chlorobenzilate+TX, chloromebuform+TX, chloromethiuron+TX, chloropropylate+TX, chlorthiophos+TX, cinerin I+TX, cinerin II+TX, cinerins+TX, closantel+TX, coumaphos+TX, crotamiton+TX, crotoxyphos+TX, cufraneb+TX, cyanthoate+TX, DCPM+TX, DDT+TX, demephion+TX, demephion-O+TX, demephion-S+TX, demeton-methyl+TX, demeton-O+TX, demeton-O-methyl+TX, demeton-S+TX, demeton-S-methyl+TX, demeton-S-methylsulfon+TX, dichlofluanid+TX, dichlorvos+TX, dicliphos+TX, dienochlor+TX, dimefox+TX, dinex+TX, dinex-diclexine+TX, dinocap-4+TX, dinocap-6+TX, dinocton+TX, dino-penton+TX, dinosulfon+TX, dinoterbon+TX, dioxathion+TX, diphenyl sulfone+TX, disulfiram+TX, DNOC+TX, dofenapyn+TX, doramectin+TX, endothion+TX, eprinomectin+TX, ethoate-methyl+TX, etrimfos+TX, fenazaflor+TX, fenbutatin oxide+TX, fenothiocarb+TX, fenpyrad+TX, fen-pyroximate+TX, fenpyrazamine+TX, fenson+TX, fentrifanil+TX, flubenzimine+TX, flucycloxuron+TX, fluenetil+TX, fluorbenside+TX, FMC 1137+TX, formetanate+TX, formetanate hydrochloride+TX, formparanate+TX, gamma-HCH+TX, glyodin+TX, halfenprox+TX, hexadecyl cyclopropanecarboxylate+TX, isocarbophos+TX, jasmolin I+TX, jasmolin II+TX, jodfenphos+TX, lindane+TX, malonoben+TX, mecarbam+TX, mephosfolan+TX, mesulfen+TX, methacrifos+TX, methyl bromide+TX, metolcarb+TX, mexacarbate+TX, milbemycin oxime+TX, mipafox+TX, monocrotophos+TX, morphothion+TX, moxidectin+TX, naled+TX, 4-chloro-2-(2-chloro-2-methyl-propyl)-5-[(6-iodo-3-pyridyl)methoxy]pyridazin-3-one+TX, nifluridide+TX, nikkomycins+TX, nitrilacarb+TX, nitrilacarb 1:1 zinc chloride complex+TX, omethoate+TX, oxydeprofos+TX, oxydisulfoton+TX, pp′-DDT+TX, parathion+TX, permethrin+TX, phenkapton+TX, phosalone+TX, phosfolan+TX, phosphamidon+TX, polychloroterpenes+TX, polynactins+TX, proclonol+TX, promacyl+TX, propoxur+TX, prothidathion+TX, prothoate+TX, pyrethrin I+TX, pyrethrin II+TX, pyrethrins+TX, pyridaphenthion+TX, pyrimitate+TX, quinalphos+TX, quintiofos+TX, R-1492+TX, phosglycin+TX, rotenone+TX, schradan+TX, sebufos+TX, selamectin+TX, sophamide+TX, SSI-121+TX, sulfiram+TX, sulfluramid+TX, sulfotep+TX, sulfur+TX, diflovidazin+TX, tau-fluvalinate+TX, TEPP+TX, terbam+TX, tetradifon+TX, tetrasul+TX, thiafenox+TX, thiocarboxime+TX, thiofanox+TX, thiometon+TX, thioquinox+TX, thuringiensin+TX, triamiphos+TX, triarathene+TX, triazophos+TX, triazuron+TX, trifenofos+TX, trinactin+TX, vamidothion+TX, vaniliprole+TX, bethoxazin+TX, copper dioctanoate+TX, copper sulfate+TX, cybutryne+TX, dichlone+TX, dichlorophen+TX, endothal+TX, fentin+TX, hydrated lime+TX, nabam+TX, quinoclamine+TX, quinonamid+TX, simazine+TX, triphenyltin acetate+TX, triphenyltin hydroxide+TX, crufomate+TX, piperazine+TX, thiophanate+TX, chloralose+TX, fenthion+TX, pyridin-4-amine+TX, strychnine+TX, 1-hydroxy-1H-pyridine-2-thione+TX, 4-(quinoxalin-2-ylamino)benzenesulfonamide+TX, 8-hydroxyquinoline sulfate+TX, bronopol+TX, copper hydroxide+TX, cresol+TX, dipyrithione+TX, dodicin+TX, fenaminosulf+TX, formaldehyde+TX, hydrargaphen+TX, kasugamycin+TX, kasugamycin hydrochloride hydrate+TX, nickel bis(dimethyldithiocarbamate)+TX, nitrapyrin+TX, octhilinone+TX, oxolinic acid+TX, oxytetracycline+TX, potassium hydroxyquinoline sulfate+TX, probenazole+TX, streptomycin+TX, streptomycin sesquisulfate+TX, tecloftalam+TX, thiomersal+TX, Adoxophyes orana GV+TX, Agrobacterium radiobacter+TX, Amblyseius spp.+TX, Anagrapha falcifera NPV+TX, Anagrus atomus+TX, Aphelinus abdominalis+TX, Aphidius colemani+TX, Aphidoletes aphidimyza+TX, Autographa californica NPV+TX, Bacillus sphaericus Neide+TX, Beauveria brongniartii+TX, Chrysoperla carnea+TX, Cryptolaemus montrouzieri+TX, Cydia pomonella GV+TX, Dacnusa sibirica+TX, Diglyphus isaea+TX, Encarsia formosa+TX, Eretmocerus eremicus+TX, Heterorhabditis bacteriophora and H. megidis+TX, Hippodamia convergens+TX, Leptomastix dactylopii+TX, Macrolophus caliginosus+TX, Mamestra brassicae NPV+TX, Metaphycus helvolus+TX, Metarhizium anisopliae var. acridum+TX, Metarhizium anisopliae var. anisopliae+TX, Neodiprion sertifer NPV and N. lecontei NPV+TX, Orius spp.+TX, Paecilomyces fumosoroseus+TX, Phytoseiulus persimilis+TX, Steinernema bibionis+TX, Steinernema carpocapsae+TX, Steinernema feltiae+TX, Steinernema glaseri+TX, Steinernema riobrave+TX, Steinernema riobravis+TX, Steinernema scapterisci+TX, Steinernema spp.+TX, Trichogramma spp.+TX, Typhlodromus occidentalis+TX, Verticillium lecanii+TX, apholate+TX, bisazir+TX, busulfan+TX, dimatif+TX, hemel+TX, hempa+TX, metepa+TX, methiotepa+TX, methyl apholate+TX, morzid+TX, penfluron+TX, tepa+TX, thiohempa+TX, thiotepa+TX, tretamine+TX, uredepa+TX, (E)-dec-5-en-1-yl acetate with (E)-dec-5-en-1-ol+TX, (E)-tridec-4-en-1-yl acetate+TX, (E)-6-methylhept-2-en-4-ol+TX, (E,Z)-tetradeca-4,10-dien-1-yl acetate+TX, (Z)-dodec-7-en-1-yl acetate+TX, (Z)-hexadec-11-enal+TX, (Z)-hexadec-11-en-1-yl acetate+TX, (Z)-hexadec-13-en-11-yn-1-yl acetate+TX, (Z)-icos-13-en-10-one+TX, (Z)-tetradec-7-en-1-al+TX, (Z)-tetradec-9-en-1-ol+TX, (Z)-tetradec-9-en-1-yl acetate+TX, (7E,9Z)-dodeca-7,9-dien-1-yl acetate+TX, (9Z,11E)-tetradeca-9,11-dien-1-yl acetate+TX, (9Z,12E)-tetradeca-9,12-dien-1-yl acetate+TX, 14-methyloctadec-1-ene+TX, 4-methylnonan-5-ol with 4-methylnonan-5-one+TX, alpha-multistriatin+TX, brevicomin+TX, codlelure+TX, codlemone+TX, cuelure+TX, disparlure+TX, dodec-8-en-1-yl acetate+TX, dodec-9-en-1-yl acetate+TX, dodeca-8+TX, 10-dien-1-yl acetate+TX, dominicalure+TX, ethyl 4-methyloctanoate+TX, eugenol+TX, frontalin+TX, grandlure+TX, grandlure I+TX, grandlure II+TX, grandlure III+TX, grandlure IV+TX, hexalure+TX, ipsdienol+TX, ipsenol+TX, japonilure+TX, lineatin+TX, litlure+TX, looplure+TX, medlure+TX, megatomoic acid+TX, methyl eugenol+TX, muscalure+TX, octadeca-2,13-dien-1-yl acetate+TX, octadeca-3,13-dien-1-yl acetate+TX, orfralure+TX, oryctalure+TX, ostramone+TX, siglure+TX, sordidin+TX, sulcatol+TX, tetradec-11-en-1-yl acetate+TX, trimedlure+TX, trimedlure A+TX, trimedlure B₁+TX, trimedlure B₂+TX, trimedlure C+TX, trunc-call+TX, 2-(octylthio)-ethanol+TX, butopyronoxyl+TX, butoxy(polypropylene glycol)+TX, dibutyl adipate+TX, dibutyl phthalate+TX, dibutyl succinate+TX, diethyltoluamide+TX, dimethyl carbate+TX, dimethyl phthalate+TX, ethyl hexanediol+TX, hexamide+TX, methoquin-butyl+TX, methylneodecanamide+TX, oxamate+TX, picaridin+TX, 1-dichloro-1-nitroethane+TX, 1,1-dichloro-2,2-bis(4-ethylphenyl)-ethane+TX, 1,2-dichloropropane with 1,3-dichloropropene+TX, 1-bromo-2-chloroethane+TX, 2,2,2-trichloro-1-(3,4-dichloro-phenyl)ethyl acetate+TX, 2,2-dichlorovinyl 2-ethylsulfinylethyl methyl phosphate+TX, 2-(1,3-dithiolan-2-yl)phenyl dimethylcarbamate+TX, 2-(2-butoxyethoxy)ethyl thiocyanate+TX, 2-(4,5-dimethyl-1,3-dioxolan-2-yl)phenyl methylcarbamate+TX, 2-(4-chloro-3,5-xylyloxy)ethanol+TX, 2-chlorovinyl diethyl phosphate+TX, 2-imidazolidone+TX, 2-isovalerylindan-1,3-dione+TX, 2-methyl(prop-2-ynyl)aminophenyl methylcarbamate+TX, 2-thiocyanatoethyl laurate+TX, 3-bromo-1-chloroprop-1-ene+TX, 3-methyl-1-phenylpyrazol-5-yl dimethyl-carbamate+TX, 4-methyl(prop-2-ynyl)amino-3,5-xylyl methylcarbamate+TX, 5,5-dimethyl-3-oxocyclohex-1-enyl dimethylcarbamate+TX, acethion+TX, acrylonitrile+TX, aldrin+TX, allosamidin+TX, allyxycarb+TX, alpha-ecdysone+TX, aluminium phosphide+TX, aminocarb+TX, anabasine+TX, athidathion+TX, azamethiphos+TX, Bacillus thuringiensis delta endotoxins+TX, barium hexafluorosilicate+TX, barium polysulfide+TX, barthrin+TX, Bayer 22/190+TX, Bayer 22408+TX, beta-cyfluthrin+TX, beta-cypermethrin+TX, bioethanomethrin+TX, biopermethrin+TX, bis(2-chloroethyl) ether+TX, borax+TX, bromfenvinfos+TX, bromo-DDT+TX, bufencarb+TX, butacarb+TX, butathiofos+TX, butonate+TX, calcium arsenate+TX, calcium cyanide+TX, carbon disulfide+TX, carbon tetrachloride+TX, cartap hydrochloride+TX, cevadine+TX, chlorbicyclen+TX, chlordane+TX, chlordecone+TX, chloroform+TX, chloropicrin+TX, chlorphoxim+TX, chlorprazophos+TX, cis-resmethrin+TX, cismethrin+TX, clocythrin+TX, copper acetoarsenite+TX, copper arsenate+TX, copper oleate+TX, coumithoate+TX, cryolite+TX, CS 708+TX, cyanofenphos+TX, cyanophos+TX, cyclethrin+TX, cythioate+TX, d-tetramethrin+TX, DAEP+TX, dazomet+TX, decarbofuran+TX, diamidafos+TX, dicapthon+TX, dichlofenthion+TX, dicresyl+TX, dicyclanil+TX, dieldrin+TX, diethyl 5-methylpyrazol-3-yl phosphate+TX, dilor+TX, dimefluthrin+TX, dimetan+TX, dimethrin+TX, dimethylvinphos+TX, dimetilan+TX, dinoprop+TX, dinosam+TX, dinoseb+TX, diofenolan+TX, dioxabenzofos+TX, dithicrofos+TX, DSP+TX, ecdysterone+TX, EI 1642+TX, EMPC+TX, EPBP+TX, etaphos+TX, ethiofencarb+TX, ethyl formate+TX, ethylene dibromide+TX, ethylene dichloride+TX, ethylene oxide+TX, EXD+TX, fenchlorphos+TX, fenethacarb+TX, fenitrothion+TX, fenoxacrim+TX, fenpirithrin+TX, fensulfothion+TX, fenthion-ethyl+TX, flucofuron+TX, fosmethilan+TX, fospirate+TX, fosthietan+TX, furathiocarb+TX, furethrin+TX, guazatine+TX, guazatine acetates+TX, sodium tetrathiocarbonate+TX, halfenprox+TX, HCH+TX, HEOD+TX, heptachlor+TX, heterophos+TX, HHDN+TX, hydrogen cyanide+TX, hyquincarb+TX, IPSP+TX, isazofos+TX, isobenzan+TX, isodrin+TX, isofenphos+TX, isolane+TX, isoprothiolane+TX, isoxathion+TX, juvenile hormone I+TX, juvenile hormone II+TX, juvenile hormone III+TX, kelevan+TX, kinoprene+TX, lead arsenate+TX, leptophos+TX, lirimfos+TX, lythidathion+TX, m-cumenyl methylcarbamate+TX, magnesium phosphide+TX, mazidox+TX, mecarphon+TX, menazon+TX, mercurous chloride+TX, mesulfenfos+TX, metam+TX, metam-potassium+TX, metam-sodium+TX, methanesulfonyl fluoride+TX, methocrotophos+TX, methoprene+TX, methothrin+TX, methoxychlor+TX, methyl isothiocyanate+TX, methylchloroform+TX, methylene chloride+TX, metoxadiazone+TX, mirex+TX, naftalofos+TX, naphthalene+TX, NC-170+TX, nicotine+TX, nicotine sulfate+TX, nithiazine+TX, nornicotine+TX, O-5-dichloro-4-iodophenyl O-ethyl ethylphosphonothioate+TX, O,O-diethyl 0-4-methyl-2-oxo-2H-chromen-7-yl phosphorothioate+TX, O,O-diethyl 0-6-methyl-2-propylpyrimidin-4-yl phosphorothioate+TX, O,O,O′,O′-tetrapropyl dithiopyrophosphate+TX, oleic acid+TX, para-dichlorobenzene+TX, parathion-methyl+TX, pentachlorophenol+TX, pentachlorophenyl laurate+TX, PH 60-38+TX, phenkapton+TX, phosnichlor+TX, phosphine+TX, phoxim-methyl+TX, pirimetaphos+TX, polychlorodicyclopentadiene isomers+TX, potassium arsenite+TX, potassium thiocyanate+TX, precocene I+TX, precocene II+TX, precocene III+TX, primidophos+TX, profluthrin+TX, promecarb+TX, prothiofos+TX, pyrazophos+TX, pyresmethrin+TX, quassia+TX, quinalphos-methyl+TX, quinothion+TX, rafoxanide+TX, resmethrin+TX, rotenone+TX, kadethrin+TX, ryania+TX, ryanodine+TX, sabadilla+TX, schradan+TX, sebufos+TX, SI-0009+TX, thiapronil+TX, sodium arsenite+TX, sodium cyanide+TX, sodium fluoride+TX, sodium hexafluorosilicate+TX, sodium pentachlorophenoxide+TX, sodium selenate+TX, sodium thiocyanate+TX, sulcofuron+TX, sulcofuron-sodium+TX, sulfuryl fluoride+TX, sulprofos+TX, tar oils+TX, tazimcarb+TX, TDE+TX, tebupirimfos+TX, temephos+TX, terallethrin+TX, tetrachloroethane+TX, thicrofos+TX, thiocyclam+TX, thiocyclam hydrogen oxalate+TX, thionazin+TX, thiosultap+TX, thiosultap-sodium+TX, tralomethrin+TX, transpermethrin+TX, triazamate+TX, trichlormetaphos-3+TX, trichloronat+TX, trimethacarb+TX, tolprocarb+TX, triclopyricarb+TX, triprene+TX, veratridine+TX, veratrine+TX, XMC+TX, zetamethrin+TX, zinc phosphide+TX, zolaprofos+TX, meperfluthrin+TX, tetramethylfluthrin+TX, bis(tributyltin) oxide+TX, bromoacetamide+TX, ferric phosphate+TX, niclosamide-olamine+TX, tributyltin oxide+TX, pyrimorph+TX, trifenmorph+TX, 1,2-dibromo-3-chloropropane+TX, 1,3-dichloropropene+TX, 3,4-dichlorotetrahydrothio-phene 1,1-dioxide+TX, 3-(4-chlorophenyl)-5-methylrhodanine+TX, 5-methyl-6-thioxo-1,3,5-thiadiazinan-3-ylacetic acid+TX, 6-isopentenylaminopurine+TX, anisiflupurin+TX, benclothiaz+TX, cytokinins+TX, DCIP+TX, furfural+TX, isamidofos+TX, kinetin+TX, Myrothecium verrucaria composition+TX, tetrachlorothiophene+TX, xylenols+TX, zeatin+TX, potassium ethylxanthate+TX, acibenzolar+TX, acibenzolar-S-methyl+TX, Reynoutria sachalinensis extract+TX, alpha-chlorohydrin+TX, antu+TX, barium carbonate+TX, bisthiosemi+TX, brodifacoum+TX, bromadiolone+TX, bromethalin+TX, chlorophacinone+TX, cholecalciferol+TX, coumachlor+TX, coumafuryl+TX, coumatetralyl+TX, crimidine+TX, difenacoum+TX, difethialone+TX, diphacinone+TX, ergocalciferol+TX, flocoumafen+TX, fluoroacetamide+TX, flupropadine+TX, flupropadine hydrochloride+TX, norbormide+TX, phosacetim+TX, phosphorus+TX, pindone+TX, pyrinuron+TX, scilliroside+TX, -sodium fluoroacetate+TX, thallium sulfate+TX, warfarin+TX, -2-(2-butoxyethoxy)ethyl piperonylate+TX, 5-(1,3-benzodioxol-5-yl)-3-hexylcyclohex-2-enone+TX, farnesol with nerolidol+TX, verbutin+TX, MGK 264+TX, piperonyl butoxide+TX, piprotal+TX, propyl isomer+TX, S421+TX, sesamex+TX, sesasmolin+TX, sulfoxide+TX, anthraquinone+TX, copper naphthenate+TX, copper oxychloride+TX, dicyclopentadiene+TX, thiram+TX, zinc naphthenate+TX, ziram+TX, imanin+TX, ribavirin+TX, chloroinconazide+TX, mercuric oxide+TX, thiophanate-methyl+TX, azaconazole+TX, bitertanol+TX, bromuconazole+TX, cyproconazole+TX, difenoconazole+TX, diniconazole-+TX, epoxiconazole+TX, fenbuconazole+TX, fluquinconazole+TX, flusilazole+TX, flutriafol+TX, furametpyr+TX, hexaconazole+TX, imazalil-+TX, imiben-conazole+TX, ipconazole+TX, metconazole+TX, myclobutanil+TX, paclobutrazole+TX, pefurazoate+TX, penconazole+TX, prothioconazole+TX, pyrifenox+TX, prochloraz+TX, propiconazole+TX, pyrisoxazole+TX, -simeconazole+TX, tebucon-azole+TX, tetraconazole+TX, triadimefon+TX, triadimenol+TX, triflumizole+TX, triticonazole+TX, ancymidol+TX, fenarimol+TX, nuarimol+TX, bupirimate+TX, dimethirimol+TX, ethirimol+TX, dodemorph+TX, fenpropidin+TX, fenpropimorph+TX, spiroxamine+TX, tridemorph+TX, cyprodinil+TX, mepanipyrim+TX, pyrimethanil+TX, fenpiclonil+TX, fludioxonil+TX, benalaxyl+TX, furalaxyl+TX, -metalaxyl-+TX, Rmetalaxyl+TX, ofurace+TX, oxadixyl+TX, carbendazim+TX, debacarb+TX, fuberidazole-+TX, thiabendazole+TX, chlozolinate+TX, dichlozoline+TX, myclozoline-+TX, procymidone+TX, vinclozoline+TX, boscalid+TX, carboxin+TX, fenfuram+TX, flutolanil+TX, mepronil+TX, oxycarboxin+TX, penthiopyrad+TX, thifluzamide+TX, dodine+TX, iminoctadine+TX, azoxystrobin+TX, dimoxystrobin+TX, enestroburin+TX, fenaminstrobin+TX, flufenoxystrobin+TX, fluoxastrobin+TX, kresoxim—methyl+TX, metominostrobin+TX, trifloxystrobin+TX, orysastrobin+TX, picoxystrobin+TX, pyraclostrobin+TX, pyrametostrobin+TX, pyraoxystrobin+TX, ferbam+TX, mancozeb+TX, maneb+TX, metiram+TX, propineb+TX, zineb+TX, captafol+TX, captan+TX, fluoroimide+TX, folpet+TX, tolylfluanid+TX, bordeaux mixture+TX, copper oxide+TX, mancopper+TX, oxine-copper+TX, nitrothal-isopropyl+TX, edifenphos+TX, iprobenphos+TX, phosdiphen+TX, tolclofos-methyl+TX, anilazine+TX, benthiavalicarb+TX, blasticidin-S+TX, chloroneb-+TX, chloro-tha-lonil+TX, cyflufenamid+TX, cymoxanil+TX, cyclobutrifluram+TX, diclocymet+TX, diclomezine-+TX, dicloran+TX, diethofencarb+TX, dimethomorph-+TX, flumorph+TX, dithianon+TX, ethaboxam+TX, etridiazole+TX, famoxadone+TX, fenamidone+TX, fenoxanil+TX, ferimzone+TX, fluazinam+TX, flumetylsulforim+TX, fluopicolide+TX, fluoxytioconazole+TX, flusulfamide+TX, fluxapyroxad+TX, -fenhexamid+TX, fosetyl-aluminium-+TX, hymexazol+TX, iprovalicarb+TX, cyazofamid+TX, methasulfocarb+TX, metrafenone+TX, pencycuron+TX, phthalide+TX, polyoxins+TX, propamocarb+TX, pyribencarb+TX, proquinazid+TX, pyroquilon+TX, pyriofenone+TX, quinoxyfen+TX, quintozene+TX, tiadinil+TX, triazoxide+TX, tricyclazole+TX, triforine+TX, validamycin+TX, valifenalate+TX, zoxamide+TX, mandipropamid+TX, flubeneteram+TX, isopyrazam+TX, sedaxane+TX, benzovindiflupyr+TX, pydiflumetofen+TX, 3-difluoromethyl-1-methyl-1H-pyrazole-4-carboxylic acid (3′,4′,5′-trifluoro-biphenyl-2-yl)-amide+TX, isoflucypram+TX, isotianil+TX, dipymetitrone+TX, 6-ethyl-5,7-dioxo-pyrrolo[4,5][1,4]dithiino[1,2-c]isothiazole-3-carbonitrile+TX, 2-(difluoromethyl)-N-[3-ethyl-1,1-dimethyl-indan-4-yl]pyridine-3-carboxamide+TX, 4-(2,6-difluorophenyl)-6-methyl-5-phenyl-pyridazine-3-carbonitrile+TX, (R)-3-(difluoromethyl)-1-methyl-N-[1,1,3-trimethylindan-4-yl]pyrazole-4-carboxamide+TX, 4-(2-bromo-4-fluoro-phenyl)-N-(2-chloro-6-fluoro-phenyl)-2,5-dimethyl-pyrazol-3-amine+TX, 4-(2-bromo-4-fluorophenyl)-N-(2-chloro-6-fluorophenyl)-1,3-dimethyl-1H-pyrazol-5-amine+TX, fluindapyr+TX, coumethoxystrobin (jiaxiangjunzhi)+TX, Ivbenmixianan+TX, dichlobentiazox+TX, mandestrobin+TX, 3-(4,4-difluoro-3,4-dihydro-3,3-dimethylisoquinolin-1-yl)quinolone+TX, 2-[2-fluoro-6-[(8-fluoro-2-methyl-3-quinolyl)oxy]phenyl]propan-2-ol+TX, oxathiapiprolin+TX, tert-butyl N-[6-[[[(1-methyltetrazol-5-yl)-phenyl-methylene]amino]oxymethyl]-2-pyridyl]carbamate+TX, pyraziflumid+TX, inpyrfluxam+TX, trolprocarb+TX, mefentrifluconazole+TX, ipfentrifluconazole+TX, 2-(difluoromethyl)-N-[(3R)-3-ethyl-1,1-dimethyl-indan-4-yl]pyridine-3-carboxamide+TX, N′-(2,5-dimethyl-4-phenoxy-phenyl)-N-ethyl-N-methyl-formamidine+TX, N′-[4-(4,5-dichlorothiazol-2-yl)oxy-2,5-dimethyl-phenyl]-N-ethyl-N-methyl-formamidine+TX, [2-[3-[2-[1-[2-[3,5-bis(difluoromethyl)pyrazol-1-yl]acetyl]-4-piperidyl]thiazol-4-yl]-4,5-dihydroisoxazol-5-yl]-3-chloro-phenyl] methanesulfonate+TX, but-3-ynyl N-[6-[[(Z)-[(1-methyltetrazol-5-yl)-phenyl-methylene]amino]oxymethyl]-2-pyridyl]carbamate+TX, methyl N-[[5-[4-(2,4-dimethylphenyl)triazol-2-yl]-2-methyl-phenyl]methyl]carbamate+TX, 3-chloro-6-methyl-5-phenyl-4-(2,4,6-trifluorophenyl)pyridazine+TX, pyridachlometyl+TX, 3-(difluoromethyl)-1-methyl-N-[1,1,3-trimethylindan-4-yl]pyrazole-4-carboxamide+TX, 1-[2-[[1-(4-chlorophenyl)pyrazol-3-yl]oxymethyl]-3-methyl-phenyl]-4-methyl-tetrazol-5-one+TX, 1-methyl-4-[3-methyl-2-[[2-methyl-4-(3,4,5-trimethylpyrazol-1-yl)phenoxy]methyl]phenyl]tetrazol-5-one+TX, aminopyrifen+TX, ametoctradin+TX, amisulbrom+TX, penflufen+TX, (Z,2E)-5-[1-(4-chlorophenyl)pyrazol-3-yl]oxy-2-methoxyimino-N,3-dimethyl-pent-3-enamide+TX, florylpicoxamid+TX, fenpicoxamid+TX, metarylpicoxamid+TX, tebufloquin+TX, ipflufenoquin+TX, quinofumelin+TX, isofetamid+TX, ethyl 1-[[4-[[2-(trifluoromethyl)-1,3-dioxolan-2-yl]methoxy]phenyl]methyl]pyrazole-3-carboxylate (may be prepared from the methods described in WO 2020/056090)+TX, ethyl 1-[[4-[(Z)-2-ethoxy-3,3,3-trifluoro-prop-1-enoxy]phenyl]methyl]pyrazole-3-carboxylate (may be prepared from the methods described in WO 2020/056090)+TX, methyl N-[[4-[1-(4-cyclopropyl-2,6-difluoro-phenyl)pyrazol-4-yl]-2-methyl-phenyl]methyl]carbamate (may be prepared from the methods described in WO 2020/097012)+TX, methyl N-[[4-[1-(2,6-difluoro-4-isopropyl-phenyl)pyrazol-4-yl]-2-methyl-phenyl]methyl]carbamate (may be prepared from the methods described in WO 2020/097012)+TX, N-[2-[2,4-dichloro-phenoxy]phenyl]-3-(difluoromethyl)-1-methyl-pyrazole-4-carboxamide+TX, N-[2-[2-chloro-4-(trifluoromethyl)phenoxy]phenyl]-3-(difluoromethyl)-1-methyl-pyrazole-4-carboxamide+TX, benzothiostrobin+TX, phenamacril+TX, 5-amino-1,3,4-thiadiazole-2-thiol zinc salt (2:1)+TX, fluopyram+TX, flufenoxadiazam+TX, flutianil+TX, fluopimomide+TX, pyrapropoyne+TX, picarbutrazox+TX, 2-(difluoromethyl)-N-(3-ethyl-1,1-dimethyl-indan-4-yl)pyridine-3-carboxamide+TX, 2-(difluoromethyl)-N-((3R)-1,1,3-trimethylindan-4-yl) pyridine-3-carboxamide+TX, 4-[[6-[2-(2,4-difluorophenyl)-1,1-difluoro-2-hydroxy-3-(1,2,4-triazol-1-yl)propyl]-3-pyridyl]oxy]benzonitrile+TX, metyltetraprole+TX, 2-(difluoromethyl)-N-((3R)-1,1,3-trimethylindan-4-yl) pyridine-3-carboxamide+TX, α-(1,1-dimethylethyl)-α-[4′-(trifluoromethoxy) [1,1′-biphenyl]-4-yl]-5-pyrimidinemethanol+TX, fluoxapiprolin+TX, enoxastrobin+TX, 4-[[6-[2-(2,4-difluorophenyl)-1,1-difluoro-2-hydroxy-3-(1,2,4-triazol-1-yl)propyl]-3-pyridyl]oxy] benzonitrile+TX, 4-[[6-[2-(2,4-difluorophenyl)-1,1-difluoro-2-hydroxy-3-(5-sulfanyl-1,2,4-triazol-1-yl)propyl]-3-pyridyl]oxy] benzonitrile+TX, 4-[[6-[2-(2,4-difluorophenyl)-1,1-difluoro-2-hydroxy-3-(5-thioxo-4H-1,2,4-triazol-1-yl)propyl]-3-pyridyl]oxy]benzonitrile+TX, trinexapac+TX, coumoxystrobin+TX, zhongshengmycin+TX, thiodiazole copper+TX, zinc thiazole+TX, amectotractin+TX, iprodione+TX, seboctylamine+TX; N′-[5-bromo-2-methyl-6-[(1S)-1-methyl-2-propoxy-ethoxy]-3-pyridyl]-N-ethyl-N-methyl-formamidine+TX, N′-[5-bromo-2-methyl-6-[(1R)-1-methyl-2-propoxy-ethoxy]-3-pyridyl]-N-ethyl-N-methyl-formamidine+TX, N′-[5-bromo-2-methyl-6-(1-methyl-2-propoxy-ethoxy)-3-pyridyl]-N-ethyl-N-methyl-formamidine+TX, N′-[5-chloro-2-methyl-6-(1-methyl-2-propoxy-ethoxy)-3-pyridyl]-N-ethyl-N-methyl-formamidine+TX, N′-[5-bromo-2-methyl-6-(1-methyl-2-propoxy-ethoxy)-3-pyridyl]-N-isopropyl-N-methyl-formamidine+TX (these compounds may be prepared from the methods described in WO2015/155075); N′-[5-bromo-2-methyl-6-(2-propoxypropoxy)-3-pyridyl]-N-ethyl-N-methyl-formamidine+TX (this compound may be prepared from the methods described in IPCOM000249876D); N-isopropyl-N′-[5-methoxy-2-methyl-4-(2,2,2-trifluoro-1-hydroxy-1-phenyl-ethyl)phenyl]-N-methyl-formamidine+TX, N′-[4-(1-cyclopropyl-2,2,2-trifluoro-1-hydroxy-ethyl)-5-methoxy-2-methyl-phenyl]-N-isopropyl-N-methyl-formamidine+TX (these compounds may be prepared from the methods described in WO2018/228896); N-ethyl-N′-[5-methoxy-2-methyl-4-[(2-trifluoromethyl)oxetan-2-yl]phenyl]-N-methyl-formamidine+TX, N-ethyl-N′-[5-methoxy-2-methyl-4-[(2-trifuoromethyl)tetrahydrofuran-2-yl]phenyl]-N-methyl-formamidine+TX (these compounds may be prepared from the methods described in WO2019/110427); N-[(1R)-1-benzyl-3-chloro-1-methyl-but-3-enyl]-8-fluoro-quinoline-3-carboxamide+TX, N-[(1S)-1-benzyl-3-chloro-1-methyl-but-3-enyl]-8-fluoro-quinoline-3-carboxamide+TX, N-[(1R)-1-benzyl-3,3,3-trifluoro-1-methyl-propyl]-8-fluoro-quinoline-3-carboxamide+TX, N-[(1S)-1-benzyl-3,3,3-trifluoro-1-methyl-propyl]-8-fluoro-quinoline-3-carboxamide+TX, N-[(1R)-1-benzyl-1,3-dimethyl-butyl]-7,8-difluoro-quinoline-3-carboxamide+TX, N-[(1S)-1-benzyl-1,3-dimethyl-butyl]-7,8-difluoro-quinoline-3-carboxamide+TX, 8-fluoro-N-[(1R)-1-[(3-fluorophenyl)methyl]-1,3-dimethyl-butyl]quinoline-3-carboxamide+TX, 8-fluoro-N-[(1S)-1-[(3-fluorophenyl)methyl]-1,3-dimethyl-butyl]quinoline-3-carboxamide+TX, N-[(1R)-1-benzyl-1,3-dimethyl-butyl]-8-fluoro-quinoline-3-carboxamide+TX, N-[(1S)-1-benzyl-1,3-dimethyl-butyl]-8-fluoro-quinoline-3-carboxamide+TX, N-((1R)-1-benzyl-3-chloro-1-methyl-but-3-enyl)-8-fluoro-quinoline-3-carboxamide+TX, N-((1S)-1-benzyl-3-chloro-1-methyl-but-3-enyl)-8-fluoro-quinoline-3-carboxamide+TX (these compounds may be prepared from the methods described in WO2017/153380); 1-(6,7-dimethylpyrazolo[1,5-a]pyridin-3-yl)-4,4,5-trifluoro-3,3-dimethyl-isoquinoline+TX, 1-(6,7-dimethylpyrazolo[1,5-a]pyridin-3-yl)-4,4,6-trifluoro-3,3-dimethyl-isoquinoline+TX, 4,4-difluoro-3,3-dimethyl-1-(6-methylpyrazolo[1,5-a]pyridin-3-yl)isoquinoline+TX, 4,4-difluoro-3,3-dimethyl-1-(7-methylpyrazolo[1,5-a]pyridin-3-yl)isoquinoline+TX, 1-(6-chloro-7-methyl-pyrazolo[1,5-a]pyridin-3-yl)-4,4-difluoro-3,3-dimethyl-isoquinoline+TX (these compounds may be prepared from the methods described in WO2017/025510); 1-(4,5-dimethylbenzimidazol-1-yl)-4,4,5-trifluoro-3,3-dimethyl-isoquinoline+TX, 1-(4,5-dimethylbenzimidazol-1-yl)-4,4-difluoro-3,3-dimethyl-isoquinoline+TX, 6-chloro-4,4-difluoro-3,3-dimethyl-1-(4-methylbenzimidazol-1-yl)isoquinoline+TX, 4,4-difluoro-1-(5-fluoro-4-methyl-benzimidazol-1-yl)-3,3-dimethyl-isoquinoline+TX, 3-(4,4-difluoro-3,3-dimethyl-1-isoquinolyl)-7,8-dihydro-6H-cyclopenta[e]benzimidazole+TX (these compounds may be prepared from the methods described in WO2016/156085); N-methoxy-N-[[4-[5-(trifluoromethyl)-1,2,4-oxadiazol-3-yl]phenyl]methyl]cyclopropanecarboxamide+TX, N,2-dimethoxy-N-[[4-[5-(trifluoromethyl)-1,2,4-oxadiazol-3-yl]phenyl]methyl]propanamide+TX, N-ethyl-2-methyl-N-[[4-[5-(trifluoromethyl)-1,2,4-oxadiazol-3-yl]phenyl]methyl]propanamide+TX, 1-methoxy-3-methyl-1-[[4-[5-(trifluoromethyl)-1,2,4-oxadiazol-3-yl]phenyl]methyl]urea+TX, 1,3-dimethoxy-1-[[4-[5-(trifluoromethyl)-1,2,4-oxadiazol-3-yl]phenyl]methyl]urea+TX, 3-ethyl-1-methoxy-1-[[4-[5-(trifluoromethyl)-1,2,4-oxadiazol-3-yl]phenyl]methyl]urea+TX, N-[[4-[5-(trifluoromethyl)-1,2,4-oxadiazol-3-yl]phenyl]methyl]propanamide+TX, 4,4-dimethyl-2-[[4-[5-(trifluoromethyl)-1,2,4-oxadiazol-3-yl]phenyl]methyl]isoxazolidin-3-one+TX, 5,5-dimethyl-2-[[4-[5-(trifluoromethyl)-1,2,4-oxadiazol-3-yl]phenyl]methyl]isoxazolidin-3-one+TX, ethyl 1-[[4-[5-(trifluoromethyl)-1,2,4-oxadiazol-3-yl]phenyl]methyl]pyrazole-4-carboxylate+TX, N,N-dimethyl-1-[[4-[5-(trifluoromethyl)-1,2,4-oxadiazol-3-yl]phenyl]methyl]-1,2,4-triazol-3-amine+TX. The compounds in this paragraph may be prepared from the methods described in WO 2017/055473, WO 2017/055469, WO 2017/093348 and WO 2017/118689; 2-[6-(4-chlorophenoxy)-2-(trifluoromethyl)-3-pyridyl]-1-(1,2,4-triazol-1-yl)propan-2-ol+TX (this compound may be prepared from the methods described in WO 2017/029179); 2-[6-(4-bromophenoxy)-2-(trifluoromethyl)-3-pyridyl]-1-(1,2,4-triazol-1-yl)propan-2-ol+TX (this compound may be prepared from the methods described in WO 2017/029179); 3-[2-(1-chlorocyclopropyl)-3-(2-fluorophenyl)-2-hydroxy-propyl]imidazole-4-carbonitrile+TX (this compound may be prepared from the methods described in WO 2016/156290); 3-[2-(1-chlorocyclopropyl)-3-(3-chloro-2-fluoro-phenyl)-2-hydroxy-propyl]imidazole-4-carbonitrile+TX (this compound may be prepared from the methods described in WO 2016/156290); (4-phenoxyphenyl)methyl 2-amino-6-methyl-pyridine-3-carboxylate+TX (this compound may be prepared from the methods described in WO 2014/006945); 2,6-Dimethyl-1H,5H-[1,4]dithiino[2,3-c:5,6-c′]dipyrrole-1,3,5,7(2H,6H)-tetrone+TX (this compound may be prepared from the methods described in WO 2011/138281); N-methyl-4-[5-(trifluoromethyl)-1,2,4-oxadiazol-3-yl]benzenecarbothioamide+TX; N-methyl-4-[5-(trifluoromethyl)-1,2,4-oxadiazol-3-yl]benzamide+TX; (Z,2E)-5-[1-(2,4-dichlorophenyl)pyrazol-3-yl]oxy-2-methoxyimino-N,3-dimethyl-pent-3-enamide+TX (this compound may be prepared from the methods described in WO 2018/153707); N′-(2-chloro-5-methyl-4-phenoxy-phenyl)-N-ethyl-N-methyl-formamidine+TX; N′-[2-chloro-4-(2-fluorophenoxy)-5-methyl-phenyl]-N-ethyl-N-methyl-formamidine+TX (this compound may be prepared from the methods described in WO 2016/202742); 2-(difluoromethyl)-N-[(3S)-3-ethyl-1,1-dimethyl-indan-4-yl]pyridine-3-carboxamide+TX (this compound may be prepared from the methods described in WO 2014/095675); (5-methyl-2-pyridyl)-[4-[5-(trifluoromethyl)-1,2,4-oxadiazol-3-yl]phenyl]methanone+TX, (3-methylisoxazol-5-yl)-[4-[5-(trifluoromethyl)-1,2,4-oxadiazol-3-yl]phenyl]methanone+TX (these compounds may be prepared from the methods described in WO 2017/220485); 2-oxo-N-propyl-2-[4-[5-(trifluoromethyl)-1,2,4-oxadiazol-3-yl]phenyl]acetamide+TX (this compound may be prepared from the methods described in WO 2018/065414); ethyl 1-[[5-[5-(trifluoromethyl)-1,2,4-oxadiazol-3-yl]-2-thienyl]methyl]pyrazole-4-carboxylate+TX (this compound may be prepared from the methods described in WO 2018/158365); 2,2-difluoro-N-methyl-2-[4-[5-(trifluoromethyl)-1,2,4-oxadiazol-3-yl]phenyl]acetamide+TX, N-[(E)-methoxyiminomethyl]-4-[5-(trifluoromethyl)-1,2,4-oxadiazol-3-yl]benzamide+TX, N-[(Z)-methoxyiminomethyl]-4-[5-(trifluoromethyl)-1,2,4-oxadiazol-3-yl]benzamide+TX, N-[N-methoxy-C-methyl-carbonimidoyl]-4-[5-(trifluoromethyl)-1,2,4-oxadiazol-3-yl]benzamide+TX (these compounds may be prepared from the methods described in WO 2018/202428); microbials including: Acinetobacter lwoffii+TX, Acremonium alternatum+TX+TX, Acremonium cephalosporium+TX+TX, Acremonium diospyri+TX, Acremonium obclavatum+TX, Adoxophyes orana granulovirus (AdoxGV) (Capex®)+TX, Agrobacterium radiobacter strain K84 (Galltrol-A®)+TX, Alternaria alternate+TX, Alternaria cassia+TX, Alternaria destruens (Smolder®)+TX, Ampelomyces quisqualis (AQ10®)+TX, Aspergillus flavus AF36 (AF36®)+TX, Aspergillus flavus NRRL 21882 (Aflaguard®)+TX, Aspergillus spp.+TX, Aureobasidium pullulans+TX, Azospirillum+TX, (MicroAZ®+TX, TAZO B®)+TX, Azotobacter+TX, Azotobacter chroocuccum (Azotomeal®)+TX, Azotobacter cysts (Bionatural Blooming Blossoms®)+TX, Bacillus amyloliquefaciens+TX, Bacillus cereus+TX, Bacillus chitinosporus strain CM-1+TX, Bacillus chitinosporus strain AQ746+TX, Bacillus licheniformis strain HB-2 (Biostart™ Rhizoboost®)+TX, Bacillus licheniformis strain 3086 (EcoGuard®+TX, Green Releaf®)+TX, Bacillus circulans+TX, Bacillus firmus (BioSafe®+TX, BioNem-WP®+TX, VOTiVO®)+TX, Bacillus firmus strain 1-1582+TX, Bacillus macerans+TX, Bacillus marismortui+TX, Bacillus megaterium+TX, Bacillus mycoides strain AQ726+TX, Bacillus papillae (Milky Spore Powder®)+TX, Bacillus pumilus spp.+TX, Bacillus pumilus strain GB34 (Yield Shield®)+TX, Bacillus pumilus strain AQ717+TX, Bacillus pumilus strain QST 2808 (Sonata®+TX, Ballad Plus®)+TX, Bacillus spahericus (VectoLex®)+TX, Bacillus spp.+TX, Bacillus spp. strain AQ175+TX, Bacillus spp. strain AQ177+TX, Bacillus spp. strain AQ178+TX, Bacillus subtilis strain QST 713 (CEASE®+TX, Serenade®+TX, Rhapsody®)+TX, Bacillus subtilis strain QST 714 (JAZZ®)+TX, Bacillus subtilis strain AQ153+TX, Bacillus subtilis strain AQ743+TX, Bacillus subtilis strain QST3002+TX, Bacillus subtilis strain QST3004+TX, Bacillus subtilis var. amyloliquefaciens strain FZB24 (Taegro®+TX, Rhizopro®)+TX, Bacillus thuringiensis Cry 2Ae+TX, Bacillus thuringiensis Cry1Ab+TX, Bacillus thuringiensis aizawai GC 91 (Agree®)+TX, Bacillus thuringiensis israelensis (BMP123®+TX, Aquabac®+TX, VectoBac®)+TX, Bacillus thuringiensis kurstaki (Javelin®+TX, Deliver®+TX, CryMax®+TX, Bonide®+TX, Scutella WP®+TX, Turilav WP®+TX, Astuto®+TX, Dipel WP®+TX, Biobit®+TX, Foray®)+TX, Bacillus thuringiensis kurstaki BMP 123 (Baritone®)+TX, Bacillus thuringiensis kurstaki HD-1 (Bioprotec-CAF/3P®)+TX, Bacillus thuringiensis strain BD #32+TX, Bacillus thuringiensis strain AQ52+TX, Bacillus thuringiensis var. aizawai (XenTari®+TX, DiPel®)+TX, bacteria spp. (GROWMEND® +TX, GROWSWEET®+TX, Shootup®)+TX, bacteriophage of Clavipacter michiganensis (AgriPhage®)+TX, Bakflor®+TX, Beauveria bassiana (Beaugenic®+TX, Brocaril WP®)+TX, Beauveria bassiana GHA (Mycotrol ES®+TX, Mycotrol O®+TX, BotaniGuard®)+TX, Beauveria brongniartii (Engerlingspilz®+TX, Schweizer Beauveria®+TX, Melocont®)+TX, Beauveria spp.+TX, Botrytis cineria+TX, Bradyrhizobium japonicum (TerraMax®)+TX, Brevibacillus brevis+TX, Bacillus thuringiensis tenebrionis (Novodor®)+TX, BtBooster+TX, Burkholderia cepacia (Deny®+TX, Intercept®+TX, Blue Circle®)+TX, Burkholderia gladii+TX, Burkholderia gladioli+TX, Burkholderia spp.+TX, Canadian thistle fungus (CBH Canadian Bioherbicide®)+TX, Candida butyri+TX, Candida famata+TX, Candida fructus+TX, Candida glabrata+TX, Candida guilliermondii+TX, Candida melibiosica+TX, Candida oleophila strain O+TX, Candida parapsilosis+TX, Candida pelliculosa+TX, Candida pulcherrima+TX, Candida reukaufii+TX, Candida saitoana (Bio-Coat®+TX, Biocure®)+TX, Candida sake+TX, Candida spp.+TX, Candida tenius+TX, Cedecea dravisae+TX, Cellulomonas flavigena+TX, Chaetomium cochliodes (Nova-Cide®)+TX, Chaetomium globosum (Nova-Cide®)+TX, Chromobacterium subtsugae strain PRAA4-1T (Grandevo®)+TX, Cladosporium cladosporioides+TX, Cladosporium oxysporum+TX, Cladosporium chlorocephalum+TX, Cladosporium spp.+TX, Cladosporium tenuissimum+TX, Clonostachys rosea (EndoFine®)+TX, Colletotrichum acutatum+TX, Coniothyrium minitans (Cotans WG®)+TX, Coniothyrium spp.+TX, Cryptococcus albidus (YIELDPLUS®)+TX, Cryptococcus humicola+TX, Cryptococcus infirmominiatus+TX, Cryptococcus laurentii+TX, Cryptophlebia leucotreta granulovirus (Cryptex®)+TX, Cupriavidus campinensis+TX, Cydia pomonella granulovirus (CYD-X®)+TX, Cydia pomonella granulovirus (Madex®+TX, Madex Plus®+TX, Madex Max/Carpovirusine®)+TX, Cylindrobasidium laeve (Stumpout®)+TX, Cylindrocladium+TX, Debaryomyces hansenii+TX, Drechslera hawaiinensis+TX, Enterobacter cloacae+TX, Enterobacteriaceae+TX, Entomophtora virulenta (Vektor®)+TX, Epicoccum nigrum+TX, Epicoccum purpurascens+TX, Epicoccum spp.+TX, Filobasidium floriforme+TX, Fusarium acuminatum+TX, Fusarium chlamydosporum+TX, Fusarium oxysporum (Fusaclean®/Biofox C®)+TX, Fusarium proliferatum+TX, Fusarium spp.+TX, Galactomyces geotrichum+TX, Gliocladium catenulatum (Primastop®+TX, Prestop®)+TX, Gliocladium roseum+TX, Gliocladium spp. (SoilGard®)+TX, Gliocladium virens (Soilgard®)+TX, Granulovirus (Granupom®)+TX, Halobacillus halophilus+TX, Halobacillus litoralis+TX, Halobacillus trueperi+TX, Halomonas spp.+TX, Halomonas subglaciescola+TX, Halovibrio variabilis+TX, Hanseniaspora uvarum+TX, Helicoverpa armigera nucleopolyhedrovirus (Helicovex®)+TX, Helicoverpa zea nuclear polyhedrosis virus (Gemstar®)+TX, Isoflavone—formononetin (Myconate®)+TX, Kloeckera apiculata+TX, Kloeckera spp.+TX, Lagenidium giganteum (Laginex®)+TX, Lecanicillium longisporum (Vertiblast®)+TX, Lecanicillium muscarium (Vertikil®)+TX, Lymantria Dispar nucleopolyhedrosis virus (Disparvirus®)+TX, Marinococcus halophilus+TX, Meira geulakonigii+TX, Metarhizium anisopliae (Met52®)+TX, Metarhizium anisopliae (Destruxin WP®)+TX, Metschnikowia fruticola (Shemer®)+TX, Metschnikowia pulcherrima+TX, Microdochium dimerum (Antibot®)+TX, Micromonospora coerulea+TX, Microsphaeropsis ochracea+TX, Muscodor albus 620 (Muscudor®)+TX, Muscodor roseus strain A3-5+TX, Mycorrhizae spp. (AMykor®+TX, Root Maximizer®)+TX, Myrothecium verrucaria strain AARC-0255 (DiTera®)+TX, BROS PLUS®+TX, Ophiostoma piliferum strain D97 (Sylvanex®)+TX, Paecilomyces farinosus+TX, Paecilomyces fumosoroseus (PFR-97®+TX, PreFeRal®)+TX, Paecilomyces linacinus (Biostat WP®)+TX, Paecilomyces lilacinus strain 251 (MeloCon WG®)+TX, Paenibacillus polymyxa+TX, Pantoea agglomerans (BlightBan C9-1®)+TX, Pantoea spp.+TX, Pasteuria spp. (Econem®)+TX, Pasteuria nishizawae+TX, Penicillium aurantiogriseum+TX, Penicillium billai (Jumpstart®+TX, TagTeam®)+TX, Penicillium brevicompactum+TX, Penicillium frequentans+TX, Penicillium griseofulvum+TX, Penicillium purpurogenum+TX, Penicillium spp.+TX, Penicillium viridicatum+TX, Phlebiopsis gigantean (Rotstop®)+TX, phosphate solubilizing bacteria (Phosphomeal®)+TX, Phytophthora cryptogea+TX, Phytophthora palmivora (Devine®)+TX, Pichia anomala+TX, Pichia guilermondii+TX, Pichia membranaefaciens+TX, Pichia onychis+TX, Pichia stipites+TX, Pseudomonas aeruginosa+TX, Pseudomonas aureofasciens (Spot-Less Biofungicide®)+TX, Pseudomonas cepacia+TX, Pseudomonas chlororaphis (AtEze®)+TX, Pseudomonas corrugate+TX, Pseudomonas fluorescens strain A506 (BlightBan A506®)+TX, Pseudomonas putida+TX, Pseudomonas reactans+TX, Pseudomonas spp.+TX, Pseudomonas syringae (Bio-Save®)+TX, Pseudomonas viridiflava+TX, Pseudomons fluorescens (Zequanox®)+TX, Pseudozyma flocculosa strain PF-A22 UL (Sporodex L®)+TX, Puccinia canaliculata+TX, Puccinia thlaspeos (Wood Warrior®)+TX, Pythium paroecandrum+TX, Pythium oligandrum (Polygandron®+TX, Polyversum®)+TX, Pythium periplocum+TX, Rhanella aquatilis+TX, Rhanella spp.+TX, Rhizobia (Dormal®+TX, Vault®)+TX, Rhizoctonia+TX, Rhodococcus globerulus strain AQ719+TX, Rhodosporidium diobovatum+TX, Rhodosporidium toruloides+TX, Rhodotorula spp.+TX, Rhodotorula glutinis+TX, Rhodotorula graminis+TX, Rhodotorula mucilagnosa+TX, Rhodotorula rubra+TX, Saccharomyces cerevisiae+TX, Salinococcus roseus+TX, Sclerotinia minor+TX, Sclerotinia minor (SARRITOR®)+TX, Scytalidium spp.+TX, Scytalidium uredinicola+TX, Spodoptera exigua nuclear polyhedrosis virus (Spod-X®+TX, Spexit®)+TX, Serratia marcescens+TX, Serratia plymuthica+TX, Serratia spp.+TX, Sordaria fimicola+TX, Spodoptera littoralis nucleopolyhedrovirus (Littovir®)+TX, Sporobolomyces roseus+TX, Stenotrophomonas maltophilia+TX, Streptomyces ahygroscopicus+TX, Streptomyces albaduncus+TX, Streptomyces exfoliates+TX, Streptomyces galbus+TX, Streptomyces griseoplanus+TX, Streptomyces griseoviridis (Mycostop®)+TX, Streptomyces lydicus (Actinovate®)+TX, Streptomyces lydicus WYEC-108 (ActinoGrow®)+TX, Streptomyces violaceus+TX, Tilletiopsis minor+TX, Tilletiopsis spp.+TX, Trichoderma asperellum (T34 Biocontrol®)+TX, Trichoderma gamsii (Tenet®)+TX, Trichoderma atroviride (Plantmate®)+TX, Trichoderma hamatum TH 382+TX, Trichoderma harzianum rifai (Mycostar®)+TX, Trichoderma harzianum T-22 (Trianum-P®+TX, PlantShield HCO+TX, RootShield®+TX, Trianum-G®)+TX, Trichoderma harzianum T-39 (Trichodex®)+TX, Trichoderma inhamatum+TX, Trichoderma koningii+TX, Trichoderma spp. LC 52 (Sentinel®)+TX, Trichoderma lignorum+TX, Trichoderma longibrachiatum+TX, Trichoderma polysporum (Binab T®)+TX, Trichoderma taxi+TX, Trichoderma virens+TX, Trichoderma virens (formerly Gliocladium virens GL-21) (SoilGuard®)+TX, Trichoderma viride+TX, Trichoderma viride strain ICC 080 (Remedier®)+TX, Trichosporon pullulans+TX, Trichosporon spp.+TX, Trichothecium spp.+TX, Trichothecium roseum+TX, Typhula phacorrhiza strain 94670+TX, Typhula phacorrhiza strain 94671+TX, Ulocladium atrum+TX, Ulocladium oudemansii (Botry-Zen®)+TX, Ustilago maydis+TX, various bacteria and supplementary micronutrients (Natural II®)+TX, various fungi (Millennium Microbes®)+TX, Verticillium chlamydosporium+TX, Verticillium lecanii (Mycotal®+TX, Vertalec®)+TX, Vip3Aa20 (VIPtera®)+TX, Virgibaclillus marismortui+TX, Xanthomonas campestris pv. Poae (Camperico®)+TX, Xenorhabdus bovienii+TX, Xenorhabdus nematophilus;

Plant extracts including: pine oil (Retenol®)+TX, azadirachtin (Plasma Neem Oil®+TX, AzaGuard®+TX, MeemAzal®+TX, Molt-X®+TX, Botanical IGR (Neemazad®+TX, Neemix®)+TX, canola oil (Lilly Miller Vegol®)+TX, Chenopodium ambrosioides near ambrosioides (Requiem®)+TX, Chrysanthemum extract (Crisant®)+TX, extract of neem oil (Trilogy®)+TX, essentials oils of Labiatae (Botania®)+TX, extracts of clove rosemary peppermint and thyme oil (Garden insect Killer®)+TX, Glycinebetaine (Greenstim®)+TX, garlic+TX, lemongrass oil (GreenMatch®)+TX, neem oil+TX, Nepeta cataria (Catnip oil)+TX, Nepeta catarina+TX, nicotine+TX, oregano oil (MossBuster®)+TX, Pedaliaceae oil (Nematon®)+TX, pyrethrum+TX, Quillaja saponaria (NemaQ®)+TX, Reynoutria sachalinensis (Regalia®+TX, Sakalia®)+TX, rotenone (Eco Roten®)+TX, Rutaceae plant extract (Soleo®)+TX, soybean oil (Ortho Ecosense®)+TX, tea tree oil (Timorex Gold®)+TX, thymus oil+TX, AGNIQUE® MMF+TX, BugOil®+TX, mixture of rosemary sesame pepermint thyme and cinnamon extracts (EF 300®)+TX, mixture of clove rosemary and peppermint extract (EF 400®)+TX, mixture of clove pepermint garlic oil and mint (Soil Shot®)+TX, kaolin (Screen®)+TX, storage glucam of brown algae (Laminarin®);
pheromones including: blackheaded fireworm pheromone (3M Sprayable Blackheaded Fireworm Pheromone®)+TX, Codling Moth Pheromone (Paramount dispenser-(CM)/Isomate C-Plus®)+TX, Grape Berry Moth Pheromone (3M MEC-GBM Sprayable Pheromone®)+TX, Leafroller pheromone (3M MEC—LR Sprayable Pheromone®)+TX, Muscamone (Snip7 Fly Bait®+TX, Starbar Premium Fly Bait®)+TX, Oriental Fruit Moth Pheromone (3M oriental fruit moth sprayable Pheromone®)+TX, Peachtree Borer Pheromone (Isomate-P®)+TX, Tomato Pinworm Pheromone (3M Sprayable Pheromone®)+TX, Entostat powder (extract from palm tree) (Exosex CM®)+TX, (E+TX,Z+TX,Z)-3+TX,8+TX,11 Tetradecatrienyl acetate+TX, (Z+TX,Z+TX,E)-7+TX,11+TX,13-Hexadecatrienal+TX, (E+TX,Z)-7+TX,9-Dodecadien-1-yl acetate+TX, 2-Methyl-1-butanol+TX, Calcium acetate+TX, Scenturion®+TX, Biolure®+TX, Check-Mate®+TX, Lavandulyl senecioate; Macrobials including: Aphelinus abdominalis+TX, Aphidius ervi (Aphelinus-System®)+TX, Acerophagus papaya+TX, Adalia bipunctata (Adalia-System®)+TX, Adalia bipunctata (Adaline®)+TX, Adalia bipunctata (Aphidalia®)+TX, Ageniaspis citricola+TX, Ageniaspis fuscicollis+TX, Amblyseius andersoni (Anderline®+TX, Andersoni-System®)+TX, Amblyseius californicus (Amblyline®+TX, Spical®)+TX, Amblyseius cucumeris (Thripex®+TX, Bugline cucumeris®)+TX, Amblyseius fallacis (Fallacis®)+TX, Amblyseius swirskii (Bugline Swirskii®+TX, Swirskii-Mite®)+TX, Amblyseius womersleyi (WomerMite®)+TX, Amitus hesperidum+TX, Anagrus atomus+TX, Anagyrus fusciventris+TX, Anagyrus kamali+TX, Anagyrus loecki+TX, Anagyrus pseudococci (Citripar®)+TX, Anicetus benefices+TX, Anisopteromalus calandrae+TX, Anthocoris nemoralis (Anthocoris-System®)+TX, Aphelinus abdominalis (Apheline®+TX, Aphiline®)+TX, Aphelinus asychis+TX, Aphidius colemani (Aphipar®)+TX, Aphidius ervi (Ervipar®)+TX, Aphidius gifuensis+TX, Aphidius matricariae (Aphipar-M®)+TX, Aphidoletes aphidimyza (Aphidend®)+TX, Aphidoletes aphidimyza (Aphidoline®)+TX, Aphytis lingnanensis+TX, Aphytis melinus+TX, Aprostocetus hagenowii+TX, Atheta coriaria (Staphyline®)+TX, Bombus spp.+TX, Bombus terrestris (Natupol Beehive®)+TX, Bombus terrestris (Beeline®+TX, Tripol®)+TX, Cephalonomia stephanoderis+TX, Chilocorus nigritus+TX, Chrysoperla carnea (Chrysoline®)+TX, Chrysoperla carnea (Chrysopa®)+TX, Chrysoperla rufilabris+TX, Cirrospilus ingenuus+TX, Cirrospilus quadristriatus+TX, Citrostichus phyllocnistoides+TX, Closterocerus chamaeleon+TX, Closterocerus spp.+TX, Coccidoxenoides perminutus (Planopar®)+TX, Coccophagus cowperi+TX, Coccophagus lycimnia+TX, Cotesia flavipes+TX, Cotesia plutellae+TX, Cryptolaemus montrouzieri (Cryptobug®+TX, Cryptoline®)+TX, Cybocephalus nipponicus+TX, Dacnusa sibirica+TX, Dacnusa sibirica (Minusa®)+TX, Diglyphus isaea (Diminex®)+TX, Delphastus catalinae (Delphastus®)+TX, Delphastus pusillus+TX, Diachasmimorpha krausii+TX, Diachasmimorpha longicaudata+TX, Diaparsis jucunda+TX, Diaphorencyrtus aligarhensis+TX, Diglyphus isaea+TX, Diglyphus isaea (Miglyphus®+TX, Digline®)+TX, Dacnusa sibirica (DacDigline®+TX, Minex®)+TX, Diversinervus spp.+TX, Encarsia citrina+TX, Encarsia formosa (Encarsia max®+TX, Encarline®+TX, En-Strip®)+TX, Eretmocerus eremicus (Enermix®)+TX, Encarsia guadeloupae+TX, Encarsia haitiensis+TX, Episyrphus balteatus (Syrphidend®)+TX, Eretmoceris siphonini+TX, Eretmocerus californicus+TX, Eretmocerus eremicus (Ercal®+TX, Eretline e®)+TX, Eretmocerus eremicus (Bemimix®)+TX, Eretmocerus hayati+TX, Eretmocerus mundus (Bemipar®+TX, Eretline m®)+TX, Eretmocerus siphonini+TX, Exochomus quadripustulatus+TX, Feltiella acarisuga (Spidend®)+TX, Feltiella acarisuga (Feltiline®)+TX, Fopius arisanus+TX, Fopius ceratitivorus+TX, Formononetin (Wirless Beehome®)+TX, Franklinothrips vespiformis (Vespop®)+TX, Galendromus occidentalis+TX, Goniozus legneri+TX, Habrobracon hebetor+TX, Harmonia axyridis (HarmoBeetle®)+TX, Heterorhabditis spp. (Lawn Patrol®)+TX, Heterorhabditis bacteriophora (NemaShield HB®+TX, Nemaseek®+TX, Terranem-Nam®+TX, Terranem®+TX, Larvanem®+TX, B-Green®+TX, NemAttack®+TX, Nematop®)+TX, Heterorhabditis megidis (Nemasys H®+TX, BioNem H®+TX, Exhibitline hm®+TX, Larvanem-M®)+TX, Hippodamia convergens+TX, Hypoaspis aculeifer (Aculeifer-System®+TX, Entomite-A®)+TX, Hypoaspis miles (Hypoline m®+TX, Entomite-M®)+TX, Lbalia leucospoides+TX, Lecanoideus floccissimus+TX, Lemophagus errabundus+TX, Leptomastidea abnormis+TX, Leptomastix dactylopii (Leptopar®)+TX, Leptomastix epona+TX, Lindorus lophanthae+TX, Lipolexis oregmae+TX, Lucilia caesar (Natufly®)+TX, Lysiphlebus testaceipes+TX, Macrolophus caliginosus (Mirical-N®+TX, Macroline c®+TX, Mirical®)+TX, Mesoseiulus longipes+TX, Metaphycus flavus+TX, Metaphycus lounsburyi+TX, Micromus angulatus (Milacewing®)+TX, Microterys flavus+TX, Muscidifurax raptorellus and Spalangia cameroni (Biopar®)+TX, Neodryinus typhlocybae+TX, Neoseiulus californicus+TX, Neoseiulus cucumeris (THRYPEX®)+TX, Neoseiulus fallacis+TX, Nesideocoris tenuis (NesidioBug®+TX, Nesibug®)+TX, Ophyra aenescens (Biofly®)+TX, Orius insidiosus (Thripor-I®+TX, Oriline i®)+TX, Orius laevigatus (Thripor-L®+TX, Oriline I®)+TX, Orius majusculus (Oriline m®)+TX, Orius strigicollis (Thripor-S®)+TX, Pauesia juniperorum+TX, Pediobius foveolatus+TX, Phasmarhabditis hermaphrodita (Nemaslug®)+TX, Phymastichus coffea+TX, Phytoseiulus macropilus+TX, Phytoseiulus persimilis (Spidex®+TX, Phytoline p®)+TX, Podisus maculiventris (Podisus®)+TX, Pseudacteon curvatus+TX, Pseudacteon obtusus+TX, Pseudacteon tricuspis+TX, Pseudaphycus maculipennis+TX, Pseudleptomastix mexicana+TX, Psyllaephagus pilosus+TX, Psyttalia concolor (complex)+TX, Quadrastichus spp.+TX, Rhyzobius lophanthae+TX, Rodolia cardinalis+TX, Rumina decollate+TX, Semielacher petiolatus+TX, Sitobion avenae (Ervibank®)+TX, Steinernema carpocapsae (Nematac C®+TX, Millenium®+TX, BioNem C®+TX, NemAttack®+TX, Nemastar®+TX, Capsanem®)+TX, Steinernema feltiae (NemaShield®+TX, Nemasys F®+TX, BioNem F®+TX, Steinernema-System®+TX, NemAttack®+TX, Nemaplus®+TX, Exhibitline sf®+TX, Scia-rid®+TX, Entonem®)+TX, Steinernema kraussei (Nemasys L®+TX, BioNem L®+TX, Exhibitline srb®)+TX, Steinernema riobrave (BioVector®+TX, BioVektor®)+TX, Steinernema scapterisci (Nematac S®)+TX, Steinernema spp.+TX, Steinernematid spp. (Guardian Nematodes®)+TX, Stethorus punctillum (Stethorus®)+TX, Tamarixia radiate+TX, Tetrastichus setifer+TX, Thripobius semiluteus+TX, Torymus sinensis+TX, Trichogramma brassicae (Tricholine b®)+TX, Trichogramma brassicae (Tricho-Strip®)+TX, Trichogramma evanescens+TX, Trichogramma minutum+TX, Trichogramma ostriniae+TX, Trichogramma platneri+TX, Trichogramma pretiosum+TX, Xanthopimpla stemmator,
other biologicals including: abscisic acid+TX, bioSea®+TX, Chondrostereum purpureum (Chontrol Paste®)+TX, Colletotrichum gloeosporioides (Collego®)+TX, Copper Octanoate (Cueva®)+TX, Delta traps (Trapline d®)+TX, Erwinia amylovora (Harpin) (ProAct®+TX, Ni-HIBIT Gold CST®)+TX, Ferri-phosphate (Ferramol®)+TX, Funnel traps (Trapline y®)+TX, Gallex®+TX, Grower's Secret®+TX, Homo-brassonolide+TX, Iron Phosphate (Lilly Miller Worry Free Ferramol Slug & Snail Bait®)+TX, MCP hail trap (Trapline f®)+TX, Microctonus hyperodae+TX, Mycoleptodiscus terrestris (Des-X®)+TX, BioGain®+TX, Aminomite®+TX, Zenox®+TX, Pheromone trap (Thripline ams®)+TX, potassium bicarbonate (MilStop®)+TX, potassium salts of fatty acids (Sanova®)+TX, potassium silicate solution (Sil-Matrix®)+TX, potassium iodide+potassiumthiocyanate (Enzicur®)+TX, SuffOil-X®+TX, Spider venom+TX, Nosema locustae (Semaspore Organic Grasshopper Control®)+TX, Sticky traps (Trapline YF®+TX, Rebell Amarillo®)+TX and Traps (Takitrapline y+b®)+TX;
(1) antibacterial agents selected from the group of:
(1.1) bacteria, examples of which are Bacillus mojavensis strain R₃B (Accession No. NCAIM (P) B001389) (WO 2013/034938) from Certis USA LLC, a subsidiary of Mitsui & Co.+TX; Bacillus pumilus, in particular strain BU F-33, having NRRL Accession No. 50185 (available as part of the CARTISSA® product from BASF, EPA Reg. No. 71840-19)+TX; Bacillus subtilis, in particular strain QST713/AQ713 (available as SERENADE OPTI or SERENADE ASO from Bayer CropScience LP, US, having NRRL Accession No. B21661, U.S. Pat. No. 6,060,051)+TX; Bacillus subtilis strain BU1814, (available as VELONDIS® PLUS, VELONDIS® FLEX and VELONDIS® EXTRA from BASF SE)+TX; Bacillus subtilis var. amyloliquefaciens strain FZB24 having Accession No. DSM 10271 (available from Novozymes as TAEGRO® or TAEGRO® ECO (EPA Registration No. 70127-5))+TX; Bacillus subtilis CX-9060 from Certis USA LLC, a subsidiary of Mitsui & Co.+TX; Bacillus sp., in particular strain D747 (available as DOUBLE NICKEL® from Kumiai Chemical Industry Co., Ltd.), having Accession No. FERM BP-8234, U.S. Pat. No. 7,094,592+TX; Paenibacillus sp. strain having Accession No. NRRL B-50972 or Accession No. NRRL B-67129, WO 2016/154297+TX; Paenibacillus polymyxa, in particular strain AC-1 (e.g. TOPSEED® from Green Biotech Company Ltd.)+TX; Pantoea agglomerans, in particular strain E325 (Accession No. NRRL B-21856) (available as BLOOMTIME BIOLOGICAL™ FD BIOPESTICIDE from Northwest Agri Products)+TX; Pseudomonas proradix (e.g. PRORADIX® from Sourcon Padena)+TX; and
(1.2) fungi, examples of which are Aureobasidium pullulans, in particular blastospores of strain DSM14940, blastospores of strain DSM 14941 or mixtures of blastospores of strains DSM14940 and DSM14941 (e.g., BOTECTOR® and BLOSSOM PROTECT® from bio-ferm, CH)+TX; Pseudozyma aphidis (as disclosed in WO2011/151819 by Yissum Research Development Company of the Hebrew University of Jerusalem)+TX; Saccharomyces cerevisiae, in particular strains CNCM No. 1-3936, CNCM No. 1-3937, CNCM No. 1-3938 or CNCM No. 1-3939 (WO 2010/086790) from Lesaffre et Compagnie, FR;
(2) biological fungicides selected from the group of:
(2.1) bacteria, examples of which are Agrobacterium radiobacter strain K84 (e.g. GALLTROL-A® from AgBioChem, CA)+TX; Agrobacterium radiobacter strain K1026 (e.g. NOGALL™ from BASF SE)+TX; Bacillus subtilis var. amyloliquefaciens strain FZB24 having Accession No. DSM 10271 (available from Novozymes as TAEGRO® or TAEGRO® ECO (EPA Registration No. 70127-5))+TX; Bacillus amyloliquefaciens, in particular strain D747 (available as Double Nickel™ from Kumiai Chemical Industry Co., Ltd., having accession number FERM BP-8234, U.S. Pat. No. 7,094,592)+TX; Bacillus amyloliquefaciens strain F727 (also known as strain MB1110) (NRRL Accession No. B-50768, WO 2014/028521) (STARGUS® from Marrone Bio Innovations)+TX; Bacillus amyloliquefaciens strain FZB42, Accession No. DSM 23117 (available as RHIZOVITAL® from ABiTEP, DE)+TX; Bacillus amyloliquefaciens isolate B246 (e.g. AVOGREEN™ from University of Pretoria)+TX; Bacillus licheniformis, in particular strain SB3086, having Accession No. ATCC 55406, WO 2003/000051 (available as ECOGUARD® Biofungicide and GREEN RELEAF™ from Novozymes)+TX+TX; Bacillus licheniformis FMCH001 and Bacillus subtilis FMCH002 (QUARTZO® (WG) and PRESENCE® (WP) from FMC Corporation)+TX; Bacillus methylotrophicus strain BAC-9912 (from Chinese Academy of Sciences' Institute of Applied Ecology)+TX; Bacillus mojavensis strain R3B (Accession No. NCAIM (P) B001389) (WO 2013/034938) from Certis USA LLC, a subsidiary of Mitsui & Co.+TX; Bacillus mycoides, isolate, having Accession No. B-30890 (available as BMJ TGAI® or WG and LifeGard™ from Certis USA LLC, a subsidiary of Mitsui & Co.)+TX; Bacillus pumilus, in particular strain QST2808 (available as SONATA® from Bayer CropScience LP, US, having Accession No. NRRL B-30087 and described in U.S. Pat. No. 6,245,551)+TX; Bacillus pumilus, in particular strain GB34 (available as Yield Shield® from Bayer AG, DE)+TX; Bacillus pumilus, in particular strain BU F-33, having NRRL Accession No. 50185 (available as part of the CARTISSA product from BASF, EPA Reg. No. 71840-19)+TX; Bacillus subtilis, in particular strain QST713/AQ713 (available as SERENADE OPTI or SERENADE ASO from Bayer CropScience LP, US, having NRRL Accession No. B21661 and described in U.S. Pat. No. 6,060,051)+TX; Bacillus subtilis Y1336 (available as BIOBAC® WP from Bion-Tech, Taiwan, registered as a biological fungicide in Taiwan under Registration Nos. 4764, 5454, 5096 and 5277)+TX; Bacillus subtilis strain MBI 600 (available as SUBTILEX from BASF SE), having Accession Number NRRL B-50595, U.S. Pat. No. 5,061,495+TX; Bacillus subtilis strain GB03 (available as Kodiak® from Bayer AG, DE)+TX; Bacillus subtilis strain BU1814, (available as VELONDIS® PLUS, VELONDIS® FLEX and VELONDIS® EXTRA from BASF SE)+TX; Bacillus subtilis CX-9060 from Certis USA LLC, a subsidiary of Mitsui & Co.+TX; Bacillus subtilis KTSB strain (FOLIACTIVE® from Donaghys)+TX; Bacillus subtilis IAB/BS03 (AVIV™ from STK Bio-Ag Technologies, PORTENTO® from Idai Nature)+TX; Bacillus subtilis strain Y1336 (available as BIOBAC® WP from Bion-Tech, Taiwan, registered as a biological fungicide in Taiwan under Registration Nos. 4764, 5454, 5096 and 5277)+TX; Paenibacillus epiphyticus (WO 2016/020371) from BASF SE+TX; Paenibacillus polymyxa ssp. plantarum (WO 2016/020371) from BASF SE+TX; Paenibacillus sp. strain having Accession No. NRRL B-50972 or Accession No. NRRL B-67129, WO 2016/154297+TX; Pseudomonas chlororaphis strain AFS009, having Accession No. NRRL B-50897, WO 2017/019448 (e.g., HOWLER™ and ZIO® from AgBiome Innovations, US)+TX; Pseudomonas chlororaphis, in particular strain MA342 (e.g. CEDOMON®, CERALL®, and CEDRESS® by Bioagri and Koppert)+TX; Pseudomonas fluorescens strain A506 (e.g. BLIGHTBAN® A506 by NuFarm)+TX; Pseudomonas proradix (e.g. PRORADIX® from Sourcon Padena)+TX; Streptomyces griseoviridis strain K61 (also known as Streptomyces galbus strain K61) (Accession No. DSM 7206) (MYCOSTOP® from Verdera, PREFENCE® from BioWorks, cf. Crop Protection 2006, 25, 468-475)+TX; Streptomyces lydicus strain WYEC108 (also known as Streptomyces lydicus strain WYCD108US) (ACTINO-IRON® and ACTINOVATE® from Novozymes)+TX; and
(2.2) fungi, examples of which are Ampelomyces quisqualis, in particular strain AQ 10 (e.g. AQ 10® by IntrachemBio Italia)+TX; Ampelomyces quisqualis strain AQ10, having Accession No. CNCM 1-807 (e.g., AQ 10® by IntrachemBio Italia)+TX; Aspergillus flavus strain NRRL 21882 (products known as AFLA-GUARD® from Syngenta/ChemChina)+TX; Aureobasidium pullulans, in particular blastospores of strain DSM14940+TX; Aureobasidium pullulans, in particular blastospores of strain DSM 14941+TX; Aureobasidium pullulans, in particular mixtures of blastospores of strains DSM14940 and DSM 14941 (e.g. Botector® by bio-ferm, CH)+TX; Chaetomium cupreum (Accession No. CABI 353812) (e.g. BIOKUPRUM™ by AgriLife)+TX; Chaetomium globosum (available as RIVADIOM® by Rivale)+TX; Cladosporium cladosporioides, strain H39, having Accession No. CBS122244, US 2010/0291039 (by Stichting Dienst Landbouwkundig Onderzoek)+TX; Coniothyrium minitans, in particular strain CON/M/91-8 (Accession No. DSM9660, e.g. Contans® from Bayer CropScience Biologics GmbH)+TX; Cryptococcus flavescens, strain 3C (NRRL Y-50378), (B2.2.99)+TX; Dactylaria candida+TX; Dilophosphora alopecuri (available as TWIST FUNGUS®)+TX; Fusarium oxysporum, strain Fo47 (available as FUSACLEAN® by Natural Plant Protection)+TX; Gliocladium catenulatum (Synonym: Clonostachys rosea f. catenulate) strain J1446 (e.g. Prestop® by Lallemand)+TX; Gliocladium roseum (also known as Clonostachys rosea f rosea), in particular strain 321U from Adjuvants Plus, strain ACM941 as disclosed in Xue (Efficacy of Clonostachys rosea strain ACM941 and fungicide seed treatments for controlling the root tot complex of field pea, Can Jour Plant Sci 83(3): 519-524), or strain IK726 (Jensen D F, et al. Development of a biocontrol agent for plant disease control with special emphasis on the near commercial fungal antagonist Clonostachys rosea strain ‘IK726’, Australas Plant Pathol. 2007,36:95-101)+TX; Lecanicillium lecanii (formerly known as Verticillium lecanii) conidia of strain KV01 (e.g. Vertalec® by Koppert/Arysta)+TX; Metschnikowia fructicola, in particular strain NRRL Y-30752, (B2.2.3)+TX; Microsphaeropsis ochracea+TX; Muscodor roseus, in particular strain A3-5 (Accession No. NRRL 30548)+TX; Penicillium steckii (DSM 27859, WO 2015/067800) from BASF SE+TX; Penicillium vermiculatum+TX; Phlebiopsis gigantea strain VRA 1992 (ROTSTOP® C from Danstar Ferment)+TX; Pichia anomala, strain WRL-076 (NRRL Y-30842), U.S. Pat. No. 7,579,183+TX; Pseudozyma flocculosa, strain PF-A22 UL (available as SPORODEX® L by Plant Products Co., CA)+TX; Saccharomyces cerevisiae, in particular strain LASO2 (from Agro-Levures et Derives), strain LAS117 cell walls (CEREVISANE® from Lesaffre, ROMEO® from BASF SE), strains CNCM No. 1-3936, CNCM No. 1-3937, CNCM No. 1-3938, CNCM No. 1-3939 (WO 2010/086790) from Lesaffre et Compagnie, FR+TX; Simplicillium lanosoniveum+TX; Talaromyces flavus, strain V117b+TX; Trichoderma asperelloides JM41R (Accession No. NRRL B-50759) (TRICHO PLUS® from BASF SE)+TX; Trichoderma asperellum, in particular, strain kd (e.g. T-Gro from Andermatt Biocontrol)+TX; Trichoderma asperellum, in particular strain SKT-1, having Accession No. FERM P-16510 (e.g. ECO-HOPE® from Kumiai Chemical Industry), strain T34 (e.g. T34 Biocontrol by Biocontrol Technologies S.L., ES) or strain ICC 012 from Isagro+TX; Trichoderma atroviride, in particular strain SC1 (having Accession No. CBS 122089, WO 2009/116106 and U.S. Pat. No. 8,431,120 (from Bi-PA)), strain 77B (T77 from Andermatt Biocontrol) or strain LU132 (e.g. Sentinel from Agrimm Technologies Limited)+TX; Trichoderma atroviride, strain CNCM 1-1237 (e.g. Esquive® WP from Agrauxine, FR)+TX; Trichoderma atroviride, strain no. V08/002387+TX; Trichoderma atroviride, strain NMI no. V08/002388+TX; Trichoderma atroviride, strain NMI no. V08/002389+TX; Trichoderma atroviride, strain NMI no. V08/002390+TX; Trichoderma atroviride, strain LC52 (e.g. Tenet by Agrimm Technologies Limited)+TX; Trichoderma atroviride, strain ATCC 20476 (IMI 206040)+TX; Trichoderma atroviride, strain T11 (IM1352941/CECT20498)+TX; Trichoderma atroviride, strain SKT-1 (FERM P-16510), JP Patent Publication (Kokai) 11-253151 A+TX; Trichoderma atroviride, strain SKT-2 (FERM P-16511), JP Patent Publication (Kokai) 11-253151 A+TX; Trichoderma atroviride, strain SKT-3 (FERM P-17021), JP Patent Publication (Kokai) 11-253151 A+TX; Trichoderma fertile (e.g. product TrichoPlus from BASF)+TX; Trichoderma gamsii (formerly T. viride), strain ICC080 (IMI CC 392151 CABI, e.g. BioDerma by AGROBIOSOL DE MEXICO, S.A. DE C.V.)+TX; Trichoderma gamsii (formerly T. viride), strain ICC 080 (IMI CC 392151 CABI) (available as BIODERMA® by AGROBIOSOL DE MEXICO, S.A. DE C.V.)+TX; Trichoderma harmatum+TX; Trichoderma harmatum, having Accession No. ATCC 28012+TX; Trichoderma harzianum strain T-22 (e.g. Trianum-P from Andermatt Biocontrol or Koppert) or strain Cepa SimbT5 (from Simbiose Agro)+TX; Trichoderma harzianum+TX; Trichoderma harzianum rifai T39 (e.g. Trichodex® from Makhteshim, US)+TX; Trichoderma harzianum, strain ITEM 908 (e.g. Trianum-P from Koppert)+TX; Trichoderma harzianum, strain TH35 (e.g. Root-Pro by Mycontrol)+TX; Trichoderma harzianum, strain DB 103 (available as T-GRO® 7456 by Dagutat Biolab)+TX; Trichoderma polysporum, strain IMI 206039 (e.g. Binab TF WP by BINAB Bio-Innovation AB, Sweden)+TX; Trichoderma stromaticum, having Accession No. Ts3550 (e.g. Tricovab by CEPLAC, Brazil)+TX; Trichoderma virens (also known as Gliocladium virens), in particular strain GL-21 (e.g. SoilGard by Certis, US)+TX; Trichoderma virens strain G-41, formerly known as Gliocladium virens (Accession No. ATCC 20906) (e.g., ROOTSHIELD® PLUS WP and TURFSHIELD® PLUS WP from BioWorks, US)+TX; Trichoderma viride, strain TV1 (e.g. Trianum-P by Koppert)+TX; Trichoderma viride, in particular strain B35 (Pietr et al., 1993, Zesz. Nauk. A R w Szczecinie 161: 125-137)+TX; mixtures of Trichoderma asperellum strain ICC 012 (also known as Trichoderma harzianum ICC012), having Accession No. CABI CC IMI 392716 and Trichoderma gamsii (formerly T. viride) strain ICC 080, having Accession No. IMI 392151 (e.g., BIO-TAM™ from Isagro USA, Inc. and BIODERMA® by Agrobiosol de Mexico, S.A. de C.V.)+TX; Ulocladium oudemansii strain U3, having Accession No. NM 99/06216 (e.g., BOTRY-ZEN® by Botry-Zen Ltd, New Zealand and BOTRYSTOP® from BioWorks, Inc.)+TX; Verticillium albo-atrum (formerly V. dahliae), strain WCS850 having Accession No. WCS850, deposited at the Central Bureau for Fungi Cultures (e.g., DUTCH TRIG® by Tree Care Innovations)+TX; Verticillium chlamydosporium+TX;
(3) biological control agents having an effect for improving plant growth and/or plant health selected from the group of: (3.1) bacteria, examples of which are Azospirillum brasilense (e.g., VIGOR® from KALO, Inc.)+TX; Azospirillum lipoferum (e.g., VERTEX-IF™ from TerraMax, Inc.)+TX; Azorhizobium caulinodans, in particular strain ZB-SK-5+TX; Azotobacter chroococcum, in particular strain H23+TX; Azotobacter vinelandii, in particular strain ATCC 12837+TX; a mixture of Azotobacter vinelandii and Clostridium pasteurianum (available as INVIGORATE® from Agrinos)+TX; Bacillus amyloliquefaciens pm414 (LOLI-PEPTA® from Biofilm Crop Protection)+TX; Bacillus amyloliquefaciens SB3281 (ATCC #PTA-7542, WO 2017/205258)+TX; Bacillus amyloliquefaciens TJ1000 (available as QUIKROOTS® from Novozymes)+TX; Bacillus amyloliquefaciens, in particular strain IN937a+TX; Bacillus amyloliquefaciens, in particular strain FZB42 (e.g. RHIZOVITAL® from ABiTEP, DE)+TX; Bacillus amyloliquefaciens BS27 (Accession No. NRRL B-5015)+TX; Bacillus cereus family member EE128 (NRRL No. B-50917)+TX; Bacillus cereus family member EE349 (NRRL No. B-50928)+TX; Bacillus cereus, in particular strain BP01 (ATCC 55675, e.g. MEPICHLOR® from Arysta Lifescience, US)+TX; Bacillus firmus, in particular strain CNMC 1-1582 (e.g. VOTIVO® from BASF SE)+TX; Bacillus mycoides BT155 (NRRL No. B-50921)+TX; Bacillus mycoides EE118 (NRRL No. B-50918)+TX; Bacillus mycoides EE141 (NRRL No. B-50916)+TX; Bacillus mycoides BT46-3 (NRRL No. B-50922)+TX; Bacillus pumilus, in particular strain QST2808 (having Accession No. NRRL No. B-30087)+TX; Bacillus pumilus, in particular strain GB34 (e.g. YIELD SHIELD® from Bayer Crop Science, DE)+TX; Bacillus siamensis, in particular strain KCTC 13613T+TX; Bacillus subtilis, in particular strain QST713/AQ713 (having NRRL Accession No. B-21661 and described in U.S. Pat. No. 6,060,051, available as SERENADE® OPTI or SERENADE® ASO from Bayer CropScience LP, US)+TX; Bacillus subtilis, in particular strain AQ30002 (having Accession Nos. NRRL B-50421 and described in U.S. patent application Ser. No. 13/330,576)+TX; Bacillus subtilis, in particular strain AQ30004 (and NRRL B-50455 and described in U.S. patent application Ser. No. 13/330,576)+TX; Bacillus subtilis strain BU1814, (available as TEQUALIS® from BASF SE), Bacillus subtilis rm303 (RHIZOMAX® from Biofilm Crop Protection)+TX; Bacillus thuringiensis BT013A (NRRL No. B-50924) also known as Bacillus thuringiensis 4Q7+TX; a mixture of Bacillus licheniformis FMCH001 and Bacillus subtilis FMCH002 (available as QUARTZO® (WG), PRESENCE® (WP) from FMC Corporation)+TX; Bacillus subtilis, in particular strain MBI 600 (e.g. SUBTILEX® from BASF SE)+TX; Bacillus tequilensis, in particular strain NII-0943+TX; Bradyrhizobium japonicum (e.g. OPTIMIZE® from Novozymes)+TX; Delftia acidovorans, in particular strain RAY209 (e.g. BIOBOOST® from Brett Young Seeds)+TX; Mesorhizobium cicer (e.g., NODULATOR from BASF SE)+TX; Lactobacillus sp. (e.g. LACTOPLANT® from LactoPAFI)+TX; Rhizobium leguminosarium biovar viciae (e.g., NODULATOR from BASF SE)+TX; Pseudomonas proradix (e.g. PRORADIX® from Sourcon Padena)+TX; Pseudomonas aeruginosa, in particular strain PN1+TX; Rhizobium leguminosarum, in particular bv. viceae strain Z25 (Accession No. CECT 4585)+TX; Paenibacillus polymyxa, in particular strain AC-1 (e.g. TOPSEED® from Green Biotech Company Ltd.)+TX; Serratia marcescens, in particular strain SRM (Accession No. MTCC 8708)+TX; Sinorhizobium meliloti strain NRG-185-1 (NITRAGIN® GOLD from Bayer CropScience)+TX; Thiobacillus sp. (e.g. CROPAID® from Cropaid Ltd UK)+TX; and (3.2) fungi, examples of which are Purpureocillium lilacinum (previously known as Paecilomyces lilacinus) strain 251 (AGAL 89/030550, e.g. BioAct from Bayer CropScience Biologics GmbH)+TX; Penicillium bilaii, strain ATCC 22348 (e.g. JumpStart® from Acceleron BioAg), Talaromyces flavus, strain V1117b+TX; Trichoderma atroviride strain CNCM 1-1237 (e.g. Esquive® WP from Agrauxine, FR), Trichoderma viride, e.g. strain B35 (Pietr et al., 1993, Zesz. Nauk. A R w Szczecinie 161: 125-137)+TX; Trichoderma atroviride strain LC52 (also known as Trichoderma atroviride strain LU132, e.g. Sentinel from Agrimm Technologies Limited)+TX; Trichoderma atroviride strain SC1 described in International Application No. PCT/IT2008/000196)+TX; Trichoderma asperellum strain kd (e.g. T-Gro from Andermatt Biocontrol)+TX; Trichoderma asperellum strain Eco-T (Plant Health Products, ZA), Trichoderma harzianum strain T-22 (e.g. Trianum-P from Andermatt Biocontrol or Koppert)+TX; Myrothecium verrucaria strain AARC-0255 (e.g. DiTera™ from Valent Biosciences)+TX; Penicillium bilaii strain ATCC ATCC20851+TX; Pythium oligandrum strain M1 (ATCC 38472, e.g. Polyversum from Bioprepraty, CZ)+TX; Trichoderma virens strain GL-21 (e.g. SoilGard® from Certis, USA)+TX; Verticillium albo-atrum (formerly V. dahliae) strain WCS850 (CBS 276.92, e.g. Dutch Trig from Tree Care Innovations)+TX; Trichoderma atroviride, in particular strain no. V08/002387, strain no. NMI No. V08/002388, strain no. NMI No. V08/002389, strain no. NMI No. V08/002390+TX; Trichoderma harzianum strain ITEM 908, Trichoderma harzianum, strain TSTh20+TX; Trichoderma harzianum strain 1295-22+TX; Pythium oligandrum strain DV74+TX; Rhizopogon amylopogon (e.g. comprised in Myco-Sol from Helena Chemical Company)+TX; Rhizopogon fulvigleba (e.g. comprised in Myco-Sol from Helena Chemical Company)+TX; Trichoderma virens strain GI-3+TX;
(4) insecticidally active biological control agents selected from
(4.1) bacteria, examples of which are Agrobacterium radiobacter strain K84 (Galltrol from AgBiochem Inc.)+TX; Bacillus amyloliquefaciens, in particular strain PTS-4838 (e.g. AVEO from Valent Biosciences, US)+TX; Bacillus firmus, in particular strain CNMC 1-1582 (e.g. VOTIVO® from BASF SE)+TX; Bacillus mycoides, isolate J. (e.g. BmJ from Certis USA LLC, a subsidiary of Mitsui & Co.)+TX; Bacillus sphaericus, in particular Serotype H5a5b strain 2362 (strain ABTS-1743) (e.g. VECTOLEX® from Valent BioSciences, US)+TX; Bacillus thuringiensis subsp. aizawai, in particular strain ABTS-1857 (SD-1372, e.g. XENTARI® from Valent BioSciences)+TX; Bacillus thuringiensis subsp. aizawai, in particular serotype H-7 (e.g. FLORBAC® WG from Valent BioSciences, US)+TX; Bacillus thuringiensis israelensis strain BMP 144 (e.g. AQUABAC® by Becker Microbial Products IL)+TX; Bacillus thuringiensis subsp. israelensis (serotype H-14) strain AM65-52 (Accession No. ATCC 1276) (e.g. VECTOBAC® by Valent BioSciences, US)+TX; Bacillus thuringiensis subsp. aizawai strain GC-91+TX; Bacillus thuringiensis var. Colmeri (e.g. TIANBAOBTC by Changzhou Jianghai Chemical Factory)+TX; Bacillus thuringiensis var. japonensis strain Buibui+TX; Bacillus thuringiensis subsp. kurstaki strain BMP 123 from Becker Microbial Products, IL+TX; Bacillus thuringiensis subsp. kurstaki strain BMP 123 by Becker Microbial Products, IL, e.g. BARITONE from Bayer CropScience+TX; Bacillus thuringiensis subsp. kurstaki strain HD-1 (e.g. DIPEL® ES from Valent BioSciences, US)+TX; Bacillus thuringiensis var. kurstaki strain EVB-113-19 (e.g., BIOPROTEC® from AEF Global)+TX; Bacillus thuringiensis subsp. kurstaki strain ABTS 351+TX; Bacillus thuringiensis subsp. kurstaki strain PB 54+TX; Bacillus thuringiensis subsp. kurstaki strain SA 11, (JAVELIN from Certis, US)+TX; Bacillus thuringiensis subsp. kurstaki strain SA 12 (THURICIDE from Certis, US)+TX; Bacillus thuringiensis subsp. kurstaki strain EG 2348 (LEPINOX from Certis, US)+TX; Bacillus thuringiensis subsp. kurstaki strain EG 7841 (CRYMAX from Certis, US)+TX; Bacillus thuringiensis subsp. tenebrionis strain NB 176 (SD-5428, e.g. NOVODOR® FC from BioFa DE)+TX; Brevibacillus laterosporus (LATERAL from Ecolibrium Biologicals)+TX; Burkholderia spp., in particular Burkholderia rinojensis strain A396 (also known as Burkholderia rinojensis strain MBI 305) (Accession No. NRRL B-50319+TX; WO 2011/106491 and WO 2013/032693+TX; e.g. MB1206 TGAI and ZELTO® from Marrone Bio Innovations)+TX; Chromobacterium subtsugae, in particular strain PRAA4-1T (MBI-203+TX; e.g. GRANDEVO® from Marrone Bio Innovations)+TX; Lecanicillium muscarium Ve6 (MYCOTAL from Koppert)+TX; Paenibacillus popilliae (formerly Bacillus popilliae+TX; e.g. MILKY SPORE POWDER™ and MILKY SPORE GRANULAR™ from St. Gabriel Laboratories)+TX; Pasteuria nishizawae strain Pn1 (CLARIVA from Syngenta/ChemChina)+TX; Serratia entomophila (e.g. INVADE® by Wrightson Seeds)+TX; Serratia marcescens, in particular strain SRM (Accession No. MTCC 8708)+TX; Trichoderma asperellum (TRICHODERMAX from Novozymes)+TX; Wolbachia pipientis ZAP strain (e.g., ZAP MALES® from MosquitoMate)+TX; and
(4.2) fungi, examples of which are Beauveria bassiana strain ATCC 74040 (e.g. NATURALIS® from Intrachem Bio Italia)+TX; Beauveria bassiana strain GHA (Accession No. ATCC74250, e.g.

BOTANIGUARD® ES and MYCONTROL-O® from Laverlam International Corporation)+TX; Beauveria bassiana strain ATPO2 (Accession No. DSM 24665)+TX; Isaria fumosorosea (previously known as Paecilomyces fumosoroseus) strain Apopka 97) PREFERAL from SePRO+TX; Metarhizium anisopliae 3213-1 (deposited under NRRL accession number 67074) (WO 2017/066094+TX; Pioneer Hi-Bred International)+TX; Metarhizium robertsii 15013-1 (deposited under NRRL accession number 67073)+TX; Metarhizium robertsii 23013-3 (deposited under NRRL accession number 67075)+TX; Paecilomyces lilacinus strain 251 (MELOCON from Certis, US)+TX; Zoophtora radicans+TX;
(5) Viruses selected from the group consisting of Adoxophyes orana (summer fruit tortrix) granulosis virus (GV)+TX; Cydia pomonella (codling moth) granulosis virus (GV)+TX; Helicoverpa armigera (cotton bollworm) nuclear polyhedrosis virus (NPV)+TX; Spodoptera exigua (beet armyworm) mNPV+TX; Spodoptera frugiperda (fall armyworm) mNPV+TX; Spodoptera littoralis (African cotton leafworm) NPV+TX;
(6) Bacteria and fungi which can be added as ‘inoculant’ to plants or plant parts or plant organs and which, by virtue of their particular properties, promote plant growth and plant health selected from Agrobacterium spp.+TX; Azorhizobium caulinodans+TX; Azospirillum spp.+TX; Azotobacter spp.+TX; Bradyrhizobium spp.+TX; Burkholderia spp., in particular Burkholderia cepacia (formerly known as Pseudomonas cepacia)+TX; Gigaspora spp., or Gigaspora monosporum+TX; Glomus spp.+TX; Laccaria spp.+TX; LactoBacillus buchneri+TX; Paraglomus spp.+TX; Pisolithus tinctorus+TX; Pseudomonas spp.+TX; Rhizobium spp., in particular Rhizobium trifolii+TX; Rhizopogon spp.+TX; Scleroderma spp.+TX; Suillus spp.+TX; Streptomyces spp.+TX;
(7) Plant extracts and products formed by microorganisms including proteins and secondary metabolites which can be used as biological control agents, selected from Allium sativum (NEMGUARD from Eco-Spray+TX; BRALIC from ADAMA)+TX; Armour-Zen+TX; Artemisia absinthium+TX; Azadirachtin (e.g. AZATIN XL from Certis, US)+TX; Biokeeper WP+TX; Brassicaceae extract, in particular oilseed rape powder or mustard powder+TX; Cassia nigricans+TX; Celastrus angulatus+TX; Chenopodium anthelminticum+TX; Chitin+TX; Dryopteris filix-mas+TX; Equisetum arvense+TX; Fortune Aza+TX; Fungastop+TX; Heads Up (Chenopodium quinoa saponin extract)+TX; PROBLAD (naturally occurring Blad polypeptide from Lupin seeds), Certis EU+TX; FRACTURE (naturally occurring Blad polypeptide from Lupin seeds), FMC+TX; Pyrethrum/Pyrethrins+TX; Quassia amara+TX; Quercus+TX; Quillaja extract (QL AGRI 35 from BASF)+TX; Reynoutria sachalinensis extract (REGALLIA/REGALIA MAXX from Marrone Bio)+TX; “Requiem™ Insecticide”+TX; Rotenone+TX; ryania/ryanodine+TX; Symphytum officinale+TX; Tanacetum vulgare+TX; Thymol+TX; Thymol mixed with Geraniol (CEDROZ from Eden Research)+TX; Thymol mixed with Geraniol and Eugenol (MEVALONE from Eden Research)+TX; Triact 70+TX; TriCon+TX; Tropaeulum majus+TX; Melaleuca alternifolia extract (TIMOREX GOLD from STK)+TX; Urtica dioica+TX; Veratrin+TX; and Viscum album+TX; and
a safener, such as benoxacor+TX, cloquintocet (including cloquintocet-mexyl)+TX, cyprosulfamide+TX, dichlormid+TX, fenchlorazole (including fenchlorazole-ethyl)+TX, fenclorim+TX, fluxofenim+TX, furilazole+TX, isoxadifen (including isoxadifen-ethyl)+TX, mefenpyr (including mefenpyr-diethyl)+TX, metcamifen+TX and oxabetrinil+TX.

The references in brackets behind the active ingredients, e.g. [3878-19-1] refer to the Chemical Abstracts Registry number. The above described mixing partners are known. Where the active ingredients are included in “The Pesticide Manual” [The Pesticide Manual—A World Compendium; Thirteenth Edition; Editor: C. D. S. TomLin; The British Crop Protection Council], they are described therein under the entry number given in round brackets hereinabove for the particular compound; for example, the compound “abamectin” is described under entry number (1). Where “[CCN]” is added hereinabove to the particular compound, the compound in question is included in the “Compendium of Pesticide Common Names”, which is accessible on the internet [A. Wood; Compendium of Pesticide Common Names, Copyright © 1995-2004]; for example, the compound “acetoprole” is described under the internet address http://www.alanwood.net/pesticides/acetoprole.htm.

Most of the active ingredients described above are referred to hereinabove by a so-called “common name”, the relevant “ISO common name” or another “common name” being used in individual cases. If the designation is not a “common name”, the nature of the designation used instead is given in round brackets for the particular compound; in that case, the IUPAC name, the IUPAC/Chemical Abstracts name, a “chemical name”, a “traditional name”, a “compound name” or a “develoment code” is used or, if neither one of those designations nor a “common name” is used, an “alternative name” is employed. “CAS Reg. No” means the Chemical Abstracts Registry Number.

The active ingredient mixture of the compounds of formula I selected from Tables A-1 to A-60, Tables B-1 to B-60, Tables C-1 to C-60, Tables D-1 to D-60 and Table P with active ingredients described above comprises a compound selected from Tables A-1 to A-60, Tables B-1 to B-60, Tables C-1 to C-60, Tables D-1 to D-60 and Table P and an active ingredient as described above preferably in a mixing ratio of from 100:1 to 1:6000, especially from 50:1 to 1:50, more especially in a ratio of from 20:1 to 1:20, even more especially from 10:1 to 1:10, very especially from 5:1 and 1:5, special preference being given to a ratio of from 2:1 to 1:2, and a ratio of from 4:1 to 2:1 being likewise preferred, above all in a ratio of 1:1, or 5:1, or 5:2, or 5:3, or 5:4, or 4:1, or 4:2, or 4:3, or 3:1, or 3:2, or 2:1, or 1:5, or 2:5, or 3:5, or 4:5, or 1:4, or 2:4, or 3:4, or 1:3, or 2:3, or 1:2, or 1:600, or 1:300, or 1:150, or 1:35, or 2:35, or 4:35, or 1:75, or 2:75, or 4:75, or 1:6000, or 1:3000, or 1:1500, or 1:350, or 2:350, or 4:350, or 1:750, or 2:750, or 4:750. Those mixing ratios are by weight.

The mixtures as described above can be used in a method for controlling pests, which comprises applying a composition comprising a mixture as described above to the pests or their environment, with the exception of a method for treatment of the human or animal body by surgery or therapy and diagnostic methods practised on the human or animal body.

The mixtures comprising a compound of formula I selected from Tables A-1 to A-60, Tables B-1 to B-60, Tables C-1 to C-60, Tables D-1 to D-60 and Table P and one or more active ingredients as described above can be applied, for example, in a single “ready-mix” form, in a combined spray mixture composed from separate formulations of the single active ingredient components, such as a “tank-mix”, and in a combined use of the single active ingredients when applied in a sequential manner, i.e. one after the other with a reasonably short period, such as a few hours or days. The order of applying the compounds of formula I selected from Tables A-1 to A-60, Tables B-1 to B-60, Tables C-1 to C-60, Tables D-1 to D-60 and Table P and the active ingredients as described above is not essential for working the present invention.

The compositions according to the invention can also comprise further solid or liquid auxiliaries, such as stabilizers, for example unepoxidized or epoxidized vegetable oils (for example epoxidized coconut oil, rapeseed oil or soya oil), antifoams, for example silicone oil, preservatives, viscosity regulators, binders and/or tackifiers, fertilizers or other active ingredients for achieving specific effects, for example bactericides, fungicides, nematocides, plant activators, molluscicides or herbicides.

The compositions according to the invention are prepared in a manner known per se, in the absence of auxiliaries for example by grinding, screening and/or compressing a solid active ingredient and in the presence of at least one auxiliary for example by intimately mixing and/or grinding the active ingredient with the auxiliary (auxiliaries). These processes for the preparation of the compositions and the use of the compounds I for the preparation of these compositions are also a subject of the invention.

The application methods for the compositions, that is the methods of controlling pests of the abovementioned type, such as spraying, atomizing, dusting, brushing on, dressing, scattering or pouring—which are to be selected to suit the intended aims of the prevailing circumstances—and the use of the compositions for controlling pests of the abovementioned type are other subjects of the invention. Typical rates of concentration are between 0.1 and 1000 ppm, preferably between 0.1 and 500 ppm, of active ingredient. The rate of application per hectare is generally 1 to 2000 g of active ingredient per hectare, in particular 10 to 1000 g/ha, preferably 10 to 600 g/ha.

A preferred method of application in the field of crop protection is application to the foliage of the plants (foliar application), it being possible to select frequency and rate of application to match the danger of infestation with the pest in question. Alternatively, the active ingredient can reach the plants via the root system (systemic action), by drenching the locus of the plants with a liquid composition or by incorporating the active ingredient in solid form into the locus of the plants, for example into the soil, for example in the form of granules (soil application). In the case of paddy rice crops, such granules can be metered into the flooded paddy-field.

The compounds of the invention and compositions thereof are also be suitable for the protection of plant propagation material, for example seeds, such as fruit, tubers or kernels, or nursery plants, against pests of the abovementioned type. The propagation material can be treated with the compound prior to planting, for example seed can be treated prior to sowing. Alternatively, the compound can be applied to seed kernels (coating), either by soaking the kernels in a liquid composition or by applying a layer of a solid composition. It is also possible to apply the compositions when the propagation material is planted to the site of application, for example into the seed furrow during drilling. These treatment methods for plant propagation material and the plant propagation material thus treated are further subjects of the invention. Typical treatment rates would depend on the plant and pest/fungi to be controlled and are generally between 1 to 200 grams per 100 kg of seeds, preferably between 5 to 150 grams per 100 kg of seeds, such as between 10 to 100 grams per 100 kg of seeds.

The term seed embraces seeds and plant propagules of all kinds including but not limited to true seeds, seed pieces, suckers, corns, bulbs, fruit, tubers, grains, rhizomes, cuttings, cut shoots and the like and means in a preferred embodiment true seeds.

The present invention also comprises seeds coated or treated with or containing a compound of formula I. The term “coated or treated with and/or containing” generally signifies that the active ingredient is for the most part on the surface of the seed at the time of application, although a greater or lesser part of the ingredient may penetrate into the seed material, depending on the method of application. When the said seed product is (re)planted, it may absorb the active ingredient. In an embodiment, the present invention makes available a plant propagation material adhered thereto with a compound of formula (I). Further, it is hereby made available, a composition comprising a plant propagation material treated with a compound of formula (I).

Seed treatment comprises all suitable seed treatment techniques known in the art, such as seed dressing, seed coating, seed dusting, seed soaking and seed pelleting. The seed treatment application of the compound formula (I) can be carried out by any known methods, such as spraying or by dusting the seeds before sowing or during the sowing/planting of the seeds.

### Biological Examples

The Examples which follow serve to illustrate the invention. Certain compounds of the invention can be distinguished from known compounds by virtue of greater efficacy at low application rates, which can be verified by the person skilled in the art using the experimental procedures outlined in the Examples, using lower application rates if necessary, for example 50 ppm, 12.5 ppm, 6 ppm, 3 ppm, 1.5 ppm, 0.8 ppm or 0.2 ppm.

### Example B1: Activity Against Bemisia tabaci (Cotton White Fly)

Cotton leaf discs were placed on agar in 24-well microtiter plates and sprayed with aqueous test solutions prepared from 10′000 ppm DMSO stock solutions. After drying the leaf discs were infested with adult white flies. The samples were checked for mortality 6 days after incubation.

The following compounds resulted in at least 80% mortality at an application rate of 200 ppm: P2, P3, P11, P12.

### Example B2: Activity Against Chilo suppressalis (Striped Rice Stemborer)

24-well microtiter plates with artificial diet were treated with aqueous test solutions prepared from 10′000 ppm DMSO stock solutions by pipetting. After drying, the plates were infested with L2 larvae (6-8 per well). The samples were assessed for mortality, anti-feeding effect, and growth inhibition in comparison to untreated samples 6 days after infestation. Control of Chilo suppressalis by a test sample is given when at least one of the categories mortality, anti-feedant effect, and growth inhibition is higher than the untreated sample.

The following compounds resulted in at least 80% control at an application rate of 200 ppm: P1, P2, P3, P4, P5, P6, P8, P9, P10, P11, P12, P13, P14, P15, P16, P17, P18, P19, P20, P21, P22, P23, P24, P25, P26, P27, P29, P30, P31, P33.

### Example B3: Activity Against Diabrotica Balteata (Corn Root Worm)

Maize sprouts placed onto an agar layer in 24-well microtiter plates were treated with aqueous test solutions prepared from 10′000 ppm DMSO stock solutions by spraying. After drying, the plates were infested with L2 larvae (6 to 10 per well). The samples were assessed for mortality and growth inhibition in comparison to untreated samples 4 days after infestation.

The following compounds gave an effect of at least 80% in at least one of the two categories (mortality or growth inhibition) at an application rate of 200 ppm: P1, P2, P3, P4, P5, P6, P9, P10, P11, P12, P13, P14, P15, P16, P17, P19, P20, P21, P22, P23, P24, P25, P26, P27, P30, P31, P32, P33, P34, P35.

### Example B4: Activity Against Euschistus heros (Neotropical Brown Stink Bug)

Soybean leaves on agar in 24-well microtiter plates were sprayed with aqueous test solutions prepared from 10′000 ppm DMSO stock solutions. After drying the leaves were infested with N2 nymphs. The samples were assessed for mortality and growth inhibition in comparison to untreated samples 5 days after infestation.

The following compounds gave an effect of at least 80% in at least one of the two categories (mortality or growth inhibition) at an application rate of 200 ppm: P1, P2, P3, P4, P5, P6, P8, P11, P12, P15, P20, P21, P22, P23, P24, P27, P33.

### Example B5: Activity Against Frankliniella occidentalis (Western Flower Thrips)

Sunflower leaf discs were placed on agar in 24-well microtiter plates and sprayed with aqueous test solutions prepared from 10′000 DMSO stock solutions. After drying the leaf discs were infested with a Frankliniella population of mixed ages. The samples were assessed for mortality 7 days after infestation.

The following compounds resulted in at least 80% mortality at an application rate of 200 ppm: P4.

### Example B6: Activity Against Myzus persicae (Green Peach Aphid) Feeding/Contact Activity

Sunflower leaf discs were placed onto agar in a 24-well microtiter plate and sprayed with aqueous test solutions prepared from 10′000 ppm DMSO stock solutions. After drying, the leaf discs were infested with an aphid population of mixed ages. The samples were assessed for mortality 6 days after infestation.

The following compounds resulted in at least 80% mortality at an application rate of 200 ppm: P1, P2, P3, P4, P7, P8, P9, P10, P11, P12, P13, P14, P15, P16, P17, P18, P20, P22, P24, P29, P34.

### Example B7: Activity Against Myzus persicae (Green Peach Aphid) Systemic Activity

Roots of pea seedlings infested with an aphid population of mixed ages were placed directly into aqueous test solutions prepared from 10′000 DMSO stock solutions. The samples were assessed for mortality 6 days after placing seedlings into test solutions.

The following compounds resulted in at least 80% mortality at a test rate of 24 ppm: P1, P2, P8, P11, P12, P15, P18, P22, P34.

### Example B8: Activity Against Plutella xylostella (Diamond Back Moth)

24-well microtiter plates with artificial diet were treated with aqueous test solutions prepared from 10′000 ppm DMSO stock solutions by pipetting. After drying, Plutella eggs were pipetted through a plastic stencil onto a gel blotting paper and the plate was closed with it. The samples were assessed for mortality and growth inhibition in comparison to untreated samples 8 days after infestation.

The following compounds gave an effect of at least 80% in at least one of the two categories (mortality or growth inhibition) at an application rate of 200 ppm: P1, P2, P3, P4, P5, P6, P8, P9, P10, P11, P12, P13, P14, P15, P16, P17, P18, P19, P20, P21, P22, P23, P24, P25, P26, P27, P28, P29, P30, P31, P32, P34, P35.

### Example B9: Activity Against Spodoptera littoralis (Egyptian Cotton Leaf Worm)

Cotton leaf discs were placed onto agar in 24-well microtiter plates and sprayed with aqueous test solutions prepared from 10′000 ppm DMSO stock solutions. After drying the leaf discs were infested with five L1 larvae. The samples were assessed for mortality, anti-feeding effect, and growth inhibition in comparison to untreated samples 3 days after infestation. Control of Spodoptera littoralis by a test sample is given when at least one of the categories mortality, anti-feedant effect, and growth inhibition is higher than the untreated sample.

The following compounds resulted in at least 80% control at an application rate of 200 ppm: P1, P2, P3, P4, P5, P6, P8, P9, P10, P11, P12, P13, P14, P15, P16, P17, P18, P19, P20, P21, P22, P23, P24, P25, P27, P29, P30, P31, P32.

### Example B10: Activity Against Tetranychus urticae (Two-Spotted Spider Mite)

Bean leaf discs on agar in 24-well microtiter plates were sprayed with aqueous test solutions prepared from 10′000 ppm DMSO stock solutions. After drying the leaf discs were infested with a mite population of mixed ages. The samples were assessed for mortality on mixed population (mobile stages) 8 days after infestation.

The following compounds resulted in at least 80% mortality at an application rate of 200 ppm: P23.

### Example B11: Activity Against Carpocapsa (Cydia) Pomonella (Codling Moth) Larvicide, Feeding/Contact

Diet cubes coated with paraffin were sprayed with diluted test solutions in an application chamber. After drying off the treated cubes (10 replicates) were infested with 1 L1 larvae. Samples were incubated at 26-27° C. and checked 14 days after infestation for mortality and growth inhibition. The following compounds gave an effect of at least 80% in at least one of the two categories (mortality or growth inhibition) at an application rate of 12.5 ppm: P1, P2, P3, P5, P9, P10, P11, P12, P14, P17, P19, P20, P21, P23, P24, P30, P31.

### Example B12: Activity Against Nilaparvata lugens (Brown Plant Hopper)

Larvicide, systemic into water Rice plants cultivated in a nutritive solution were treated with the diluted test solutions into nourishing cultivation system. 1 day after application plants were infested with ˜20 N3 nymphs. 7 days after infestation samples were assessed for mortality and growth regulation.

The following compounds resulted in at least 80% mortality at an application rate of 12.5 ppm: P1, P2, P3, P9, P10, P11, P12, P14, P17, P19, P20, P23, P24, P30, P31.

1. A compound of formula (I): 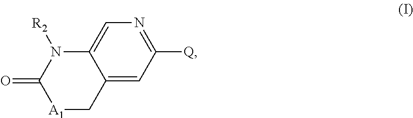
wherein
R₂ is C₁-C₆haloalkyl;
A₁ is CH₂ or O;
Q is a radical selected from the group consisting of formula Qa and Qb 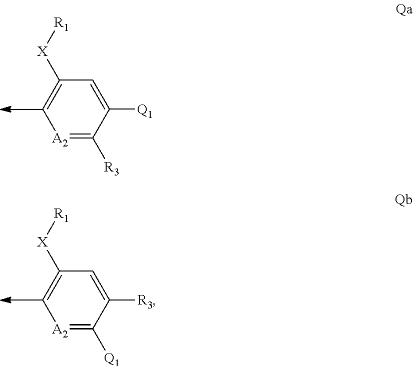
wherein the arrow denotes the point of attachment to the carbon atom of the bicyclic ring;
and wherein A₂ represents CH or N;
X is S, SO, or SO₂;
R₁ is C₁-C₄alkyl or C₃-C₆cycloalkyl-C₁-C₄alkyl;
Q₁ is hydrogen, halogen, C₁-C₆haloalkyl, C₃-C₆cycloalkyl, C₃-C₆cycloalkyl monosubstituted by cyano, C₁-C₆cyanoalkyl, C₁-C₆cyanoalkoxy, C₁-C₆haloalkoxy, —N(R₄)₂, —N(R₄)COR₅, —N(R₄)CON(R₄)₂, (oxazolidin-2-one)-3-yl or 2-pyridyloxy; or
Q₁ is a five- to six-membered aromatic ring system linked via a ring carbon atom to the ring which contains the substituent A₂, said ring system is unsubstituted or is mono- or polysubstituted by substituents selected from the group consisting of halogen, cyano, C₁-C₄alkyl, C₁-C₄haloalkyl, C₁-C₄alkoxy, C₁-C₄haloalkoxy, C₁-C₄alkylsulfanyl, C₁-C₄alkylsulfinyl and C₁-C₄alkylsulfonyl; and said ring system can contain 1, 2 or 3 ring heteroatoms selected from the group consisting of nitrogen, oxygen and sulphur, where said ring system may not contain more than one ring oxygen atom and not more than one ring sulfur atom; or
Q₁ is a five-membered aromatic ring system linked via a ring nitrogen atom to the ring which contains the substituent A₂, said ring system is unsubstituted or is mono- or polysubstituted by substituents selected from the group consisting of halogen, cyano, C₁-C₄alkyl, C₁-C₄haloalkyl, C₁-C₄alkoxy, C₁-C₄haloalkoxy, C₁-C₄alkylsulfanyl, C₁-C₄alkylsulfinyl and C₁-C₄alkylsulfonyl;
and said ring system contains 1, 2 or 3 ring heteroatoms selected from the group consisting of nitrogen, oxygen and sulphur, where said ring system contains at least one ring nitrogen atom and may not contain more than one ring oxygen atom and not more than one ring sulfur atom;
R₃ is hydrogen, halogen or C₁-C₄alkyl;
each R₄ is independently hydrogen, C₁-C₄alkyl or C₃-C₆cycloalkyl; and
R₅ is C₁-C₆alkyl, C₁-C₆haloalkyl or C₃-C₆cycloalkyl;
or an agrochemically acceptable salt, stereoisomer, enantiomer, tautomer or N-oxide of a compound of formula I.

2. A compound of formula I according to claim 1, represented by the compounds of formula (I-1): 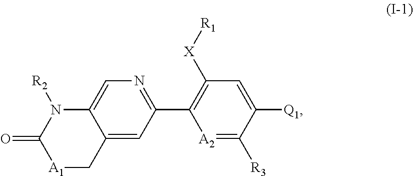
wherein A₁, A₂, X, R₁, and R₂ are as defined under formula I in claim 1, or an agrochemically acceptable salt, stereoisomer, enantiomer, tautomer or N-oxide of a compound of formula I-1, and wherein
Q₁ is hydrogen, halogen, C₁-C₆haloalkyl, C₃-C₆cycloalkyl, C₃-C₆cycloalkyl monosubstituted by cyano, C₁-C₆cyanoalkyl, C₁-C₆cyanoalkoxy, C₁-C₆haloalkoxy, —N(R₄)₂, —N(R₄)COR₅, or —N(R₄)CON(R₄)₂, (oxazolidin-2-one)-3-yl or 2-pyridyloxy; or
Q₁ is a five- to six-membered aromatic ring system linked via a ring carbon atom to the ring which contains the substituent A₂, said ring system is unsubstituted or is mono-substituted by a substituent selected from the group consisting of halogen, cyano and C₁-C₄haloalkyl; and said ring system can contain 1 or 2 ring nitrogen atoms; or
Q₁ is a five-membered aromatic ring system linked via a ring nitrogen atom to the ring which contains the substituent A₂, said ring system is unsubstituted or is mono-substituted by a substituent selected from the group consisting of halogen, cyano and C₁-C₄haloalkyl; and said ring system contains 2 or 3 ring nitrogen atoms;
R₃ is hydrogen or C₁-C₄alkyl;
each R₄ is independently hydrogen or C₁-C₄alkyl; and
R₅ is preferably C₁-C₆alkyl or C₃-C₆cycloalkyl.

3. A compound of formula I according to claim 1, represented by the compounds of formula (I-2): 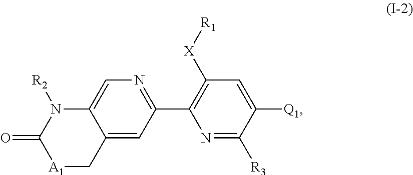
wherein A₁, X, R₁ and R₂ are as defined under formula I in claim 1, or an agrochemically acceptable salt, stereoisomer, enantiomer, tautomer or N-oxide of a compound of formula I-2, and wherein
Q₁ is hydrogen, halogen, C₁-C₆haloalkyl, C₃-C₆cycloalkyl, C₃-C₆cycloalkyl monosubstituted by cyano, C₁-C₆cyanoalkyl, C₁-C₆cyanoalkoxy, C₁-C₆haloalkoxy, —N(R₄)₂, —N(R₄)COR₅, or —N(R₄)CON(R₄)₂, (oxazolidin-2-one)-3-yl or 2-pyridyloxy; or
Q₁ is a five- to six-membered aromatic ring system linked via a ring carbon atom to the pyridyl ring substituted by X—R₁, said ring system is unsubstituted or is mono-substituted by a substituent selected from the group consisting of halogen, cyano and C₁-C₄haloalkyl; and said ring system can contain 1 or 2 ring nitrogen atoms; or
Q₁ is a five-membered aromatic ring system linked via a ring nitrogen atom to the pyridyl ring substituted by X—R₁, said ring system is unsubstituted or is mono-substituted by a substituent selected from the group consisting of halogen, cyano and C₁-C₄haloalkyl; and said ring system contains 2 or 3 ring nitrogen atoms;
R-3 is hydrogen or C₁-C₄alkyl;
each R₄ is independently hydrogen or C₁-C₄alkyl; and
R₅ is C₁-C₆alkyl or C₃-C₆cycloalkyl.

4. A compound of formula I according to claim 1, represented by the compounds of formula (I-3): 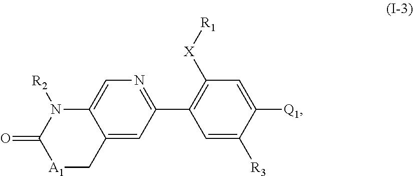
wherein A₁, X, R₁ and R₂ are as defined under formula I in claim 1, or an agrochemically acceptable salt, stereoisomer, enantiomer, tautomer or N-oxide of a compound of formula I-3, and wherein
Q₁ is hydrogen, halogen, C₁-C₆haloalkyl, C₃-C₆cycloalkyl, C₃-C₆cycloalkyl monosubstituted by cyano, C₁-C₆cyanoalkyl, C₁-C₆cyanoalkoxy, C₁-C₆haloalkoxy, —N(R₄)₂, —N(R₄)COR₅, or —N(R₄)CON(R₄)₂, (oxazolidin-2-one)-3-yl or 2-pyridyloxy; or
Q₁ is a five- to six-membered aromatic ring system linked via a ring carbon atom to the phenyl ring substituted by X—R₁, said ring system is unsubstituted or is mono-substituted by a substituent selected from the group consisting of halogen, cyano and C₁-C₄haloalkyl; and said ring system can contain 1 or 2 ring nitrogen atoms; or
Q₁ is a five-membered aromatic ring system linked via a ring nitrogen atom to the phenyl ring substituted by X—R₁, said ring system is unsubstituted or is mono-substituted by a substituent selected from the group consisting of halogen, cyano and C₁-C₄haloalkyl; and said ring system contains 2 or 3 ring nitrogen atoms;
R₃ is hydrogen or C₁-C₄alkyl;
each R₄ is independently hydrogen or C₁-C₄alkyl; and
R₅ is C₁-C₆alkyl or C₃-C₆cycloalkyl.

5. A compound of formula I according to claim 1, represented by the compounds of formula (I-4): 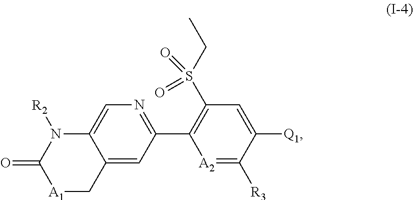
wherein
A₁ is CH₂ or O;
A₂ is CH or N, or A₂ is N;
R₂ is C₁-C₆haloalkyl, or R₂ is C₁-C₆fluoroalkyl, or R₂ is —CH₂CF₂CF₃, —CH₂CF₂CHF₂, —CH₂CF₃, —CH₂CHF₂ or —CH₂CF₂CHFCF₃;
R₃ is hydrogen or C₁-C₄alkyl, or R₃ is hydrogen or methyl;
Q₁ is hydrogen, halogen, C₁-C₆haloalkyl, C₃-C₆cycloalkyl, C₃-C₆cycloalkyl monosubstituted by cyano, C₁-C₆cyanoalkyl, C₁-C₆cyanoalkoxy, C₁-C₆haloalkoxy, —N(R₄)₂, —N(R₄)COR₅, or —N(R₄)CON(R₄)₂, (oxazolidin-2-one)-3-yl or 2-pyridyloxy; or
Q₁ is a five- to six-membered aromatic ring system linked via a ring carbon atom to the ring which contains the substituent A₂, said ring system is unsubstituted or is mono-substituted by a substituent selected from the group consisting of halogen, cyano and C₁-C₄haloalkyl; and said ring system can contain 1 or 2 ring nitrogen atoms; or
Q₁ is a five-membered aromatic ring system linked via a ring nitrogen atom to the ring which contains the substituent A₂, said ring system is unsubstituted or is mono-substituted by a substituent selected from the group consisting of halogen, cyano and C₁-C₄haloalkyl; and said ring system contains 2 or 3 ring nitrogen atoms;
each R₄ is independently hydrogen or C₁-C₄alkyl, or each R₄ is hydrogen or methyl; and
R₅ is C₁-C₆alkyl or C₃-C₆cycloalkyl, or R₅ is methyl, ethyl or cyclopropyl, or R₅ is methyl or cyclopropyl; or
an agrochemically acceptable salt, stereoisomer, enantiomer, tautomer or N-oxide of a compound of formula I-4.

6. A compound of formula I according to claim 1, represented by the compounds of formula (I-5): 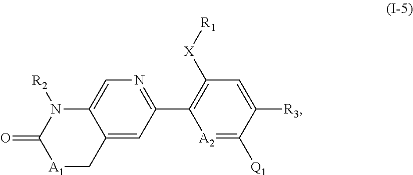
wherein A₁, A₂, X, R₁, and R₂ are as defined under formula I in claim 1, or an agrochemically acceptable salt, stereoisomer, enantiomer, tautomer or N-oxide of a compound of formula I-5, and wherein
Q₁ is hydrogen, halogen, C₁-C₆haloalkyl, C₃-C₆cycloalkyl, C₃-C₆cycloalkyl monosubstituted by cyano, C₁-C₆cyanoalkyl, C₁-C₆cyanoalkoxy, C₁-C₆haloalkoxy, —N(R₄)₂, —N(R₄)COR₅, or —N(R₄)CON(R₄)₂, (oxazolidin-2-one)-3-yl or 2-pyridyloxy; or
Q₁ is a five- to six-membered aromatic ring system linked via a ring carbon atom to the ring which contains the substituent A₂, said ring system is unsubstituted or is mono-substituted by a substituent selected from the group consisting of halogen, cyano and C₁-C₄haloalkyl; and said ring system can contain 1 or 2 ring nitrogen atoms; or
Q₁ is a five-membered aromatic ring system linked via a ring nitrogen atom to the ring which contains the substituent A₂, said ring system is unsubstituted or is mono-substituted by a substituent selected from the group consisting of halogen, cyano and C₁-C₄haloalkyl; and said ring system contains 2 or 3 ring nitrogen atoms;
R₃ is hydrogen or C₁-C₄alkyl;
each R₄ is independently hydrogen or C₁-C₄alkyl; and
R₅ is C₁-C₆alkyl or C₃-C₆cycloalkyl.

7. A compound of formula I according to claim 1, represented by the compounds of formula (I-6): 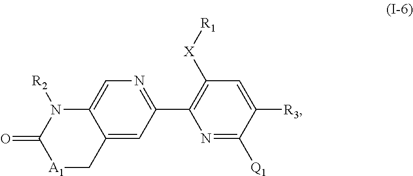
wherein A₁, X, R₁ and R₂ are as defined under formula I in claim 1, or an agrochemically acceptable salt, stereoisomer, enantiomer, tautomer or N-oxide of a compound of formula I-6, and wherein
Q₁ is hydrogen, halogen, C₁-C₆haloalkyl, C₃-C₆cycloalkyl, C₃-C₆cycloalkyl monosubstituted by cyano, C₁-C₆cyanoalkyl, C₁-C₆cyanoalkoxy, C₁-C₆haloalkoxy, —N(R₄)₂, —N(R₄)COR₅, or —N(R₄)CON(R₄)₂, (oxazolidin-2-one)-3-yl or 2-pyridyloxy; or
Q₁ is a five- to six-membered aromatic ring system linked via a ring carbon atom to the pyridyl ring substituted by X—R₁, said ring system is unsubstituted or is mono-substituted by a substituent selected from the group consisting of halogen, cyano and C₁-C₄haloalkyl; and said ring system can contain 1 or 2 ring nitrogen atoms; or
Q₁ is a five-membered aromatic ring system linked via a ring nitrogen to the pyridyl ring substituted by X-R₁, said ring system is unsubstituted or is mono-substituted by a substituent selected from the group consisting of halogen, cyano and C₁-C₄haloalkyl; and said ring system contains 2 or 3 ring nitrogen atoms;
R₃ is hydrogen or C₁-C₄alkyl;
each R₄ is independently hydrogen or C₁-C₄alkyl; and
R₅ is C₁-C₆alkyl or C₃-C₆cycloalkyl.

8. A compound of formula I according to claim 1, represented by the compounds of formula (I-7): 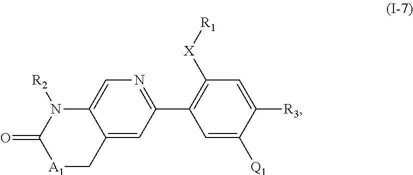
wherein A₁, X, R₁ and R₂ are as defined under formula I in claim 1, or an agrochemically acceptable salt, stereoisomer, enantiomer, tautomer or N-oxide of a compound of formula I-7, and wherein
Q₁ is hydrogen, halogen, C₁-C₆haloalkyl, C₃-C₆cycloalkyl, C₃-C₆cycloalkyl monosubstituted by cyano, C₁-C₆cyanoalkyl, C₁-C₆cyanoalkoxy, C₁-C₆haloalkoxy, —N(R₄)₂, —N(R₄)COR₅, or —N(R₄)CON(R₄)₂, (oxazolidin-2-one)-3-yl or 2-pyridyloxy; or
Q₁ is a five- to six-membered aromatic ring system linked via a carbon atom to the phenyl ring substituted by X—R₁, said ring system is unsubstituted or is mono-substituted by a substituent selected from the group consisting of halogen, cyano and C₁-C₄haloalkyl; and said ring system can contain 1 or 2 ring nitrogen atoms; or
Q₁ is a five-membered aromatic ring system linked via a nitrogen atom to the phenyl ring substituted by X—R₁, said ring system is unsubstituted or is mono-substituted by a substituent selected from the group consisting of halogen, cyano and C₁-C₄haloalkyl; and said ring system contains 2 or 3 ring nitrogen atoms;
R₃ is hydrogen or C₁-C₄alkyl;
each R₄ is independently hydrogen or C₁-C₄alkyl; and
R₅ is C₁-C₆alkyl or C₃-C₆cycloalkyl.

9. A compound of formula I according to claim 1, represented by the compounds of formula (I-8): 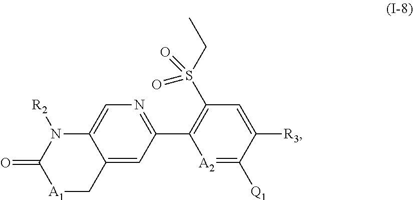
wherein
A₁ is CH₂ or O;
A₂ is CH or N, or A₂ is N;
R₂ is C₁-C₆haloalkyl, or R₂ is C₁-C₆fluoroalkyl, or R₂ is —CH₂CF₂CF₃, —CH₂CF₂CHF₂, —CH₂CF₃, —CH₂CHF₂ or —CH₂CF₂CHFCF₃;
R₃ is hydrogen or C₁-C₄alkyl, or R₃ is hydrogen or methyl;
Q₁ is hydrogen, halogen, C₁-C₆haloalkyl, C₃-C₆cycloalkyl, C₃-C₆cycloalkyl monosubstituted by cyano, C₁-C₆cyanoalkyl, C₁-C₆cyanoalkoxy, C₁-C₆haloalkoxy, —N(R₄)₂, —N(R₄)COR₅, or —N(R₄)CON(R₄)₂, (oxazolidin-2-one)-3-yl or 2-pyridyloxy; or
Q₁ is a five- to six-membered aromatic ring system linked via a ring carbon atom to the ring which contains the substituent A₂, said ring system is unsubstituted or is mono-substituted by a substituent selected from the group consisting of halogen, cyano and C₁-C₄haloalkyl; and said ring system can contain 1 or 2 ring nitrogen atoms; or
Q₁ is a five-membered aromatic ring system linked via a ring nitrogen atom to the ring which contains the substituent A₂, said ring system is unsubstituted or is mono-substituted by a substituent selected from the group consisting of halogen, cyano and C₁-C₄haloalkyl; and said ring system contains 2 or 3 ring nitrogen atoms;
each R₄ is independently hydrogen or C₁-C₄alkyl, or each R₄ is hydrogen or methyl; and
R₅ is C₁-C₆alkyl or C₃-C₆cycloalkyl; or R₅ is methyl, ethyl or cyclopropyl, or R₅ is methyl or cyclopropyl; or an agrochemically acceptable salt, stereoisomer, enantiomer, tautomer or N-oxide of a compound of formula I-8.

10. A compound of formula I according to claim 1, represented by the compounds of formula (I-9): 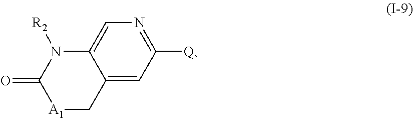
wherein
A₁ is CH₂ or O;
R₂ is C₁-C₆haloalkyl, or R₂ is C₁-C₆fluoroalkyl, or R₂ is —CH₂CF₂CF₃, —CH₂CF₂CHF₂, —CH₂CF₃, —CH₂CHF₂ or —CH₂CF₂CHFCF₃;
Q is a radical selected from the group consisting of formula Qa1 and Qb1 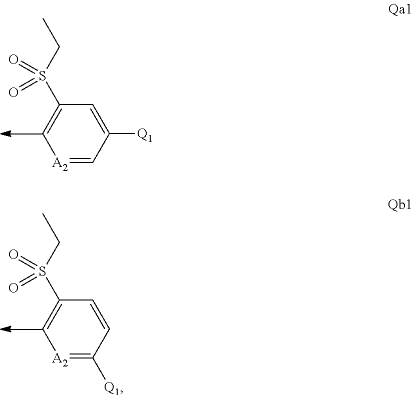
wherein the arrow denotes the point of attachment to the carbon atom of the bicyclic ring;
and wherein
A₂ is CH or N, or A₂ is N; and
Q₁ is hydrogen, halogen, trifluoromethyl, fluoroisopropyl, cyclopropyl, cyanocyclopropyl, cyanoisopropyl, cyanoisopropoxy, trifluoroethoxy, difluoropropoxy, —N(R₄)₂, —N(R₄)COR₅, or —N(R₄)CON(R₄)₂, in each of which R₄ is independently either hydrogen or methyl and R₅ is methyl, ethyl or cyclopropyl; or R₅ is methyl or cyclopropyl, or Q₁ is (oxazolidin-2-one)-3-yl, 2-pyridyloxy, N-linked pyrazolyl which is unsubstituted or is mono-substituted by chloro, cyano or trifluoromethyl, or Q₁ is N-linked triazolyl or C-linked pyrimidinyl; or an agrochemically acceptable salt, stereoisomer, enantiomer, tautomer or N-oxide of a compound of formula I-9.

11. A compound according to claim 1, wherein R₂ is —CH₂CF₂CF₃, —CH₂CF₂CHF₂, —CH₂CF₃, —CH₂CHF₂ or —CH₂CF₂CHFCF₃

12. A compound according to claim 1, wherein R₃ is hydrogen or methyl; preferably R₃ is hydrogen.

13. A compound according to claim 1, wherein Q₁ is hydrogen, chlorine, bromine, trifluoromethyl, 1-fluoro-1-methyl-ethyl, cyclopropyl, 1-cyanocyclopropyl, 1-cyano-1-methyl-ethyl, 1-cyano-1-methyl-ethoxy, 2,2,2-trifluoroethoxy, 2,2-difluoropropoxy, —NH₂, —NH(CH₃), —N(CH₃)₂, —NHCOCH₃, —N(CH₃)COCH₃, —N(CH₃)COCH₂CH₃, —NHCO(cyclopropyl), —N(CH₃)CO(cyclopropyl), —N(H)CONH₂, —N(H)CONH(CH₃), —N(H)CON(CH₃)₂, —N(CH₃)CONH₂, —N(CH₃)CONH(CH₃), —N(CH₃)CON(CH₃)₂, (oxazolidin-2-one)-3-yl, 2-pyridyloxy, pyrazol-1-yl, 3-chloro-pyrazol-1-yl, 3-cyano-pyrazol-1-yl, 3-trifluoromethyl-pyrazol-1-yl, 1,2,4-triazol-1-yl or pyrimidin-2-yl.

14. A compound according to claim 1, wherein Q₁ is hydrogen, chlorine, bromine, trifluoromethyl, 1-fluoro-1-methyl-ethyl, cyclopropyl, 1-cyanocyclopropyl, 1-cyano-1-methyl-ethyl, 1-cyano-1-methyl-ethoxy, 2,2,2-trifluoroethoxy, 2,2-difluoropropoxy, —NH(CH₃), —N(CH₃)COCH₃, —N(CH₃)CO(cyclopropyl), —N(H)CONH(CH₃), —N(CH₃)CONH(CH₃), (oxazolidin-2-one)-3-yl, 2-pyridyloxy, pyrazol-1-yl, 3-chloro-pyrazol-1-yl, 3-cyano-pyrazol-1-yl, 3-trifluoromethyl-pyrazol-1-yl, 1,2,4-triazol-1-yl or pyrimidin-2-yl.

15. A compound according to claim 1, wherein A₁ is CH₂ and R₂ is —CH₂CF₃, —CH₂CF₂CHF₂ or —CH₂CF₂CF₃.

16. A compound according to claim 1, A₁ is O and R₂ is —CH₂CF₃, —CH₂CF₂CHF₂ or —CH₂CF₂CF₃.

17. A compound of formula I according to claim 1, selected from the group consisting of:
6-(3-ethylsulfonyl-2-pyridyl)-1-(2,2,3,3,3-pentafluoropropyl)-3,4-dihydro-1,7-naphthyridin-2-one (compound P1); 1-[5-ethylsulfonyl-6-[2-oxo-1-(2,2,2-trifluoroethyl)-3,4-dihydro-1,7-naphthyridin-6-yl]-3-pyridyl]cyclopropanecarbonitrile (compound P2); 1-[5-ethylsulfonyl-6-[2-oxo-1-(2,2,3,3,3-pentafluoropropyl)-4H-pyrido[3,4-d][1,3]oxazin-6-yl]-3-pyridyl]cyclopropanecarbonitrile (compound P3); 1-[5-ethylsulfanyl-6-[2-oxo-1-(2,2,3,3,3-pentafluoropropyl)-4H-pyrido[3,4-d][1,3]oxazin-6-yl]-3-pyridyl]cyclopropanecarbonitrile (compound P4); 6-[5-(3-chloropyrazol-1-yl)-3-ethylsulfonyl-2-pyridyl]-1-(2,2,3,3,3-pentafluoropropyl)-3,4-dihydro-1,7-naphthyridin-2-one (compound P5); 1-[5-ethylsulfanyl-6-[2-oxo-1-(2,2,2-trifluoroethyl)-3,4-dihydro-1,7-naphthyridin-6-yl]-3-pyridyl]cyclopropanecarbonitrile (compound P6); N-[5-ethylsulfanyl-6-[2-oxo-1-(2,2,3,3,3-pentafluoropropyl)-3,4-dihydro-1,7-naphthyridin-6-yl]-3-pyridyl]-N-methyl-acetamide (compound P7); and N-[5-ethylsulfonyl-6-[2-oxo-l-(2,2,3,3,3-pentafluoropropyl)-3,4-dihydro-1,7-naphthyridin-6-yl]-3-pyridyl]-N-methyl-acetamide (compound P8); 6-[3-ethylsulfonyl-5-(trifluoromethyl)-2-pyridyl]-1-(2,2,3,3,3-pentafluoropropyl)-3,4-dihydro-1,7-naphthyridin-2-one (compound P9); 6-[3-ethylsulfonyl-5-(trifluoromethyl)-2-pyridyl]-1-(2,2,3,3,3-pentafluoropropyl)-4H-pyrido[3,4-d][1,3]oxazin-2-one (compound P10); 2-[5-ethylsulfonyl-6-[2-oxo-1-(2,2,3,3,3-pentafluoropropyl)-3,4-dihydro-1,7-naphthyridin-6-yl]-3-pyridyl]-2-methyl-propanenitrile (compound P11); 1-[5-ethylsulfonyl-6-[2-oxo-l-(2,2,3,3,3-pentafluoropropyl)-3,4-dihydro-1,7-naphthyridin-6-yl]-3-pyridyl]cyclopropanecarbonitrile (compound P12); 6-[3-ethylsulfonyl-6-(1,2,4-triazol-1-yl)-2-pyridyl]-1-(2,2,3,3,3-pentafluoropropyl)-3,4-dihydro-1,7-naphthyridin-2-one (compound P13); 6-(6-cyclopropyl-3-ethylsulfonyl-2-pyridyl)-1-(2,2,3,3,3-pentafluoropropyl)-3,4-dihydro-1,7-naphthyridin-2-one (compound P14); 2-[5-ethylsulfonyl-6-[2-oxo-1-(2,2,3,3,3-pentafluoropropyl)-4H-pyrido[3,4-d][1,3]oxazin-6-yl]-3-pyridyl]-2-methyl-propanenitrile (compound P15); 6-[3-ethylsulfonyl-6-(1,2,4-triazol-1-yl)-2-pyridyl]-1-(2,2,3,3,3-pentafluoropropyl)-4H-pyrido[3,4-d][1,3]oxazin-2-one (compound P16): 6-(6-cyclopropyl-3-ethylsulfonyl-2-pyridyl)-1-(2,2,3,3,3-pentafluoropropyl)-4H-pyrido[3,4-d][1,3]oxazin-2-one (compound P17); N-[5-ethylsulfonyl-6-[2-oxo-I-(2,2,3,3,3-pentafluoropropyl)-4H-pyrido[3,4-d][1,3]oxazin-6-yl]-3-pyridyl]-N-methyl-acetamide (compound P18); 6-(5-cyclopropyl-3-ethylsulfonyl-2-pyridyl)-1-(2,2,3,3,3-pentafluoropropyl)-3,4-dihydro-1,7-naphthyridin-2-one (compound P19): 2-[[5-ethylsulfonyl-6-[2-oxo-1-(2,2,3,3,3-pentafluoropropyl)-3,4-dihydro-1,7-naphthyridin-6-yl]-3-pyridyl]oxy]-2-methyl-propanenitrile (compound P20); 6-[3-ethylsulfonyl-5-(2-pyridyloxy)-2-pyridyl]-1-(2,2,3,3,3-pentafluoropropyl)-3,4-dihydro-1,7-naphthyridin-2-one (compound P21); 6-(3-ethylsulfonyl-6-pyrimidin-2-yl-2-pyridyl)-1-(2,2,3,3,3-pentafluoropropyl)-3,4-dihydro-1,7-naphthyridin-2-one (compound P22); 6-[3-ethylsulfonyl-5-(1-fluoro-1-methyl-ethyl)-2-pyridyl]-1-(2,2,3,3,3-pentafluoropropyl)-3,4-dihydro-1,7-naphthyridin-2-one (compound P23); 6-[5-(2,2-difluoropropoxy)-3-ethylsulfonyl-2-pyridyl]-1-(2,2,3,3,3-pentafluoropropyl)-3,4-dihydro-1,7-naphthyridin-2-one (compound P24); 6-[5-(2,2-difluoropropoxy)-3-ethylsulfanyl-2-pyridyl]-1-(2,2,3,3,3-pentafluoropropyl)-3,4-dihydro-1,7-naphthyridin-2-one (compound P25); 6-[3-ethylsulfanyl-5-(trifluoromethyl)-2-pyridyl]-1-(2,2,3,3,3-pentafluoropropyl)-4H-pyrido[3,4-d][1,3]oxazin-2-one (compound P26); 6-[5-(2,2-difluoropropoxy)-3-ethylsulfanyl-2-pyridyl]-1-(2,2,3,3,3-pentafluoropropyl)-4H-pyrido[3,4-d][1,3]oxazin-2-one (compound P27); 6-[3-ethylsulfanyl-5-(methylamino)-2-pyridyl]-1-(2,2,3,3,3-pentafluoropropyl)-4H-pyrido[3,4-d][1,3]oxazin-2-one (compound P28); N-[5-ethylsulfanyl-6-[2-oxo-1-(2,2,3,3,3-pentafluoropropyl)-4H-pyrido[3,4-d][1,3]oxazin-6-yl]-3-pyridyl]-N-methyl-acetamide (compound P29); 6-[3-ethylsulfonyl-5-(1-fluoro-1-methyl-ethyl)-2-pyridyl]-1-(2,2,3,3,3-pentafluoropropyl)-4H-pyrido[3,4-d][1,3]oxazin-2-one (compound P30); 6-[5-(2,2-difluoropropoxy)-3-ethylsulfonyl-2-pyridyl]-1-(2,2,3,3,3-pentafluoropropyl)-4H-pyrido[3,4-d][1,3]oxazin-2-one (compound P31): 6-[5-(2,2-difluoropropoxy)-3-ethylsulfinyl-2-pyridyl]-1-(2,2,3,3,3-pentafluoropropyl)-4H-pyrido[3,4-d][1,3]oxazin-2-one (compound P32); 2-[5-ethylsulfanyl-6-[2-oxo-1-(2,2,3,3,3-pentafluoropropyl)-4H-pyrido[3,4-d][1,3]oxazin-6-yl]-3-pyridyl]-2-methyl-propanenitrile (compound P33); 1-[5-ethylsulfonyl-6-[2-oxo-1-(2,2,2-trifluoroethyl)-4H-pyrido[3,4-d][1,3]oxazin-6-yl]-3-pyridyl]cyclopropanecarbonitrile (compound P34); 1-[5-ethylsulfanyl-6-[2-oxo-1-(2,2,2-trifluoroethyl)-4H-pyrido[3,4-d][1,3]oxazin-6-yl]-3-pyridyl]cyclopropanecarbonitrile (compound P35).

18. A composition comprising an insecticidally, acaricidally, nematicidally or molluscicidally effective amount of a compound of formula (I), or an agrochemically acceptable salt, stereoisomer, enantiomer, tautomer or N-oxide thereof, as defined in claim 1 and, optionally, an auxiliary or diluent.

19. A method of combating and controlling insects, acarines, nematodes or molluscs which comprises applying to a pest, to a locus of a pest, or to a plant susceptible to attack by a pest an insecticidally, acaricidally, nematicidally or molluscicidally effective amount of a compound of formula (I), or an agrochemically acceptable salt, stereoisomer, enantiomer, tautomer or N-oxide thereof, as defined in claim 1.

20. A method for the protection of plant propagation material from the attack by insects, acarines, nematodes or molluscs, which comprises treating the propagation material or the site, where the propagation material is planted, with a composition according to claim 18.

21. A compound of formula XXIXa 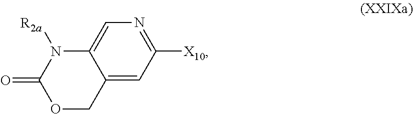
wherein
R₂ₐ is hydrogen or C₁-C₆haloalkyl; and
X₁₀ is a halogen or a pseudo-halogen leaving group.
